(19) United States
(12) Patent Application Publication
(10) Pub. No.: US 2021/0155671 A1
(43) Pub. Date: May 27, 2021

(54) USES OF CD20-BINDING MOLECULES AND ADDITIONAL THERAPEUTIC AGENTS

(71) Applicant: Molecular Templates, Inc., Austin, TX (US)

(72) Inventors: Eric Poma, New York, NY (US); Roger Waltzman, New York, NY (US); Thomas Strack, New Rochelle, NY (US)

(21) Appl. No.: 17/103,304

(22) Filed: Nov. 24, 2020

Related U.S. Application Data
(60) Provisional application No. 63/061,062, filed on Aug. 4, 2020.
(60) Provisional application No. 62/939,673, filed on Nov. 24, 2019.

(51) Int. Cl. C07K 14/705; A61K 31/454; A61K 47/26

(52) U.S. Cl. CPC C07K 14/70596; A61K 9/0053; A61K 47/26; A61K 31/454

(57) ABSTRACT

Provided herein are uses of CD20-binding molecules and one or more additional therapeutic agents. Certain CD20-binding molecules useful in the methods disclosed herein comprise 1) two or more CD20 binding regions and 2) one or more Shiga toxin effector polypeptides derived from an A Subunit of a member of the Shiga toxin family. Also disclosed herein are uses of CD20-binding molecules, and compositions thereof, (such as in conjunction with one or more additional therapeutic agents) for selective killing of specific cell types (such as a CD20-expressing tumor cell) and/or treating a variety of conditions, including cancers and tumors involving a CD20-expressing cell.

### CROSS REFERENCE TO RELATED APPLICATIONS

This application claims priority to U.S. Provisional Application No. 63/061,062, filed on Aug. 4, 2020 and U.S. Provisional Application No. 62/939,673, filed on Nov. 24, 2019, each of which is incorporated by reference herein in its entirety.

### SEQUENCE LISTING

The Sequence Listing associated with this application is provided in text format in lieu of a paper copy, and is hereby incorporated by reference into the specification. The name of the text file containing the Sequence Listing is MTEM-012-03US-Sequence-Listing.txt. The text file is about 1.19 megabytes in size, was created on Nov. 24, 2020, and is being submitted electronically via EFS-Web.

### TECHNICAL FIELD

The present application relates to CD20-binding molecules comprising a CD20-binding region and a toxin effector region, such as, e.g., a Shiga toxin effector polypeptide derived from the A1 fragment of the A Subunit of a member(s) of the Shiga toxin family, and compositions comprising the same. This application also relates to compositions and methods for treating a variety of diseases, disorders, and conditions, including compositions and methods for treating cancer and/or tumors.

### BACKGROUND

The development of synthetic fusion proteins from toxins that are effective as therapeutics has challenged scientists for decades. Naturally occurring toxins or truncated toxin fragments have been linked or fused to immunoglobulin domains or receptor ligands through chemical conjugation or recombinant protein engineering techniques with the hope of creating cell-targeted therapeutic molecules. The aim of such molecular engineering techniques is to design chimeric molecules with the dual functionality of: 1) delivering toxins to specific cell types or places within an organism after systemic administration; and 2) effectuating a targeted cytotoxicity to specific cells using potent cytotoxicity mechanisms, such as toxin catalytic activities.

CD20 is an attractive target for therapies, such as for lymphocyte proliferation disorders and/or autoimmune diseases. There is a need in the art to develop effective compositions, therapeutic molecules, and therapeutic methods which target cells expressing cell-surface CD20. In particular, there is a need in the art to develop therapies using CD20-targeted molecules that trigger rapid and/or efficient cellular internalization of cell-surface CD20 molecules as anti-neoplastic therapeutics. Such cell-targeted therapies may be used for the targeted killing of CD20-expressing cells, such as, e.g., certain malignant cells, B-lymphocytes (B-cells), and T-lymphocytes (T-cells). New therapies are especially needed for patients who are insensitive or develop resistance to current CD20-targeted therapies relying on extracellular mechanisms, such as, e.g., immune mechanisms based on signaling function(s) of an immunoglobulin domain like a fragment crystallizable Fc region (Fc region) interaction(s) with a Fc receptor(s) or the complement system.

There accordingly remains a need in the art for therapies using CD20-binding molecules which exhibit efficient and effective cellular internalization, intracellular-routing, and potent cytotoxicity toward CD20-expressing cells for the treatment of a variety of diseases, such as, e.g., cancers and tumors involving CD20-expressing cells.

### BRIEF SUMMARY

Provided herein are methods for treating or slowing progression of a disease, disorder, or condition, comprising administering to a subject in need thereof an effective amount of (i) a CD20-binding molecule and (ii) at least one additional therapeutic agent, wherein the CD20-binding molecule comprises (a) a CD20-binding region, wherein the CD20-binding region is capable of specifically binding an extracellular portion of a CD20 protein, and (b) a Shiga toxin effector polypeptide.

In some embodiments, a method for treating or slowing the progression of a disease, disorder, or condition comprises administering to a subject in need thereof an effective amount of (i) a CD20-binding molecule; and (ii) one or more additional therapeutic agents; wherein the CD20-binding molecule comprises a polypeptide having the sequence of SEQ ID NO: 54. The one or more additional therapeutic agents may be, for example, a nucleoside analog (e.g. gemcitabine), a platinum-based chemotherapeutic agent (e.g. oxaliplatin), an immunomodulatory drug (e.g. lenalidomide), a Bruton's tyrosine kinase (BTK) inhibitor, a PI3K inhibitor, or sirolimus (i.e., rapamycin).

These and other features, aspects and advantages of the invention will become better understood in view of the following description, appended claims, and accompanying figures. The features, aspects and advantages of the invention may be individually combined or removed freely.

### BRIEF DESCRIPTION OF THE FIGURES

FIGS. 1A, 1B, and 1C show schematic representations of illustrative CD20-binding molecules as described herein. FIG. 1A shows the general architecture of illustrative CD20-binding proteins. FIG. 1B shows schematic representations of illustrative CD20-binding molecules that each comprise two CD20 binding regions where a small, vertical line may represent any suitable type of molecular association, such as, e.g., a single covalent bond like a disulfide bond or a linker, whether flexible or rigid; and a curved line may represent any suitable type of molecular association, such as, e.g., a flexible linker. FIG. 1C shows schematic representations of illustrative CD20-binding molecules, which each comprise two CD20-binding regions derived from an immunoglobulin(s), and with examples of non-covalent, intermolecular associations as a result of intermolecular domain swapping between immunoglobulin-derived, CD20-binding regions. In FIG. 1C, the heavier weight lines represent any suitable type of molecular association, such as, e.g., a covalent bond or linker; and the lighter weight lines represent connections between immunoglobulin-derived domains of a CD20 binding region component, such as, e.g., a single covalent bond or a fifty amino acid residue linker. The schematic representations in FIG. 1A-1C show illustrative forms of the CD20-binding molecules that may represent different structural forms, including monomeric, heterodimeric, and/or homodimeric forms, such as, e.g., a homodimeric form stabilized by an inter-polypeptide disulfide bond(s) between two components of the molecule (e.g., Shiga toxin A Subunit effector regions and/or CD20 binding regions).

FIG. 2 graphically shows the sizes of different, illustrative CD20-binding molecules in different, illustrative compositions as described herein, analyzed by size exclusion chromatography (SEC). In addition, FIG. 2 shows the purity of different, illustrative compositions as described herein, analyzed by SEC. For the SEC analysis of three different samples, the absorbance of ultraviolet light at 280 nanometers (nm) of the material eluted after flowing through a SEC column in milli-absorbance units (mAU) was plotted over the elution volume (mL).

FIG. 3 shows a Coomassie-stained, sodium dodecyl sulfate, polyacrylamide gel after electrophoresis of illustrative CD20-binding molecules from illustrative CD20-binding molecules compositions prepared in either reducing or non-reducing conditions. FIG. 3 shows the sizes of different, illustrative CD20-binding molecules analyzed by gel electrophoresis, and the relative amounts or purity of proteinaceous molecules present in different, illustrative compositions.

FIG. 4 graphically shows the binding affinity characteristics of illustrative CD20-binding molecule compositions, including a monovalent CD20-binding protein composition, to CD20 positive (CD20+) human tumor-derived, cells. The mean fluorescent intensity representing the amount of cell-bound, CD20-binding protein was plotted over the sample concentration of multivalent CD20-binding protein in nanograms per milliliter (ng/mL) analyzed.

FIG. 5 graphically shows the ribosomal inhibitory activity of different, illustrative CD20-binding molecules, including a monovalent CD20-binding protein, provided as percent of zero inhibition of protein synthesis in an in vitro translation assay. The percentage of protein synthesis of zero protein synthesis activity was plotted over the logarithm to base 10 of the molar concentration in picomolar of Shiga toxin component(s) present in each sample analyzed.

FIG. 6 graphically shows the cytotoxicities of illustrative multivalent CD20-binding molecule compositions to CD20+ human derived tumor cells as compared to the cytotoxicity of a composition comprising the monovalent, CD20-binding protein component of the illustrative CD20-binding molecules of those illustrative CD20-binding molecule compositions. The percentage of viable cells was plotted over the logarithm to base 10 of CD20-binding protein concentration in nanograms per milliliter (ng/mL).

FIG. 7 graphically shows the cytotoxicity of an illustrative multivalent CD20-binding molecule composition to CD20+ human tumor-derived, cells as compared to a composition comprising the monovalent, CD20-binding protein component of the illustrative CD20-binding molecule(s) of that illustrative CD20-binding molecule composition. In addition, FIG. 7 shows the cytotoxicity of a composition comprising a mixture of monovalent and multivalent CD20-binding molecules. The percent viability of cells was plotted over the logarithm to base 10 of CD20-binding protein concentration in nanograms per milliliter (ng/mL).

FIG. 8 graphically shows the cytotoxicities of various, fixed-ratio mixtures of purified, CD20-binding protein compositions to CD20+ human tumor-derived, cells. The percent viability of cells was plotted over the logarithm to base 10 of CD20-binding protein concentration in nanograms per milliliter (ng/mL).

FIG. 9 graphically shows the cytotoxicities of an illustrative CD20-binding protein composition and fixed-ratio mixtures of purified, CD20-binding protein compositions to CD20 negative (CD20-) human tumor-derived, cells. The percent viability of cells was plotted over the logarithm to base 10 of CD20-binding protein concentration in nanograms per milliliter (ng/mL).

FIG. 10 graphically shows the cytotoxicities (in CD₅₀ concentrations) to CD20+ human tumor-derived, cells of different CD20-binding protein compositions which varied in their proportions of multivalent CD20-binding molecule(s) to monovalent CD20-binding protein. CD₅₀ values in nanograms per milliliter (ng/mL) of different, fixed-ratio, CD20-binding protein mixtures were plotted over the protein concentration percentages of (αCD20-scFv::SLT-1A)2 composition present in the sample tested. In addition, FIG. 10 shows a line which was fit to the data points using linear regression statistical modeling and the resulting coefficient of determination (R squared) of that line fit.

FIG. 11 graphically shows the cytotoxicities (in CD₅₀ concentrations) to CD20+ human tumor-derived, cells of different CD20-binding protein compositions which varied in their proportions of multivalent CD20-binding molecule(s) to monovalent CD20-binding protein. CD₅₀ values in nanograms per milliliter (ng/mL) of different, fixed ratio, CD20-binding protein mixtures were graphed over the protein concentration percentages of (αCD20-scFv::SLT-1A)2 composition present in the sample tested.

FIGS. 12A, 12B, and 12C graphically show the sizes and proportions of molecules present in different, illustrative CD20-binding molecule compositions as described herein analyzed by size exclusion chromatography (SEC). For the SEC analysis, the absorbance of ultraviolet light at 280 nm of the material eluted after flowing through a SEC column was plotted in milli-absorbance units (mAU) over the elution time (minutes). Software was used to identify individual peaks in the 280 nm trace and the retention time of each peak's maximum absorbance of ultraviolet light at 280 nm.

FIG. 13 graphically shows anti-Molecule 001 antibody levels in mice on study days 17 and 38.

FIG. 14 graphically shows anti-Molecule 001 antibody levels in primates on study days 10 and 17.

FIG. 15 shows anti-Molecule 001 antibody levels in primates. Anti-Molecule 001 antibody titer levels are plotted versus the day of study for the two primate treatment groups (Molecule 001 only, Molecule 001 plus sirolimus).

FIG. 16 graphically shows pharmacokinetic data from primates treated with Molecule 001 alone, or Molecule 001 plus sirolimus. Serum concentration of Molecule 001 (ng/mL) was plotted versus the day of study for the two primate treatment groups (Molecule 001 only, Molecule 001 plus sirolimus).

FIG. 17 graphically shows peripheral B-cell depletion in primates over the course of the study for the two primate treatment groups (Molecule 001 only, Molecule 001 plus sirolimus).

FIGS. 18A and 18B show the results of an in vitro experiment wherein various cancer cell lines were treated with Molecule 001, a chemotherapeutic agent, or a combination thereof. Fraction affected (Fa) represents the fractional response (cytotoxicity) measured in the presence of a given combination. Combination Index (CI) measures the degree of drug interaction. CA and Fa are plotted to graphically depict combinatorial responses observed.

FIG. 19A shows cytotoxicity of Molecule 001 alone or in combination with LEN on Daudi cells. FIG. 19B shows cytotoxicity of LEN alone or in combination with Molecule 001 on Daudi cells. FIG. 19C shows cell viability after treatment with LEN, Molecule 001, or a combination thereof. FIG. 19D is an isobologram depicting the synergistic toxicity of Molecule 001 on Daudi cells pre-treated with lenalidomide for 24 hours. FIG. 19E shows cytotoxicity of lenalidomide alone, Molecule 001 alone, or lenalidomide in combination with Molecule 001 on ST-486 cells.

FIG. 20 shows the design of a clinical study using Molecule 001 in combination with lenalidomide. “*” indicates that in Cohorts 4 and 5 (25 and 50 μg/kg/dose), Molecule 001 will be dosed biweekly for 2 weeks (Days 1, 5, 8, 12) for Cycles 1-2 and then once weekly for subsequent cycles. Abbreviations: LEN, lenalidomide; MTD, maximum tolerated dose; qd, once daily; TC, telephone call.

FIG. 21 provides a summary of best response and time on study for a clinical study of Molecule 001 in combination with lenalidomide. Among 5 subjects with response data available, one subject had progressive disease, one had stable disease, one had a partial response, and 2 had complete responses. Abbreviations: DLBCL, diffuse large B-cell lymphoma; FL, follicular lymphoma; GCB, germinal center B-cell; MCL, mantle cell lymphoma; NOS, not otherwise specified; SLL, small lymphocytic lymphoma.

FIG. 22A shows cytotoxicity of gemcitabine alone or in combination with different concentrations of Molecule 001 on SU-DHL-4, HBL-1, L-82, and MV-4-11 cells. FIG. 22B shows cytotoxicity of gemcitabine alone or in combination with different concentrations of Molecule 001 on Daudi, Raji, and Toledo cells.

FIG. 23 shows the minimum observed CD19+ percent change from baseline upon treatment with Molecule 001 and GemOx. Baseline is the value observed at screening. If no screening value was available, baseline was considered the value at day 8 of Cycle 1.

### DETAILED DESCRIPTION

The present invention is described more fully hereinafter using illustrative, non-limiting embodiments, and references to the accompanying figures. This invention may, however, be embodied in many different forms and should not be construed as to be limited to the embodiments set forth below. Rather, these embodiments are provided so that this disclosure is thorough and conveys the scope described herein to those skilled in the art.

In order that the present invention may be more readily understood, certain terms are defined below. Additional definitions may be found within the detailed description described herein.

As used in the specification and the appended claims, the terms “a,” “an” and “the” include both singular and the plural referents unless the context clearly dictates otherwise.

As used in the specification and the appended claims, the term “and/or” when referring to two species, A and B, means at least one of A and B. As used in the specification and the appended claims, the term “and/or” when referring to greater than two species, such as A, B, and C, means at least one of A, B, or C, or at least one of any combination of A, B, or C (with each species in singular or multiple possibility).

The term “amino acid residue” or “amino acid” includes reference to an amino acid that is incorporated into a protein, polypeptide, or peptide. The term “polypeptide” includes any polymer of amino acids or amino acid residues. The term “polypeptide sequence” refers to a series of amino acids or amino acid residues from which a polypeptide is physically composed. A “protein” is a macromolecule comprising one or more polypeptides or polypeptide “chains.” A “peptide” is a small polypeptide of sizes less than about 15 to 20 amino acid residues. The term “amino acid sequence” refers to a series of amino acids or amino acid residues which physically comprise a peptide or polypeptide depending on the length. Unless otherwise indicated, polypeptide and protein sequences disclosed herein are written from left to right representing their order from an amino terminus to a carboxy terminus.

The terms “amino acid,” “amino acid residue,” “amino acid sequence,” or polypeptide sequence include naturally occurring amino acids (including L and D isosteriomers) and, unless otherwise limited, also include known analogs of natural amino acids that can function in a similar manner as naturally occurring amino acids, such as, e.g., selenocysteine, pyrrolysine, N-formylmethionine, gamma-carboxyglutamate, hydroxyprolinehypusine, pyroglutamic acid, and selenomethionine (see e.g. Nagata K et al., Bioinformatics 30: 1681-9 (2014)). The amino acids referred to herein are described by shorthand designations as follows in Table 1.

TABLE 1
Amino Acid Nomenclature

| Name | 3-letter | 1-letter |
|---|---|---|
| Alanine | Ala | A |
| Arginine | Arg | R |
| Asparagine | Asn | N |
| Aspartic Acid or Aspartate | Asp | D |
| Cysteine | Cys | C |
| Glutamic Acid or Glutamate | Glu | E |
| Glutamine | Gln | Q |
| Glycine | Gly | G |
| Histidine | His | H |
| Isoleucine | Ile | I |
| Leucine | Leu | L |
| Lysine | Lys | K |
| Methionine | Met | M |
| Phenylalanine | Phe | F |
| Proline | Pro | P |
| Serine | Ser | S |
| Threonine | Thr | T |
| Tryptophan | Trp | W |
| Tyrosine | Tyr | Y |
| Valine | Val | V |

The phrase “conservative substitution” with regard to a polypeptide, refers to a change in the amino acid composition of the polypeptide that does not substantially alter the function and structure of the overall polypeptide (see Creighton, Proteins: Structures and Molecular Properties (W. H. Freeman and Company, New York (2nd ed., 1992))).

As used herein, the terms “expressed,” “expressing,” or “expresses,” and grammatical variants thereof, refer to translation of a polynucleotide or nucleic acid into a polypeptide and/or protein. The expressed polypeptides or proteins may remain intracellular, become a component of the cell surface membrane or be secreted into an extracellular space.

As used herein, the meaning of the phrase “CD20-expressing cell” encompasses any cell that expresses, at a cellular surface, a CD20 molecule which comprises a transmembrane domain.

As used herein, cells which express a significant amount of CD20 at least one cellular surface are “CD20 positive cells” or “CD20+ cells” and are cells physically coupled to the extracellular target biomolecule CD20.

As used herein, the symbol “a” is shorthand for an immunoglobulin-type binding region capable of binding to the biomolecule following the symbol. The symbol “a” is used to refer to the functional characteristic of an immunoglobulin-type binding region based on its capability of binding to the biomolecule following the symbol.

The terms “associated,” “associating,” “linked,” or “linking” as used herein refers to the state of two or more components of a molecule being joined, attached, connected, or otherwise coupled to form a single molecule or the act of making two molecules associated with each other to form a single molecule by creating an association, linkage, attachment, and/or any other connection between the two molecules. For example, the term “linked” may refer to two or more components associated by one or more atomic interactions such that a single molecule is formed and wherein the atomic interactions may be covalent and/or non-covalent. Non-limiting examples of covalent associations between two components include peptide bonds and cysteine-cysteine disulfide bonds. Non-limiting examples of non-covalent associations between two molecular components include ionic bonds.

For purposes as described herein, the term “linked” refer to two or more molecular components associated by one or more atomic interactions such that a single molecule is formed and wherein the atomic interaction includes at least one covalent bond. For purposes as described herein, the term “linking” refers to the act of creating a linked molecule as described above.

For purposes as described herein, the term “fused” refers to two or more proteinaceous components associated by at least one covalent bond which is a peptide bond, regardless of whether the peptide bond involves the carbon of a carboxyl acid group or involves another carbon, such as, e.g., the α-carbon, β-carbon, γ-carbon, σ-carbon, etc. Non-limiting examples of two proteinaceous components fused together include, e.g., an amino acid, peptide, or polypeptide fused to a polypeptide via a peptide bond such that the resulting molecule is a single, continuous polypeptide. For purposes as described herein, the term “fusing” refers to the act of creating a fused molecule as described above, such as, e.g., a fusion protein generated from the recombinant fusion of genetic regions.

The symbol “::” means the polypeptide regions before and after it are physically linked together to form a continuous polypeptide.

For purposes as described herein, the term “effector” means providing a biological activity, such as cytotoxicity, biological signaling, enzymatic catalysis, subcellular routing, and/or intermolecular binding resulting in the recruitment of one or more factors and/or allosteric effect(s).

As used herein, the phrase “multivalent CD20-binding molecule” refers to a CD20-binding molecule or plurality of CD20-binding molecules comprising two or more high-affinity CD20 binding regions, such as, e.g. a protein comprising two or more CD20 binding regions where each individual binding region has a dissociation constant of 10⁻⁵ to 10⁻¹² moles per liter toward an extracellular part of CD20.

As used herein, the phrase “multivalent CD20-binding protein” refers to a CD20-binding protein molecule or plurality of CD20-binding protein molecules comprising two or more high-affinity CD20 binding regions, such as, e.g. a protein comprising two or more CD20 binding regions where each individual binding region has a dissociation constant of 10⁻⁵ to 10⁻¹² moles/liter toward an extracellular part of CD20.

With regard to a Shiga toxin protein sequence, the term “wild-type” as used herein refers to a naturally occurring, Shiga toxin protein sequence(s) found in a living species, such as, e.g., a pathogenic bacterium, wherein that Shiga toxin protein sequence(s) is one of the most frequently occurring variants. This is in contrast to infrequently occurring Shiga toxin protein sequences that, while still naturally occurring, are found in less than one percent of individual organisms of a given species out of individual organisms of that same species when sampling a statistically powerful number of naturally occurring individual organisms of that species which comprise at least one Shiga toxin protein variant. A clonal expansion of a natural isolate outside its natural environment (regardless of whether the isolate is an organism or molecule comprising biological sequence information) does not alter the naturally occurring requirement as long as the clonal expansion does not introduce new sequence variety not present in naturally occurring populations of that species and/or does not change the relative proportions of sequence variants to each other.

For purposes as described herein, the phrase “derived from” means that the polypeptide region comprises amino acid sequences originally found in a protein and which may now comprise additions, deletions, truncations, rearrangements, or other alterations relative to the original sequence as long as the overall function and structure are substantially conserved.

For purposes as described herein, a Shiga toxin effector function is a biological activity conferred by a polypeptide region derived from a Shiga toxin A Subunit. Non-limiting examples of Shiga toxin effector functions include cellular internalization, subcellular routing, catalytic activity, and cytotoxicity. Shiga toxin catalytic activities include, for example, ribosome inactivation, protein synthesis inhibition, N-glycosidase activity, polynucleotide:adenosine glycosidase activity, RNAase activity, and DNAase activity. Shiga toxins are ribosome inactivating proteins (RIPs). RIPs can depurinate nucleic acids, polynucleosides, polynucleotides, rRNA, ssDNA, dsDNA, mRNA (and polyA), and viral nucleic acids (see e.g. Brigotti M et al., Toxicon 39: 341-8 (2001); Brigotti M et al., FASEB J 16: 365-72 (2002)). Some RIPs show antiviral activity and superoxide dismutase activity. Shiga toxin catalytic activities have been observed both in vitro and in vivo. Non-limiting examples of assays for Shiga toxin effector activity measure protein synthesis inhibitory activity, depurination activity, inhibition of cell growth, cytotoxicity, supercoiled DNA relaxation activity, and nuclease activity.

The term “IC50” or “IC₅₀” is used herein to refer to the half-maximal inhibitory concentration as measured using in an in vitro ribosome function assay. The term “CD₅₀” or “CD₅₀” is used herein to refer to the half-maximal cytotoxicity concentration in an in vitro cell killing and/or survival assay. The skilled artisan will readily understand the meaning of each of these terms, when taken in context. Each of IC₅₀, and CD₅₀ may be measured by generating a multiple data points using different molecule concentrations or a concentration series. For some samples, accurate values for either IC₅₀ or CD₅₀ might be unobtainable due to the inability to collect the required data points for an accurate curve fit. For example, theoretically, neither an IC₅₀ nor CD₅₀ can be determined if greater than 50% ribosome inhibition or cell death, respectively, does not occur in a concentration series for a given sample. Data insufficient to accurately fit a curve should not be considered as representative of actual molecule activity.

As used herein, the retention of Shiga toxin effector function refers to a level of Shiga toxin functional activity, as measured by an appropriate quantitative assay with reproducibility comparable to a wild-type Shiga toxin effector polypeptide region control. For ribosome inhibition, Shiga toxin effector function is exhibiting an IC₅₀ of 10,000 picomolar (pM) or less. For cytotoxicity in a target positive cell kill assay, Shiga toxin effector function is exhibiting a CD₅₀ of 1,000 nanomolar (nM) or less, depending on the cell type and its expression of the appropriate extracellular CD20 target biomolecule.

As used herein, the retention of “significant” Shiga toxin effector function refers to a level of Shiga toxin functional activity, as measured by an appropriate quantitative assay with reproducibility comparable to a wild-type Shiga toxin effector polypeptide control. For in vitro ribosome inhibition, significant Shiga toxin effector function is exhibiting an IC₅₀ of 300 pM or less depending on the source of the ribosomes (e.g. bacteria, archaea, or eukaryote (algae, fungi, plants, or animals)). This is significantly greater inhibition as compared to the approximate IC₅₀ of 100,000 pM for the catalytically inactive SLT-1A 1-251 double mutant (Y77S/E167D). For cytotoxicity in a target positive cell kill assay in laboratory cell culture, significant Shiga toxin effector function is exhibiting a CD₅₀ of 100, 50, or 30 nM or less, depending on the cell line and its expression of the appropriate extracellular CD20 target biomolecule. This is significantly greater cytotoxicity to the appropriate target cell line as compared to an SLT-1A subunit alone, without a cell targeting binding region, which has a CD₅₀ of 100-10,000 nM, depending on the cell line.

For some samples, accurate values for either IC₅₀ or CD₅₀ might be unobtainable due to the inability to collect the required data points for an accurate curve fit. For example, theoretically, neither an IC₅₀ nor CD₅₀ can be determined if greater than 50% ribosome inhibition or cell death, respectively, does not occur in a concentration series for a given sample. Inaccurate IC₅₀ and/or CD₅₀ values should not be considered when determining significant Shiga toxin effector function activity. Data insufficient to accurately fit a curve as described in the analysis of the data from illustrative Shiga toxin effector function assays, such as, e.g., assays described in the Examples, infra, should not be considered as representative of actual Shiga toxin effector function.

A failure to detect activity in Shiga toxin effector function may be due to improper expression, polypeptide folding, and/or polypeptide stability rather than a lack of cell entry, subcellular routing, and/or enzymatic activity. Assays for Shiga toxin effector functions may not require much CD20-binding molecule described herein to measure significant amounts of Shiga toxin effector function activity. To the extent that an underlying cause of low or no effector function is determined empirically to relate to protein expression or stability, one of skill in the art may be able to compensate for such factors using protein chemistry and molecular engineering techniques known in the art, such that a Shiga toxin functional effector activity may be restored and measured. As examples, improper cell-based expression may be compensated for by using different expression control sequences; improper polypeptide folding and/or stability may benefit from stabilizing terminal sequences, or compensatory mutations in non-effector regions which stabilize the three-dimensional structure of the protein, etc. When new assays for individual Shiga toxin functions become available, Shiga toxin effector regions or polypeptides may be analyzed for any level of those Shiga toxin effector functions, such as for being within a certain-fold activity of a wild-type Shiga toxin effector polypeptide. Examples of meaningful activity differences are, e.g., Shiga toxin effector polypeptide regions that have 1000-fold or 100-fold or less the activity of a wild-type Shiga toxin effector polypeptide; or that have 3-fold to 30-fold or more activity compared to a functional knock-down or knockout Shiga toxin effector polypeptide.

Certain Shiga toxin effector functions are not easily measurable, e.g. subcellular routing functions. Currently there is no routine, quantitative assay to distinguish whether the failure of a Shiga toxin effector polypeptide to be cytotoxic is due to improper subcellular routing, but at a time when tests are available, Shiga toxin effector polypeptides may be analyzed for any significant level of subcellular routing as compared to the appropriate wild-type Shiga toxin effector polypeptide region.

It should be noted that even if the cytotoxicity of a Shiga toxin effector polypeptide is reduced relative to wild-type, in practice, applications using attenuated, Shiga toxin effector polypeptides may be equally or more effective than those using wild-type, Shiga toxin effector polypeptides because the highest potency variants might exhibit undesirable effects which are minimized or reduced in reduced-potency variants. Wild-type Shiga toxin effector polypeptides are very potent, being able to kill with only one molecule reaching the cytosol or perhaps 40 molecules being internalized (Tam P, Lingwood C, Microbiology 153: 2700-10 (2007)). Shiga toxin effector polypeptides with even considerably reduced Shiga toxin effector functions, such as, e.g., subcellular routing or cytotoxicity, as compared to wild-type Shiga toxin effector polypeptides may still be potent enough for practical applications involving targeted cell killing and/or detection of certain subcellular compartments of specific cell types. And such effector polypeptides may also be useful for delivering cargos (e.g. additional exogenous material) to certain intracellular locations or subcellular compartments.

The term “selective cytotoxicity” with regard to the cytotoxic activity of a cytotoxic CD20-binding molecule refers to the relative levels of cytotoxicity between a targeted cell population and a non-targeted bystander cell population, which can be expressed as a ratio of the half-maximal cytotoxic concentration (CD₅₀) for a targeted cell type over the CD₅₀ for an untargeted cell type to show the preferentiality of cell killing of the targeted cell type as a metric for selectivity.

As used in the specification and the claims herein, the phrase “physiological temperature appropriate for the cell” refers to temperatures known in the art and/or identifiable by the skilled worker which fall within a range suitable for healthy growth, propagation, and/or function of that particular cell or cell type; corresponding to the core temperature of the species from which the cell is derived; and/or corresponding to a healthy, living organism comprising the cell. For example, temperatures around 37° C. are appropriate for many mammalian cells depending on the species.

For purposes as described herein, the phrase “internalization of a molecular complex comprising the CD20-binding molecule bound to CD20” means the cellular internalization of the CD20-binding molecule is CD20-mediated in that the internalization begins with CD20-binding molecule and cell-surface CD20 forming a complex at an extracellular position and ends with both the CD20-binding molecule and CD20 molecule(s) entering the cell prior to dissociation of the CD20-binding molecule from CD20 molecule(s) to which the CD20-binding molecule has bound.

For purposes as described herein, the phrase “CD20 natively present on the surface of a cell” means a cell expresses the CD20 molecule using its own protein synthesis machinery and localizes the CD20 molecule to a cellular surface using its own intracellular routing machinery such that the CD20 molecule is physically coupled to said cell and at least a part of the CD20 molecule is accessible from an extracellular space, i.e. on the surface of a cell.

For the purposes of certain embodiments as described herein, cellular internalization is considered rapid if the time for internalization to occur due to the binding of the CD20-binding molecule as described herein is reduced as compared to the time for internalization of a prior art reference molecule at the same percent CD20 occupancy as determined by the same assay using the same cell type at the same temperature.

As used in the specification and the claims herein, the phrase “rapid cellular internalization” refers to the ability of a CD20-binding molecule as described herein to decrease the time on average for cellular internalization of an extracellular CD20 antigen or cell surface localized CD20 molecule as compared to the time on average required for cellular internalization of an extracellular CD20 antigen or cell surface localized CD20 molecule, as measured by any one of a number of cell internalization assays known in the art or described herein.

As used in the specification and the claims herein, the phrase “rapid internalization” includes internalization which may be assayed as compared to a basal CD20 internalization rate and/or molecular binding induced internalization rate for CD20 after administration of an immunoglobulin-type binding molecule (e.g. a monoclonal antibody) known in the art to bind an extracellular part of CD20. The scope of the phrase “rapid cellular internalization” is intended to encompass internalization rates, on average, faster than those observed when testing a CD20-specific antibody or immunoglobulin-derived protein molecule with an Fc region. In general, an internalization rate constant may be defined as the time after administration of a CD20-specific binding molecule of interest to CD20 positive cells at which 50% of cell surface CD20 antigens, CD20 molecules, and/or the CD20-specific binding molecule is internalized at a given administered concentration, mass, molarity, or CD20 occupancy-adjusted concentration, to a particular cell type, and at a particular temperature. Cell-surface CD20 internalization, whether basally or in response to administration of a CD20-binding molecule, may be assayed by various methods known to the skilled worker.

For the purposes of certain embodiments as described herein, cellular internalization is considered rapid if the time for internalization to occur due to the binding of the CD20-binding molecule as described herein is reduced as compared to the time for internalization of the target CD20 molecule with the binding of a well-characterized antibody recognizing a CD20 antigen, such as the αCD20 monoclonal antibody 1H4 (Haisma H et al., Blood 92: 184-90 (1999)). For example, internalization timing for the CD20 antigen, although variable for cell type and antibody type, does not typically begin to reach maximal levels until approximately six hours or more after binding. Thus the term “rapid” as used throughout the present description is intended to indicate that a CD20-binding molecule as described herein enters one or more CD20-expressing and/or CD20 positive cells in less than six hours. In some embodiments, rapid can be as quickly as less than about thirty minutes, but can also encompass a range of from about 1 hour to about 2 hours, to about 3 hours, to about 4 hours, to about 5 hours; a range of about 2 hours to about 3 hours, to about 4 hours, to about 5 hours; a range of about 3 hours to about 4 hours, to about 5 hours; and a range of about 4 hours to about 5 hours.

For purposes as described herein, the phrase “one or more non-covalent linkages,” with regard to a molecule comprising two or more components linked together, includes the types of linkages connecting the components that in certain molecules may be observed as being eliminated (i.e., no longer connecting two or more components) when changing the molecule from native protein-folding conditions to protein-denaturing conditions. For example, when using techniques known in the art and/or described herein, such as, e.g., electrophoretic and/or chromatographic assays, for assaying the sizes of proteinaceous molecules, a multi-component molecule that appears as a single-sized species under native protein-folding conditions (e.g. pH-buffered environments intended to be similar to the lumen of the endoplasmic reticulum of a eukaryotic cell or to an extracellular environment within an organism), can also be observed as being composed of two or more smaller-sized, proteinaceous molecules under denaturing conditions and/or after being subjected to a denaturing condition. “Protein-denaturing” conditions are known to the skilled worker and include conditions markedly different from native protein-folding conditions, such as, e.g., environments with a high temperature (e.g., greater than 50 degrees Celsius) and/or those characterized by the presence of chemical denaturants and/or detergents, such as, e.g., 1-10% sodium dodecyl sulfate, polysorbates, Triton® X-100, sarkosyl, and other detergents whether ionic, non-ionic, zwitterionic, and/or chaotropic.

As used herein, the term “monomeric” with regard to describing a protein and/or proteinaceous molecule refers to a molecule comprising only one polypeptide component consisting of a single, continuous polypeptide, regardless of its secondary or tertiary structure, which may be synthesized by a ribosome from a single polynucleotide template, including a continuous linear polypeptide which later forms a cyclic structure. In contrast, a multimeric molecule may comprise two or more polypeptides (e.g. subunits) which together do not form a single, continuous polypeptide that may be synthesized by a ribosome from a single polynucleotide template is multimeric.

As used herein, the term “multimeric” with regard to describing a protein and/or proteinaceous molecule refers to a molecule that comprises two or more, individual, polypeptide components associated together and/or linked together, such as, e.g., a molecule consisting of two components each of which is its own continuous polypeptide. For example, the association or linkage between components of a molecule may include 1) one or more non-covalent interactions; 2) one or more post-translational, covalent interactions; 3) one or more, covalent chemical conjugations; and/or 4) one or more covalent interactions resulting in a single molecule comprising a non-linear polypeptide, such as, e.g., a branched or cyclic polypeptide structure, resulting from the arrangement of the two or more polypeptide components. A molecule comprising two, discontinuous polypeptides as a result of the proteolytic cleavage of one or more peptide bonds in a single, continuous polypeptide synthesized by a ribosome from a single polynucleotide templates is “multimeric” and not “monomeric.”

As used herein, the phrase “CD20-binding molecule composition” refers to a composition comprising at least one type of CD20-binding molecule, and, may commonly comprise two or more types of CD20-binding molecule, wherein each type of CD20-binding molecule has a reproducibly measurable representation in the composition, e.g., of at least one percent (by mass) of the most abundant type of CD20-binding molecule. A composition comprising only one type of CD20-binding molecule with no other type of proteinaceous molecule present (e.g. a composition comprising one hundred percent of a single type of CD20-binding molecule of the total proteinaceous molecule(s) present) is encompassed by the phrase “CD20-binding molecule composition.”

The term “about” when immediately preceding a numerical value means±up to 20% of the numerical value. For example, “about” a numerical value means±up to 20% of the numerical value, in some embodiments, ±up to 19%, ±up to 18%, ±up to 17%, ±up to 16%, ±up to 15%, ±up to 14%, ±up to 13%, ±up to 12%, ±up to 11%, ±up to 10%, ±up to 9%, ±up to 8%, ±up to 7%, ±up to 6%, ±up to 5%, ±up to 4%, ±up to 3%, ±up to 2%, ±up to 1%, ±up to less than 1%, or any other value or range of values therein.

The term “pharmaceutically acceptable salt” means an acid addition salt of a basic active agent, including a basic antimicrobial agent, or a base addition salt of an acidic active agent, including an acidic antimicrobial agent.

Basic molecules that form an acid addition salt include, for example, those comprising an amino group. Illustrative inorganic acids that form acid addition salts with basic molecules include hydrochloric, hydrobromic, sulfuric, nitric and phosphoric acids, as well as acidic metal salts such as sodium monohydrogen orthophosphate and potassium hydrogen sulfate. Illustrative organic acids that form acid addition salts with basic molecules include mono-, di- and tricarboxylic acids. Illustrative of such organic acids are, for example, acetic, trifluoroacetic, propionic, glycolic, lactic, pyruvic, malonic, succinic, glutaric, fumaric, malic, oxalic, tartaric, citric, ascorbic, maleic, hydroxymaleic, benzoic, hydroxybenzoic, phenylacetic, cinnamic, mandelic, salicylic, 2-phenoxybenzoic, isethionic, p-toluenesulfonic acid and other sulfonic acids such as methanesulfonic acid, ethanesulfonic acid and 2-hydroxyethanesulfonic acid. Either the mono- or di-acid salts can be formed, and such salts can exist in either a hydrated, solvated or substantially anhydrous form.

Acidic molecules that form a base addition salt include, for example, compounds comprising a carboxylic acid group. Illustrative inorganic bases that form base addition salts with acidic acid agents include lithium, sodium, potassium, calcium, magnesium or barium hydroxides, carbonates and bicarbonates, as well as ammonia. Illustrative organic bases that form base addition salts with acidic molecules include aliphatic, alicyclic or aromatic organic amines such as isopropylamine, methylamine, trimethylamine, picoline, diethylamine, triethylamine, tripropylamine, ethanolamine, 2-dimethylaminoethanol, 2-diethylaminoethanol, dicyclohexylamine, lysine, arginine, histidine, caffeine, procaine, hydrabamine, choline, EGFRaine, ethylenediamine, glucosamine, methylglucamine, theobromine, purines, piperazine, piperidine, N-ethylpiperidine, polyamine resins, and the like. Illustrative organic bases are isopropylamine, diethylamine, ethanolamine, trimethylamine, dicyclohexylamine, choline, and caffeine (see e.g. Berge S et al., J Pharm Sci 66, 1-19 (1977)).

The formation of a desired compound salt is achieved using standard techniques. For example, the neutral compound is treated with an acid or base in a suitable solvent and the formed salt is isolated by filtration, extraction or any other suitable method.

### CD20-Binding Molecules and Methods of Use Thereof

Provided herein are methods for treating or slowing the progression of a disease, disorder, or condition, comprising administering to a subject in need thereof an effective amount of (i) a CD20-binding molecule and (ii) at least one additional therapeutic agent, wherein the CD20-binding molecule comprises (a) a CD20-binding region, wherein the CD20-binding region is capable of specifically binding an extracellular portion of a CD20 protein, and b) a Shiga toxin effector polypeptide region (the CD20-binding molecule being a “CD20-binding molecule as described herein”). In some embodiments, the CD20-binding molecule as described herein is multivalent. In certain other embodiments, the CD20-binding molecule as described herein is monovalent.

In some embodiments, a method for treating or slowing the progression of a disease, disorder, or condition comprises administering to a subject in need thereof an effective amount of: (i) a CD20-binding molecule; and (ii) one or more additional therapeutic agents; wherein the CD20-binding molecule comprises a polypeptide having the sequence of SEQ ID NO: 54. In some embodiments, the CD20-binding molecule is a homodimer comprising two identical polypeptides. In some embodiments, each identical polypeptide has the amino acid sequence of SEQ ID NO: 54; and wherein there is a disulfide bond at each cysteine residue at amino acid position 503 of SEQ ID NO: 54.

In addition, provided are compositions comprising an effective amount of a CD20-binding molecule as described herein and an additional therapeutic agent. Described herein is the discovery that illustrative CD20-binding molecules and CD20-binding molecule compositions as described herein are cytotoxic to CD20-expressing cells (see Examples, infra). The ability to deliver a therapeutic into a target cell and kill it is convincing proof of cellular internalization (Mazor Y et al., J Immunol Methods 321: 41-59 (2007)). Furthermore, the potency of target cell kill can be correlated with the efficiency of cellular internalization (Pirie C et al., J Biol Chem 286: 4165-72 (2011)).

As described in more detail in the Examples, certain illustrative multivalent CD20-binding molecules, and enriched compositions thereof, exhibit unexpectedly cytotoxic potencies compared to what was measured using equivalent amounts of a monovalent CD20-binding variant, and certain compositions thereof. Without being bound by theory, multivalent CD20-binding molecules as described herein, and compositions thereof, may possess the improved ability(ies) of: internalizing into CD20-expressing cells, intracellularly routing to a certain subcellular compartment(s), and/or delivering an active toxin effector polypeptide region (e.g. a Shiga toxin A Subunit effector polypeptide) to the cytosol as compared to certain 1) monovalent CD20-binding molecules and compositions thereof, 2) multivalent CD20-binding molecules lacking a toxin effector region(s) (e.g. a Shiga toxin A Subunit effector polypeptide), and/or compositions comprising another CD20-binding molecule comprising a Shiga toxin A Subunit effector polypeptide to total CD20-binding molecule.

### I. The General Structure of the CD20-Binding Molecules as Described Herein

Provided herein are various CD20-binding molecules for targeted cellular internalization into CD20-expressing cell types. In some embodiments, a CD20-binding molecule as described herein comprises 1) a CD20 binding region capable of specifically binding an extracellular part of CD20 and 2) at least one Shiga toxin effector region comprising a polypeptide derived from the amino acid sequence of an A Subunit of at least one member of the Shiga toxin family. Also provided herein are various compositions comprising an effective amount of a CD20-binding molecule and an additional therapeutic agent.

In some embodiments, the binding molecule is a homo-dimer or a hetero-dimer. In some embodiments, the binding molecule is a homo-dimer comprising two monomers, wherein each monomer comprises a CD20 binding region and a Shiga toxin effector polypeptide. In some embodiments, a dimeric binding molecule exhibits properties which are more favorable than the properties of a monomeric variant comprising identical binding region and toxin region.

Some of the CD20-binding molecules described herein can induce rapid cellular internalization of CD20 (e.g., CD20 natively present on the surface of a cell). Upon binding a CD20 antigen on the surface of a cell, the CD20-binding molecules described herein are capable of inducing rapid cellular internalization of the complex comprising of the CD20-binding molecule and a CD20 antigen into the interior of a eukaryotic cell. In some embodiments, the CD20-binding molecules are capable of inducing, in less than about one hour, cellular internalization of a CD20 natively present on the surface of a cell. In some embodiments, the CD20-binding molecules are capable of inducing, in less than about one hour, cellular internalization of a CD20 natively present on the surface of a member of a B-cell lineage.

The linking of CD20 binding regions with Shiga-toxin-Subunit-A-derived polypeptides enables the engineering of cytotoxic Shiga-toxin based molecules that are capable of inducing rapid cellular internalization of natively expressed CD20, as well as capable of delivering additional exogenous materials into the interior of CD20 expressing cells. The CD20-binding molecules have uses, e.g., for targeted killing of CD20 positive cell types, delivering exogenous materials, as diagnostic agents, and as therapeutics for the treatment of a variety of conditions in subjects, such as cancers, tumors, and immune disorders related to B-cell lineages and/or involving a CD20-expressing cell type.

Some of the CD20-binding molecules described herein, and compositions thereof, are cytotoxic and others are not, such as, e.g., for labeling the interiors of CD20-expressing cells. Certain CD20-binding molecules as described herein, and compositions thereof, can deliver additional exogenous materials into CD20-expressing cells and may or may not result in cytotoxicity independent of the activity of the Shiga toxin effector polypeptide region(s).

In some embodiments, upon administration of the CD20-binding molecule to a cell which expresses CD20 on a cellular surface, the CD20-binding molecules are capable of causing the death of the cell. In certain other embodiments, the CD20-binding molecules comprise Shiga toxin effector regions that lack catalytic activity and are not capable of causing the death of a cell.

In some embodiments, upon administration of the CD20-binding molecule to a first populations of cells whose members express CD20, and a second population of cells whose members do not express CD20, the cytotoxic effect of the CD20-binding molecule to members of the first population of cells relative to members of the second population of cells is at least 3-fold greater.

### A. CD20 Binding Regions of the CD20-Binding Molecules as Described Herein

The CD20-binding molecule as described herein comprises a CD20 binding region wherein each binding region comprises a peptide or polypeptide region capable of binding specifically to an extracellular part of a CD20 molecule. In some embodiments, the CD20-binding molecule comprises two or more CD20 binding regions wherein each binding region comprises a peptide or polypeptide region capable of binding specifically to an extracellular part of a CD20 molecule in physical association with a cell. The CD20 binding region may comprise one or more various peptidic or polypeptide moieties, such as randomly generated peptide sequences, naturally occurring ligands or derivatives thereof, immunoglobulin-derived domains, engineered scaffolds as alternatives to immunoglobulin domains, and the like.

In some embodiments, the CD20 binding region comprises the polypeptide comprising, consisting essentially of, or consisting of the binding region that includes autonomous V_{H} domains, single-domain antibody domains (sdAbs), heavy-chain antibody domains derived from camelids (V_{H}H fragments or V_{H} domain fragments), heavy-chain antibody domains derived from camelid V_{H}H fragments or V_{H} domain fragments, heavy-chain antibody domains derived from cartilaginous fishes, immunoglobulin new antigen receptors (IgNARs), V_{NAR} fragments, single-chain variable (scFv) fragments, nanobodies, Fd fragments consisting of the heavy chain and C_{H}1 domains, permutated Fvs (pFv), single chain Fv-C_{H}3 minibodies, dimeric C_{H}2 domain fragments (C_{H}2D), Fc antigen binding domains (Fcabs), isolated complementary determining region 3 (CDR3) fragments, constrained framework region 3, CDR3, framework region 4 (FR3-CDR3-FR4) polypeptides, small modular immunopharmaceutical (SMIP) domains, scFv-Fc fusions, multimerizing scFv fragments (diabodies, triabodies, tetrabodies), disulfide stabilized antibody variable (Fv) fragments, disulfide stabilized antigen-binding (Fab) fragments consisting of the V_{L}, V_{H}, C_{L} and C_{H}1 domains, bivalent nanobodies, bivalent minibodies, bivalent F(ab′)₂ fragments (Fab dimers), bispecific tandem V_{H}H fragments, bispecific tandem scFv fragments, bispecific nanobodies, bispecific minibodies, and any genetically manipulated counterparts of the foregoing that retains its binding functionality (see e.g. Wörn A, Plückthun A, J Mol Biol 305: 989-1010 (2001); Xu L et al., Chem Biol 9: 933-42 (2002); Wikman M et al., Protein Eng Des Sel 17: 455-62 (2004); Binz H et al., Nat Biotechnol 23: 1257-68 (2005); Hey T et al., Trends Biotechnol 23:514-522 (2005); Holliger P, Hudson P, Nat Biotechnol 23: 1126-36 (2005); Gill D, Damle N, Curr Opin Biotech 17: 653-8 (2006); Koide A, Koide S, Methods Mol Biol 352: 95-109 (2007); Byla P et al., J Biol Chem 285: 12096 (2010); Zoller F et al., Molecules 16: 2467-85 (2011); Alfarano P et al., Protein Sci 21: 1298-314 (2012); Madhurantakam C et al., Protein Sci 21: 1015-28 (2012); Varadamsetty G et al., J Mol Biol 424: 68-87 (2012); Reichen C et al., J Struct Biol 185: 147-62 (2014)).

In some embodiments, the CD20-binding molecule comprises a CD20 binding region which is an scFv comprising a V_{H} domain and a V_{L} domain. In some embodiments, the scFv comprises a linker which connects the V_{H} domain and the V_{L} domain. In some embodiments, the CD20 binding region is an anti-CD20 scFv comprising an amino acid sequence with at least 90%, at least 95%, at least 96%, at least 97%, at least 98%, or at least 99% identity to SEQ ID NO: 375. In some embodiments, the CD20 binding region is an anti-CD20 scFv comprising the amino acid sequence of SEQ ID NO: 375. In some embodiments, the CD20 binding region is an anti-CD20 scFv comprising the amino acid sequence of SEQ ID NO: 375 with one or more mutations relative thereto. For example, in some embodiments, the CD20 binding region comprises 1, 2, 3, 4, 5, 6, 7, 8, 9, 10, or more mutations relative to SEQ ID NO: 375. In some embodiments, the CD20 binding region is an anti-CD20 scFv comprising the amino acid sequence of SEQ ID NO: 375 with 1-5, 5-10, 11-5, 15-20, 10-25, 25-30, or more than 30 mutations. In some embodiments, the V_{H} domain of the anti-CD20 scFv comprises an amino acid sequence with at least 90%, at least 95%, at least 96%, at least 97%, at least 98%, or at least 99% identity to SEQ ID NO: 376. In some embodiments, the V_{H} domain of the anti-CD20 scFv comprises the amino acid sequence of SEQ ID NO: 376. In some embodiments, the V_{H} domain of the anti-CD20 scFv comprises the amino acid sequence of SEQ ID NO: 376 with one or more mutations relative thereto. For example, in some embodiments, the V_{H} domain comprises 1, 2, 3, 4, 5, 6, 7, 8, 9, 10, or more mutations relative to SEQ ID NO: 376. In some embodiments, the V_{H} domain of anti-CD20 scFv comprises the amino acid sequence of SEQ ID NO: 376 with 1-5, 5-10, 11-5, 15-20, 10-25, 25-30, or more than 30 mutations. In some embodiments, the V_{L} domain of the anti-CD20 scFv comprises an amino acid sequence with at least 90%, at least 95%, at least 96%, at least 97%, at least 98%, or at least 99% identity to SEQ ID NO: 377. In some embodiments, the V_{L} domain of the anti-CD20 scFv comprises the amino acid sequence of SEQ ID NO: 377. In some embodiments, the V_{L} domain of the anti-CD20 scFv comprises the amino acid sequence of SEQ ID NO: 377 with one or more mutations relative thereto. For example, in some embodiments, the V_{L} domain comprises 1, 2, 3, 4, 5, 6, 7, 8, 9 or 10 relative to SEQ ID NO: 377. In some embodiments, the V_{L} domain of the anti-CD20 scFv comprises the amino acid sequence of SEQ ID NO: 377 with 1-5, 5-10, 11-5, 15-20, 10-25, 25-30, or more than 30 mutations.

As used herein, the term “CD20 binding region” refers to a proteinaceous (e.g., peptidic and/or polypeptide) region of a molecule as described herein which is capable of specifically binding an extracellular part of a CD20 molecule with high affinity, such as, e.g., having a dissociation constant with regard to CD20 of 10⁻⁵ to 10⁻¹² moles per liter.

In some embodiments, the binding region of a CD20-binding molecule comprises a polypeptide capable of selectively and specifically binding an extracellular part of a CD20 expressed at a cellular surface and in physical association with a cell. In some embodiments, the CD20 binding region comprises a naturally occurring ligand of a CD20 molecule or derivative thereof that retains binding functionality to an extracellular part of CD20. In some embodiments, the CD20 binding region comprises a synthetic ligand capable of binding to an extracellular part of CD20 with high affinity.

While the name CD20 may refer to multiple proteins with related structures and polypeptide sequences from various species, as used herein, the term “CD20” refers to the B-lymphocyte antigen CD20 proteins present in mammals whose exact sequence might vary slightly based on the isoform and from individual to individual. Alternative names for CD20, as recognized in the art, include B-lymphocyte surface antigen Bl, Leu-16 and Bp35. For example, in humans CD20 typically refers to the protein represented by the predominant polypeptide sequence UnitProt P11836 and NCBI accession NP 690605.1; however, different isoforms and variants may exist. The polypeptide sequences of CD20 proteins from various species have been described, such as from bats, cats, cattle, dogs, mice, marmosets, and rats, and can be predicted by bioinformatics in numerous other species based on genetic homology (e.g. CD20 has been predicted in various primates, including baboons, macaques, gibbons, chimpanzees, and gorillas) (see NCBI protein database (National Center for Biotechnology Information, U.S.)). A skilled worker will be able to identify a CD20 related protein in mammals, even if it differs from the referenced sequences.

CD20 is expressed by B-cells within certain cell developmental stages that give rise to non-Hodgkins lymphoma (NHL) and chronic lymphocytic leukemia (CLL); however, CD20 is not expressed on hematopoietic stem cells or on mature plasma cells (van Meerten T et al., Clin Cancer Res 12: 4027-35 (2006)). An attractive characteristic of CD20 is that it represents a quasi-universal target of lymphoma cells for being expressed on approximately 90% of B-cell non-Hodgkin lymphomas (Anderson K et al., Blood 63: 2825-33 (1984); Press O et al., Cancer Res 49: 4906-12 (1989); Press O et al., Blood. 83: 1390-7 (1994); Manches O et al., Blood 101: 949-54 (2003)). Additional attractive characteristics of CD20 are its high expression on the plasma membrane of lymphoma cells and its multiple extracellular CD20 antigenic epitopes in close proximity to the plasma membrane (Teeling J et al., J Immunol 177: 362-71 (2006); Lim S et al., Haematologica 95: 135-43 (2010)).

An extracellular part of a CD20 molecule refers to a portion of its structure exposed to the extracellular environment when the CD20 molecule is present in a cell membrane, such as, e.g., CD20 molecules natively expressed at a cellular surface. In this context, exposed to the extracellular environment means that part of the CD20 molecule is accessible by, e.g., an antibody or at least a binding moiety smaller than an antibody such as a single-domain antibody domain, a nanobody, a heavy-chain antibody domain derived from camelids or cartilaginous fishes, a single-chain variable fragment, or any number of engineered alternative scaffolds to immunoglobulins (see below). The exposure to the extracellular environment of or accessibility to a part of CD20 physically coupled to a cell may be empirically determined by the skilled worker using methods well known in the art. Note that some portion of CD20, which was predicted not to be accessible to an antibody in the extracellular space based on its epitope location within CD20, was empirically shown to be accessible by a monoclonal antibody (Teeling J et al., J. Immunol. 177: 362-71 (2006)).

CD20 binding regions may be derived from antibody or antibody-like structures; however, alternative scaffolds from other sources are contemplated as a source of CD20 binding regions within the scope as described herein. In some embodiments, the CD20 binding region is derived from an immunoglobulin-derived binding region, such as an antibody paratope. In some embodiments, the CD20 binding region comprises an immunoglobulin-type binding region that is an engineered polypeptide not derived from any immunoglobulin domain.

In some embodiments, the CD20 binding region may comprise an immunoglobulin-type binding region. The term “immunoglobulin-type binding region” as used herein refers to a polypeptide region capable of binding one or more target biomolecules, such as an antigen or epitope. Immunoglobulin-type binding regions are functionally defined by their ability to bind to target molecules, such as CD20. Immunoglobulin-type binding regions are commonly derived from antibody or antibody-like structures; however, alternative scaffolds from other sources are contemplated within the scope of the term.

Immunoglobulin (Ig) proteins have a structural domain known as an Ig domain. Ig domains range in length from about 70-110 amino acid residues and possess a characteristic Ig-fold, in which typically 7 to 9 antiparallel beta strands arrange into two beta sheets which form a sandwich-like structure. The Ig fold is stabilized by hydrophobic amino acid interactions on inner surfaces of the sandwich and highly conserved disulfide bonds between cysteine residues in the strands. Ig domains may be variable (IgV or V-set), constant (IgC or C-set) or intermediate (IgI or I-set). Some Ig domains may be associated with a complementarity determining region or complementary determining region (CDR), also referred to as antigen binding region (ABR), which is important for the specificity of antibodies binding to their epitopes. Ig-like domains are also found in non-immunoglobulin proteins and are classified on that basis as members of the Ig superfamily of proteins. The HUGO Gene Nomenclature Committee (HGNC) provides a list of members of the Ig-like domain containing family.

As used herein, the term “heavy chain variable (V_{H}) domain” or “light chain variable (V_{L}) domain” respectively refer to any antibody V_{H} or V_{L} domain (e.g. a human V_{H} or V_{L} domain) as well as any derivative thereof retaining at least qualitative antigen binding ability of the corresponding native antibody (e.g. a humanized V_{H} or V_{L} domain derived from a native murine V_{H} or V_{L} domain). A V_{H} or V_{L} domain consists of a “framework” region interrupted by the three CDRs or ABRs. The framework regions serve to align the CDRs for specific binding to an epitope of an antigen. From amino-terminus to carboxyl-terminus, both V_{H} and V_{L} domains comprise the following framework (FR) and CDR regions: FR1, CDR1, FR2, CDR2, FR3, CDR3, and FR4. For camelid V_{H}H fragments, IgNARs of cartilaginous fish, V_{NAR} fragments, and derivatives thereof, there is a single heavy chain variable domain comprising the same basic arrangement: FR1, CDR1, FR2, CDR2, FR3, CDR3, and FR4.

An immunoglobulin-type binding region may be a polypeptide sequence of antibody or antigen-binding fragment thereof wherein the amino acid sequence has been varied from that of a native antibody or an Ig-like domain of a non-immunoglobulin protein, for example by molecular engineering or library screening. Because of the relevance of recombinant DNA techniques and in vitro library screening in the generation of immunoglobulin-type binding regions, antibodies can be redesigned to obtain desired characteristics, such as smaller size, cell entry, or other therapeutic improvements. The possible variations are many and may range from the changing of just one amino acid to the complete redesign of, for example, a variable region. Typically, changes in the variable region will be made in order to improve the antigen-binding characteristics, improve variable region stability, or reduce the potential for immunogenic responses.

There are numerous immunoglobulin-type binding regions that bind an extracellular part of CD20 contemplated herein. In some embodiments, the immunoglobulin-type binding region is derived from an immunoglobulin binding region, such as an antibody paratope capable of binding an extracellular part of CD20. In some embodiments, the immunoglobulin-type binding region comprises an engineered polypeptide not derived from any immunoglobulin domain but that functions like an immunoglobulin binding region by providing high-affinity binding to an extracellular part of CD20. This engineered polypeptide may optionally include polypeptide scaffolds comprising, consisting essentially of, or consisting of complementary determining regions from immunoglobulins as described herein.

There are numerous immunoglobulin-derived binding regions and non-immunoglobulin engineered polypeptides in the prior art that are useful for targeting the CD20-binding molecules as described herein to CD20-expressing cells. In some embodiments, the immunoglobulin-type binding region of the CD20-binding molecule described herein comprises, consists essentially of, or consists of one or more autonomous V_{H} domains, single-domain antibody domains (sdAbs), heavy-chain antibody domains derived from camelids (V_{H}H fragments or V_{H} domain fragments), heavy-chain antibody domains derived from camelid V_{H}H fragments or V_{H} domain fragments, heavy-chain antibody domains derived from cartilaginous fishes, immunoglobulin new antigen receptors (IgNARs), V_{NAR} fragments, single-chain variable (scFv) fragments, nanobodies, Fd fragments consisting of the heavy chain and C_{H}1 domains, permutated Fvs (pFvs), single chain Fv-C_{H}3 minibodies, dimeric C_{H}2 domain fragments (C_{H}2D), Fc antigen binding domains (Fcabs), isolated complementary determining region 3 (CDR3) fragments, constrained framework region 3, CDR3, framework region 4 (FR3-CDR3-FR4) polypeptides, small modular immunopharmaceutical (SMIP) domains, scFv-Fc fusions, multimerizing scFv fragments (diabodies, triabodies, tetrabodies), disulfide stabilized antibody variable (Fv) fragments, disulfide stabilized antigen-binding (Fab) fragments consisting of the V_{L}, V_{H}, C_{L} and C_{H}1 domains, bivalent nanobodies, bivalent minibodies, bivalent F(ab′)2 fragments (Fab dimers), bispecific tandem V_{H}H fragments, bispecific tandem scFv fragments, bispecific nanobodies, bispecific minibodies, and any genetically manipulated counterparts of the foregoing that retain its paratope and binding function (see Ward E et al., Nature 341: 544-6 (1989); Davies J, Riechmann L, Biotechnology (NY) 13: 475-9 (1995); Brinkmann U et al., J Mol Biol 268: 107-17 (1997); Reiter Y et al., Mol Biol 290: 685-98 (1999); Riechmann L, Muyldermans S, J Immunol Methods 231: 25-38 (1999); Tanha J et al., J Immunol Methods 263: 97-109 (2002); Vranken W et al., Biochemistry 41: 8570-9 (2002); Jespers L et al., J Mol Biol 337: 893-903 (2004); Jespers L et al., Nat Biotechnol 22: 1161-5 (2004); To R et al., J Biol Chem 280: 41395-403 (2005); Saerens D et al., Curr Opin Pharmacol 8: 600-8 (2008); Dimitrov D, MAbs 1: 26-8 (2009); Weiner L, Cell 148: 1081-4 (2012); Ahmad Z et al., Clin Dev Immunol 2012: 980250 (2012)).

There are a variety of binding regions comprising polypeptides derived from the constant regions of immunoglobulins, such as, e.g., engineered dimeric Fc domains, monomeric Fcs (mFcs), scFv-Fcs, V_{H}H-Fcs, C_{H}2 domains, monomeric C_{H}3s domains (mC_{H}3s), synthetically reprogrammed immunoglobulin domains, and/or hybrid fusions of immunoglobulin domains with ligands (Hofer T et al., Proc Natl Acad Sci USA 105: 12451-6 (2008); Xiao J et al., J Am Chem Soc 131: 13616-13618 (2009); Xiao X et al., Biochem Biophys Res Commun 387: 387-92 (2009); Wozniak-Knopp G et al., Protein Eng Des Sel 23 289-97 (2010); Gong R et al., PLoS ONE 7: e42288 (2012); Wozniak-Knopp G et al., PLoS ONE 7: e30083 (2012); Ying T et al., J Biol Chem 287: 19399-408 (2012); Ying T et al., J Biol Chem 288: 25154-64 (2013); Chiang M et al., J Am Chem Soc 136: 3370-3 (2014); Rader C, Trends Biotechnol 32: 186-97 (2014) Ying T et al., Biochimica Biophys Acta 1844: 1977-82 (2014)).

In some embodiments, the binding region comprises an engineered, alternative scaffold to immunoglobulin domains. Engineered alternative scaffolds are known in the art which exhibit similar functional characteristics to immunoglobulin-derived structures, such as high-affinity and specific binding of target biomolecules, and may provide improved characteristics to certain immunoglobulin domains, such as, e.g., greater stability or reduced immunogenicity. Generally, alternative scaffolds to immunoglobulins are less than 20 kilodaltons (kDa), consist of a single polypeptide chain, lack cysteine residues, and exhibit relatively high thermodynamic stability.

In some embodiments, the immunoglobulin-type binding region comprises, consists essentially of, or consists of one or more engineered, Armadillo repeat polypeptides (ArmRPs); engineered, fibronectin-derived, 10^{th} fibronectin type III (10Fn3) domains (monobodies, AdNectins™, or AdNexins™); engineered, tenascin-derived, tenascin type III domains (Centryns™); engineered, ankyrin repeat motif containing polypeptides (DARPins™); engineered, low-density-lipoprotein-receptor-derived, A domains (LDLR-A) (Avimers™); lipocalins (anticalins); engineered, protease inhibitor-derived, Kunitz domains; engineered, Protein-A-derived, Z domains (Affibodies™); engineered, gamma-B crystalline-derived scaffold or engineered, ubiquitin-derived scaffolds (Affilins); Sac7d-derived polypeptides (Nanoffitins® or affitins); engineered, Fyn-derived, SH2 domains (Fynomers®); and engineered antibody mimics and any genetically manipulated counterparts of the foregoing that retains its binding functionality (Wörn A, Plückthun A, J Mol Biol 305: 989-1010 (2001); Xu L et al., Chem Biol 9: 933-42 (2002); Wikman M et al., Protein Eng Des Sel 17: 455-62 (2004); Binz H et al., Nat Biotechnol 23: 1257-68 (2005); Hey T et al., Trends Biotechnol 23:514-522 (2005); Holliger P, Hudson P, Nat Biotechnol 23: 1126-36 (2005); Gill D, Damle N, Curr Opin Biotech 17: 653-8 (2006); Koide A, Koide S, Methods Mol Biol 352: 95-109 (2007); Byla P et al., J Biol Chem 285: 12096 (2010); Zoller F et al., Molecules 16: 2467-85 (2011); Alfarano P et al., Protein Sci 21: 1298-314 (2012); Madhurantakam C et al., Protein Sci 21: 1015-28 (2012); Varadamsetty G et al., J Mol Biol 424: 68-87 (2012)). For example, the engineered Fn3(CD20) is an engineered, alternative scaffold CD20 binding region which exhibits high-affinity binding to CD20 expressing cells (Natarajan A et al., Clin Cancer Res 19: 6820-9 (2013)).

Among certain embodiments as described herein, the immunoglobulin-type binding region is derived from a nanobody or single domain immunoglobulin-derived region V_{H}H. Generally, nanobodies are constructed from fragments of naturally occurring single, monomeric variable domain antibodies (sdAbs) of the sort found in camelids and cartilaginous fishes (Chondrichthyes). Nanobodies are engineered from these naturally occurring antibodies by truncating the single, monomeric variable domain to create smaller and more stable molecules, such as, e.g., IgNAR, V_{H}H, and V_{NAR} constructs. Due to their small size, nanobodies are able to bind to antigens that are not accessible to whole antibodies. Among certain embodiments as described herein, the immunoglobulin-type binding region is derived from a nanobody or single domain immunoglobulin-derived region V_{H}H which exhibits high-affinity binding specifically to an extracellular part of CD20.

In some embodiments, the immunoglobulin-type binding region of the CD20-binding molecules as described herein comprises an immunoglobulin-derived binding region that does not comprise an Fc region or any Fc region effector domain which retains an Fc region effector function. In some embodiments, the CD20-binding molecule does not comprise an Fc region or Fc region effector domain which retains an Fc function (see examples of Fc functions below).

As used herein, the phrase “Fc region” refers to the fragment crystallizable region or Fc (Fragment, crystallizable region) which is a polypeptide domain present in immunoglobulins, such as, e.g., the immunoglobulin isotypes IgA, IgD, IgE, IgG, and IgM. Fc regions interact with the complement system of the immune system and/or Fc receptors present on immune cells, such as, e.g., T-cells, basophils, eosinophils, macrophagocytes (macrophages), mast cells, neutrophils, and natural killer cells (NK cells). Fc region effector functions include activating T-cells, stimulating the release of inflammatory mediators such as cytokines like TNF-alpha, initiating complement dependent cytotoxicity (CDC), antibody-dependent cytotoxicity (ADCC), eventual phagocytosis, and possible immunization effects. Fc regions may be engineered into recombinant polypeptides and proteins, such as, e.g., fusions of antigen-binding fragments and Fc regions in synthetic F(ab′)2 and Fcabs.

The CD20-binding molecules as described herein that do not comprise any Fc region or Fc region effector domain which retains an Fc region effector function may function equally well in subjects with impaired Fc-FcyR-dependent mechanisms, such as immunocompromised patients, as in other subjects, such as immunocompetent patients.

Any of the above CD20 binding regions may be used as a component as described herein as long as the CD20 binding region component has a dissociation constant of 10⁻⁵ to 10⁻¹² moles per liter, preferably less than 200 nM, towards an extracellular part of CD20. In some embodiments, the CD20-binding molecule as described herein comprises a toxin effector region derived from a proteinaceous toxin, such as, e.g., a Shiga toxin A Subunit of the Shiga toxin family.

### B. Shiga Toxin Effector Polypeptide of a CD20-Binding Molecule as Described Herein

For purposes as described herein, the phrase “Shiga toxin effector region”, “Shiga toxin effector polypeptide”, or “Shiga toxin effector polypeptide region” refers to a polypeptide derived from a Shiga toxin A Subunit of at least one member of the Shiga toxin family wherein the Shiga toxin effector polypeptide is capable of exhibiting at least one Shiga toxin function. Shiga toxin functions include, e.g., promoting cell entry, deforming lipid membranes, stimulating clathrin-mediated endocytosis, directing retrograde transport, directing subcellular routing, avoiding intracellular degradation, catalytically inactivating ribosomes, effectuating cytotoxicity, and effectuating cytostatic effects.

A member of the Shiga toxin family refers to any member of a family of naturally occurring protein toxins which are structurally and functionally related, notably, toxins isolated from S. dysenteriae and E. coli (Johannes L, Römer W, Nat Rev Microbiol 8: 105-16 (2010)). For example, the Shiga toxin family encompasses true Shiga toxin (Stx) isolated from S. dysenteriae serotype 1, Shiga-like toxin 1 variants (SLT1 or Stx1 or SLT-1 or Slt-I) isolated from serotypes of enterohemorrhagic E. coli, and Shiga-like toxin 2 variants (SLT2 or Stx2 or SLT-2) isolated from serotypes of enterohemorrhagic E. coli. SLT1 differs by only one residue from Stx, and both have been referred to as Verocytotoxins or Verotoxins (VTs) (O'Brien A et al., Curr Top Microbiol Immunol 180: 65-94 (1992)). Although SLT1 and SLT2 variants are only about 53-60% similar to each other at the amino acid sequence level, they share mechanisms of enzymatic activity and cytotoxicity common to the members of the Shiga toxin family (Johannes, Nat Rev Microbiol 8: 105-16 (2010)). Over 39 different Shiga toxins have been described, such as the defined subtypes Stx1a, Stx1c, Stx1d, and Stx2a-g (Scheutz F et al., J Clin Microbiol 50: 2951-63 (2012)). Members of the Shiga toxin family are not naturally restricted to any bacterial species because Shiga-toxin-encoding genes can spread among bacterial species via horizontal gene transfer. As an example of interspecies transfer, a Shiga toxin was discovered in a strain of A. haemolyticus isolated from a patient (Grotiuz G et al., J Clin Microbiol 44: 3838-41 (2006)). Once a Shiga toxin encoding polynucleotide enters a new subspecies or species, the Shiga toxin amino acid sequence is presumed to be capable of developing slight sequence variations due to genetic drift and/or selective pressure while still maintaining a mechanism of cytotoxicity common to members of the Shiga toxin family.

Shiga toxin effector polypeptides of the CD20-binding molecules as described herein comprise, consist essentially of, or consist of a polypeptide derived from a Shiga toxin A Subunit dissociated from any form of its native Shiga toxin B Subunit. In addition, the CD20-binding molecules as described herein do not comprise any polypeptide comprising, consisting essentially of, or consisting of a functional binding domain of a native Shiga toxin B subunit. Rather, the Shiga toxin A Subunit derived regions of the CD20-binding molecules as described herein are functionally associated with heterologous binding regions to effectuate cell targeting.

In some embodiments, a Shiga toxin effector polypeptide of the CD20-binding molecules as described herein may comprise, consist essentially of, or consist of a full-length Shiga toxin A Subunit (e.g. SLT-1A (SEQ ID NO:1), StxA (SEQ ID NO:2), or SLT-2A (SEQ ID NO:3)), noting that naturally occurring Shiga toxin A Subunits may comprise precursor forms containing signal sequences of about 22 amino acids at their amino-terminals which are removed to produce mature Shiga toxin A Subunits and are recognizable to the skilled worker. In other embodiments, the Shiga toxin effector polypeptide described herein comprises, consists essentially of, or consists of a truncated Shiga toxin A Subunit which is shorter than a full-length Shiga toxin A Subunit.

Shiga-like toxin 1 A Subunit truncations are catalytically active, capable of enzymatically inactivating ribosomes in vitro, and cytotoxic when expressed within a cell. The smallest Shiga toxin A Subunit fragment exhibiting full enzymatic activity was shown to be a polypeptide composed of residues 1-239 of Slt1A. Although the smallest fragment of the Shiga toxin A Subunit reported to retain substantial catalytic activity was residues 75-247 of StxA, a StxA truncation expressed de novo within a eukaryotic cell requires only up to residue 240 to reach the cytosol and exert catalytic inactivation of ribosomes.

Shiga toxin effector polypeptides may commonly be smaller than a full-length Shiga toxin A Subunit. In some embodiments, the Shiga toxin effector polypeptide maintain the polypeptide region from amino acid position 77 to 239 (SLT-1A (SEQ ID NO:1) or StxA (SEQ ID NO:2)) or the equivalent in other A Subunits of members of the Shiga toxin family (e.g. 77 to 238 of (SEQ ID NO:3)). For example, in some embodiments described herein, a Shiga toxin effector polypeptide region derived from SLT-1A may comprise, consist essentially of, or consist of amino acids 75 to 251 of SEQ ID NO:1, 1 to 241 of SEQ ID NO:1, 1 to 251 of SEQ ID NO:1, or amino acids 1 to 261 of SEQ ID NO:1. In some embodiments, the Shiga toxin effector region derived from SLT-1A comprises a sequence with at least 90%, at least 95%, at least 96%, at least 97%, at least 98%, or at least 99% identity to amino acids 75 to 251 of SEQ ID NO:1, 1 to 241 of SEQ ID NO:1, 1 to 251 of SEQ ID NO:1, or amino acids 1 to 261 of SEQ ID NO:1.

In some embodiments, a Shiga toxin effector region may be derived from StxA. In some embodiments, a Shiga toxin effector region derived from StxA may comprise, consist essentially of, or consist of amino acids 75 to 251 of SEQ ID NO:2, 1 to 241 of SEQ ID NO:2, 1 to 251 of SEQ ID NO:2, or amino acids 1 to 261 of SEQ ID NO:2. In some embodiments, the Shiga toxin effector region derived from StxA comprises a sequence with at least 90%, at least 95%, at least 96%, at least 97%, at least 98%, or at least 99% identity to amino acids 75 to 251 of SEQ ID NO:2, 1 to 241 of SEQ ID NO:2, 1 to 251 of SEQ ID NO:2, or amino acids 1 to 261 of SEQ ID NO:2.

In some embodiments, a Shiga toxin effector region may be derived from SLT-2. In some embodiments, a Shiga toxin effector region derived from SLT-2 may comprise, consist essentially of, or consist of amino acids 75 to 251 of SEQ ID NO:3, 1 to 241 of SEQ ID NO:3, 1 to 251 of SEQ ID NO:3, or amino acids 1 to 261 of SEQ ID NO:3. In some embodiments, the Shiga toxin effector region derived from SLT-2 comprises a sequence with at least 90%, at least 95%, at least 96%, at least 97%, at least 98%, or at least 99% identity to amino acids 75 to 251 of SEQ ID NO:3, 1 to 241 of SEQ ID NO:3, 1 to 251 of SEQ ID NO:3, or amino acids 1 to 261 of SEQ ID NO:3.

In some embodiments, the binding molecules described herein comprise a Shiga toxin effector polypeptide comprising the sequence of any one of SEQ ID NOs: 1-3 and 380-391. In some embodiments, the binding molecules described herein comprise a Shiga toxin effector polypeptide comprising a sequence with one or more mutations relative to any one of SEQ ID NOs: 1-3 and 380-391. In some embodiments, the binding molecules described herein comprise a Shiga toxin effector polypeptide comprising a sequence with at least 90%, at least 95%, at least 96%, at least 97%, at least 98%, or at least 99% identity to any one of SEQ ID NOs: 1-3 and 380-391. In some embodiments, the binding molecules described herein comprise a Shiga toxin effector polypeptide with a sequence of any one of SEQ ID NOs: 1-3 and 380-391 with one or more mutations, such as 2, 3, 4, 5, 6, 7, 8, or 10 mutations. In some embodiments, the Shiga toxin effector comprises any one of SEQ ID NOs: 1-3 and 380-391 with 1-5, 5-10, 11-5, 15-20, 10-25, 25-30, or more than 30 mutations. In some embodiments, the binding molecules described herein comprise a Shiga toxin effector polypeptide comprising a sequence with one or more mutations relative to any one of SEQ ID NOs: 1-3 and 380-391, wherein the sequence comprises a S45C mutation. In some embodiments, mutations in the Shiga toxin effector polypeptide render the polypeptide catalytically inactive. In some embodiments, mutations in the Shiga toxin effector polypeptide do not affect the catalytic activity of the polypeptide. In some embodiments, mutations in the Shiga toxin effector polypeptide increase the catalytic activity of the polypeptide. In some embodiments, mutations in the Shiga toxin effector polypeptide decrease the catalytic activity of the polypeptide.

In some embodiments, the binding molecules described herein comprise a Shiga toxin effector polypeptide comprising amino acids 1 to 251 of SEQ ID NO: 1. In some embodiments, the binding molecules described herein comprise a Shiga toxin effector polypeptide that has one or more mutations relative to amino acids 1 to 251 of SEQ ID NO: 1. In some embodiments, the binding molecules described herein comprise a Shiga toxin effector polypeptide comprising a sequence with at least 90%, at least 95%, at least 96%, at least 97%, at least 98%, or at least 99% identity to amino acids 1 to 251 of SEQ ID NO: 1. In some embodiments, the binding molecules described herein comprise a Shiga toxin effector polypeptide comprising amino acids 1 to 251 of SEQ ID NO: 1 with one or more mutations, such as 2, 3, 4, 5, 6, 7, 8, or 10 mutations. In some embodiments, the Shiga toxin effector comprises amino acids 1 to 251 of SEQ ID NO: 1, with 1-5, 5-10, 11-5, 15-20, 10-25, 25-30, or more than 30 mutations. In some embodiments, mutations in the Shiga toxin effector polypeptide render the polypeptide catalytically inactive. In some embodiments, mutations in the Shiga toxin effector polypeptide do not affect the catalytic activity of the polypeptide. In some embodiments, mutations in the Shiga toxin effector polypeptide increase the catalytic activity of the polypeptide. In some embodiments, mutations in the Shiga toxin effector polypeptide decrease the catalytic activity of the polypeptide. In some embodiments, mutations in the Shiga toxin effector polypeptide reduce immunogenicity of the CD20-binding molecules disclosed herein.

In some embodiments, the binding molecules described herein comprise a Shiga toxin effector polypeptide comprising the amino acid sequence of SEQ ID NO: 382. In some embodiments, the binding molecules described herein comprise a Shiga toxin effector polypeptide that has one or more mutations relative to the amino acid sequence of SEQ ID NO: 382. In some embodiments, the binding molecules described herein comprise a Shiga toxin effector polypeptide comprising a sequence with at least 90%, at least 95%, at least 96%, at least 97%, at least 98%, or at least 99% identity to SEQ ID NO: 382. In some embodiments, the binding molecules described herein comprise a Shiga toxin effector polypeptide comprising the amino acid sequence of SEQ ID NO: 382, with one or more mutations, such as 2, 3, 4, 5, 6, 7, 8, or 10 mutations. In some embodiments, the Shiga toxin effector comprises the amino acid sequence of SEQ ID NO: 382, with 1-5, 5-10, 11-5, 15-20, 10-25, 25-30, or more than 30 mutations. In some embodiments, mutations in the Shiga toxin effector polypeptide render the polypeptide catalytically inactive. In some embodiments, mutations in the Shiga toxin effector polypeptide do not affect the catalytic activity of the polypeptide. In some embodiments, mutations in the Shiga toxin effector polypeptide increase the catalytic activity of the polypeptide. In some embodiments, mutations in the Shiga toxin effector polypeptide decrease the catalytic activity of the polypeptide. In some embodiments, mutations in the Shiga toxin effector polypeptide reduce immunogenicity of the CD20-binding molecules disclosed herein.

In some embodiments, the Shiga toxin effector polypeptide differs from a naturally occurring Shiga toxin A Subunit by up to 1, 2, 3, 4, 5, 6, 7, 8, 9, 10, 15, 20, 25, 30, 35, 40 or more amino acid residues (but by no more than that which retains at least 85%, 90%, 95%, 99%, or more amino acid sequence identity).

In some embodiments, the CD20-binding molecule comprises a toxin effector region derived from a proteinaceous toxin other than a Shiga toxin(s). In some embodiments, the CD20-binding molecule comprises a catalytically inactive Shiga toxin effector region. In some embodiments, the CD20-binding molecule does not comprise a Shiga toxin effector region. In some embodiments, the CD20-binding molecule comprises a toxin effector region, whether catalytically active or inactive, derived from a toxin(s) other than a member of the Shiga toxin family, such as, e.g., from an ABx toxin other than Shiga toxin, a ribosome inactivating protein toxin other than Shiga toxin, abrin, anthrax toxin, Aspf1, bouganin, bryodin, cholix toxin, claudin, diphtheria toxin, gelonin, heat-labile enterotoxin, mitogillin, pertussis toxin, pokeweed antiviral protein, pulchellin, Pseudomonas exotoxin A, restrictocin, ricin, saporin, sarcin, and subtilase cytotoxin (see e.g., WO 2015/113005; WO 2015/120058). In some embodiments, the CD20-binding molecule does not comprise either a toxin effector region or any polypeptide derived from a toxin.

In some embodiments, the CD20 binding regions and toxin effector polypeptide region(s) (which may be cytotoxic and/or harbor one or more mutations altering, reducing, or eliminating catalytic activity and/or cytotoxicity) may be directly linked to each other and/or suitably linked to each other via one or more linkers well known in the art and/or described herein, such as, e.g., proteinaceous linkers capable of being genetically fused between other proteinaceous components of the CD20-binding molecules.

Optionally, a CD20-binding molecule can further comprise a carboxy-terminal endoplasmic retention/retrieval signal motif, such as, e.g., the amino acids KDEL (SEQ ID NO:305) at the carboxy-terminus of a proteinaceous component (e.g. a protein component) of the CD20-binding molecule.

### C. Linkages Connecting Components of the CD20-Binding Molecules

Individual peptide, polypeptide and/or protein components of the CD20-binding molecules, e.g., CD20 binding regions and Shiga toxin effector polypeptides, may be suitably linked to each other via one or more linkers well known in the art and/or described herein. Protein and polypeptide components of the CD20-binding molecules, e.g., multi-chain binding regions, may be suitably linked to each other or other polypeptide components of the CD20-binding molecules described herein via one or more linkers well known in the art. Peptide components of the CD20-binding molecules, e.g., antigenic peptides and KDEL family endoplasmic reticulum retention/retrieval signal motifs, may be suitably linked to another component described herein via one or more linkers, such as a proteinaceous linker, which are well known in the art.

Suitable linkers are generally those which allow each polypeptide component of the CD20-binding molecule as described herein to fold with a three-dimensional structure very similar to the polypeptide components produced individually without any linker or other component. Suitable linkers include single amino acids, peptides, polypeptides, and linkers lacking any of the aforementioned, such as various non-proteinaceous carbon chains, whether branched or cyclic (see e.g. Alley S et al., Bioconjug Chem 19: 759-65 (2008); Ducry L, Stump B, Bioconjug Chem 21: 5-13 (2010)).

Suitable linkers may be proteinaceous and comprise one or more amino acids, peptides, and/or polypeptides. Proteinaceous linkers are suitable for both recombinant fusion proteins and chemically linked conjugates. A proteinaceous linker typically has from about 2 to about 50 amino acid residues, such as, e.g., from about 5 to about 30 or from about 6 to about 25 amino acid residues. The length of the linker can depend upon a variety of factors, such as, e.g., the desired property or properties for which the linker is being selected. In some embodiments, the proteinaceous linker may promote formation of higher order structures, such as dimers, e.g., homodimers and heterodimers, as well as multimeric forms. In other embodiments, the proteinaceous linker may prevent the formation of higher order structures, such as dimers (including homodimers and heterodimers) and multimers.

Suitable linkers may be non-proteinaceous, such as, e.g. chemical linkers. Various non-proteinaceous linkers known in the art may be used to link CD20 binding regions to a Shiga toxin effector polypeptide(s), such as linkers commonly used to conjugate immunoglobulin polypeptides to heterologous polypeptides. For example, components of the CD20-binding molecules as described herein may be linked together using the functional side chains of their amino acid residues and carbohydrate moieties such as, e.g., a carboxy, amine, sulfhydryl, carboxylic acid, carbonyl, hydroxyl, and/or cyclic ring group. For example, disulfide bonds and thioether bonds may be used to link two or more proteins. In addition, non-natural amino acid residues may be used with other functional side chains, such as ketone groups (see e.g. Axup J et al., Proc Natl Acad Sci USA 109: 16101-6 (2012)). Examples of non-proteinaceous chemical linkers include but are not limited to N-succinimidyl (4-iodoacetyl)-aminobenzoate, S—(N-succinimidyl) thioacetate (SATA), N-succinimidyl-oxycarbonyl-cu-methyl-α-(2-pyridyldithio) toluene (SMPT), N-succinimidyl 4-(2-pyridyldithio)-pentanoate (SPP), succinimidyl 4-(N-maleimidomethyl) cyclohexane carboxylate (SMCC or MCC), sulfosuccinimidyl (4-iodoacetyl)-aminobenzoate, 4-succinimidyl-oxycarbonyl-α-(2-pyridyldithio) toluene, sulfosuccinimidyl-6-(α-methyl-α-(pyridyldithiol)-toluamido) hexanoate, N-succinimidyl-3-(-2-pyridyldithio)-proprionate (SPDP), succinimidyl 6(3(−(-2-pyridyldithio)-proprionamido) hexanoate, sulfosuccinimidyl 6(3(−(-2-pyridyldithio)-propionamido) hexanoate, maleimidocaproyl (MC), maleimidocaproyl-valine-citrulline-p-aminobenzyloxycarbonyl (MC-vc-PAB), 3-maleimidobenzoic acid N-hydroxysuccinimide ester (MBS), alpha-alkyl derivatives, sulfoNHS-ATMBA (sulfosuccinimidyl N-[3-(acetylthio)-3-methylbutyryl-beta-alanine]), sulfodicholorphenol, 2-iminothiolane, 3-(2-pyridyldithio)-propionyl hydrazide, Ellman's reagent, dichlorotriazinic acid, and S-(2-thiopyridyl)-L-cysteine.

Suitable linkers, whether proteinaceous or non-proteinaceous, may include, e.g., protease sensitive, environmental redox potential sensitive, pH sensitive, acid cleavable, photocleavable, and/or heat sensitive linkers (see e.g., Zarling D et al., J Immunol 124: 913-20 (1980); Jung S, Moroi M, Biochem Biophys Acta 761: 152-62 (1983); Bouizar Z et al., Eur J Biochem 155: 141-7 (1986); Park L et al., J Biol Chem 261: 205-10 (1986); Browning J, Ribolini A, J Immunol 143: 1859-67 (1989); Joshi S, Burrows R, J Biol Chem 265: 14518-25 (1990); Doronina S et al., Bioconjug Chem 17: 114-24 (2003); Saito G et al., Adv Drug Deliv Rev 55: 199-215 (2003); Jeffrey S et al., J Med Chem 48: 1344-58 (2005); Sanderson R et al., Clin Cancer Res 11: 843-52 (2005); Erickson H et al., Cancer Res 66: 4426-33 (2006); Chen X et al., Adv Drug Deliv Rev 65: 1357-69 (2013)).

Proteinaceous linkers may be chosen for incorporation into the CD20-binding molecules, to form recombinant, fusion proteins. For example, the proteinaceous components of a CD20-binding protein may be joined by one or more linkers comprising one or more amino acids, peptides, and/or polypeptides. For recombinant fusion CD20-binding proteins, linkers typically comprise about 1 to 50 amino acid residues, preferably about 5 to 30 amino acid residues. Commonly, proteinaceous linkers comprise a majority of amino acid residues with polar, uncharged, and/or charged residues, such as, e.g., threonine, proline, glutamine, glycine, and alanine. Non-limiting examples of proteinaceous linkers include alanine-serine-glycine-glycine-proline-glutamate (ASGGPE) (SEQ ID NO:353), valine-methionine (VM), alanine-methionine (AM), AM(G_{2 to 4}S)ₓAM (SEQ ID NO:354) where G is glycine, S is serine, and x is an integer from 1 to 10.

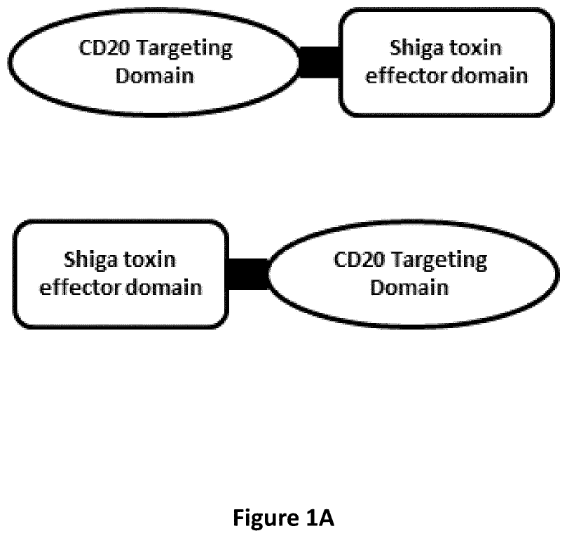

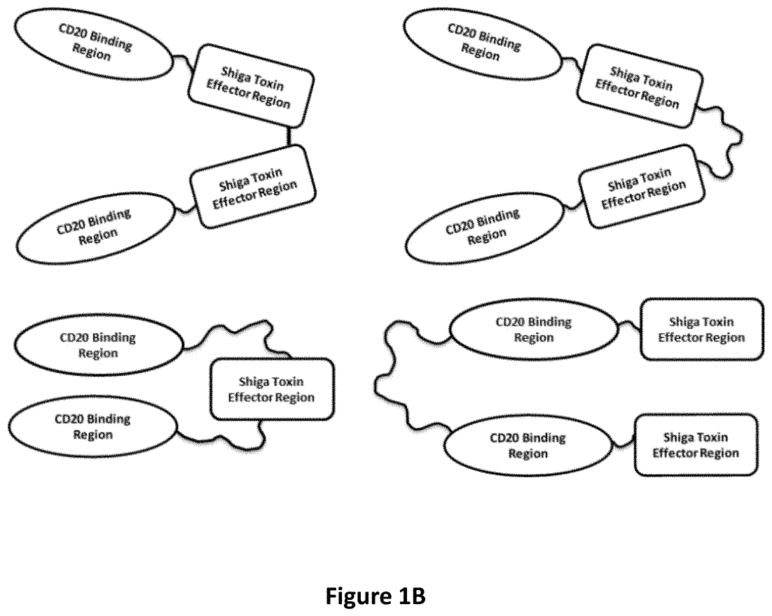

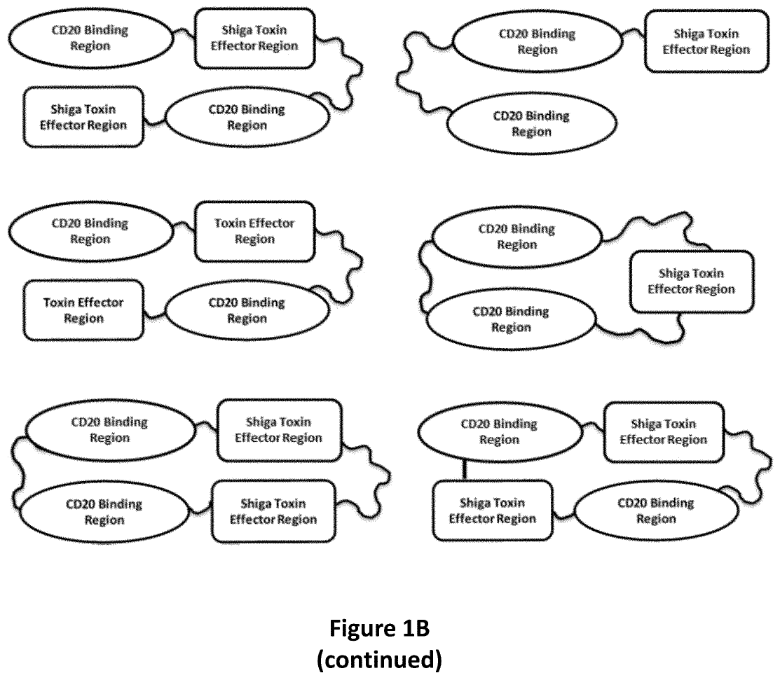

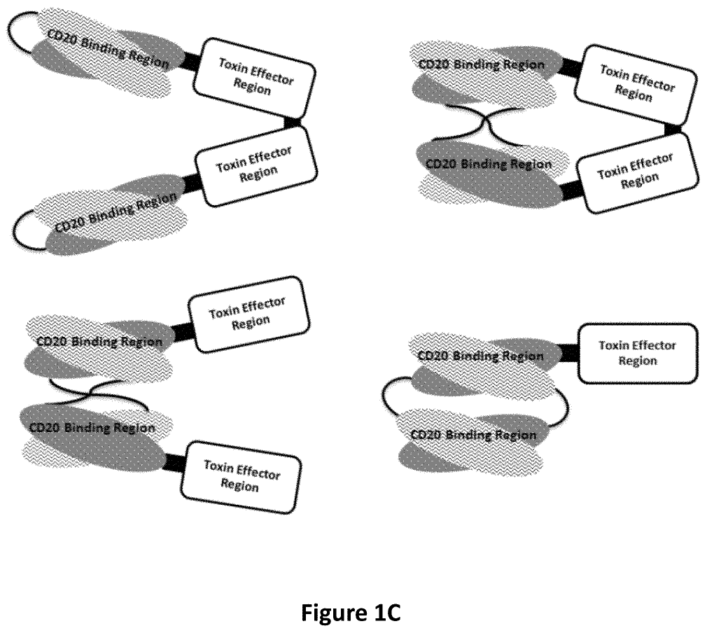

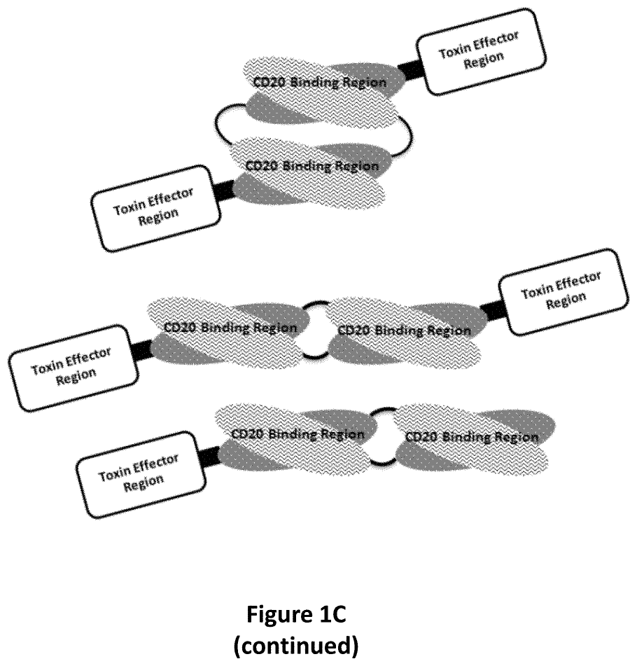

Proteinaceous linkers may be selected based upon the properties desired. Proteinaceous linkers may be chosen by the skilled worker with specific features in mind, such as to optimize one or more of the fusion protein's folding, stability, expression, solubility, pharmacokinetic properties, pharmacodynamic properties, and/or the activity of the fused domains in the context of a fusion construct as compared to the activity of the same domain by itself. For example, proteinaceous linkers may be selected based on flexibility, rigidity, and/or cleavability. The skilled worker may use databases and linker design software tools when choosing linkers. Certain linkers may be chosen to optimize expression. Certain linkers may be chosen to promote intermolecular interactions between identical CD20-binding molecules to form homomultimers or different CD20-binding molecules to form heteromultimers (see e.g. FIG. 1). For example, proteinaceous linkers may be selected which allow for desired non-covalent interactions between proteinaceous components of the CD20-binding molecules as described herein, such as, e.g., interactions related to the formation of dimers and other higher order multimers (see e.g. FIG. 1).

By “linker” herein is meant a domain linker that joins two protein domains together, such as are used in scFv and/or other protein and protein fusion structures. For example, a “binding region linker” may be used to link a Shiga Toxin A subunit effector polypeptide with a binding region, and a “scFv linker” may be used to link the V_{H} and the V_{L} in an scFv. A “cleavable spacer” is a type of linker that contains a cleavage site for one or more proteases. Generally, there are a number of suitable linkers that can be used, including traditional peptide bonds, generated by recombinant techniques that allows for recombinant attachment of the two domains with sufficient length and flexibility to allow each domain to retain its biological function. In some embodiments, the linker peptide can predominantly include the following amino acid residues: Gly, Ser, Ala, or Thr. The linker peptide should have a length that is adequate to link two molecules in such a way that they assume the correct conformation relative to one another so that they retain the desired activity. In some embodiments, the linker is from about 1 to about 50 amino acids in length. In some embodiments, the linker is from about 1 to about 30 amino acids in length. In one embodiment, linkers of 1 to 20 amino acids in length can be used, with from about 5 to about 10 amino acids finding use in some embodiments. Flexible proteinaceous linkers are often greater than twelve amino acid residues long and rich in small, non-polar amino acid residues; polar amino acid residues; and/or hydrophilic amino acid residues, such as, e.g., glycines, serines, and threonines. Flexible proteinaceous linkers may be chosen to increase the spatial separation between components and/or to allow for intramolecular interactions between components. For example, various “GS” linkers are known to the skilled artisan and are composed of multiple glycines and/or one or more serines, sometimes in repeating units, such as, e.g., (GₓS)ₙ (SEQ ID NO:355), (SₓG)ₙ (SEQ ID NO:356), (GGGGS)ₙ (SEQ ID NO:357), and (G)ₙ (SEQ ID NO:358), in which x is 1 to 6 and n is 1 to 30 (see e.g. WO 96/06641). Non-limiting examples of flexible proteinaceous linkers include GKSSGSGSESKS (SEQ ID NO:359), EGKSSGSGSESKEF (SEQ ID NO:360), GSTSGSGKSSEGKG (SEQ ID NO:361), GSTSGSGKSSEGSGSTKG (SEQ ID NO:362), GSTSGSGKPGSGEGSTKG (SEQ ID NO:363), SRSSG (SEQ ID NO:364), SGSSC (SEQ ID NO:365), GSTSGSGKPGSGEGS (SEQ ID NO: 378), and EFPKPSTPPGSSGGAP (SEQ ID NO: 379). In some embodiments, the scFv linker is GSTSGSGKPGSGEGS (SEQ ID NO: 378). In some embodiments, the binding region linker is EFPKPSTPPGSSGGAP (SEQ ID NO: 379).

Rigid proteinaceous linkers are often stiff alpha-helical structures and rich in proline residues and/or one or more strategically placed prolines. Rigid linkers may be chosen to prevent intramolecular interactions between linked components.

Suitable linkers may be chosen to allow for in vivo separation of components, such as, e.g., due to cleavage and/or environment-specific instability. In vivo cleavable proteinaceous linkers are capable of unlinking by proteolytic processing and/or reducing environments often at a specific site within an organism or inside a certain cell type. In vivo cleavable proteinaceous linkers often comprise protease sensitive motifs and/or disulfide bonds formed by one or more cysteine pairs. In vivo cleavable proteinaceous linkers may be designed to be sensitive to proteases that exist only at certain locations in an organism, compartments within a cell, and/or become active only under certain physiological or pathological conditions (such as, e.g., involving proteases with abnormally high levels, proteases overexpressed at certain disease sites, and proteases specifically expressed by a pathogenic microorganism). For example, there are proteinaceous linkers known in the art which are cleaved by proteases present only intracellularly, proteases present only within specific cell types, and proteases present only under pathological conditions like cancer or inflammation, such as, e.g., R-x-x-R motif and AMGRSGGGCAGNRVGSSLSCGGLNLQAM (SEQ ID NO:366).

In some embodiments, a linker may be used which comprises one or more protease sensitive sites to provide for cleavage by a protease present within a target cell. In some embodiments, a linker may be used which is not cleavable to reduce unwanted toxicity after administration to a vertebrate organism.

Suitable linkers may include, e.g., protease sensitive, environmental redox potential sensitive, pH sensitive, acid cleavable, photocleavable, and/or heat sensitive linkers, whether proteinaceous or non-proteinaceous.

Suitable cleavable linkers may include linkers comprising cleavable groups which are known in the art such as, e.g., linkers noted by Zarling D et al., J Immunol 124: 913-20 (1980); Jung S, Moroi M, Biochem Biophys Acta 761: 152-62 (1983); Bouizar Z et al., Eur J Biochem 155: 141-7 (1986); Park L et al., J Biol Chem 261: 205-10 (1986); Browning J, Ribolini A, J Immunol 143: 1859-67 (1989); Joshi S, Burrows R, J Biol Chem 265: 14518-25 (1990).

Suitable linkers may include pH sensitive linkers. For example, certain suitable linkers may be chosen for their instability in lower pH environments to provide for dissociation inside a subcellular compartment of a target cell (see e.g. van Der Velden V et al., Blood 97: 3197-204 (2001); Ulbrich K, Subr V, Adv Drug Deliv Rev 56: 1023-50 (2004)). For example, linkers that comprise one or more trityl groups, derivatized trityl groups, bismaleimideothoxy propane groups, adipic acid dihydrazide groups, and/or acid labile transferrin groups, may provide for release of components of the CD20-binding molecules as described herein, e.g. a polypeptide component, in environments with specific pH ranges (see e.g. Welhoner H et al., J Biol Chem 266: 4309-14 (1991); Fattom A et al., Infect Immun 60: 584-9 (1992)). Certain linkers may be chosen which are cleaved in pH ranges corresponding to physiological pH differences between tissues, such as, e.g., the pH of tumor tissue is lower than in healthy tissues (see e.g. U.S. Pat. No. 5,612,474).

Photocleavable linkers are linkers that are cleaved upon exposure to electromagnetic radiation of certain wavelength ranges, such as light in the visible range. Photocleavable linkers may be used to release a component of a CD20-binding molecule as described herein, e.g. a polypeptide component, upon exposure to light of certain wavelengths. Non-limiting examples of photocleavable linkers include a nitrobenzyl group as a photocleavable protective group for cysteine, nitrobenzyloxycarbonyl chloride cross-linkers, hydroxypropylmethacrylamide copolymer, glycine copolymer, fluorescein copolymer, and methylrhodamine copolymer. Photocleavable linkers may have particular uses in linking components to form CD20-binding molecules described herein designed for treating diseases, disorders, and conditions that can be exposed to light using fiber optics.

In some embodiments, a CD20 binding region is linked to a Shiga toxin effector polypeptide using any number of means known to the skilled worker, including either or both covalent and noncovalent linkages. Individual, polypeptide subcomponents of the CD20 binding regions, e.g. an immunoglobulin CDR, ABR, heavy chain variable region (V_{H}), light chain variable region (V_{L}), and/or V_{H}H region, may be suitably linked to each other via one or more linkers well known in the art and/or described herein.

In some embodiments, the molecule comprises a CD20 binding region which is a scFv with a linker connecting a heavy chain variable (V_{H}) domain and a light chain variable (V_{L}) domain. There are numerous linkers known in the art suitable for this purpose, such as, e.g., the 15-residue (Gly4Ser)₃ peptide (SEQ ID NO:367). Suitable scFv linkers which may be used in forming non-covalent structures include GGS, GGGS (Gly3Ser or G3 S) (SEQ ID NO:368), GGGGS (Gly4Ser or G4S) (SEQ ID NO:369), GGGGSGGG (SEQ ID NO:370), GGSGGGG (SEQ ID NO:371), GSTSGGGSGGGSGGGGSS (SEQ ID NO:372), GSTSGSGKPGSSEGSTKG (SEQ ID NO:373), and GSTSGSGKPGSGEGS (SEQ ID NO: 378). In some embodiments, the scFv linker is GSTSGSGKPGSGEGS (SEQ ID NO: 378).

Suitable methods for linking components of the CD20-binding molecules may be by any method presently known in the art for accomplishing such, as long as the attachment does not substantially impede the binding capability of the CD20 binding regions, the cellular internalization of the CD20-binding molecule, and/or desired, Shiga toxin effector function(s) of the Shiga toxin effector region as measured by an appropriate assay, including by assays described herein.

### D. Structural Examples of CD20-Binding Molecules

The general structure of the CD20-binding molecules described herein is modular, in that various, diverse CD20 binding regions may be used with the same or different Shiga toxin effector polypeptide regions to provide for cell-targeting of cytotoxicity, cytostasis, diagnostic agents, and/or exogenous material delivery to various diverse CD20-expressing cell types. It will be appreciated by the skilled worker that any two or more CD20 binding regions, each capable of binding an extracellular part of CD20, may be associated with a Shiga toxin effector polypeptide(s) to produce CD20-binding molecules as described herein.

In some embodiments, the CD20-binding molecules comprise 1, 2, 3, 4, 5, 6, 7, 8, 9, 10, 11, 12, 13, 14, 15, 16, 17, 18, 19, 20, or 22 individual CD20 binding regions.

The CD20-binding molecules can comprise various structures (see e.g. FIG. 1). For example, structures can be created using covalent and/or non-covalent interactions to associate together various components to form a CD20-binding molecule. In some embodiments, the CD20-binding molecules are multimeric complexes of two or more proteinaceous subunits, such as, e.g. dimers, trimers, tetramers, diabodies, triabodies, tetrabodies, etc.

In some embodiments, a CD20-binding molecule comprises two or more CD20 binding regions because two or more components of the CD20-binding molecule are chemically linked or conjugated together. For example, chemical linkers may be used to conjugate two or more CD20-binding proteins together to form a CD20-binding molecule (see e.g. Wolff E et al., Cancer Res 53: 2560-5 (1993); Ghetie M et al., Proc Natl Acad Sci USA 94: 7509-14 (1997); Ghetie M et al., Blood 97: 1392-8 (2001)).

Certain embodiments of the CD20-binding molecules comprise a multimeric structure comprising two or more component molecules, which may be identical or non-identical. As used herein, the nomenclature (X), refers to a molecule comprising or consisting of integer number (n) copies of a component (X). For example, a dimeric protein comprising two identical, monovalent, CD20-binding polypeptide subunits may be referred to as a homodimer or (CD20-binding monomer)₂. Another example is a mixture of multivalent proteins, each protein comprising three or more identical polypeptide “X” subunits, which is referred to herein as (X)₂₊ₙ, where “n” refers to a positive integer and the value of “2+n” representing the number of CD20 binding regions per protein of the protein molecules present, thus describing a plurality of different multivalent protein species present in a single protein composition.

In some embodiments, the CD20-binding molecules are multimeric, being comprised of two or more CD20-binding molecules, such as, e.g., homodimers, homotrimers, and homotetramers, and the like. For example, two or more monovalent CD20-binding polypeptides may be combined to form a multivalent CD20-binding molecule as described herein (see e.g. FIG. 1).

In some embodiments, a CD20-binding molecule comprises two or more components, each comprising at least one CD20 binding region, because of a non-covalent intermolecular association(s) resulting from domain swapping between the two or more components which results in a CD20-binding molecule with a multimeric structure (see e.g. FIG. 1B). For example, protein domain swapping between immunoglobulin domains can be engineered and optimized as a mechanism of producing precise multimeric structures (see e.g. Arndt K et al., Biochemistry 37: 12918-26 (1998); Holliger P et al., Proc Natl Acad Sci USA 90: 6444-8 (1993).

The skilled worker can engineer multimeric CD20-binding molecules using various scFv-based polypeptide interactions, such as, e.g. scFv-based dimeric, trimeric, tetrameric complexes, etc. For example, the length of the linker in the scFv can affect the spontaneous assembly of non-covalent based, multimeric structures. Generally, linkers of twelve amino acids or less, including the absence of any linker, promote the multimerization of polypeptides or proteins comprising scFvs into higher molecular weight species via favoring intermolecular domain swapping over intra-chain domain pairing (see e.g., Huston J et al., Methods Enzymol 203: 46-88 (1991); Holliger P et al., Proc Natl Acad Sci USA 90: 6444-8 (1993); Stemmer W et al., Biotechniques 14: 256-65 (1993); Whitlow M et al., Protein Eng 6: 989-95 (1993); Desplancq D et al., Protein Eng 7: 1027-33 (1994); Whitlow M et al., Protein Eng 7: 1017-26 (1994); Alfthan K et al., Protein Eng 8: 725-31 (1995); Iiiades P et al., FEBS Lett 409: 437-41 (1997); Kortt A et al., Biomol Eng 18: 95-108 (2001); Todorovska A et al., J Immunol Methods 248: 47-66 (2001); Tomlinson I, Holliger P et al., Methods Enzymol 326: 461-79 (2001); Dolezal O et al., Protein Eng 16: 47-56 (2003)). However, scFvs with no linker at all or a linker with an illustrative length of 15 amino acid residues may multimerize (Whitlow M et al., Protein Eng 6: 989-95 (1993); Desplancq D et al., Protein Eng 7: 1027-33 (1994); Whitlow M et al., Protein Eng 7, 1017-26 (1994); Alfthan K et al., Protein Eng 8: 725-31 (1995)). The skilled worker can identify the multimeric structure(s) created and/or purified using techniques known in the art and/or described herein.

In addition, engineered structures with additional covalent bonds can be used to stabilize multimeric structures that spontaneously assemble (see e.g. Glockshuber R et al., Biochemistry 29: 1362-7 (1990)). For example, the introduction of cysteine residues at specific locations may be used to create disulfide stabilized structures like Cys-diabodies, scFv′ multimers, V_{H}H multimers, V_{NAR} multimers, and IgNAR multimers such as, e.g., by adding the following amino acid residues: GGGGC (SEQ ID NO:374) and SGGGGC (SEQ ID NO:375) (Tai M et al., Biochemistry 29: 8024-30 (1990); Caron P et al., J Exp Med 176: 1191-5 (1992); Shopes B, J Immunol 148: 2918-22 (1992); Adams G et al., Cancer Res 53: 4026-34 (1993); McCartney J et al., Protein Eng 18: 301-14 (1994); Perisic O et al., Structure 2: 1217-26 (1994); George A et al., Proc Natl Acad Sci USA 92: 8358-62 (1995); Tai M et. al.., Cancer Res (Suppl) 55: 5983-9 (1995); Olafsen et al., Protein Eng Des Sel 17: 21-7 (2004)). Thus, the skilled worker can create or stabilize CD20-binding molecules as described herein using disulfide bridge(s) and/or by adding or removing cysteine residue(s) at certain positions to control the position(s) of certain disulfide bridges.

In some embodiments, the structure of a CD20-binding molecule as described herein comprises two or more immunoglobulin domains that binding an extracellular part of CD20. In some embodiments, the CD20-binding molecule may comprise or consist of a single, continuous, polypeptide chain. For example, single-chain bivalent scFvs, sometimes referred to as tandem scFvs (taFvs), single chain diabodies (scDbs), and tandem diabodies (tanDbs or Tandabs), represent binding proteins which are created from a single continuous polypeptide (see e.g. Mack M et al., Proc Natl Acad Sci USA 92: 7021-5 (1995); Kipriyanov S et al., J Mol Biol 293: 41-56 (1999); Cochlovius, B et al., Cancer Res 60: 4336-41 (2000); Volkel T et al., Protein Eng 14: 815-23 (2001); Jendreyko N et al., J Biol Chem 278: 47812-9 (2003); Kipriyanov S et al., J Mol Biol 330: 99-111 (2003); Miller K et al., J Immunol 170: 4854-61 (2003); Meng R et al., Clin Cancer Res 10: 1274-81 (2004); Schlereth B et al., Cancer Res 65: 2882-9 (2005); Huang T, Morrison S, J Pharmacol Exp Ther 316: 983-91 (2006); Liu X et al., Int Immunopharmacol 6: 791-9 (2006); Shen J et al., J Biol Chem 281: 10706-14 (2006); Shen J et al., J Immunol Methods 318: 65-74 (2007); Wu C et al., Nat Biotech 25: 1290-7 (2007); Li B et al., Cancer Res 68: 2400-8 (2008)).

In some embodiments, a CD20-binding molecule comprises both a linker(s) between two or more CD20 binding regions as well as one or more disulfide bonds between components of the CD20 binding regions, whether proximal or distal to the linker, such as a disulfide bond between two immunoglobulin regions which requires an immunoglobulin domain swapping association between those two immunoglobulin regions (see e.g. Glockshuber R et al., Biochemistry. 29: 1362-7 (1990)).

Alternatively, two or more polypeptide chains may be linked together using polypeptide domains which self-associate or multimerize with each other (see e.g. U.S. Pat. No. 6,329,507). For example, the addition of carboxy-terminal multimerization domains has been used to construct proteins comprising immunoglobulin domains, such as, e.g., scFvs, autonomous V_{H} domains, V_{H}Hs, V_{NAR}s, and IgNARs. Examples of self-associating domains known to the skilled worker include immunoglobulin constant domains (such as knobs-into-holes, electrostatic steering, and IgG/IgA strand exchange), immunoglobulin Fab chains (e.g. (Fab-scFv)₂ and (Fab′ scFv)₂), immunoglobulin Fc domains (e.g. (scDiabody-Fc)₂, (scFv-Fc)₂ and scFv-Fc-scFv), immunoglobulin CHX domains, immunoglobulin CH1-3 regions, immunoglobulin CH3 domains (e.g. (scDiabody-CH3)₂, LD minibody, and Flex-minibody), immunoglobulin CH4 domains, CHCL domains, amphiphilic helix bundles (e.g. scFv-HLX), helix-turn-helix domains (e.g. scFv-dHlx), coiled-coil structures including leucine zippers and cartilage oligometric matrix proteins (e.g. scZIP), cAMP-dependent protein kinase (PKA) dimerization and docking domains (DDDs) combined with an A kinase anchor protein (AKAP) anchoring domain (AD) (also referred to as “dock-and-lock” or “DNL”), streptavidin, verotoxin B multimerization domains, tetramerization regions from p53, and barnase-barstar interaction domains (Pack P, Plückthun A, Biochemistry 1579-84 (1992); Holliger P et al., Proc Natl Acad Sci USA 90: 6444-8 (1993); Kipriyanov S et al., Hum Antibodies Hybridomas 6: 93-101 (1995); de Kruif J, Logtenberg T, J Biol Chem 271: 7630-4 (1996); Flu S et al., Cancer Res 56: 3055-61 (1996); Kipriyanov S et al., Protein Eng 9: 203-11 (1996); Rheinnecker M et al., J Immunol 157: 2989-97 (1996); Tershkikh A et al., Proc Natl Acad Sci USA 94: 1663-8 (1997); Müller K et al., FEBS Lett 422: 259-64 (1998); Cloutier S et al., Mol. Immunol 37: 1067-77 (2000); Li S et al., Cancer Immunol Immunother 49: 243-52 (2000); Schmiedl A et al., Protein Eng 13: 725-34 (2000); Schoonjans R et al., J Immunol 165: 7050-7 (2000); Borsi L et al., Int J Cancer 102: 75-85 (2002); Deyev S et al., Nat Biotechnol 21: 1486-92 (2003); Wong W, Scott J, Nat Rev Mol Cell Biol. 5: 959-70 (2004); Zhang J et al., J Mol Biol. 335: 49-56 (2004); Baillie G et al., FEBS Letters 579: 3264-70 (2005); Rossi E et al., Proc Natl Acad Sci USA 103: 6841-6 (2006); Simmons D et al., J Immunol Methods 315: 171-84 (2006); Braren I et al., Biotechnol Appl Biochem 47: 205-14 (2007); Chang C et al., Clin Cancer Res 13: 5586--91s (2007); Liu M et al., Biochem J 406: 237-46 (2007); Zhang J et al., Protein Expr Purif 65: 77-82 (2009); Bell A et al., Cancer Lett 289: 81-90 (2010); Iqbal U et al., Br J Pharmacol 160: 1016-28 (2010); Asano R et al., FEBS J 280: 4816-26 (2013); Gil D, Schrum A, Adv Biosci Biotechnol 4: 73-84 (2013)).

In some embodiments, the structure of a CD20-binding molecule is engineered from an antibody or Fab fragment. For example, CD20-binding molecules may be engineered using approaches known to the skilled worker (see e.g. Shuford W et al., Science 252: 724-7 (1991); Caron P et. al.., J Exp Med 176: 1191-5 (1992); Shopes B, J Immunol 148: 2918-22 (1992); Wolff E et al., Cancer Res 53: 2560-5 (1993)).

In some embodiments, all the cell-targeting binding regions of a CD20-binding molecules are identical and/or share the same binding specificities. In such embodiments, the CD20-binding molecule is monospecific—meaning it comprises CD20 binding regions that bind with high affinity to the same extracellular CD20 target biomolecule, overlapping extracellular epitopes in the same CD20 target biomolecule, and/or the same extracellular epitope in a CD20 target biomolecule. Whether two binding regions are binding to the same extracellular part of a CD20 target biomolecule may be determined by the skilled worker with available methods, such as, e.g., empirically using competitive binding assays or predictively based on the overlap of known epitope and/or immunized peptide sequences.

In some embodiments, the CD20-binding molecule may comprise binding regions that bind with high affinity to non-identical epitopes, whether non-overlapping or overlapping. The CD20-binding molecules may comprise binding regions with high binding affinity to non-overlapping epitopes. Multispecific CD20-binding molecules as described herein may be created using two or more different binding regions, such as, e.g., two different scFvs, V_{H}Hs, V_{NAR}s, and/or IgNARs in diabodies, triabodies, tandem formats (including tandem di-scFv, tandem tri-scFv, and scFv-Fc tandems), single-chain diabodies (scDb), tandem Fvs, bispecific scFv (Bis-scFv), scFv2, (Fab′)₃, tetrameric (scFv2)₂, scFv2-Fc, and combinations of scFvs, V_{H}Hs, V_{NAR}s, and/or IgNARs with different specificities (Adams G et al., Cancer Res 53: 4026-34 (1993); Mallender W et al. J Biol Chem 269: 199-206 (1994); Todorovska A et al., J Immunol Methods 248: 47-66 (2001); Korn T et al., J Gene Med 6: 642-51 (2004); Lu D et al., J Biol. Chem 280: 19665-72 (2005); Schneider M et al., Eur J Immunol 35: 987-95 (2005); Wittel U et al., Nucl Med Biol. 32: 157-64 (2005); Semenyuk E et al., Biochimie 89: 31-8 (2007)).

In some embodiments, a CD20-binding molecule may comprise a single, continuous polypeptide component which is multimerized with itself or another protein to form a multimeric structure. For example, single-chain bivalent scFvs, sometimes referred to as tandem scFvs (taFvs), single chain diabodies (scDbs), and tandem diabodies (tanDbs or Tandabs), can be expressed as single continuous polypeptide chain (Mack M et al., Proc Natl Acad Sci USA 92: 7021-5 (1995); Kipriyanov S et al., J Mol Biol 293: 41-56 (1999); Cochlovius, B et al., Cancer Res 60: 4336-41 (2000); Volkel T et al., Protein Eng 14: 815-23 (2001); Kipriyanov S et al., J Mol Biol 330: 99-111 (2003); Schlereth B et al., Cancer Res 65: 2882-9 (2005)). These structures may be engineered to multimerize into higher-order, higher-valence structures, such as, e.g. a tetravalent F(ab′)₂, (taFv)₂, and (scDb)₂ structures (see Todorovska A et al., J Immunol Methods 248: 47-66 (2001)).

Structures comprising two scFvs linked by non-covalent interactions due to the intermolecular pairing of variable regions are known to the skilled worker, such as, e.g., diabodies, mini-antibodies, and bivalent mini-antibodies, all of which may be either monospecific or bispecific (Holliger, P et al., Proc Natl Acad Sci USA 90: 6444-8 (1993); Pack P et al., Biotechnology (NY) 11: 1217-7 (1993); Tai M et al., Cancer Res (Suppl) 55: 5983-9 (1995); Atwell J et al., Mol Immunol 33: 1301-12 (1996); Rheinnecker M et al., J Immunol 157: 2989-97 (1996); Schier R et al., J Mol Biol 255: 28-43 (1996); Adams G et al., Br J Cancer 77: 1405-12 (1998); Todorovska A et al., J Immunol Methods 248: 47-66 (2001); Baler P et al., Cancer Immunol Immunother 57: 43-52 (2008)). Numerous scFv monomers have been observed to naturally form multimers or oligomers (e.g. diabodies, triabodies, and tetrabodies) due to self-association, with the majority form being dimeric for scFv structures comprising linkers of 3-12 amino acid residues (Essig N et al., J Mol Biol 234: 897-901 (1993); Griffiths A et al., EMBO J 12: 725-34 (1993); Holliger P et al., Proc Natl Acad Sci USA 90: 6444-8 (1993); Whitlow M et al., Protein Eng 6: 989-95 (1993); Desplancq D et al., Protein Eng 7: 1027-33 (1994); Whitlow M et al., Protein Eng 7, 1017-26 (1994); Kortt A et al., Protein Eng 10: 423-33 (1997); Arndt K et al., Biochemistry 37: 12918-26 (1998); Atwell J et al., Protein Eng 12: 597-604 (1999)).

In general, scFv structures with a relatively short linker of five to ten amino acid residues or less have a greater propensity for homo-dimerization (Arndt K et al., Biochemistry 37: 12918-26 (1988); Holliger P et al., Proc Natl Acad Sci USA 90: 6444-8 (1993); Perisic O et al., Structure 2: 1217-26 (1994); Atwell J et al., Mol Immunol 33: 1301-12 (1996); Iliades P et al., FEBS Lett 409: 437-41 (1997); Kortt A et al., Protein Eng 10: 423-33 (1997); Metzger D et al., Protein Eng 10: 423-33 (1997); Pei X et al., Proc Natl Acad Sci USA 94: 9637-42 (1997); Atwell J et al., Protein Eng 12: 597-604 (1999); Denton G et al., Cancer Immunol Immunother 48: 29-38 (1999); Le Gall F et al., FEBS Lett 453: 164-8 (1999); Atwell J et al., Protein Eng 12: 597-604 (1999); Dolezal, O et al., Protein Eng 13: 565-74 (2000); Nielsen U et al., Cancer Res 60: 6434-40 (2000); Todorovska A et al., J Immunol Methods 248: 47-66 (2001); Wu A et al., Protein Eng 14: 1025-33 (2001); Arndt M et al., FEBS Lett 578: 257-61 (2004); Le Gall F et al., J Immunol Methods 285: 111-27 (2004)). In contrast, scFvs with linkers comprising at least 12 amino acid residues predominantly form monomers with only a minority fraction undergoing spontaneous multimerization (Nielsen U et al., Cancer Res 60: 6434-40 (2000); Denton G et al., Cancer Immunol Immunother 48: 29-38 (1999); Kortt A et al., Biomol Eng 18: 95-108 (2001); Volkel T et al., Protein Eng 14: 815-23 (2001)).

The use of linkers of three amino acid residues or fewer may promote multimerization to higher order structures larger than dimeric forms. If an scFv has a linker of less than 3 residues, then trimerization may be favored (Iliades P et al., FEBS Lett 409: 437-41 (1997)); Kortt A et al., Biomol Eng 18: 95-108 (2001); Todorovska A et al., J Immunol Methods 248: 47-66 (2001); Arndt M et al., FEBS Lett 578: 257-61 (2004)). Furthermore, scFvs with very short linkers, e.g., linkers of 2 amino acid residues or less, often form trimers and/or mixtures of trimers and tetramers (Pei X et al., Proc Natl Acad Sci USA 94: 9637-42 (1997); Hudson P, Kortt A, J Immunol Methods 231: 177-89 (1999); Dolezal O et al., Protein Eng 13: 565-74 (2000); Power B et al., Protein Sci 12: 734-47 (2003); Le Gall F et al., J Immunol Methods 285: 111-27 (2004)). In certain arrangements with short linkers, tetramers may be favored (Dolezal O et al., Protein Eng 13: 565-74 (2003); Arndt M et al., FEBS Lett 578: 257-61 (2004)). Multimeric structures can be formed by scFvs lacking any linker, i.e. having a linker length of zero amino acid residues. For example, the direct linkage of variable domains with V_{L} before V_{H} may favor the formation of tetrabodies (Iliades P et al., FEBS Lett 409: 437-41 (1997)) whereas V_{H} before V_{L} may favor trimers (Kortt A et al., Protein Eng 10: 423-33 (1997)).

In addition to the linker length, the orientation of the variable domains may affect multimerization characteristics (Huston J et al., Proc Natl Acad Sci USA 85, 5879-83 (1988); Padlan E, Mol Immunol 31: 169-217 (1994); Kortt A et al., Protein Eng 10: 423-33 (1997); Dolezal, O et al., Protein Eng 13: 565-74 (2000); Carmichael J et al., J Mol Biol 326: 341-51 (2003); Arndt M et al., FEBS Lett 578: 257-61 (2004)). It has been suggested that the V_{L}-V_{H} orientation exhibits a greater tendency to form higher molecular weight oligomers than does the reverse orientation because the V_{L}-V_{H} orientation is more constrained (Kortt A et al., Protein Eng 10: 423-33 (1997); Dolezal, O et al., Protein Eng 13: 565-74 (2000); Plückthun A, Pack P, Immunotechnology 3: 83-105 (1997)).

The same linker has shown variability in its effect on scFv multimerization depending on the V_{H} and V_{L} orientation, such as, e.g., affecting the relative proportions of dimeric to trimeric forms (Le Gall F et al., FEBS Lett 453: 164-8 (1999); Arndt Metal., FEBS Lett 578: 257-61 (2004); Le Gall F et al., J Immunol Methods 285: 111-27 (2004)).

Camelid V_{H}H immunoglobulin domains have been multimerized using particular hinges and covalently linked multi V_{H}H chains (tandem) (Fraile S et al., Mol Microbiol 53: 1109-21 (2004); Zhang J et al., J Mol Biol 335: 49-56 (2004)). Immunoglobulin domains from Chondrichthyes, such as IgNARs, have been multimerized using certain hinges or cysteine-mediated disulfide bond stabilization (see e.g. Simmons et al., J Immunol Methods 315: 171-84 (2006)).

Thus, the generation of CD20-binding molecules comprising various immunoglobulin domains may be controlled by molecular engineering strategies which are either covalent or non-covalent, such as, e.g., covalent strategies involving single-chain tandem arrangements, covalent strategies involving cysteine-mediated, disulfide bond stabilized multimers, and/or non-covalent strategies involving dimerization domains, linker choice, and/or variable domain order. Multiple strategies (e.g., linker-related non-covalent multimerization and covalent disulfide bond stabilization) may be combined when creating structures that are CD20-binding molecules as described herein (see e.g. Lu D et al., J Immunol Methods 279: 219-32 (2003)).

For the purposes as described herein, the specific order or orientation is not fixed for the toxin effector region(s) and the two or more CD20 binding regions in relation to each other or the entire CD20-binding molecule as described herein. The components of the CD20-binding molecules as described herein may be arranged in any order provided that the desired activities of the CD20 binding regions and the toxin effector region(s) are not eliminated. Desired activities include providing the CD20-binding molecule with the ability to, e.g., bind CD20-expressing cells; rapidly induce cellular internalization; cause efficient internalization; intracellularly route to a desired subcellular compartment(s); cause cytostasis; cause cytotoxicity; selectively kill CD20-expressing cells; deliver exogenous materials into the interior of a cell; diagnosis a disease, disorder, or condition; and/or treat a disease, disorder, or condition in a patient in need thereof.

In some embodiments, the CD20-binding molecule described herein comprises (i) an anti-CD20 scFv; (ii) a binding region linker; and (iii) a Shiga toxin effector polypeptide. In some embodiments, the CD20-binding molecule comprises an amino acid sequence with at least 90%, at least 95%, at least 96%, at least 97%, at least 98%, or at least 99% identity to SEQ ID NO: 54. In some embodiments, the CD20-binding molecule comprises an amino acid sequence of SEQ ID NO: 54. In some embodiments, the CD20-binding molecule comprises an amino acid sequence of SEQ ID NO: 54 with one or more mutations, such as 2, 3, 4, 5, 6, 7, 8, or 10 mutations. In some embodiments, the CD20-binding molecule comprises an amino acid sequence of SEQ ID NO: 54 with 1-5, 5-10, 11-5, 15-20, 10-25, 25-30, or more than 30 mutations.

### E. Determining the Relative Proportion(s) of a CD20-Binding Molecule to One or More Other CD20-Binding Molecule(s) Present in a Composition as Described Herein

The ratios, percentages, and/or relative proportions of different molecular species within a composition as described herein may be determined by the skilled worker using a technique well-known in the art and/or described herein, such as, e.g., chromatographic, electrophoretic, electrochromatographic, capillary, centrifugation, isoelectric focusing, and microfluidic techniques for analyzing proteinaceous molecules. For example, the size and/or intensity of different “peaks” or “bands” resulting from subjecting a protein sample to any of the aforementioned methods can be used to calculate the relative ratio of different-sized CD20-binding molecules within a composition.

Because all compositions as described herein comprise at least one CD20-binding molecule which comprises at least one proteinaceous component, the skilled worker may use techniques known in the art to determine relative proportions of proteinaceous molecules. For example, the proportions of different proteinaceous molecular species within a composition as described herein may be determined by amino acid analysis/amino acid quantification techniques known to the skilled worker (see e.g. Bio-Synthesis, Inc., Lewisville, Tex., U.S.). In another example, the relative proportions of different-sized proteinaceous molecules within a composition as described herein may be determined using chromatographic, electrophoretic, electrochromatographic, and/or density-gradient ultra-centrifugation techniques known to the skilled worker and/or described herein, such as, e.g., via gel electrophoresis and densitometry analysis of the results.

The skilled worker may use software methods known in the art and/or described herein to perform analysis of data obtained from, inter alia, amino acid quantification, chromatographic, electrophoretic, electrochromatographic, and density-gradient ultra-centrifugation assays to determine the proportions of different molecular species present in a composition as described herein. For example, the skilled worker may use software methods known in the art to perform peak-integration analysis of chromatographic, electrophoretic, and/or electrochromatographic data, such as, e.g., size-exclusion chromatographic (SEC) data, in order to compare the relative proportion(s) of different molecular species present in a composition as described herein. Through the use of molecular-size migration standards (e.g., gel filtration and ion-exchange standards) and knowledge of possible molecular species present in a composition as described herein, the size of molecular species in a peak may be estimated and the identity of the molecular species in a peak may be inferred. Alternatively or in addition, complimentary methods (e.g. sodium dodecyl sulfate, polyacrylamide gel electrophoresis (SDS-PAGE) or mass spectroscopy) known to the skilled worker and/or described herein may be used to determine the molecule(s) composed in certain peaks.

Peak integration calculations can be used to determine various curve characteristics including peak areas, retention times, peak heights, peak widths, and percentage of peak area to total peak areas. For any peak integration analysis, a baseline may be calculated first, such as, e.g., using an automatic calculation combined with a blank curve (e.g. data collected from a solvent or mobile phase blank run), blank curve subtraction, or zero baseline. Certain settings, such as, e.g., structure width, baseline noise parameter(s), baseline slope limit or threshold slope setting, maximum baseline limit, and/or minimum distance between data points, may be adjusted in certain situations. For any chromatographic, electrophoretic, and/or electrochromatographic data analysis, the scope of analysis can be limited to certain retention time ranges (e.g., to avoid the column inclusion volume and/or to avoid data from retention times beyond the exclusion limit). User editing of peak window limits and rejection of peak assignments may be performed where appropriate or via changing settings like minimum area and/or minimum height.

Amino acid quantification, chromatographic, electrophoretic, electrochromatographic, and/or density-gradient ultra-centrifugation methods known to the skilled worker and/or described herein may be used to determine: 1) relative concentration ratios of different CD20-binding molecules within a composition as described herein, 2) relative molar ratios of different CD20-binding molecules within a composition as described herein, 3) relative mass ratios of different CD20-binding molecules within a composition as described herein, and/or 4) relative molal concentration ratios of different CD20-binding molecules within a composition as described herein. For example, the relative proportion of CD20-binding molecule in a composition as described herein can be expressed as a percentage (whether of concentrations, molarities, masses, or molalities) calculated from the total CD20-binding molecule divided by the total proteinaceous species multiplied by 100. Alternatively, the relative proportion of CD20-binding molecule in a composition as described herein can be expressed as a ratio of concentrations, molarities, masses, or molalities using the measurement of a total CD20-binding molecule species to the measurement of another molecular species regardless of it being a different CD20-binding molecule species or a non-CD20-binding molecular species.

In the Examples below, fast protein liquid chromatography size exclusion (FPLC-SEC) and high performance liquid chromatography size exclusion (HPLC-SEC) analyses of CD20-binding molecule compositions were used to determine the relative amounts of CD20-binding molecules of different sizes present in a composition as well as the relative amounts of multivalent CD20-binding molecules to other molecules like monovalent CD20-binding molecules, e.g., ratios between monovalent, divalent, and higher-valence CD20-binding proteinaceous species present in the compositions were determined.

One example of a method known in the art with which the skilled worker may use to determine ratios and/or percentages of different molecular species within a composition as described herein is dynamic light scattering or photon correlation spectroscopy (see e.g., Lamkemeyer T et al., FEBS J 273: 3393-410 (2006); Rousselot Metal., FEBS J 273: 4055-71 (2006); Bruneaux M et al., Curr Protein Pept Sci 9: 15-80 (2008)).

Another example of a method known in the art with which the skilled worker may use to determine ratios and/or percentages of different molecular species within a composition as described herein is the Protein 230 Assay using the Agilent Bioanalyzer running Agilent 2100 Expert software (Agilent Technologies, Inc., Santa Clara, Calif., U.S.). The Protein 230 Assay can be used to estimate the quantity, molecular weight, and purity of a CD20-binding molecule composition as described herein. The Protein 230 Assay produces data presented as gel-like images with “bands” and/or electrophenograms with “peaks.” A standard ladder of known marker sizes may be used to create standard gel-like and electrophenogram profiles for each analysis. Then the migration behavior of a sample in the assay is used to predict, inter alia, its size. Laser-induced fluorescence intensity may be used to estimate protein quantity in a sample, individual band, and/or peak.

In some embodiments, the multivalent CD20-binding molecule compositions as described herein comprise a multivalent CD20-binding molecule as described herein, wherein the composition comprises a ratio of monovalent CD20-binding protein concentration to total CD20-binding molecule concentration of less than one to three; and wherein each monovalent CD20-binding protein comprises only one CD20 binding region capable of specifically binding an extracellular part of a CD20 and comprises at least one Shiga toxin effector polypeptide. In some embodiments, the multivalent CD20-binding molecule composition comprises the ratio of monovalent CD20-binding protein concentration to total CD20-binding molecule concentration of less than the ratio selected from the following: 1:5, 1:6, 1:7, 1:8, 1:9, 1:10, and 1:11. In some embodiments, the multivalent CD20-binding molecule composition as described herein comprises a ratio of multivalent CD20-binding protein concentration to total CD20-binding protein concentration of more than two to three.

In some embodiments, the CD20-binding molecule compositions as described herein comprise a ratio of relatively large valence CD20-binding protein concentration to total CD20-binding protein concentration of less than the ratio selected from the following: 1:4, 1:7, 1:11, 1:21, 1:41, 1:71, 1:111, and 1:161; wherein each relatively large-valence CD20-binding protein comprises three or more CD20 binding regions capable of specifically binding an extracellular part of a CD20 and comprises at least one Shiga toxin effector polypeptide.

In some embodiments, the CD20-binding molecule compositions as described herein comprise a ratio of bivalent CD20-binding molecule concentration to total CD20-binding molecule concentration of more than a ratio selected from the following: 1:2, 2:3, 3:4, 4:5, 5:6, 7:8, 8:9, 9:10, 10:11, 11:12, 12:13, 13:14, and 14:15; wherein each bivalent CD20-binding molecule comprises (1) only two CD20 binding regions capable of specifically binding an extracellular part of a CD20 and (2) one or more Shiga toxin effector polypeptides.

In some embodiments, the CD20-binding molecule compositions comprise a multivalent CD20-binding molecule, wherein the composition comprises a ratio of monovalent CD20-binding molecule mass to total CD20-binding molecule mass of less than one to three; and wherein each monovalent CD20-binding molecule comprises only one CD20 binding region capable of specifically binding an extracellular part of a CD20 and comprises at least one Shiga toxin effector polypeptide. In some embodiments, the CD20-binding molecule composition comprises the ratio of monovalent CD20-binding molecule mass to total CD20-binding protein mass of less than the ratio selected from the following: 1:5, 1:6, 1:7, 1:8, 1:9, 1:10, and 1:11. In some embodiments, the CD20-binding molecule composition as described herein comprises a ratio of multivalent CD20-binding molecule mass to total CD20-binding molecule mass of more than two to three.

In some embodiments, the CD20-binding molecule composition comprises a ratio of relatively large valence CD20-binding molecule mass to total CD20-binding molecule mass of less than the ratio selected from the following: 1:4, 1:7, 1:11, 1:21, 1:41, 1:71, 1:111, and 1:161; wherein each relatively large-valence CD20-binding molecule comprises three or more CD20 binding regions capable of specifically binding an extracellular part of a CD20 and comprises at least one Shiga toxin effector polypeptide.

In some embodiments, the CD20-binding molecule composition comprises a ratio of bivalent CD20-binding molecule mass to total CD20-binding molecule mass of more than a ratio selected from the following: 1:2, 2:3, 3:4, 4:5, 5:6, 7:8, 8:9, 9:10, 10:11, 11:12, 12:13, 13:14, and 14:15; wherein each bivalent CD20-binding molecule comprises (1) only two CD20 binding regions capable of specifically binding an extracellular part of a CD20 and (2) one or more Shiga toxin effector polypeptides.

In some embodiments, the CD20-binding molecule composition as described herein comprises a multivalent CD20-binding molecule as described herein, wherein the composition comprises a ratio of monovalent CD20-binding molecule molarity to total CD20-binding molecule molarity of less than one to 1.5; and wherein each monovalent CD20-binding molecule comprises only one CD20 binding region capable of specifically binding an extracellular part of a CD20 and comprises at least one Shiga toxin effector polypeptide. In some embodiments, the CD20-binding molecule composition comprises the ratio of monovalent CD20-binding molecule molarity to total CD20-binding protein molarity of less than the ratio selected from the following: 1:2, 1:3, 1:4, 1:5, 1:6, 1:7, and 1:8. In some embodiments, the CD20-binding molecule composition as described herein comprises a ratio of multivalent CD20-binding molecule molarity to total CD20-binding molecule molarity of more than one to 1.5.

In some embodiments, the CD20-binding molecule composition as described herein comprises a ratio of relatively large valence CD20-binding molecule molarity to total CD20-binding molecule molarity of less than the ratio selected from the following: 1:2, 1:3.5, 1:5, 1:11, 1:21, 1:36, 1:55, and 1:59; wherein each relatively large-valence CD20-binding molecule comprises three or more CD20 binding regions capable of specifically binding an extracellular part of a CD20 and comprises at least one Shiga toxin effector polypeptide.

In some embodiments, the CD20-binding molecule composition as described herein comprises a ratio of bivalent CD20-binding molecule molarity to total CD20-binding molecule molarity of more than a ratio selected from the following: 1:1.5, 2:3, 3:4, 4:5, 5:6, 7:8, 8:9, 9:10, 10:11, 11:12, 12:13, 13:14, and 14:15; wherein each bivalent CD20-binding molecule comprises (1) only two CD20 binding regions capable of specifically binding an extracellular part of a CD20 and (2) one or more Shiga toxin effector polypeptides.

In some embodiments, the CD20-binding molecule composition as described herein comprises a multivalent CD20-binding molecule as described herein, wherein the composition comprises a ratio of monovalent CD20-binding molecule molality to total CD20-binding molecule molality of less than one to 1.5; and wherein each monovalent CD20-binding molecule comprises only one CD20 binding region capable of specifically binding an extracellular part of a CD20 and comprises at least one Shiga toxin effector polypeptide. In some embodiments, the CD20-binding molecule composition comprises the ratio of monovalent CD20-binding molecule molality to total CD20-binding protein molality of less than the ratio selected from the following: 1:2, 1:3, 1:4, 1:5, 1:6, 1:7, and 1:8. In some embodiments, the CD20-binding molecule composition as described herein comprises a ratio of multivalent CD20-binding molecule molality to total CD20-binding molecule molality of more than one to 1.5.

In some embodiments, the CD20-binding molecule composition as described herein comprises a ratio of relatively large valence CD20-binding molecule molality to total CD20-binding molecule molality of less than the ratio selected from the following: 1:2, 1:3.5, 1:5, 1:11, 1:21, 1:36, 1:55, and 1:59; wherein each relatively large-valence CD20-binding molecule comprises three or more CD20 binding regions capable of specifically binding an extracellular part of a CD20 and comprises at least one Shiga toxin effector polypeptide.

In some embodiments, the CD20-binding molecule composition as described herein comprises a ratio of bivalent CD20-binding molecule molality to total CD20-binding molecule molality of more than a ratio selected from the following: 1:1.5, 2:3, 3:4, 4:5, 5:6, 7:8, 8:9, 9:10, 10:11, 11:12, 12:13, 13:14, and 14:15; wherein each bivalent CD20-binding molecule comprises (1) only two CD20 binding regions capable of specifically binding an extracellular part of a CD20 and (2) one or more Shiga toxin effector polypeptides.

### F. Molecular Stability, Composition Stability, Controlling Multimerization, and Minimizing Aggregation

For certain applications, the stability of the relative proportion of multivalent CD20-binding molecule(s) to total CD20-binding molecules in a composition as described herein may be important to the composition's effectiveness. For example, in certain medical applications, the stability of the relative proportions of multivalent CD20-binding molecule(s) as described herein to monovalent CD20-binding molecule(s) may be important. In certain applications, the stability of the relative proportions of bivalent CD20-binding molecules to higher-valence CD20-binding molecules may be important. In certain applications the stability of the relative proportion of bivalent CD20-binding molecules to non-bivalent CD20-binding molecules may be important.

For certain embodiments, a one or more steps of controlled multimerization of some or all of the components of a CD20-binding molecule as described herein may be used to produce a composition as described herein.

For some applications, the minimization or otherwise controlling of unwanted aggregation and/or multimerization of CD20-binding molecules may be important for certain compositions as described herein. For example, with certain proteinaceous therapeutics, the aggregation and/or multimerization of the therapeutic molecule can in certain situations increase the risk for unwanted immune responses in recipients of the proteinaceous therapeutic. In particular, aggregation and/or multimerization of CD20-binding molecules to higher molecular weight complexes may increase the risk of unwanted immune responses after administration of certain CD20-binding molecule compositions to certain recipients. In addition, misfolded proteins and degraded protein products can exhibit increased immunogenicity as compared to their properly folded counterparts.

For all of these reasons and depending on the specific application, the skilled worker will appreciate whether there is a need to consider 1) the stability of CD20-binding molecules of the compositions as described herein and 2) the stability of the ratios of different CD20-binding molecules present in compositions as described herein. For example, in some embodiments, the CD20-binding molecule as described herein and compositions thereof are the result of controlled multimerization and/or certain purification steps. Similarly, in some embodiments, the CD20-binding molecule as described herein will be engineered to eliminate or reduce certain multimerization possibilities. In some embodiments, the CD20-binding molecule as described herein will be designed to avoid the formation of unwanted aggregates, such as, e.g., under certain storage conditions like in an aqueous solution at 8, 4, 2, −4, −10, −20, or −25° C.

For certain applications of the compositions as described herein, it may be desirable to minimize in the composition as described herein the amount of: 1) high molecular weight CD20-binding molecules (e.g. molecules greater than 175, 180, 190, 200, or 250 kDa or larger); 2) greatly CD20-binding molecules (i.e. molecules comprising five or more CD20 binding regions); 3) multimers of CD20-binding molecules which are high molecular weight CD20-binding molecules representing #1 and/or greatly CD20-binding molecules representing #2 (e.g. certain, large, noncovalent multimers of CD20-binding molecules); 3) misfolded proteins (e.g., misfolded CD20-binding proteins or protein components thereof); and/or 4) degradation products (e.g. unwanted protein fragments of a proteinaceous component of a CD20-binding molecule, such as, e.g., a polypeptide fragment of a Shiga toxin effector region or CD20 binding region). For example, a rationale to minimize the amount of any of the types of molecules listed in #1 to #4 above might be for medical applications where the presence of a certain amounts of these molecules might increase the potential for unwanted antigenic and/or immunogenic reactions in a recipient of a compositions as described herein, such as, e.g., by the presence of these molecules revealing new epitopes or by forming repetitive motifs more readily identified by a recipient's immune system as foreign.

The skilled worker may use routine methods to assess multimerization states of the CD20-binding molecules as described herein and/or molecules present in the compositions as described herein. The skilled worker may use routine methods to minimize the presence or relative proportion of CD20-binding molecule aggregates, high molecular weight CD20-binding protein multimers, misfolded CD20-binding proteins, and CD20-binding protein degradation products in the compositions as described herein.

In some embodiments of the compositions as described herein, the relative proportion of bivalent, trivalent, and/or tetravalent forms of multivalent CD20-binding molecule(s) is maximized, such as by further purifying away from monovalent CD20-binding protein(s), higher molecular weight CD20-binding molecule(s), misfolded CD20-binding protein(s), and/or protein degradation product(s).

The skilled worker may use routine methods to create a CD20-binding molecule as described herein, and compositions thereof. The skilled worker may use routine methods to stabilize the relative proportions of certain CD20-binding molecules to other molecules in a composition as described herein, including the proportions of different multimeric forms of CD20-binding molecules, such as, e.g., the proportions of covalently linked, multimeric CD20-binding molecules to non-covalently linked, multimeric CD20-binding molecules (see e.g. Gil D, Schrum A, Adv Biosci Biotechnol 4: 73-84 (2013); WO2005000898). For example, the multimerization of CD20-binding molecule(s) in compositions as described herein may be controlled and/or minimized, such as, e.g., by choosing certain linkers to link and/or associate different components and/or subunits of the CD20-binding molecule(s) present in the compositions as described herein. For example, in some embodiments, the CD20 binding region of the CD20-binding molecule as described herein is engineered to minimize the formation of unwanted, intermolecular associations, multimers, and/or aggregates, such as, e.g., by using disulfide-stabilized scFvs, Fv fragments, or Fabs (see e.g. Reiter Y et al., J Biol Chem 269: 18327-31 (1994); Kuan C, Pastan I, Biochemistry 35: 2872-7 (1996); Almog O et al., Proteins 31: 128-38 (1998); Schoonjans R et al., J Immunol 165: 7050-7 (2000); Olafsen T et al., Protein Eng Des Sel 17: 21-7 (2004); Gil D, Schrum A, Adv Biosci Biotechnol 4: 73-84 (2013); U.S. 20120283418); base loop connections (see e.g. Brinkmann U et al., J Mol Biol 268: 107-17 (1997)); and/or other modifications, such as the addition of charged resides, glycans, and/or immunoglobulin-domain truncations (see e.g. Gong R et al., Mol Pharm 10: 2642-52 (2013); Lee C et al., Trends Biotechnol 31: 612-20 (2013)).

In some embodiments as described herein, the CD20-binding molecule as described herein comprises a CD20 binding region which is an scFv engineered not to aggregate, such as, e.g., by using a shorter linker (typically less than twelve amino acid residues) and/or disulfide-stabilized linker that links the heavy and light chain regions of the scFv (see e.g., Brinkmann U et al., Proc Natl Acad Sci USA 90: 7538-42 (1993); Whitlow M et al., Protein Engineering 6: 989-95 (1993); Reiter Y et al., Biochemistry 33: 5451-9 (1994); Gong R et al., Molecular Pharmaceutics 10: 2642-52 (2013)).

In some embodiments, the CD20-binding molecule composition as described herein minimizes the proportion relative to other CD20-binding molecules of certain CD20-binding molecule(s) with a valence greater than two. In some embodiments, the CD20-binding molecule composition as described herein comprises a relative percentage of CD20-binding molecules with a valence of greater than four which is 15%, 10%, 7.5%, 5%, 2%, 1%, or less of the total CD20-binding molecules in the composition. In some embodiments, a CD20-binding molecule composition as described herein comprises a relative percentage of CD20-binding molecules with a valence of greater than three to other CD20-binding molecules which is 15%, 10%, 7.5%, 5%, 2%, 1%, or less of the total CD20-binding molecules in the composition. In some embodiments, a CD20-binding molecule composition as described herein comprises a percentage of CD20-binding molecules with a valence greater than two which is 15%, 10%, 7.5%, 5%, 2%, 1%, or less of the total CD20-binding molecules in the composition.

In some embodiments, the composition as described herein maximizes the relative proportion of CD20-binding molecule(s) with exactly two CD20 binding regions to total CD20-binding molecules. Thus, in some embodiments, a composition as described herein comprises a proportion of CD20-binding molecule with only two CD20 binding regions which is 80%, 85%, 88%, 90%, 92%, 93%, or more of the total CD20-binding molecules in the composition.

For certain applications, it may be desirable to maintain stability (e.g., the stability of associations and/or linkages between components and/or subunits of the CD20-binding molecules) of CD20-binding molecule(s) in a composition as described herein, such as, e.g., to minimize degradation during formulation, storage (such as, e.g., storage in an aqueous solution at 8, 4, 2, −4, −10, −20, or −25° C.), and/or after administration to a recipient. The skilled worker may use well known methods to minimize component or subunit separation for a CD20-binding molecule as described herein, such as, e.g., by using high-stability linkages between the Shiga toxin effector polypeptide(s) and binding region(s) and/or by engineering disulfide linkages between components, regions, or sub-regions of a CD20-binding molecule or between monovalent CD20-binding proteins to generate multivalent CD20-binding protein(s) as described herein (see e.g. Gil D, Schrum A, Adv Biosci Biotechnol 4: 73-84 (2013)). The skilled worker may use the addition or maintenance of intermolecular disulfide bonds to stabilize certain CD20 binding regions of the CD20-binding molecules as described herein.

In some embodiments, the CD20-binding molecule comprises a CD20 binding region(s) which comprises an immunoglobulin domain and/or Ig-fold structure having an intra-domain disulfide bond, such as, e.g., the disulfide bond found natively between the B and F β strands of certain immunoglobulins and/or a disulfide bond between their heavy and light chains of or derived from an immunoglobulin. However, in some embodiments of the CD20-binding molecules as described herein, the molecules are very stable even though they do not comprise an intra-domain disulfide bond or any intra-domain disulfide bond within one or more CD20 binding regions.

In some embodiments, the composition as described herein comprises a CD20-binding molecule with one or more disulfide bonds between two or more cysteine residues contained within Shiga toxin effector polypeptide regions of different polypeptide chains. In some embodiments, the composition as described herein comprises a proteinaceous, dimeric CD20-binding molecule with five disulfide bonds, such as, e.g., the dimeric CD20-binding molecule comprising: 1) four, intramolecular, disulfide bonds representing two disulfide bonds per immunoglobulin-derived CD20 binding region and where each disulfide bond involves a pair of cysteine residues and wherein one cysteine residue of each pair is within an immunoglobulin heavy chain derived domain and the other cysteine residue of the pair is within an immunoglobulin light chain derived domain; and 2) one, intermolecular, disulfide bond bridging two, Shiga toxin effector regions wherein the disulfide bond occurs between a pair of cysteine residues where each cysteine residue of the pair is within a Shiga toxin effector region but the Shiga toxin effector regions are within different polypeptide chains representing different subunits of a CD20-binding protein as described herein.

In some embodiments, the CD20-binding molecule as described herein comprises a CD20 binding region derived from an immunoglobulin which has been engineered with certain camelid V_{H}H “tetrad” mutations to improve solubility, to improve stability, and/or otherwise “camelize” the binding region.

### II. Examples of Specific Structural Variations of the CD20-Binding Molecules

In some embodiments, the CD20-binding molecules described herein comprises 1) two or more proteinaceous CD20 binding regions, each capable, on its own, of specifically binding an extracellular part of CD20; and 2) one or more Shiga toxin effector regions comprising a polypeptide derived from the amino acid sequence of the A Subunit of at least one member of the Shiga toxin family.

In some embodiments, the CD20-binding molecule as described herein comprises two or more CD20 binding regions comprising an immunoglobulin-type polypeptide selected for specific and high-affinity binding to the cellular surface of a CD20+ cell.

In some embodiments of the CD20-binding molecule as described herein, the CD20 binding region comprises a polypeptide(s), the polypeptide(s) comprising: a) a heavy chain variable (V_{H}) domain comprising i) a HCDR1 comprising, consisting essentially of, or consisting of the amino acid sequence as shown in SEQ ID NO:5, SEQ ID NO:11, SEQ ID NO:17, SEQ ID NO:23, SEQ ID NO:29, or SEQ ID NO:35; ii) a HCDR2 comprising, consisting essentially of, or consisting of the amino acid sequence as shown in SEQ ID NO:6, SEQ ID NO:12, SEQ ID NO:18, SEQ ID NO:24, SEQ ID NO:30, or SEQ ID NO:36; and iii) a HCDR3 comprising, consisting essentially of, or consisting of the amino acid sequence as shown in SEQ ID NO:7, SEQ ID NO:13, SEQ ID NO:19, SEQ ID NO:25, SEQ ID NO:31, or SEQ ID NO:37; or b) a light chain variable (V_{L}) domain comprising i) a LCDR1 comprising, consisting essentially of, or consisting of the amino acid sequence as shown in SEQ ID NO:8, SEQ ID NO:14, SEQ ID NO:20, SEQ ID NO:26, SEQ ID NO:32, or SEQ ID NO:38; ii) a LCDR2 comprising, consisting essentially of, or consisting of the amino acid sequence as shown in SEQ ID NO:9, SEQ ID NO:15, SEQ ID NO:21, SEQ ID NO:27, SEQ ID NO:33, or SEQ ID NO:39; and iii) a LCDR3 comprising, consisting essentially of, or consisting of the amino acid sequence as shown in SEQ ID NO:10, SEQ ID NO:16, SEQ ID NO:22, SEQ ID NO:28, SEQ ID NO:34, or SEQ ID NO:40. In some embodiments, the CD20-binding molecule as described herein comprises the CD20 binding region comprising, consisting essentially of, or consisting of amino acids 1-232, 1-233, 1-234, 1-235, 1-236, 1-242, 1-243, 1-244, 1-245, 1-246, 1-252, 1-253, 1-254, 1-255, or 1-256 of any one of SEQ ID NOs: 47-119 and 176-248.

In some embodiments, the CD20-binding molecule as described herein comprises one or more Shiga toxin effector polypeptides(s), each comprising, consisting essentially of, or consisting of the polypeptide: (a) amino acids 75 to 251 of SEQ ID NO:1, SEQ ID NO:2, or SEQ ID NO:3; (b) amino acids 1 to 241 SEQ ID NO:1, SEQ ID NO:2, or SEQ ID NO:3; (c) amino acids 1 to 251 of SEQ ID NO:1, SEQ ID NO:2, or SEQ ID NO:3; or (d) amino acids 1 to 261 of SEQ ID NO:1, SEQ ID NO:2, or SEQ ID NO:3.

In some embodiments, the CD20-binding molecules comprise the Shiga toxin effector region comprising or consisting essentially of amino acids 75 to 251 of SEQ ID NO:1, SEQ ID NO:2, or SEQ ID NO:3. Some embodiments are CD20-binding molecules in which the Shiga toxin effector region comprises or consists essentially of amino acids 1 to 241 of SEQ ID NO:1, SEQ ID NO:2, or SEQ ID NO:3; amino acids 1 to 251 of SEQ ID NO:1, SEQ ID NO:2, or SEQ ID NO:3; and/or amino acids 1 to 261 of SEQ ID NO:1, SEQ ID NO:2, or SEQ ID NO:3.

In some embodiments, the CD20-binding molecule comprises or consists essentially of amino acids of SEQ ID NO:4, SEQ ID 12, SEQ ID NO:14, or SEQ ID NO:16.

In some embodiments, the CD20-binding molecule as described herein comprises one or more Shiga toxin effector polypeptides comprising one or more of the following substitutions: A231E, R75A, Y77S, Y114S, E167D, R170A, R176K, and W203A as positioned in the polypeptide shown in SEQ ID NO:1, SEQ ID NO:2, SEQ ID NO:3, and SEQ ID NO:4.

In some embodiments, the CD20-binding molecule as described herein comprises or consists essentially of two proteins and comprises at least one disulfide bond. In some embodiments, the disulfide bond is between a pair of cysteine residues wherein a first cysteine residue of the pair is positioned at amino acid residue 242 or 261 of the polypeptide shown in SEQ ID NO:1 or SEQ ID NO:2 or at amino acid residue position 241 or 260 in the polypeptide shown in SEQ ID NO:3 in a first Shiga toxin effector polypeptide region and a second cysteine residue of the pair is positioned at amino acid residue 242 or 261 of the polypeptide shown in SEQ ID NO:1 or SEQ ID NO:2 or at amino acid residue position 241 or 260 in the polypeptide shown in SEQ ID NO:3 of second Shiga toxin effector polypeptide region (see e.g. FIG. 1).

In some embodiments, the CD20-binding molecule as described herein comprises the protein shown in any one of SEQ ID NOs: 47-304, and optionally, the protein further comprises an amino-terminal methionine residue. In some embodiments, the CD20-binding molecule as described herein comprises, consists of, or consists essentially of two proteins, each protein selected from any one of the polypeptides shown in SEQ ID NOs: 47-304, and optionally, each protein may further comprises an amino-terminal methionine residue. In some embodiments, each of the two proteins has the sequence of any one of SEQ ID NOs: 47-175, and the CD20-binding molecule further comprises five disulfide bonds, each linking the sulfhydryl groups of a pair of cysteine residues; and wherein four of the five disulfide bonds involve cysteine residues which are in an immunoglobulin domain of a CD20-binding region of the protein linked to another cysteine residue in an immunoglobulin domain of the same CD20-binding region, and wherein the remaining disulfide bond of the five disulfide bonds involves a cysteine residue in a first protein of the two proteins having the sequence of SEQ ID NOs: 47-175 at position 242, 482, 483, 484, 490, 491, 492, 493, 494, 495, 499, 500, 501, 502, 503, 504, 505, 510, 511, 512, 513 or 521 linked to a cysteine residue from a second protein of the two proteins having the sequence of SEQ ID NOs: 47-175 at position 242, 482, 483, 484, 490, 491, 492, 493, 494, 495, 499, 500, 501, 502, 503, 504, 505, 510, 511, 512, 513 or 521.

In some embodiments, the CD20-binding molecule as described herein is a homodimer and consists essentially of (a) two identical polypeptides, each represented by SEQ ID NO:49, and (b) a cysteine disulfide bond linking the two identical polypeptides involving the cysteine at amino acid position 490 of each of the two identical polypeptides.

In some embodiments, the CD20-binding molecule as described herein is a homodimer and consists essentially of (a) two identical polypeptides, each represented by only one of SEQ ID NO:50, SEQ ID NO:61, SEQ ID NO:73, SEQ ID NO:96, SEQ ID NO:101, or SEQ ID NO:102, and (b) a cysteine disulfide bond linking the two identical polypeptides involving the cysteine at amino acid position 501 of each of the two identical polypeptides.

In some embodiments, the CD20-binding molecule as described herein is a homodimer and consists essentially of (a) two identical polypeptides, each represented by only one of SEQ ID NO:53, SEQ ID NO:63, SEQ ID NO:66, SEQ ID NO:75, SEQ ID NO:83, SEQ ID NO:89, or SEQ ID NO:95, and (b) a cysteine disulfide bond linking the two identical polypeptides involving the cysteine at amino acid position 512 of each of the two identical polypeptides.

In some embodiments, the CD20-binding molecule as described herein is a homodimer and consists essentially of (a) two identical polypeptides, each represented by only one of SEQ ID NO:54, SEQ ID NO:57, SEQ ID NO:69, SEQ ID NO:78, SEQ ID NO:82, SEQ ID NO:84, SEQ ID NO:87, SEQ ID NO:88, SEQ ID NO:94, SEQ ID NO:110, SEQ ID NO:111, or SEQ ID NO:115, and (b) a cysteine disulfide bond linking the two identical polypeptides involving the cysteine at amino acid position 503 of each of the two identical polypeptides.

In some embodiments, the CD20-binding molecule as described herein is a homodimer and consists essentially of (a) two identical polypeptides, each represented by SEQ ID NO:54, and (b) a cysteine disulfide bond linking the two identical polypeptides involving the cysteine at amino acid position 503 of each of the two identical polypeptides.

In some embodiments, the CD20-binding molecule as described herein is a homodimer and consists essentially of (a) two identical polypeptides, each represented by only one of SEQ ID NO:55, SEQ ID NO:64, SEQ ID NO:67, SEQ ID NO:76, SEQ ID NO:90, SEQ ID NO:92, SEQ ID NO:93, SEQ ID NO:97, or SEQ ID NO:98, and (b) a cysteine disulfide bond linking the two identical polypeptides involving the cysteine at amino acid position 502 of each of the two identical polypeptides.

In some embodiments, the CD20-binding molecule as described herein is a homodimer and consists essentially of (a) two identical polypeptides, each represented by only one of SEQ ID NO:56, SEQ ID NO:68, SEQ ID NO:91, SEQ ID NO:99, SEQ ID NO:103, or SEQ ID NO:104, and (b) a cysteine disulfide bond linking the two identical polypeptides involving the cysteine at amino acid position 492 of each of the two identical polypeptides.

In some embodiments, the CD20-binding molecule as described herein is a homodimer and consists essentially of (a) two identical polypeptides, each represented by only one of SEQ ID NO:58, SEQ ID NO:70, or SEQ ID NO:81, and (b) a cysteine disulfide bond linking the two identical polypeptides involving the cysteine at amino acid position 493 of each of the two identical polypeptides.

In some embodiments, the CD20-binding molecule as described herein is a homodimer and consists essentially of (a) two identical polypeptides, each represented by only one of SEQ ID NOs: 249-304, and (b) a cysteine disulfide bond linking the two identical polypeptides involving the cysteine at amino acid position 242 of each of the two identical polypeptides.

It is within the scope as described herein to use fragments, variants, and/or derivatives of the proteins of the CD20-binding molecules as described herein, such as, e.g., proteins which contain two or more, functional, CD20 binding regions, and even more preferably two CD20 binding regions capable of binding an extracellular part of CD20 with high affinity (e.g. as determined using the CD20 binding region's K_{D} empirically measured with a CD20-expressing cell(s) or in vitro with a CD20 target molecule(s)). For example, while polypeptides that can bind to CD20 are provided herein, any binding region that binds to an extracellular part of CD20 with a dissociation constant of 10⁻⁵ to 10⁻¹² moles per liter, preferably less than 200 nM, may be suitable for use in making CD20-binding molecules as described herein, and related compositions and methods described herein.

### III. General Functions of the CD20-Binding Molecule and Compositions Thereof

Provided herein are various CD20-binding molecules and compositions thereof wherein each CD20-binding molecule comprises 1) two or more CD20 binding regions for cell targeting; and 2) at least one Shiga toxin effector polypeptide region. The linking of multiple cell targeting, CD20 binding regions with Shiga toxin Subunit A derived polypeptides enables the cell type-specific targeting of the potent Shiga toxin cytotoxicity and/or cytostasis, as well as the ability to deliver exogenous materials into the interiors of CD20+ cell types, such as, e.g., intracellularly cytotoxic agents.

In some embodiments, the CD20-binding molecule as described herein, and compositions thereof, may be used to target potent Shiga toxin cytotoxicity, cytostasis, rapid intracellular delivery of cargo, or other cellular internalization function to various, CD20-expressing cell types. In some embodiments, the CD20-binding molecule as described herein are capable of binding extracellular CD20 molecules associated with the cell surfaces of particular cell types and rapidly entering those cells. Once internalized within a targeted cell, certain embodiments of the CD20-binding molecules as described herein are capable of routing a cytotoxic Shiga toxin effector polypeptide fragment into the cytosol of the target cell. Once in the cytosol of a targeted cell, certain embodiments of the cytotoxic CD20-binding molecules as described herein are capable of enzymatically inactivating ribosomes, interfering with cell homeostasis, and eventually killing the cell. In some embodiments, the CD20-binding molecule as described herein, and compositions thereof, may be used to deliver additional exogenous materials into CD20-expressing cells, such as, e.g., peptides, polypeptides, proteins, polynucleotides, and detection promoting agents to label the interiors of target cells for collecting diagnostically useful information.

### A. CD20 Positive Cell Kill Via Targeted Shiga Toxin Cytotoxicity

Because members of the Shiga toxin family are adapted to killing eukaryotic cells, CD20-binding molecules comprising a Shiga toxin effector region can show potent cell-kill activity. The A Subunits of members of the Shiga toxin family comprise enzymatic domains capable of killing a eukaryotic cell once in the cell's cytosol. Certain embodiments of the CD20-binding molecules as described herein, and compositions thereof, take advantage of this cytotoxic mechanism. Certain embodiments of the CD20-binding molecules as described herein, and compositions thereof, exhibit cell-targeted cytotoxicity via their Shiga toxin effector polypeptide regions.

In some embodiments of the CD20-binding molecules as described herein, and compositions thereof, upon contacting a cell physically coupled with extracellular CD20 having the extracellular part bound by the binding regions of the CD20-binding molecule as described herein, the CD20-binding molecule as described herein, and/or a composition thereof, is capable of causing the death of the cell. In some embodiments, the capability to cause the death of the cell requires an intracellular mechanism, such as, e.g., a catalytic activity of a toxin effector polypeptide region.

The killing of a CD20-expressing cell(s) may be accomplished using a cytotoxic CD20-binding molecule as described herein, and/or a composition thereof, under varied conditions of CD20-expressing target cells, such as an ex vivo manipulated target cell, a target cell cultured in vitro, a target cell within a tissue sample cultured in vitro, and/or a target cell in vivo.

The expression of CD20 at a cellular surface need not be native to achieve targeted cell killing by a cytotoxic CD20-binding molecule as described herein and/or composition thereof. Cell-surface expression of CD20 by a target cell could be the result of an infection, the presence of a pathogen, and/or the presence of an intracellular microbial pathogen. Expression of CD20 by a target cell could be artificial or engineered such as, for example, by forced or induced expression after infection with a viral expression vector, see e.g. adenoviral, adeno-associated viral, and retroviral systems. An example of inducing expression of CD20 is the up-regulation of CD20 induced by exposing a cell to ionizing radiation.

Whether a particular cell type or cell population expresses CD20 at a cellular surface can be determined by methods well known in the art. For example, both FACS methods and immunohistochemical methods using anti-CD20 antibodies are known in the art may be used to as assays to determine cells which express CD20 at a cellular surface (CD20+ cells) and, thus, determine which cells have a particular extracellular CD20 target biomolecule physically coupled to them. In addition, the density of CD20 expression at a cell surface of a CD20+ cell type may be assayed using methods known in the art, including but not limited to the methods mentioned herein.

The effectiveness and potency of CD20-binding molecules as described herein toward different target cells may be influenced by the densities of their CD20 target antigen(s) on a target cell surface, the locations of their epitope-binding interaction with CD20, the rates of CD20 internalization of cell-surface bound CD20 of different target cells, and the intracellular itinerary of different target cells.

The cell surface representation and/or density of an extracellular CD20 target may influence the applications for which certain CD20-binding molecules as described herein, or compositions thereof, may be most suitably used. Differences in cell surface representation and/or density of certain extracellular CD20 target(s) between cells may alter the internalization and/or cytotoxicity of a given CD20-binding molecule described herein, or composition thereof, both quantitatively and qualitatively. The total cell surface representation of CD20 and/or of certain CD20 epitope(s) on a particular cell or population of cells may be determined using methods known to the skilled worker, such as by using fluorescence-activated cell sorting (FACS), flow-cytometry techniques.

The cell surface representation and/or density of a given extracellular CD20 target (or extracellular epitope of a given CD20 target) may influence the applications for which certain CD20-binding molecules as described herein may be most suitably used. Differences in cell surface representation and/or density of a given CD20 target(s) or epitope(s) between cells may alter, both quantitatively and qualitatively, the efficiency of cellular internalization, and/or potency of cytotoxicity of a given CD20-binding molecule as described herein. The cell surface representation and/or density of a given extracellular CD20 target or epitope may vary greatly among CD20 positive cells or even on the same cell at different points in the cell cycle or cell differentiation. The total cell surface representation of a given extracellular CD20 target (e.g. a particular extracellular epitope of a given CD20) on a particular cell or population of cells may be determined using methods known to the skilled worker, such as methods involving fluorescence-activated cell sorting (FACS) flow cytometry.

An example of a FACS based assay for determining cell surface representation of an extracellular CD20 antigen for a particular cell type is as follows. An anti-CD20 antibody is labeled with a fluorophore, such as, e.g. a fluorescein derivative like fluorescein isothiocyanate (FITC), an Alexa Fluor® Dye like Alexa488, or some other fluorescent tag. A population of cells of the cell type of interest are grown and harvested at a density of 1×10⁶ cells per milliliter (mL) and treated with 0.1 to 1.0 milligrams (mg) per mL (mg/mL) of labeled anti-CD20 antibody for 30 minutes on ice. Then the cold, treated cells are washed twice to remove unbound antibody. Alternatively, an unlabeled anti-CD20 antibody is used and is detected by a secondary antibody, such as, e.g., an anti-mouse IgG conjugated with a fluorophore, such as, e.g., Alexa488 or FITC. Direct immunofluorescence is used to quantify the amount of extracellular CD20 such as by using a FACS device.

For example, cell-surface CD20 is usually expressed at high levels by B-cells as compared to other cell surface targets, such as at levels of 250,000 cell-surface CD20 molecules per cell, which provides a large density of extracellular CD20 targets for the CD20-binding molecules as described herein.

For certain embodiments of the CD20-binding molecule, and compositions thereof, the ability, upon contacting a cell physically coupled with extracellular CD20 having the extracellular part bound by the binding regions of the CD20-binding molecule, of killing the cell may or may not depend on the catalytic activity of one or more Shiga toxin effector regions of the CD20-binding molecule. In some embodiments of the CD20-binding molecules as described herein, and compositions thereof, upon contacting a cell physically coupled with extracellular CD20 having the extracellular part bound by the binding regions of the CD20-binding molecule, the CD20-binding molecule as described herein, and/or a composition thereof, is capable of causing the death of the cell. In some embodiments, the CD20-binding molecule as described herein either (1) comprises a Shiga toxin effector region(s) that lacks catalytic activity and/or is not capable of causing the death of a cell through a Shiga toxin effector-mediated, ribosome inactivation mechanism; or (2) does not comprise any Shiga toxin effector region.

Certain embodiments of the multivalent CD20-binding molecules described herein, and enriched compositions thereof, exhibit unexpectedly potent, cell-targeted cytotoxicity potencies compared to monovalent CD20-binding molecules and/or compositions thereof which lack CD20-binding molecule(s) or have lower proportions of multivalent CD20-binding molecule(s) to the total CD20-binding molecule than an enriched composition (i.e., a CD20-binding molecule composition comprising that multivalent CD20-binding molecule). Without being bound by any theory, the CD20-binding valence-related improvement in function(s) between monovalent CD20-binding molecules and multivalent CD20-binding molecules result from the CD20-binding structure in a way that is believed to be more than just a result of CD20-binding valency effect(s), but rather, seems to result from a de novo property(ies) of the CD20-binding structures that is not present in certain monovalent CD20-binding variants. Without being bound by any theory, multivalent CD20-binding molecules as described herein, and enriched compositions thereof, that exhibit cytotoxic potencies that are unexpectedly greater in a qualitative and/or quantitative manner compared to a monovalent CD20-binding variant may exhibit such levels of cytotoxic potency as a result of an improvement(s), such as, e.g., in the molecule's efficiency of 1) cellular internalization into a CD20-expressing cell(s), 2) intracellular routing after cellular internalization to a certain subcellular compartment(s), and/or 3) delivering a Shiga toxin effector polypeptide to the cytosol.

In some embodiments, a multivalent CD20-binding molecule comprises one or more monovalent CD20-binding molecule components; and whereby upon administration of the multivalent CD20-binding molecule, or a composition thereof, to a population of cells physically coupled with CD20 (e.g. CD20+ cells), which have the extracellular part bound by two or more CD20 binding regions of the multivalent CD20-binding molecule, the multivalent CD20-binding molecule exhibits a cytotoxic effect which is greater than a cytotoxic effect resulting from administration of an equivalent amount, mass, or molarity of any one of the monovalent CD20-binding molecule components of the multivalent CD20-binding molecule to a population of the same type of CD20 positive cells under same conditions (e.g., same temperature, cell density, and assay time duration) by a factor of 1.33, 1.5, 1.75, 2, 3, 5, 7.5, 10, 20, 100, or greater than (1) the change in CD20-binding valence between the monovalent CD20-binding component and the multivalent CD20-binding molecule; (2) the change in equilibrium binding constants (K_{D}) between the multivalent CD20-binding molecule and the monovalent CD20-binding component for binding to CD20 or CD20-expressing cell; and/or (3) the change in affinity constant (1/K_{D}) between the multivalent CD20-binding molecule and the monovalent CD20-binding component for binding to CD20 or CD20-expressing cell. In some embodiments, members of the population of cells express at a cellular surface the CD20 which (1) have the extracellular part bound by the two or more CD20 binding regions of the multivalent CD20-binding molecule wherein each CD20 binding region is tested in isolation from the multivalent CD20-binding molecule, (2) have a transmembrane domain, and (3) remain physically coupled to the cell. In some embodiments, members of the population of cells are CD20 positive cells. For certain embodiments, the members of the population of cells are physically coupled with a significant amount of extracellular CD20 which (1) have the extracellular part bound by the two or more CD20 binding regions of the multivalent CD20-binding molecule. In some embodiments, members of the population of cells are descendants or members of a B-cell lineage. In some embodiments, members of the population of cells are members of the following groups: malignant B-cell, B-cell leukemia cell, B-cell lymphoma cell, B-cell myeloma cell, acute myeloid leukemia cell, acute non-lymphocytic leukemia cell, B-cell chronic lymphocytic leukemia cell, B-cell lymphoma cell, B-cell non-Hodgkin's lymphoma cell, B-cell precursor acute lymphoblastic leukemia cell, B-cell prolymphocytic leukemia cell, Burkitt's lymphoma cell, chronic lymphocytic leukemia cell, chronic myeloid leukemia cell, diffuse large B-cell lymphoma cell, follicular lymphoma cell, hairy cell leukemia cell, Hodgkin's lymphoma cell, immunoblastic large cell lymphoma cell, mantle cell lymphoma cell, melanoma cell, multiple myeloma cell, neoplastic plasma cell, nodular lymphocyte predominant Hodgkin's lymphoma cell, non-Hodgkin's lymphoma cell, plasmablastic lymphoma cell, plasma cell myeloma cell, precursor B-lymphoblastic lymphoma cell, small lymphocytic lymphoma cell, malignant T-cell, T-cell leukemia cell, T-cell lymphoma cell, T-cell large granular lymphocyte leukemia cell, T-cell prolymphocytic leukemia, healthy B-cell lineage cell or healthy T-cell.

### B. Selective Cytotoxicity Among a Mixture of Different Cells

The cytotoxic CD20-binding molecules as described herein, and compositions thereof, are useful for the elimination of populations of specific cell types within the presence of untargeted cells or “bystander” cells. For example, the cytotoxic CD20-binding molecules described herein, and compositions thereof, are useful for the treatment of certain tumors, cancers, and/or growth abnormalities by eliminating CD20-expressing cells that express elevated levels of CD20 at one or more cellular surfaces. By targeting the delivery of enzymatically active Shiga toxin regions using multiple, high-affinity CD20 binding regions to CD20 expressing cells, Shiga toxin cell-kill activity can be restricted to preferentially killing CD20-expressing cell types that express CD20 at a cellular surface (e.g. CD20+ cells), such as, e.g., certain neoplastic or malignant plasma cells, in the presence of two or more cell types where at least one cell type population expresses more CD20 than at least one other cell type population.

In some embodiments, the cytotoxic CD20-binding molecule as described herein is capable of selectively or preferentially causing the death of a specific cell type within a mixture of two or more different cell types. This enables the targeted cytotoxic activity to specific cell types with a high preferentiality, such as a 3-fold cytotoxic effect, over “bystander” cell types that do not express extracellular CD20.

In some embodiments, administration of the CD20-binding molecule as described herein to a mixture of cell types, the CD20-binding molecule is capable of selectively killing CD20-expressing cells displaying an extracellular CD20 target compared to cell types lacking extracellular CD20 target(s) of the binding region(s) of the CD20-binding molecule.

In some embodiments, upon administration of the cytotoxic CD20-binding molecule as described herein to a mixture of cell types, the cytotoxic CD20-binding molecule is capable of selectively killing CD20+ cells expressing an extracellular CD20 target biomolecule compared to cells lacking any cell-surface expression of extracellular CD20 target biomolecules.

Certain CD20 positive, cell types may be killed by a CD20-binding molecule as described herein, or compositions thereof, in the presence of other cells, including other CD20 positive cells, based on different levels of extracellular CD20 target expression among the target cells and non-target cells. For example, cells which overexpress CD20 may be killed among healthy cells, whether the healthy cells are expressing CD20 or not.

A cell which “overexpresses” a target biomolecule includes a cell which has significantly higher levels of the target biomolecule physically coupled at its cell surface compared to a healthy cell of the same tissue type. Overexpression may be caused by a variety of circumstances, such as, e.g., gene amplification, increased transcription, increased translation, reduced CD20 shedding, and/or reduced removal of the CD20 target biomolecule. The skilled worker may determine overexpression of a particular target biomolecule using methods known in the art.

Levels of extracellular CD20 target biomolecules on the surface of cells may be determined using various methods known to the skilled worker, such as, e.g., FACS methods. As used herein, a significant amount of an extracellular CD20 expressed at a cellular surface is greater than 10,000, 20,000, 30,000, 40,000, or 50,000 mean fluorescence intensity (MFI) by FACS analysis depending on the cell type.

The cytotoxic CD20-binding molecules as described herein are useful for reducing or eliminating populations of a specific cell type(s). For example, the cytotoxic CD20-binding molecules described herein are useful for the treatment of certain tumors, cancers, and/or growth abnormalities by eliminating CD20+ cells that express elevated levels of CD20 at one or more cellular surfaces (e.g. cells characterized as overexpressing CD20).

In some embodiments, a CD20-binding molecule may be used to target cytotoxic activity to specific cell types with a high preferentiality, such as with at least a 3-fold cytotoxic effect, over “bystander” cell types that are CD20+ but express cell-surface CD20 at lower cell surface amounts or densities than target cells. The expression of CD20 may be non-exclusive to one cell type if extracellular CD20 is expressed in low enough amounts by cell types that are not to be targeted. Thus, preferential killing of one CD20 positive cell type may be accomplished in mixtures of multiple CD20+ where some CD20+ cell types are bystander cells, such as mixtures of CD20+ cell types with varying CD20 expression levels, optionally in the presence of CD20 negative cells as well. This enables the preferential cell-killing of high-expressing CD20 cell types, such as a 3-fold cytotoxic effect, over “bystander” cell types that do not express significant amounts of CD20 or do not expose significant amounts of an extracellular CD20 target of at least one of the CD20 binding regions of the cytotoxic CD20-binding molecules.

In some embodiments, the cytotoxic activity of a CD20-binding molecule toward populations of cell types physically coupled with an extracellular CD20 target is at least 3-fold higher than the cytotoxic activity toward populations of cell types not physically coupled with significant amounts of an extracellular CD20 target bound specifically by at least one of the CD20 binding regions of that CD20-binding molecule. Selective cytotoxicity may be quantified in terms of the ratio (a/b) of (a) cytotoxicity towards a population of cells physically coupled with a significant amount of an extracellular CD20 target of at least one of the CD20 binding regions of the CD20-binding molecule to (b) cytotoxicity towards a population of cells of a cell type not physically coupled with a significant amount of an extracellular CD20 target of at least one of the CD20 binding regions of the CD20-binding molecule.

In some embodiments, the cytotoxicity ratio is indicative of the selective cytotoxicity which is at least 3-fold, 5-fold, 10-fold, 15-fold, 20-fold, 25-fold, 30-fold, 40-fold, 50-fold, 75-fold, 100-fold, 250-fold, 500-fold, 750-fold, 1000-fold or higher for populations of cells or cell types expressing or physically coupled with an extracellular CD20 target of at least one of the CD20 binding regions of the CD20-binding molecule compared to populations of cells or cell types which do not express an extracellular CD20 target or that are not physically coupled with significant amounts of an extracellular CD20 target bound specifically by at least one of the CD20 binding regions of the CD20-binding molecule as described herein. For example, upon administration of a certain CD20-binding molecule as described herein to two different populations of cells which differ with respect to the presence and/or polypeptide sequence of extracellular CD20 target biomolecule, the CD20-binding molecule is capable of causing cell death to the cell type(s) physically coupled with an extracellular CD20 target biomolecule bound by at least one of the CD20-binding molecule's CD20 binding regions, e.g., at a CD₅₀ at least three times less than the CD₅₀ of binding to cell types that are not physically coupled with an extracellular CD20 target of the CD20-binding molecule's CD20 binding region.

In some embodiments of the CD20-binding molecules as described herein, upon administration of the CD20-binding molecule to two different populations of cell types, the CD20-binding molecule is capable of causing cell death as defined by the half-maximal cytotoxic concentration (CD₅₀) to a first cell population, whose members express CD20 at a cellular surface, at a dose at least three-times lower than the CD₅₀ dose of the same CD20-binding molecule to a second population of cells whose members do not express CD20, do not express a significant amount of CD20, or are not exposing a significant amount of an extracellular CD20 target of at least one of the CD20 binding regions of the CD20-binding molecule.

Selective cytotoxicity may be quantified in terms of the ratio (a/b) of (a) cytotoxicity towards a population of CD20+ cells to (b) cytotoxicity towards a population of CD20 negative cells. In some embodiments, the cytotoxicity ratio is indicative of selective cytotoxicity which is at least 3-fold, 5-fold, 10-fold, 15-fold, 20-fold, 25-fold, 30-fold, 40-fold, 50-fold, 75-fold, 100-fold, 250-fold, 500-fold, 750-fold, or 1000-fold higher for populations of CD20+ cells or CD20+ cell populations compared to CD20− cells or CD20− cell populations. For example, administration of certain embodiments of the CD20-binding molecule to two different populations of cell types with respect to the presence of an extracellular CD20 target biomolecule, the CD20-binding molecule is capable of causing cell death to the CD20 target biomolecule positive cells at a CD₅₀ at least three times less than the CD₅₀ to CD20 target biomolecule negative cells.

Particular CD20 expression levels within an organism may be limited to unique cells, tissues, cell types, conditions, disease states, disorders, and/or cellular contexts. CD20 may be overexpressed by cells involved in many disease states, such as, e.g., by malignant immune cells, tumor cells, and cancer cells. CD20 may be expressed by cells (e.g. healthy cells) which are overactive or providing a detrimental effect to the organism resulting in a condition, disease, or disorder.

In some embodiments, administration of a CD20-binding molecule composition to a mixture of cell types, the CD20-binding molecule composition is capable of selectively killing CD20-expressing cells displaying an extracellular CD20 target compared to cell types lacking an extracellular CD20 target(s) of the CD20-binding molecule of the composition.

In some embodiments, administration of a CD20-binding molecule composition to two populations of cell types which differ in the presence and/or polypeptide sequence of a extracellular CD20 target, the CD20-binding molecule composition is capable of causing cell death as defined by the half-maximal cytotoxic concentration (CD₅₀) to a population of CD20+ target cells, e.g., at a dose at least three times lower than the CD₅₀ dose of the same CD20-binding molecule composition to a CD20− cell population.

In some embodiments, the CD20-binding molecule compositions as described herein are capable of selectively or preferentially causing the death of a specific cell type within a mixture of two or more different cell types. This enables targeting cytotoxic activity to specific cell types with a high preferentiality, such as with at least a 3-fold cytotoxic effect, over “bystander” cell types that do not express any significant amount of the appropriate extracellular CD20 target(s), such as, e.g., CD20 negative cells. This enables the targeted cell-killing of specific cell types expressing CD20 on cellular surfaces with a high preferentiality, such as with at least a 3-fold cytotoxic effect, over “bystander” cell types that do not express significant amounts of the appropriate CD20 target(s) or are not exposing significant amounts of the appropriate CD20 target at a cellular surface.

Levels of extracellular CD20 expressed on the surface of a cell or cell population may be determined using various methods known to the skilled worker, such as, e.g., FACS methods. As used herein, a significant amount of an extracellular CD20 expressed at a cellular surface is greater than 10,000, 20,000, 30,000, 40,000, or 50,000 mean fluorescence intensity (MFI) by FACS analysis depending on the cell type.

Alternatively, certain CD20-binding molecules as described herein, and compositions thereof, enable targeting cytotoxic activity to specific cell types with a high preferentiality, such as with at least a 3-fold cytotoxic effect, over “bystander” cell types that are CD20+ but express CD20 at lower cell surface amounts or densities than target cells. Thus, preferential killing of one CD20 positive cell type may be accomplished in mixtures of multiple CD20+ cell types where some CD20+ cell types are bystander cells, such as mixtures of CD20+ cell types with varying CD20 expression levels, and optionally in the presence of CD20 negative cells as well.

In some embodiments, the cytotoxic activity toward populations of cell types physically coupled with an extracellular CD20 target is at least 3-fold higher than the cytotoxic activity toward populations of cell types not physically coupled with significant amounts of extracellular CD20 target(s) of at least one of the CD20 binding regions of the cytotoxic CD20-binding molecule as described herein. Selective cytotoxicity may be quantified in terms of the ratio (a/b) of (a) cytotoxicity towards a population of cells physically coupled with a significant amount of an extracellular CD20 target of at least one of the CD20 binding regions of the cytotoxic CD20-binding molecule to (b) cytotoxicity towards a population of cells of a cell type not physically coupled with a significant amount of an extracellular CD20 target of at least one of the CD20 binding regions of the cytotoxic CD20-binding molecule. In some embodiments, the cytotoxicity ratio is indicative of selective cytotoxicity which is at least 3-fold, 5-fold, 10-fold, 15-fold, 20-fold, 25-fold, 30-fold, 40-fold, 50-fold, 75-fold, 100-fold, 250-fold, 500-fold, 750-fold, or 1000-fold higher for populations of cells or cell types expressing an extracellular CD20 target or physically coupled with an extracellular CD20 target of at least one of the CD20 binding regions of the cytotoxic CD20-binding molecule compared to populations of cells or cell types which do not express an extracellular CD20 target or are not physically coupled with significant amounts of an extracellular CD20 target of at least one of the CD20 binding regions of the cytotoxic CD20-binding molecule. For example, administration of certain embodiments of the CD20-binding molecule composition as described herein to two different populations of cell types with respect to the presence of an extracellular CD20 target biomolecule, the CD20-binding molecule composition is capable of causing cell death to the cell type(s) physically coupled with an extracellular CD20 target biomolecule of one or more of its CD20 binding regions at a CD₅₀ at least three times less than the CD₅₀ to cell types which are not physically coupled with an extracellular CD20 target of its CD20 binding region.

In some embodiments of the CD20-binding molecule composition as described herein, administration of the CD20-binding molecule composition to two different populations of cell types, the CD20-binding molecule composition is capable of causing cell death as defined by the half-maximal cytotoxic concentration (CD₅₀) to a first cell population, whose members express CD20 at a cellular surface, at a dose at least three-times lower than the CD₅₀ dose of the same CD20-binding molecule compositions to a second population of cells whose members do not express CD20, do not express a significant amount of CD20, or are not exposing a significant amount of an extracellular CD20 target of at least one of the CD20 binding regions of the CD20-binding molecule composition.

In some embodiments, the cytotoxic activity of a CD20-binding molecule composition as described herein toward populations of cell types expressing CD20 at a cellular surface is at least 3-fold higher than the cytotoxic activity toward populations of cell types not physically coupled with any extracellular CD20 target bound specifically by a CD20-binding molecule of the CD20-binding molecule composition.

Selective cytotoxicity may be quantified in terms of the ratio (a/b) of (a) cytotoxicity towards a population of cells expressing an extracellular CD20 target of a CD20 binding region of the embodiment to (b) cytotoxicity towards a population of cells of a cell type not physically coupled with any extracellular CD20 target of a CD20 binding region of the embodiment. In some embodiments, the cytotoxicity ratio is indicative of selective cytotoxicity which is at least 3-fold, 5-fold, 10-fold, 15-fold, 20-fold, 25-fold, 30-fold, 40-fold, 50-fold, 75-fold, 100-fold, 250-fold, 500-fold, 750-fold, or 1000-fold higher for populations of cells or cell types expressing CD20 compared to populations of cells or cell types which do not express CD20.

In some embodiments, the cytotoxic activity of a CD20-binding molecule composition as described herein toward populations of cell types physically coupled with an extracellular CD20 target is at least 3-fold higher than the cytotoxic activity toward populations of cell types not physically coupled with significant amounts of an extracellular CD20 target bound specifically by at least one of the CD20 binding regions of a CD20-binding molecule of the CD20-binding molecule composition. Selective cytotoxicity may be quantified in terms of the ratio (a/b) of (a) cytotoxicity towards a population of cells physically coupled with a significant amount of an extracellular CD20 target of at least one of the CD20 binding regions of a cytotoxic CD20-binding molecule as described herein to (b) cytotoxicity towards a population of cells of a cell type not physically coupled with a significant amount of an extracellular CD20 target of at least one of the CD20 binding regions of the cytotoxic CD20-binding molecule. In some embodiments, the cytotoxicity ratio is indicative of selective cytotoxicity which is at least 3-fold, 5-fold, 10-fold, 15-fold, 20-fold, 25-fold, 30-fold, 40-fold, 50-fold, 75-fold, 100-fold, 250-fold, 500-fold, 750-fold, 1000-fold or higher for populations of cells or cell types expressing an extracellular CD20 target or physically coupled with an extracellular CD20 target of at least one of the CD20 binding regions of a cytotoxic CD20-binding molecule of the composition described herein compared to populations of cells or cell types which do not express an extracellular CD20 target or that are not physically coupled with significant amounts of an extracellular CD20 target bound specifically by any of the CD20 binding regions of the cytotoxic CD20-binding molecule of the composition described herein. For example, upon administration of certain CD20-binding molecule compositions described herein to two different populations of cells which differ with respect to the presence and/or polypeptide sequence of extracellular CD20 target biomolecule, the CD20-binding molecule compositions are capable of causing cell death to the cell type(s) physically coupled with an extracellular CD20 target biomolecule bound by at least one of the CD20 binding regions of a CD20-binding molecule of the composition, e.g., at a CD₅₀ at least three times less than the CD₅₀ of binding to cell types that are not physically coupled with an extracellular CD20 target of any of the CD20-binding molecules of the composition.

In some embodiments of the CD20-binding molecule composition as described herein, upon administration of the CD20-binding molecule composition to two different populations of cell types, the CD20-binding molecule composition is capable of causing cell death as defined by the half-maximal cytotoxic concentration (CD₅₀) to a first cell population, whose members express CD20 at a cellular surface, at a dose at least three-times lower than the CD₅₀ dose of the same CD20-binding molecule composition to a second population of cells whose members do not express CD20, do not express a significant amount of CD20, or are not exposing a significant amount of an extracellular CD20 target of at least one of the CD20 binding regions of any cytotoxic CD20-binding molecule of the CD20-binding molecule composition.

This preferential cell-killing function allows a targeted CD20+ cell to be killed by certain CD20-binding molecules as described herein, and compositions thereof, under varied conditions and in the presence of non-targeted CD20− bystander cells, such as ex vivo manipulated mixtures of cell types, in vitro cultured tissues with mixtures of cell types, or in vivo in the presence of multiple cell types (e.g. in situ, in a native location within a multicellular organism, or at disease locus within a multicellular organism).

In some embodiments, upon administration of the CD20-binding molecule as described herein, and/or composition thereof, to a mixture of cell types, the CD20-binding molecule, and/or composition thereof, is capable of selectively killing CD20+ cells expressing an extracellular CD20 target biomolecule compared to cells lacking any cell-surface expression of extracellular CD20 target biomolecules. By targeting the delivery of enzymatically active Shiga toxin regions to specific cell types using high-affinity CD20 binding regions, this potent and selective cell-kill activity can be restricted to killing only CD20 expressing cells within in an organism.

In some embodiments described herein, the CD20-binding molecules as described herein, and compositions thereof, have applications in killing CD20+ cells in a disease, disorder or condition involving cells with abnormally high CD20 expression and/or ectopic CD20 expression. Various types of cells which express CD20 may be targeted by the CD20-binding molecules as described herein, and compositions thereof, for cell killing and/or cytostasis. For example, there are various types of CD20+ cells which function in various biological processes including autoimmune conditions, neoplastic B-cell proliferation, and hypersensitivity responses.

C. Delivery of an Additional, Exogenous Material into the Interior of a Target Cell

In addition to CD20-binding molecules as described herein, and compositions thereof, optionally may be used for delivery of additional exogenous materials into the interiors of target cells. The delivery of additional exogenous materials may be used, e.g., for cytotoxic, cytostatic, information gathering, and/or diagnostic functions. Nontoxic and reduced-cytotoxic variants of the cytotoxic CD20-binding molecules as described herein, or optionally toxic variants, may be used to deliver additional exogenous materials to and/or label the interiors of cells physically coupled with an extracellular CD20 molecule.

For example, certain embodiments of the CD20-binding molecules as described herein do not exhibit a significant level of Shiga toxin based cytotoxicity, such as, e.g., no significant cell death is observed at concentrations of a CD20-binding molecule as described herein of less than 1,000 nM, 500 nM, 100 nM, 75 nM, 50 nM in an in vitro cell culture, cell-kill assay. Similarly, at administration doses of 1-100 μg of a cell-targeting molecule as described herein per kg of a mammalian recipient, there will be no observable toxicity. This system is modular, in that any number of diverse CD20-binding regions can be used to target a CD20-binding molecule as described herein to various, diverse cell types, such as, e.g., cells of different mammalian species.

Various types of cells and/or cell populations which express CD20 at one or more cellular surfaces may be targeted by the CD20-binding molecules as described herein for receiving exogenous materials. The functional components as described herein are modular in that various Shiga toxin effector regions and additional exogenous materials may be linked to various CD20 binding regions to create varied CD20-binding molecules as described herein that are suitable for use in diverse applications, such as non-invasive, in vivo imaging of tumor cells.

Because the CD20-binding molecules, whether cytotoxic, reduced-cytotoxic, or nontoxic, and catalytically inactive forms thereof, are capable of entering cells physically coupled with CD20 molecule, some CD20-binding molecules as described herein may be used to deliver additional exogenous materials into the interior of targeted CD20+ cell types. In one sense, the entire CD20-binding molecule is an exogenous material which will enter the target cell; thus, the “additional” exogenous materials are heterologous materials linked to but other than the core CD20-binding molecule itself, which is composed of merely of the minimum required components to achieve rapid cellular internalization and/or efficient sub-cellular routing to the desired intracellular compartment(s).

In some embodiments, the CD20-binding molecule comprises an additional exogenous material. An “additional exogenous material” as used herein refers to one or more atoms or molecules that can be transported to the interior of a cell by a binding molecule. In some embodiments, an additional exogenous material is any material transported into the interior of a cell by a binding molecule, whether or not it is typically present in the native target cell or in a native Shiga toxin. In some embodiments, an additional exogenous material is a material that is not generally present in Shiga toxins and/or native target cells.

Non-limiting examples of additional exogenous materials are cytotoxic agents, peptides, polypeptides, proteins, polynucleotides, small molecule chemotherapeutic agents, radionuclides, and detection promoting agents.

Nontoxic variants and reduced cytotoxicity variants of the cytotoxic CD20-binding molecules as described herein, and compositions thereof, or optionally toxic variants, may be used to deliver additional exogenous materials and/or label the interiors of cells physically coupled with CD20 molecules bound by the CD20-binding molecules as described herein. Various types of cells and/or cell populations which express CD20 at a cellular surface may be targeted by the CD20-binding molecules as described herein, and compositions thereof, for killing and/or receiving exogenous materials, such as detection promoting agents. The system as described herein is modular, in that various Shiga toxin effector regions and additional exogenous materials may be linked to the same binding region to provide diverse applications, such as, e.g., non-invasive in vivo imaging of the interiors of tumor cells and/or immune cells.

In some embodiments, the additional exogenous material is a cytotoxic agent, such as, e.g., a small molecule chemotherapeutic agent, cytotoxic antibiotic, alkylating agent, antimetabolite, topoisomerase inhibitor, and/or tubulin inhibitor. Non-limiting examples of cytotoxic agents include aziridines, cisplatins, tetrazines, procarbazine, hexamethylmelamine, vinca alkaloids, taxanes, camptothecins, etoposide, doxorubicin, mitoxantrone, teniposide, novobiocin, aclarubicin, anthracyclines, actinomycin, bleomycin, plicamycin, mitomycin, daunorubicin, epirubicin, idarubicin, dolastatins, maytansines, docetaxel, adriamycin, calicheamicin, auristatins, pyrrolobenzodiazepine, carboplatin, 5-fluorouracil (5-FU), capecitabine, mitomycin C, paclitaxel, 1,3-Bis(2-chloroethyl)-1-nitrosourea (BCNU), rifampicin, cisplatin, methotrexate, and gemcitabine.

In some embodiments, the additional exogenous material comprises a protein or polypeptide comprising an enzyme. In certain other embodiments, the additional exogenous material is a nucleic acid, such as, e.g. a ribonucleic acid that functions as a small inhibiting RNA (siRNA) or microRNA (miRNA). In some embodiments, the additional exogenous material is an antigen, such as antigens derived from bacterial proteins, viral proteins, proteins mutated in cancer, proteins aberrantly expressed in cancer, or T-cell complementary determining regions. For example, exogenous materials include antigens, such as those characteristic of antigen-presenting cells infected by bacteria, and T-cell complementary determining regions capable of functioning as exogenous antigens. In some embodiments, the additional exogenous material comprises a proapoptotic peptide, polypeptide, or protein, such as, e.g., BCL-2, caspases, cytochromes, granzyme B, apoptosis-inducing factor (AIF), BAX, tBid (truncated Bid), and proapoptotic fragments or derivatives thereof. Additional examples of exogenous materials include proteins larger than an antigenic peptide, such as enzymes. Exogenous materials comprising a protein may optionally comprise one or more antigens whether known or unknown to the skilled worker.

In some embodiments of the CD20-binding molecules as described herein, and compositions thereof, for use in delivery of additional exogenous materials into the interior of a cell, the additional exogenous material is one or more radionucleides, such as, e.g., ²¹¹At, ¹³¹I, ¹²⁵I, ⁹⁰Y, ¹¹¹In, ¹⁸⁶Re, ¹⁸⁸Re, ¹⁵³Sm, ²¹²Bi, ³²P, ⁶⁰C, and/or radioactive isotopes of Lu.

In some embodiments, the CD20-binding molecule as described herein comprises an additional exogenous material for delivery into a cell, and the CD20-binding molecule comprises the protein shown in any one of SEQ ID NOs: 51-52, 59-61, 64-65, 71-73, 76-77, 82-83, 88-89, 94, 100, 106, 109-112, 115-118, 124, 132, 140, 145, 150, 156, 162, 168, 171, 174, 180-181, 189-190, 193-194, 200-202, 205-206, 211-212, 217-218, 223, 229, 235, 238-241, 244-247, 253, 261, 269, 274, 279, and 285; and optionally, the protein further comprises an amino-terminal methionine residue. In some embodiments, the CD20-binding molecule as described herein comprises or consists essentially of two proteins, each protein selected from any one of the polypeptides shown in SEQ ID NOs: 51-52, 59-61, 64-65, 71-73, 76-77, 82-83, 88-89, 94, 100, 106, 109-112, 115-118, 124, 132, 140, 145, 150, 156, 162, 168, 171, 174, 180-181, 189-190, 193-194, 200-202, 205-206, 211-212, 217-218, 223, 229, 235, 238-241, 244-247, 253, 261, 269, 274, 279, and 285; and optionally, each protein further comprises an amino-terminal methionine residue. In some embodiments, the protein is selected from any one of the proteins shown in SEQ ID NOs: 51-52, 59-61, 64-65, 71-73, 76-77, 82-83, 88-89, 94, 100, 106, 109-112, 115-118, 124, 132, 140, 145, 150, 156, 162, 168, 171, and 174, and further comprises a disulfide bond involving a cysteine residue at position 242, 482, 483, 484, 490, 491, 492, 493, 494, 495, 499, 500, 501, 502, 503, 504, 505, 510, 511, 512, 513, or 521.

For certain embodiments, upon administration of the CD20-binding molecule, and/or a composition thereof, to one or more cells physically coupled with CD20, which have the extracellular part bound by the two or more CD20 binding regions of the CD20-binding molecule, the CD20-binding molecule internalizes into one or more of the cells and delivers an additional exogenous material into the interior of the cell(s) or at least some of the cells.

For certain embodiments of the CD20-binding molecule as described herein and compositions thereof, a cellular internalization rate may be measured as the time after administration (on average) at which the CD20-binding molecule as described herein is observed inside a cell(s) and/or a majority of the cells contacted with the CD20-binding molecule and/or composition thereof. For example, the anti-CD20 monoclonal antibody rituximab typically reaches maximal cellular internalization at 37° C. after approximately 16 to 18 hours, and thus, in the context as described herein, a “rapid internalization” would indicate internalization rate several hours faster than that observed for rituximab, on average at the same temperature and receptor occupancy level.

For certain embodiments, upon administration of the CD20-binding molecule, and/or a composition thereof, to one or more cells physically coupled with CD20, which have the extracellular part bound by the two or more CD20 binding regions of the CD20-binding molecule, the CD20-binding molecule internalizes into one or more of the cells and delivers an additional exogenous material into the interior of the cell in about five hours, four hours, three hours, two hours, one hour, thirty minutes, or less at a physiological temperature appropriate for the cell and/or at about 37 degrees Celsius. In some embodiments, the cell(s) expresses at a cellular surface the CD20 which (1) have the extracellular part bound by the two or more CD20 binding regions of the CD20-binding molecule, (2) have a transmembrane domain, and (3) remain physically coupled to the cell. In some embodiments, the cell(s) is a CD20 positive cell. For certain embodiments, the cell(s) is physically coupled with a significant amount of extracellular CD20 which (1) have the extracellular part bound by the two or more CD20 binding regions of the CD20-binding molecule. For certain embodiments, the cell(s) is a descendant or member of a B-cell lineage. For certain embodiments, the cell(s) is a malignant B-cell, B-cell leukemia cell, B-cell lymphoma cell, B-cell myeloma cell, acute myeloid leukemia cell, acute non-lymphocytic leukemia cell, B-cell chronic lymphocytic leukemia cell, B-cell lymphoma cell, B-cell non-Hodgkin's lymphoma cell, B-cell precursor acute lymphoblastic leukemia cell, B-cell prolymphocytic leukemia cell, Burkitt's lymphoma cell, chronic lymphocytic leukemia cell, chronic myeloid leukemia cell, diffuse large B-cell lymphoma cell, follicular lymphoma cell, hairy cell leukemia cell, Hodgkin's lymphoma cell, immunoblastic large cell lymphoma cell, mantle cell lymphoma cell, melanoma cell, multiple myeloma cell, neoplastic plasma cell, nodular lymphocyte predominant Hodgkin's lymphoma cell, non-Hodgkin's lymphoma cell, plasmablastic lymphoma cell, plasma cell myeloma cell, precursor B-lymphoblastic lymphoma cell, small lymphocytic lymphoma cell, malignant T-cell, T-cell leukemia cell, T-cell lymphoma cell, T-cell large granular lymphocyte leukemia cell, T-cell prolymphocytic leukemia, healthy B-cell lineage cell, or healthy T-cell.

For purposes of certain embodiments as described herein, the phrase “in less than about thirty minutes” means that the maximal (or half-maximal in certain contexts) observed amount of intracellular CD20, CD20 antigen, and/or CD20-binding molecule during a internalization assay time course is observed at or before thirty minutes from the step of contacting CD20 positive cell(s) with the CD20-binding molecule as described herein as determined by an appropriate assay at conditions similar to 37° C. and 50 nM of CD20-binding molecule. The time of maximal or half-maximal intracellular accumulation may be determined by comparing intracellular accumulation at different times to find a peak or plateau. If a plateau is observed, then the maximal intracellular accumulation may be determined to be the first time the plateau reaches its highest point.

The extracellular CD20 cell surface density and the K_{D} of a CD20-binding molecule may be used to calculate the percent occupancy for a given concentration of CD20-binding molecule, such as a CD20-binding molecule as described herein or a reference CD20-binding molecule (e.g. monoclonal antibody) known to the skilled worker. For example, the CD20 receptor occupancy may be determined as a function of the 1) binding interaction between the extracellular CD20 receptor and CD20-binding molecule, 2) amount (e.g., concertation or effective concentration) of extracellular CD20 receptor available for binding, and 3) the amount (e.g., mass, concertation, or molarity) of CD20-binding molecule present in a given situation.

In some embodiments, internalization rates of a CD20-binding molecule as described herein compared to a CD20 antibody known in the art may be determined using assays performed at comparable extracellular CD20 receptor occupancies, instead of being determined using assays performed at comparable concentrations of the administered CD20-binding molecules (i.e. a CD20-binding molecule as described herein and a reference, anti-CD20 antibody of prior art). The percent CD20 receptor occupancy (RO_{CD20}) may be determined using models and formulae, such as, e.g.,

$${RO}_{CD 20} = \frac{\begin{matrix}{K}_{D} + {A}_{tot} + CD {20}_{tot} - \\ \sqrt{{( - {K}_{D} - {A}_{tot} - CD {20}_{tot} )}^{2} - 4 \cdot {A}_{tot} \cdot CD {20}_{tot}}\end{matrix}}{2 \cdot CD {20}_{tot}}$$

where RO is the receptor occupancy of the extracellular CD20 in the internalization assay, K_{D} is the dissociation constant of the CD20 binding molecule of interest to the extracellular CD20 receptor, Aₜₒₜ is the total number of CD20 binding molecules in the assay, and CD20ₜₒₜ is the total number of cell surface CD20 molecules in the assay.

In some embodiments of the CD20-binding molecule as described herein, which comprises an additional exogenous material; whereby upon administration of the CD20-binding molecule, or a composition thereof, to a plurality of cells physically coupled with CD20, which have the extracellular part bound by the two or more CD20 binding regions of the CD20-binding molecule, at a concentration of CD20-binding molecule equivalent to five or thirty-eight percent to fifty percent cell-surface occupancy, the majority of the CD20-binding molecule internalizes into the plurality of cells and delivers the additional exogenous material into the interiors of the majority of the plurality of cells in about five hours, four hours, three hours, two hours, one hour, thirty minutes, or less at a physiological temperature appropriate for the cell and/or at about 37 degrees Celsius. In some embodiments, members of the plurality of cells express at a cellular surface the CD20 which (1) have the extracellular part bound by the two or more CD20 binding regions of the CD20-binding molecule, (2) have a transmembrane domain, and (3) remain physically coupled to the cell. In some embodiments, members of the plurality of cells are CD20 positive cells. For certain embodiments, the members of the plurality of cells are physically coupled with a significant amount of extracellular CD20 which (1) have the extracellular part bound by the two or more CD20 binding regions of the CD20-binding molecule. In some embodiments, members of the plurality of cells are descendants or members of a B-cell lineage. For certain embodiments, members of the plurality of cells are members of the following group of cells: malignant B-cell, B-cell leukemia cell, B-cell lymphoma cell, B-cell myeloma cell, acute myeloid leukemia cell, acute non-lymphocytic leukemia cell, B-cell chronic lymphocytic leukemia cell, B-cell lymphoma cell, B-cell non-Hodgkin's lymphoma cell, B-cell precursor acute lymphoblastic leukemia cell, B-cell prolymphocytic leukemia cell, Burkitt's lymphoma cell, chronic lymphocytic leukemia cell, chronic myeloid leukemia cell, diffuse large B-cell lymphoma cell, follicular lymphoma cell, hairy cell leukemia cell, Hodgkin's lymphoma cell, immunoblastic large cell lymphoma cell, mantle cell lymphoma cell, melanoma cell, multiple myeloma cell, neoplastic plasma cell, nodular lymphocyte predominant Hodgkin's lymphoma cell, non-Hodgkin's lymphoma cell, plasmablastic lymphoma cell, plasma cell myeloma cell, precursor B-lymphoblastic lymphoma cell, small lymphocytic lymphoma cell, malignant T-cell, T-cell leukemia cell, T-cell lymphoma cell, T-cell large granular lymphocyte leukemia cell, T-cell prolymphocytic leukemia, healthy B-cell lineage cell, or healthy T-cell.

Because certain CD20-binding molecules as described herein, and compositions thereof, exhibit specific cell-targeting and efficient cellular internalization (e.g. within thirty minutes after administration), a cytotoxic cargo (such as, e.g., a cytotoxic agent, ribonucleic acid, antigen, and/or proapoptotic peptide) conjugated to a CD20-binding molecule as described herein can be efficiently delivered into a CD20-expressing cell for the purpose of killing the cell. Because certain CD20-binding molecules as described herein, and compositions thereof, exhibit selective cell-targeting and efficient cellular internalization (e.g. within thirty minutes after administration), a cytotoxic cargo (such as, e.g., a cytotoxic agent, ribonucleic acid, antigen, and/or proapoptotic peptide) conjugated to a CD20-binding molecule as described herein can be selectively and efficiently delivered into a CD20-expressing cell for the purpose of killing the cell in the presence of other cells, including other CD20-expressing cells which express lower levels of CD20 than the targeted cell(s).

### D. Information Gathering for Diagnostic Functions

Certain CD20-binding molecules as described herein, and compositions thereof, have uses in the in vitro and/or in vivo detection of specific cells, cell types, cell populations, and/or subcellular compartments of any of the foregoing. In some embodiments, the CD20-binding molecules as described herein, and compositions thereof, are used for both diagnosis and treatment, or for diagnosis alone. When the same cytotoxic CD20-binding molecule is used for both diagnosis and treatment, the cytotoxic CD20-binding molecule variant which incorporates a detection promoting agent for diagnosis may be rendered nontoxic or to exhibit reduced cytotoxicity by catalytic inactivation of its Shiga toxin effector region(s) via one or more amino acid substitutions, such as, e.g., illustrative substitutions described herein, e.g. such that at a given dose (e.g., less than 1 mg/kg) there is no observable reduction in a CD20 target positive cell. Reduced-cytotoxic variants may still be cytotoxic at certain concentrations or dosages but exhibit reduced cytotoxicity, such as, e.g., are not capable of exhibiting a significant level of Shiga toxin cytotoxicity in a given in vitro cell-kill assay. Nontoxic or reduced-cytotoxic forms of the cytotoxic CD20-binding molecules described herein that are conjugated to detection promoting agents optionally may be used for diagnostic functions, such as for companion diagnostics used in conjunction with a therapeutic regimen comprising the same or a related CD20 binding region for cell-targeting and/or involving the same or a related CD20 epitope for targeted therapy.

The ability to conjugate detection promoting agents known in the art to various cytotoxic CD20-binding molecules as described herein provides useful compositions for the detection of cancer, tumor, and immune cells, as well as subcellular compartments of the foregoing. These diagnostic embodiments of the CD20-binding molecules as described herein, and compositions thereof, may be used for information gathering via various imaging techniques and assays known in the art. For example, diagnostic embodiments of the CD20-binding molecules as described herein may be used for information gathering via imaging of intracellular organelles (e.g. endocytotic, Golgi, endoplasmic reticulum, and cytosolic compartments) of individual cancer cells, neoplastic cells, malignant tumor cells, non-malignant tumor cells, immune cells, and/or hematological cells in a patient or biopsy sample.

Various types of information may be gathered using the diagnostic embodiments of the CD20-binding molecules as described herein, and compositions thereof, whether for diagnostic uses or other uses. This information may be useful, for example, in diagnosing CD20 positive, neoplastic cell types; determining therapeutic susceptibilities of a patient's disease; assaying the progression of anti-neoplastic therapies over time; assaying the progression of immunomodulatory therapies over time; assaying the progression of antimicrobial therapies over time; evaluating the presence of unwanted CD20+ cell types in transplantation materials; and/or evaluating the presence of residual tumor cells after surgical excision of a tumor mass.

For example, subpopulations of patients might be ascertained using information gathered using the diagnostic variants of the CD20-binding molecules as described herein, and compositions thereof, and then individual patients could be further categorized into subpopulations based on their unique characteristic(s) revealed using those diagnostic embodiments. For example, the effectiveness of specific pharmaceuticals or therapies might be one type of criterion used to define a patient subpopulation. For example, a nontoxic diagnostic variant of a particular cytotoxic CD20-binding molecule as described herein, and/or composition thereof, may be used to differentiate which patients are in a class or subpopulation of patients predicted to respond positively to a cytotoxic variant of the same cytotoxic CD20-binding molecule or other, related, therapeutic molecule. Accordingly, associated methods for patient identification, patient stratification and diagnosis using cytotoxic CD20-binding molecules and their nontoxic or reduced-cytotoxic variants, as well as compositions thereof, are considered to be within the scope as described herein.

### IV. Variations in Proteinaceous Components of the CD20-Binding Molecules

The skilled worker will recognize that variations may be made to the CD20-binding molecules as described herein (and polynucleotides encoding them and/or their components) without diminishing their biological activities, e.g., by maintaining the overall structure and function of a given CD20-binding molecule. For example, some modifications may facilitate expression, facilitate purification, improve pharmacokinetic properties, improve protein stability, and/or improve immunogenicity. Such modifications are well known to the skilled worker and include, for example, a methionine added at the amino terminus to provide an initiation site, additional amino acids placed on either terminus to create conveniently located restriction sites or termination codons, and biochemical affinity tags fused to either terminus to provide for convenient detection and/or purification. A common modification to improve the immunogenicity of a polypeptide is to remove, after the production of the polypeptide, the starting methionine residue, which may be formulated during production in a bacterial host system, because, e.g., the presence of amino-terminal formylmethionine (fMet) might induce undesirable immune responses in chordates.

In some embodiments, the CD20-binding molecule as described herein is PEGylated, such as, e.g., to improve pharmacokinetic properties, to improve immunogenicity, and/or provide other benefit(s).

Also contemplated herein is the inclusion of additional amino acid residues at the amino and/or carboxy termini, such as sequences for epitope tags or other moieties. The additional amino acid residues may be used for various purposes including, e.g., to facilitate cloning, expression, post-translational modification, synthesis, purification, detection, and/or administration. Non-limiting examples of epitope tags and moieties are: chitin binding protein domains, enteropeptidase cleavage sites, Factor Xa cleavage sites, FIAsH tags, FLAG tags, green fluorescent proteins (GFP), glutathione-S-transferase moieties, HA tags, maltose binding protein domains, myc tags, polyhistidine tags, ReAsH tags, strep-tags, strep-tag II, TEV protease sites, thioredoxin domains, thrombin cleavage site, and V5 epitope tags.

In some embodiments, the CD20-binding molecule is a variant in which there are one or more conservative amino acid substitutions introduced into a proteinaceous region(s). As used herein, the term “conservative substitution” denotes that one or more amino acids are replaced by another, biologically similar amino acid residue. Examples include substitution of amino acid residues with similar characteristics, e.g. small amino acids, acidic amino acids, polar amino acids, basic amino acids, hydrophobic amino acids and aromatic amino acids (see, for example, Table 2 below). An example of a conservative substitution with a residue normally not found in endogenous, mammalian peptides and proteins is the conservative substitution of an arginine or lysine residue with, for example, ornithine, canavanine, aminoethylcysteine, or another basic amino acid. For further information concerning phenotypically silent substitutions in peptides and proteins see, e.g., Bowie J et al., Science 247: 1306-10 (1990).

TABLE 2
Examples of Conservative Amino Acid Substitutions

| I | II | III | IV | V | VI | VII | VIII | IX | X | XI | XII | XIII | XIV |
|---|---|---|---|---|---|---|---|---|---|---|---|---|---|
| A | D | H | C | F | N | A | C | F | A | C | A | A | D |
| G | E | K | I | W | Q | G | M | H | C | D | C | C | E |
| P | Q | R | L | Y | S | I | P | W | F | E | D | D | G |
| S | N | | M | | T | L | | Y | G | H | G | E | K |
| T | | | V | | | V | | | H | K | N | G | P |
| | | | | | | | | | I | N | P | H | Q |
| | | | | | | | | | L | Q | S | K | R |
| | | | | | | | | | M | R | T | N | S |
| | | | | | | | | | R | S | V | Q | T |
| | | | | | | | | | T | T | | R | |
| | | | | | | | | | V | | | S | |
| | | | | | | | | | W | | | P | |
| | | | | | | | | | Y | | | T | |

In the conservative substitution scheme in Table 2, illustrative conservative substitutions of amino acids are grouped by physicochemical properties—I: neutral, hydrophilic; II: acids and amides; III: basic; IV: hydrophobic; V: aromatic, bulky amino acids, VI hydrophilic uncharged, VII aliphatic uncharged, VIII non-polar uncharged, IX cycloalkenyl-associated, X hydrophobic, XI polar, XII small, XIII turn-permitting, and XIV flexible. For example, conservative amino acid substitutions include the following: 1) S may be substituted for C; 2) M or L may be substituted for F; 3) Y may be substituted for M; 4) Q or E may be substituted for K; 5) N or Q may be substituted for H; and 6) H may be substituted for N.

In some embodiments, a CD20-binding molecule as described herein or CD20-binding molecule composition as described herein may comprise a protein that has, at most, 20, 15, 10, 9, 8, 7, 6, 5, 4, 3, 2, or 1 amino acid substitution(s) compared to a protein sequence recited herein, as long as the CD20-binding molecule retains the requisite biological activity(ies).

Variants of CD20-binding molecules provided herein are within the scope as described herein as a result of changing a proteinaceous component of the CD20-binding molecule as described herein by altering one or more amino acids or deleting or inserting one or more amino acids, such as, e.g., within a CD20 binding region or Shiga toxin effector region, in order to achieve desired properties, such as, e.g., changed cytotoxicity, changed cytostatic effects, changed immunogenicity, and/or changed serum half-life. A polypeptide component of a CD20-binding molecule or CD20-binding molecule composition may further be with or without a signal sequence. In some embodiments, a proteinaceous component of a CD20-binding molecule or CD20-binding molecule composition shares at least 85%, 90%, 95%, 96%, 97%, 98%, 99% or more amino acid sequence identity to any one of the amino acid sequences of a molecule described herein, as long as the proteinaceous component retains, alone and/or as a component of a CD20-binding molecule, measurable biological activity, such as cytotoxicity, extracellular CD20 target biomolecule binding, enzymatic catalysis, or subcellular routing.

In some embodiments, a proteinaceous component of a CD20-binding molecule may comprise functional fragments or variants of a polypeptide region that have, at most, 20, 15, 10, 9, 8, 7, 6, 5, 4, 3, 2, or 1 amino acid substitution(s) compared to a polypeptide sequence described herein, as long as the substituted protein retains measurable biological activity alone and/or as a component of a CD20-binding molecule.

In some embodiments, a proteinaceous component of a CD20-binding molecule of a composition as described herein shares at least 85%, 90%, 95%, 96%, 97%, 98%, 99% or more amino acid sequence identity to any one of the amino acid sequences of a protein recited herein, as long as the protein retains measurable biological activity, such as cytotoxicity, extracellular CD20 target biomolecule binding, enzymatic catalysis, or subcellular routing. The CD20 binding regions of a CD20-binding molecule may differ from the amino acid sequences of a CD20 binding region described herein, as long as the CD20-binding region retains binding functionality to an extracellular part of CD20. Binding functionality will most likely be retained if the amino acid sequences of the CDRs or ABRs are identical. For example, a CD20-binding molecule is within the claim scope wherein the CD20-binding region comprises one or more CD20 binding regions comprising, consisting essentially of, or consisting of 85% amino acid identity to a CD20 binding region recited herein which for the purposes of determining the degree of amino acid identity, the amino acid residues that form the CDRs or ABRs are disregarded. Extracellular CD20 binding functionality can be determined by the skilled worker using standard techniques.

Also provided are variants of the CD20-binding molecules, wherein the Shiga toxin effector region differs from a naturally occurring Shiga toxin A Subunit by up to 1, 2, 3, 4, 5, 6, 7, 8, 9, 10, 15, 20, 25, 30, 35, 40 or more amino acid residues (but by no more than that which retains at least 85%, 90%, 95%, 99% or more amino acid sequence identity). Thus, a polypeptide region derived from an A Subunit of a member of the Shiga toxin family may comprise additions, deletions, truncations, or other alterations from the original sequence as long as at least 85%, 90%, 95%, 99% or more amino acid sequence identity is maintained to a naturally occurring Shiga toxin A Subunit.

Accordingly, in some embodiments, the Shiga toxin effector region comprises or consists essentially of amino acid sequences having at least 55%, 60%, 65%, 70%, 75%, 80%, 85%, 90%, 95%, 97%, 98%, 99%, 99.5% or 99.7% overall sequence identity to a naturally occurring Shiga toxin A Subunit, such as SLT-1A (SEQ ID NO:1), StxA (SEQ ID NO:2), and/or SLT-2A (SEQ ID NO:3).

Optionally, either a full-length or a truncated version of the Shiga toxin A Subunit may comprise one or more mutations (e.g. substitutions, deletions, insertions or inversions). It is preferred in some embodiments described herein that the Shiga toxin effector region has sufficient sequence identity to a naturally occurring Shiga toxin A Subunit to retain cytotoxicity after entry into a cell, either by well-known methods of host cell transformation, transfection, infection or induction, or by internalization mediated by the cell targeting CD20 binding region linked with the Shiga toxin effector region. The most critical residues for enzymatic activity and/or cytotoxicity in the Shiga toxin A Subunits have been mapped to the following residue-positions: aspargine-75, tyrosine-77, glutamate-167, arginine-170, and arginine-176 among others (Di R et al., Toxicon 57: 525-39 (2011)). In any one of the embodiments described herein, the Shiga toxin effector region may preferably but not necessarily maintain one or more conserved amino acids at positions, such as those found at positions 77, 167, 170, and 176 in StxA, SLT-1A, or the equivalent conserved position in other members of the Shiga toxin family which are typically required for cytotoxic activity. The capacity of a cytotoxic CD20-binding molecule as described herein to cause cell death, e.g. its cytotoxicity, may be measured using any one or more of a number of assays well known in the art.

In some embodiments of the CD20-binding molecules as described herein, one or more amino acid residues may be mutated, inserted, or deleted in order to increase the enzymatic activity of one or more of the molecule's Shiga toxin effector regions. For example, mutating residue-position alanine-231 in Stx1A to glutamate increased Stx1A's enzymatic activity in vitro (Suhan M, Hovde C, Infect Immun 66: 5252-9 (1998)).

In some embodiments of the CD20-binding molecules as described herein, one or more amino acid residues may be mutated or deleted in order to reduce or eliminate catalytic and/or cytotoxic activity of one or more of the molecule's Shiga toxin effector regions. The catalytic and/or cytotoxic activity of the A Subunits of members of the Shiga toxin family may be reduced or eliminated by mutation or truncation. The positions labeled tyrosine-77, glutamate-167, arginine-170, tyrosine-114, and tryptophan-203 have been shown to be important for the catalytic activity of Stx, Stx1, and Stx2. Mutating both glutamate-167 and arginine-170 eliminated the enzymatic activity of Slt-I A1 in a cell-free ribosome inactivation assay. In another approach using de novo expression of Slt-I A1 in the endoplasmic reticulum, mutating both glutamate-167 and arginine-170 or truncating it to residues 1-239 eliminated Slt-I A1 fragment cytotoxicity at that expression level.

In some embodiments, the Shiga toxin effector region(s) may be altered to change its enzymatic activity and/or cytotoxicity as long as the Shiga toxin effector region(s) retains one or more other Shiga toxin effector functions. This change may or may not result in a change in the cytotoxicity of a molecule of which the altered Shiga toxin effector region(s) is a component. Possible alterations include the following mutations to the Shiga toxin effector region(s): a truncation, deletion, inversion, insertion, rearrangement, and/or substitution.

The cytotoxicity of the A Subunits of members of the Shiga toxin family may be altered, reduced, or eliminated by mutation or truncation. The positions labeled tyrosine-77, glutamate-167, arginine-170, tyrosine-114, and tryptophan-203 have been shown to be important for the catalytic activity of Stx, Stx1, and Stx2. Mutating both glutamate-167 and arginine-170 eliminated the enzymatic activity of Slt-I A1 in a cell-free ribosome inactivation assay. In another approach using de novo expression of Slt-I A1 in the endoplasmic reticulum, mutating both glutamate-167 and arginine-170 eliminated Slt-I A1 fragment cytotoxicity at that expression level. A truncation analysis demonstrated that a fragment of StxA from residues 75 to 268 still retains significant enzymatic activity in vitro. A truncated fragment of Slt-I A1 containing residues 1-239 displayed significant enzymatic activity in vitro and cytotoxicity by de novo expression in the cytosol. Expression of a Slt-I A1 fragment truncated to residues 1-239 in the endoplasmic reticulum was not cytotoxic because the Slt-I A1 truncation could not retrotranslocate to the cytosol.

The most critical residues for enzymatic activity and/or cytotoxicity in the Shiga toxin A Subunits have been mapped to the following residue-positions: aspargine-75, tyrosine-77, glutamate-167, arginine-170, and arginine-176 among others. In particular, a double-mutant construct of Stx2A containing glutamate-E1 67-to-lysine and arginine-176-to-lysine mutations was completely inactivated; whereas, many single mutations in Stx1 and Stx2 showed a 10-fold reduction in cytotoxicity. Further, truncation of Stx1A to 1-239 or 1-240 reduced its cytotoxicity, and similarly, truncation of Stx2A to a conserved hydrophobic residue reduced its cytotoxicity.

The most critical residues for binding eukaryotic ribosomes and/or eukaryotic ribosome inhibition in the Shiga toxin A Subunit have been mapped to the following residue-positions arginine-172, arginine-176, arginine-179, arginine-188, tyrosine-189, valine-191, and leucine-233 among others.

Shiga-like toxin 1 A Subunit truncations are catalytically active, capable of enzymatically inactivating ribosomes in vitro, and cytotoxic when expressed within a cell. The smallest Shiga toxin A Subunit fragment exhibiting full enzymatic activity is a polypeptide composed of residues 1-239 of Slt1A. Although the smallest fragment of the Shiga toxin A Subunit reported to retain substantial catalytic activity was residues 75-247 of StxA, a StxA truncation expressed de novo within a eukaryotic cell requires only up to residue 240 to reach the cytosol from the endoplasmic reticulum and exert catalytic inactivation of ribosomes.

In certain CD20-binding molecules as described herein which comprise a Shiga toxin effector region derived from SLT-1A (SEQ ID NO:1) or StxA (SEQ ID NO:2), these mutational changes include substitution of the asparagine at position 75, tyrosine at position 77, tyrosine at position 114, glutamate at position 167, arginine at position 170, arginine at position 176, and/or substitution of the tryptophan at position 203. Examples of such substitutions will be known to the skilled worker based on the prior art, such as asparagine at position 75 to alanine, tyrosine at position 77 to serine, substitution of the tyrosine at position 114 to serine, substitution of the glutamate at position 167 to aspartate, substitution of the arginine at position 170 to alanine, substitution of the arginine at position 176 to lysine, and/or substitution of the tryptophan at position 203 to alanine.

In some embodiments, the CD20-binding molecule as described herein is de-immunized (see e.g., WO 2015/113005 and WO 2015/113007). In some embodiments, the de-immunized CD20-binding molecule as described herein comprises the protein shown in any one of SEQ ID NOs: 49-51, 63-64, 75-76, 81-82, 87-88, 93-94, 99-100, 105-106, 111-112, 117-118, 122-124, 131-132, 139-140, 144-145, 149-150, 155-156, 161-162, 167-168, 171, 174, 178-180, 192-193, 204-205, 210-211, 216-217, 222-223, 228-229, 234-235, 240-241, 246-247, 251-253, 260-261, 268-269, 273-274, 278-279, 284-285, 290, and 296; and optionally, the protein further comprises an amino-terminal methionine residue. In some embodiments, the CD20-binding molecule as described herein comprises or consists essentially of two proteins, each protein selected from any one of the polypeptides shown in SEQ ID NOs: 49-51, 63-64, 75-76, 81-82, 87-88, 93-94, 99-100, 105-106, 111-112, 117-118, 122-124, 131-132, 139-140, 144-145, 149-150, 155-156, 161-162, 167-168, 171, 174, 178-180, 192-193, 204-205, 210-211, 216-217, 222-223, 228-229, 234-235, 240-241, 246-247, 251-253, 260-261, 268-269, 273-274, 278-279, 284-285, 290, and 296; and optionally, each protein further comprises an amino-terminal methionine residue. In some embodiments, the protein is selected from any one of the proteins shown in SEQ ID NOs: 49-51, 63-64, 75-76, 81-82, 87-88, 93-94, 99-100, 105-106, 111-112, 117-118, 122-124, 131-132, 139-140, 144-145, 149-150, 155-156, 161-162, 167-168, 171, and 174, and further comprises a disulfide bond involving a cysteine residue at position 242, 482, 483, 484, 490, 491, 492, 493, 494, 495, 499, 500, 501, 502, 503, 504, 505, 510, 511, 512, 513, or 521.

CD20-binding molecules as described herein may optionally be conjugated to one or more additional agents, such as therapeutic and/or diagnostic agents known in the art, including such agents as described herein.

### V. Production, Manufacture, and Purification of CD20-Binding Molecules as Described Herein and Compositions Thereof

The CD20-binding molecules as described herein, and compositions thereof, may be produced using biochemical engineering techniques well known to those of skill in the art. For example, CD20-binding molecules as described herein, and compositions thereof, may be manufactured by standard synthetic methods, by use of recombinant expression systems, or by any other suitable method. The CD20-binding molecules as described herein may be produced as fusion proteins, chemically coupled conjugates, and/or combinations thereof, such as, e.g., a fusion protein component covalently linked to one or more other components of the CD20-binding molecule described herein. Thus, the CD20-binding molecules as described herein may be synthesized in a number of ways, including, e.g. methods comprising: (1) synthesizing a polypeptide or polypeptide component of a CD20-binding molecule described herein using standard solid-phase or liquid-phase methodology, either stepwise or by fragment assembly, and isolating and purifying the final proteinaceous compound product; (2) expressing a polynucleotide that encodes a polypeptide or polypeptide component of a CD20-binding molecule described herein in a host cell and recovering the expression product from the host cell or host cell culture; or (3) cell-free in vitro expression of a polynucleotide encoding a polypeptide or polypeptide component of a CD20-binding molecule described herein, and recovering the expression product; or by any combination of the methods of (1), (2) or (3) to obtain fragments of a proteinaceous component of a CD20-binding molecule described herein, subsequently joining (e.g. ligating) the fragments to obtain the proteinaceous component of a CD20-binding molecule described herein, and purifying or recovering that proteinaceous component. For example, polypeptide and/or peptide components may be ligated together using coupling reagents, such as, e.g., N,N′-dicyclohexycarbodiimide and N-ethyl-5-phenyl-isoxazolium-3′-sulfonate (Woodward's reagent K).

It may be preferable to synthesize a polypeptide or polypeptide component of a CD20-binding molecule as described herein by means of solid-phase or liquid-phase peptide synthesis. CD20-binding molecules as described herein may suitably be manufactured by standard synthetic methods. Thus, polypeptides may be synthesized by, e.g. methods comprising synthesizing the polypeptide by standard solid-phase or liquid-phase methodology, either stepwise or by fragment assembly, and isolating and purifying the final polypeptide product. In this context, reference may be made to WO 1998/11125 or, inter alia, Fields G et al., Principles and Practice of Solid-Phase Peptide Synthesis (Synthetic Peptides, Grant G, ed., Oxford University Press, U.K., 2nd ed., 2002) and the synthesis examples therein.

CD20-binding molecules as described herein may be prepared (produced and purified) using recombinant techniques well known in the art. In general, methods for preparing proteins by culturing host cells transformed or transfected with a vector comprising the encoding polynucleotide and recovering the protein from cell culture are described in, e.g. Sambrook J et al., Molecular Cloning: A Laboratory Manual (Cold Spring Harbor Laboratory Press, NY, U.S., 1989); Dieffenbach C et al., PCR Primer: A Laboratory Manual (Cold Spring Harbor Laboratory Press, N.Y., U.S., 1995). Any suitable host cell may be used to produce a proteinaceous component of a CD20-binding molecule as described herein and/or a CD20-binding protein described herein. Host cells may be cells stably or transiently transfected, transformed, transduced or infected with one or more expression vectors which drive expression of a CD20-binding protein described herein and/or a proteinaceous component of a CD20-binding molecule as described herein. In addition, a CD20-binding protein as described herein may be produced by modifying the polynucleotide encoding the CD20-binding molecule described herein, or proteinaceous component thereof, described herein that result in altering one or more amino acids or deleting or inserting one or more amino acids in order to achieve desired properties, such as changed cytotoxicity, changed cytostatic effects, changed immunogenicity, and/or changed serum half-life.

There are a wide variety of expression systems which may be chosen to produce a CD20-binding protein described herein and/or a proteinaceous component of a CD20-binding molecule as described herein. For example, host organisms for expression of a CD20-binding protein described herein and/or a proteinaceous component of a CD20-binding molecule as described herein include prokaryotes, such as E. coli and B. subtilis, eukaryotic cells, such as yeast and filamentous fungi (like S. cerevisiae, P. pastoris, A. awamori, and K. lactis), algae (like C. reinhardtii), insect cell lines, mammalian cells (like CHO cells), plant cell lines, and eukaryotic organisms such as transgenic plants (like A. thaliana and N. benthamiana).

Also provided herein are methods for producing a CD20-binding molecule according to above recited methods and using (i) a polynucleotide encoding part or all of a proteinaceous component of a CD20-binding molecule as described herein and/or a CD20-binding protein as described herein, (ii) an expression vector comprising at least one polynucleotide described herein capable of encoding part or all of a CD20-binding molecule as described herein and/or a CD20-binding protein as described herein when introduced into a suitable host cell or cell-free expression system, and/or (iii) a host cell comprising a polynucleotide or expression vector as described herein.

When a protein is expressed using recombinant techniques in a host cell or cell-free system, it is advantageous to separate (or purify) the desired protein away from other components, such as host cell factors, in order to obtain preparations that are of high purity or are substantially homogeneous. Purification can be accomplished by methods well known in the art, such as centrifugation techniques, extraction techniques, chromatographic and fractionation techniques (e.g. size separation by gel filtration, charge separation by ion-exchange column, hydrophobic interaction chromatography, reverse phase chromatography, chromatography on silica or cation-exchange resins such as diethylaminoethyl (DEAE) resins and the like, chromatofocusing, and Protein A Sepharose chromatography to remove contaminants), and precipitation techniques (e.g. ethanol precipitation or ammonium sulfate precipitation). Any number of biochemical purification techniques may be used to increase the purity of a CD20-binding molecule as described herein. In some embodiments, the CD20-binding molecules as described herein may optionally be purified in homo-multimeric forms (e.g. a protein complex of two or more identical, CD20-binding proteins) or in hetero-multimeric forms (e.g. a protein complex of two or more non-identical CD20-binding proteins).

In the Examples below are descriptions of non-limiting examples of methods for producing a CD20-binding molecule as described herein and compositions thereof, as well as specific but non-limiting aspects of production of certain, disclosed, illustrative, cytotoxic CD20-binding molecules as described herein.

### VI. CD20-Binding Molecules Immobilized on Solid Substrates

Certain embodiments as described herein include a molecule as described herein (e.g., a CD20-binding molecule or any effector fragment thereof) immobilized on a solid substrate. Solid substrates contemplated herein include, but are not limited to, microbeads, nanoparticles, polymers, matrix materials, microarrays, microtiter plates, or any solid surface known in the art (see e.g. U.S. Pat. No. 7,771,955). In accordance with these embodiments, a molecule as described herein may be covalently or non-covalently linked to a solid substrate, such as, e.g., a bead, particle, or plate, using techniques known to the skilled worker (see e.g. Jung Y et al., Analyst 133: 697-701 (2008)). Immobilized molecules described herein may be used for screening applications using techniques known in the art (see e.g. Bradbury A et al., Nat Biotechnol 29: 245-54 (2011); Sutton C, Br J Pharmacol 166: 457-75 (2012); Diamante L et al., Protein Eng Des Sel 26: 713-24 (2013); Houlihan G et al., J Immunol Methods 405: 47-56 (2014)).

Non-limiting examples of solid substrates to which a molecule as described herein may be immobilized on include: microbeads, nanoparticles, polymers, nanopolymers, nanotubes, magnetic beads, paramagnetic beads, superparamagnetic beads, streptavidin coated beads, reverse-phase magnetic beads, carboxy terminated beads, hydrazine terminated beads, silica (sodium silica) beads, iminodiacetic acid (IDA)-modified beads, aldehyde-modified beads, epoxy-activated beads, diaminodipropylamine (DADPA)-modified beads (beads with primary amine surface group), biodegradable polymeric beads, polystyrene substrates, amino-polystyrene particles, carboxyl-polystyrene particles, epoxy-polystyrene particles, dimethylamino-polystyrene particles, hydroxy-polystyrene particles, colored particles, flow cytometry particles, sulfonate-polystyrene particles, nitrocellulose surfaces, reinforced nitrocellulose membranes, nylon membranes, glass surfaces, activated glass surfaces, activated quartz surfaces, polyvinylidene difluoride (PVDF) membranes, polyacrylamide-based substrates, poly-vinyl chloride substrates, poly-methyl methacrylate substrates, poly(dimethyl siloxane) substrates, and photopolymers which contain photoreactive species (such as, e.g., nitrenes, carbenes, and ketyl radicals) capable of forming covalent linkages. Other examples of solid substrates to which a molecule as described herein may be immobilized on are commonly used in molecular display systems, such as, e.g., cellular surfaces, phages, and virus particles.

### VII. Pharmaceutical and Diagnostic Compositions Comprising a CD20-Binding Molecule

Described herein are CD20-binding molecules for use, alone or in combination with one or more additional therapeutic agents, in a pharmaceutical composition, for treatment or prophylaxis of conditions, diseases, disorders, or symptoms described in further detail below (e.g., cancers, malignant tumors, non-malignant tumors, growth abnormalities, and immune disorders). Also provided herein are pharmaceutical compositions comprising a CD20-binding molecule, or a pharmaceutically acceptable salt or solvate thereof, together with at least one pharmaceutically acceptable carrier, excipient, or vehicle. In some embodiments, the pharmaceutical composition as described herein may comprise CD20-binding molecules that are homo-multimeric and/or hetero-multimeric. The pharmaceutical compositions as described herein are useful in methods of treating, ameliorating, or preventing a disease, condition, disorder, or symptom described in further detail below. Each such disease, condition, disorder, or symptom is envisioned to be a separate embodiment with respect to uses of a pharmaceutical composition as described herein. Also provided are pharmaceutical compositions for use in at least one method of treatment, as described in more detail below.

As used herein, the terms “patient” and “subject” are used interchangeably to refer to any organism, commonly vertebrates such as humans and animals, which presents symptoms, signs, and/or indications of at least one disease, disorder, or condition. These terms include mammals such as the non-limiting examples of primates, livestock animals (e.g. cattle, horses, pigs, sheep, goats, etc.), companion animals (e.g. cats, dogs, etc.) and laboratory animals (e.g. mice, rabbits, rats, etc.).

As used herein, “treat,” “treating,” or “treatment” and grammatical variants thereof refer to an approach for obtaining beneficial or desired clinical results. The terms may refer to slowing the onset or rate of development of a condition, disorder or disease, reducing or alleviating symptoms associated with it, generating a complete or partial regression of the condition, or some combination of any of the above. Beneficial or desired clinical results may include, but are not limited to, reduction or alleviation of symptoms, diminishment of extent of disease, stabilization (e.g. not worsening) of state of disease, delay or slowing of disease progression, amelioration or palliation of the disease state, and remission (whether partial or total), whether detectable or undetectable. “Treat,” “treating,” or “treatment” can also mean prolonging survival relative to expected survival time if not receiving treatment. A subject (e.g. a human) in need of treatment may thus be a subject already afflicted with the disease or disorder in question. The terms “treat,” “treating,” or “treatment” includes inhibition or reduction of an increase in severity of a pathological state or symptoms relative to the absence of treatment, and is not necessarily meant to imply complete cessation of the relevant disease, disorder, or condition. With regard to tumors and/or cancers, treatment includes reductions in overall tumor burden and/or individual tumor size.

As used herein, the terms “prevent,” “preventing,” “prevention” and grammatical variants thereof refer to an approach for preventing the development of, or altering the pathology of, a condition, disease, or disorder. Accordingly, “prevention” may refer to prophylactic or preventive measures. Beneficial or desired clinical results may include, but are not limited to, prevention or slowing of symptoms, progression or development of a disease, whether detectable or undetectable. A subject (e.g. a human) in need of prevention may thus be a subject not yet afflicted with the disease or disorder in question. The term “prevention” includes slowing the onset of disease relative to the absence of treatment, and is not necessarily meant to imply permanent prevention of the relevant disease, disorder or condition. Thus “preventing” or “prevention” of a condition may in certain contexts refer to reducing the risk of developing the condition, or preventing or delaying the development of symptoms associated with the condition.

As used herein, an “effective amount” or “therapeutically effective amount” is an amount or dose of a composition (e.g. a therapeutic composition, compound, or agent) that produces at least one desired therapeutic effect in a subject, such as preventing or treating a target condition or beneficially alleviating a symptom associated with the condition. The most desirable therapeutically effective amount is an amount that will produce a desired efficacy of a particular treatment selected by one of skill in the art for a given subject in need thereof. This amount will vary depending upon a variety of factors understood by the skilled worker, including but not limited to the characteristics of the therapeutic composition (including activity, pharmacokinetics, pharmacodynamics, and bioavailability), the physiological condition of the subject (including age, sex, disease type, disease stage, general physical condition, responsiveness to a given dosage, and type of medication), the nature of the pharmaceutically acceptable carrier or carriers in the formulation, and the route of administration. One skilled in the clinical and pharmacological arts will be able to determine a therapeutically effective amount through routine experimentation, namely by monitoring a subject's response to administration of a composition and adjusting the dosage accordingly (see e.g. Remington: The Science and Practice of Pharmacy (Gennaro A, ed., Mack Publishing Co., Easton, Pa., U.S., 19th ed., 1995)).

Production or Manufacture of a Pharmaceutical and/or Diagnostic Composition Comprising a CD20-Binding Molecule as Described Herein and/or a Composition Thereof

Pharmaceutically acceptable salts or solvates of any of the CD20-binding molecules as described herein are within the scope as described herein.

The term “solvate” in the context as described herein refers to a complex of defined stoichiometry formed between a solute (e.g. in casu, a proteinaceous compound or pharmaceutically acceptable salt thereof) and a solvent. The solvent in this connection may, for example, be water, ethanol or another pharmaceutically acceptable, typically small-molecular organic species, such as, but not limited to, acetic acid or lactic acid. When the solvent in question is water, such a solvate is normally referred to as a hydrate.

CD20-binding molecules as described herein, or salts thereof, may be formulated as pharmaceutical compositions prepared for storage or administration, which typically comprise a therapeutically effective amount of a molecule or composition as described herein, or a salt thereof, in a pharmaceutically acceptable carrier. The term “pharmaceutically acceptable carrier” includes any of the standard pharmaceutical carriers. Pharmaceutically acceptable carriers for therapeutic use are well known in the pharmaceutical art, and are described, for example, in Remington's Pharmaceutical Sciences (Mack Publishing Co. (A. Gennaro, ed., 1985)). As used herein, “pharmaceutically acceptable carrier” includes any and all physiologically acceptable, i.e. compatible, solvents, dispersion media, coatings, antimicrobial agents, isotonic, and absorption delaying agents, and the like. Pharmaceutically acceptable carriers or diluents include those used in formulations suitable for oral, rectal, nasal or parenteral (including subcutaneous, intramuscular, intravenous, intradermal, and transdermal) administration. Illustrative pharmaceutically acceptable carriers include sterile aqueous solutions or dispersions and sterile powders for the extemporaneous preparation of sterile injectable solutions or dispersions. Examples of suitable aqueous and nonaqueous carriers that may be employed in the pharmaceutical compositions described herein include water, ethanol, polyols (such as glycerol, propylene glycol, polyethylene glycol, and the like), and suitable mixtures thereof, vegetable oils, such as olive oil, and injectable organic esters, such as ethyloleate. Proper fluidity can be maintained, for example, by the use of coating materials, such as lecithin, by the maintenance of the required particle size in the case of dispersions, and by the use of surfactants. In some embodiments, the carrier is suitable for intravenous, intramuscular, subcutaneous, parenteral, spinal or epidermal administration (e.g. by injection or infusion). Depending on selected route of administration, the CD20-binding molecule as described herein or other pharmaceutical component may be coated in a material intended to protect the CD20-binding molecule and/or a compound thereof from the action of low pH and other natural inactivating conditions to which the active CD20-binding molecule described herein may encounter when administered to a patient by a particular route of administration.

The pharmaceutical compositions as described herein may also contain adjuvants such as preservatives, wetting agents, emulsifying agents and dispersing agents. Preventing the presence of microorganisms may be ensured both by sterilization procedures, and by the inclusion of various antibacterial and antifungal agents, for example, paraben, chlorobutanol, phenol sorbic acid, and the like. Isotonic agents, such as sugars, sodium chloride, and the like into the compositions, may also be desirable. In addition, prolonged absorption of the injectable pharmaceutical form may be brought about by the inclusion of agents which delay absorption such as, aluminum monostearate and gelatin.

A pharmaceutical composition as described herein also optionally includes a pharmaceutically acceptable antioxidant. Illustrative pharmaceutically acceptable antioxidants are water soluble antioxidants such as ascorbic acid, cysteine hydrochloride, sodium bisulfate, sodium metabisulfite, sodium sulfite and the like; oil-soluble antioxidants, such as ascorbyl palmitate, butylated hydroxyanisole (BHA), butylated hydroxytoluene (BHT), lecithin, propylgallate, alpha-tocopherol, and the like; and metal chelating agents, such as citric acid, ethylenediamine tetraacetic acid (EDTA), sorbitol, tartaric acid, phosphoric acid, and the like.

Also provided herein are pharmaceutical compositions comprising one or a combination of different CD20-binding molecules as described herein, or an ester, salt or amide of any of the foregoing, and at least one pharmaceutically acceptable carrier.

A pharmaceutical composition as described herein optionally includes a pharmaceutically acceptable excipient. Non-limiting examples of pharmaceutically acceptable excipients include arginine, arginine sulfate, citric acid, glycerol, hydrochloric acid, mannitol, methionine, polysorbate, sodium chloride, sodium citrate, sodium hydroxide, sorbitol, sucrose, trehalose, and/or water. In some embodiments, the pharmaceutical composition as described herein comprises an aqueous carrier and at least one pharmaceutically acceptable excipient. In certain other embodiments, the pharmaceutical composition as described herein comprises a salt and/or powder, such as, e.g. a freeze-dried, lyophilized, dehydrated, and/or cryodesiccated composition comprising at least one pharmaceutically acceptable excipient. In some embodiments of the pharmaceutical composition as described herein, the excipient functions to reduce and/or limit the immunogenicity and/or immunogenic potential of the CD20-binding molecule, such as, e.g. after administration and/or repeated administration to a mammal.

The pharmaceutical compositions as described herein may comprise one or more adjuvants such as a buffer, tonicity-adjusting agent (isotonic agent), antioxidant, surfactant, stabilizer, preservative, emulsifying agent, cryoprotective agent, wetting agent, and/or dispersing agent or other additives well known to those of skill in the art, such as, e.g. a binding agent. In some embodiments, the pharmaceutical composition as described herein comprises an aqueous carrier and a pharmaceutically acceptable adjuvant or other additive. In certain other embodiments, the pharmaceutical composition as described herein comprises a salt and/or powder, such as, e.g. a freeze-dried, lyophilized, dehydrated, and/or cryodesiccated composition comprising a pharmaceutically acceptable adjuvant or other additive. Non-limiting examples of pharmaceutically suitable stabilizers include human albumin and polysorbates such as, e.g., polyoxyethylene (20) sorbitan monolaurate (polysorbate 20), polyoxyethylene (20) sorbitan monopalmitate (polysorbate 40), polyoxyethylene (20) sorbitan monostearate (polysorbate 60), and (polyoxyethylene (20) sorbitan monooleate (polysorbate 80).

The pharmaceutical composition as described herein may comprise one or more pharmaceutically acceptable buffers. Non-limiting examples of suitable buffers include acetate, citrate, citric acid, histidine, phosphate, sodium citrate, and succinate buffers. In some embodiments, the pharmaceutical composition as described herein comprises an aqueous carrier comprising a pharmaceutically acceptable buffer. In some embodiments, the pharmaceutical composition as described herein comprises a salt and/or powder, such as, e.g. a freeze-dried, lyophilized, dehydrated, and/or cryodesiccated composition comprising a pharmaceutically acceptable buffer.

The pharmaceutical composition as described herein may comprise one or more pharmaceutically acceptable isotonic agents or tonicity-adjusting agents. Non-limiting examples of suitable isotonic agents include sugars (e.g. dextrose), sugar alcohols, sodium chloride, and the like. Further examples of suitable sugars include disaccharides like sucrose and trehalose. Illustrative, pharmaceutically acceptable sugar alcohols include glycerol, mannitol, and sorbitol. In some embodiments, the pharmaceutical composition as described herein comprises an aqueous carrier and a pharmaceutically acceptable isotonic agent. In some embodiments, the pharmaceutical composition as described herein comprises a salt and/or powder, such as, e.g. a freeze-dried, lyophilized, dehydrated, and/or cryodesiccated composition comprising a pharmaceutically acceptable isotonic agent.

The pharmaceutical compositions as described herein may comprise one or more pharmaceutically acceptable antioxidants. Illustrative pharmaceutically acceptable antioxidants include water soluble antioxidants, such as, e.g., ascorbic acid, cysteine hydrochloride, methionine, sodium bisulfate, sodium metabisulfite, sodium sulfite and the like; oil-soluble antioxidants, such as, e.g., ascorbyl palmitate, butylated hydroxyanisole (BHA), butylated hydroxytoluene (BHT), lecithin, propylgallate, alpha-tocopherol, and the like; and metal-chelating agents, such as, e.g., citric acid, ethylenediamine tetraacetic acid (EDTA), sorbitol, tartaric acid, phosphoric acid, and the like. In some embodiments, the pharmaceutical composition as described herein comprises an aqueous carrier and a pharmaceutically acceptable antioxidant. In certain other embodiments, the pharmaceutical composition as described herein comprises a salt and/or powder, such as, e.g. a freeze-dried, lyophilized, dehydrated, and/or cryodesiccated composition comprising a pharmaceutically acceptable antioxidant.

A pharmaceutical composition as described herein may comprise one or more pharmaceutically acceptable surfactants and/or emulsifying agents (emulsifiers). Non-limiting examples of suitable surfactants and/or emulsifiers include polysorbates such as, e.g., polyoxyethylene (20) sorbitan monolaurate (polysorbate 20), polyoxyethylene (20) sorbitan monopalmitate (polysorbate 40), polyoxyethylene (20) sorbitan monostearate (polysorbate 60), and (polyoxyethylene (20) sorbitan monooleate (polysorbate 80). In some embodiments, the pharmaceutical composition as described herein comprises an aqueous carrier and a pharmaceutically acceptable surfactant and/or emulsifier. In some embodiments, the pharmaceutical composition as described herein comprises a salt and/or powder, such as, e.g. a freeze-dried, lyophilized, dehydrated, and/or cryodesiccated composition comprising a pharmaceutically acceptable surfactant and/or emulsifier. One or more surfactants and/or emulsifying agents may also be desirable in a pharmaceutical composition as described herein to help prevent aggregation of the cell-targeting molecule as described herein. The pharmaceutical compositions as described herein may comprise one or more pharmaceutically acceptable preservative agents. For example, preventing the presence of microorganisms may be ensured both by sterilization procedures, and by the inclusion of various antibacterial and antifungal agents, such as, e.g., paraben, chlorobutanol, phenol sorbic acid, and the like in the compositions as described herein.

A pharmaceutical composition as described herein may comprise one or more pharmaceutically acceptable cryoprotective agents, also referred to as cryoprotectants or cryogenic protectants. Non-limiting examples of suitable cryoprotectants include ethylene glycol, glycerol, sorbitol, sucrose, and trehalose. In some embodiments, the pharmaceutical composition as described herein comprises an aqueous carrier and a pharmaceutically acceptable cryoprotectant. In certain other embodiments, the pharmaceutical composition as described herein comprises a salt and/or powder, such as, e.g. a freeze-dried, lyophilized, dehydrated, and/or cryodesiccated composition comprising a pharmaceutically acceptable cryoprotectant.

In addition, prolonged absorption of the injectable pharmaceutical form may be brought about by the inclusion of agents which delay absorption such as, e.g., a monostearate salt, aluminum monostearate, and/or gelatin.

Also provided herein are pharmaceutical compositions comprising one or a combination of different polypeptides and/or cell-targeting molecules described herein, or an ester, salt or amide of any of the foregoing, and at least one pharmaceutically acceptable carrier.

The pH of the pharmaceutical composition as described herein can be adjusted with acids or bases, such as hydrochloric acid or sodium hydroxide, or buffers with acetate, citrate, citric acid, histidine, sodium citrate, succinate, phosphate, and the like. Non-limiting examples of pharmaceutically acceptable solvents or carriers for use in a pharmaceutical composition as described herein include aqueous solutions comprising a cell-targeting molecule as described herein and a buffer such as, e.g., citrate, histidine, phosphate, or succinate adjusted to pH 5.0, 6.0, 7.0, or 4.0, respectively. Certain embodiments as described herein include compositions comprising one of the aforementioned solvents and/or carriers as described herein.

Pharmaceutical compositions as described herein that are solutions or suspensions used for intradermal or subcutaneous application typically include one or more of: a sterile diluent such as water for injection, saline solution, fixed oils, polyethylene glycols, glycerine, propylene glycol or other synthetic solvents; antibacterial agents such as benzyl alcohol or methyl parabens; antioxidants such as ascorbic acid, cysteine hydrochloride, methionine, sodium bisulfate, sodium metabisulfite, and sodium sulfite; chelating agents such as citric acid, ethylenediaminetetraacetic acid, sorbitol, tartaric acid, and phosphoric acid; surfactants such as a polysorbate; buffers such as acetate, citrate, histidine, and phosphate buffers; and tonicity adjusting agents such as, e.g., dextrose, glycerol, mannitol, sodium chloride, sorbitol, sucrose, and trehalose. Such preparations may be enclosed in ampoules, disposable syringes or multiple dose vials made of a glass or plastic.

Sterile injectable solutions may be prepared by incorporating a protein or cell-targeting molecule as described herein in the required amount in an appropriate solvent with one or a combination of ingredients described above, as required, followed by sterilization microfiltration. Dispersions may be prepared by incorporating the active compound into a sterile vehicle that contains a dispersion medium and other ingredients, such as those described above. In the case of sterile powders for the preparation of sterile injectable solutions, the methods of preparation are vacuum drying and freeze-drying (lyophilization) that yield a powder of the active ingredient in addition to any additional desired ingredient from a sterile-filtered solution thereof. In some embodiments, the pharmaceutical composition as described herein comprises a powder comprising sorbitol, trehalose, sodium citrate, and polysorbate-20, and optionally, further comprises glycerol and/or methionine. In some embodiments, the pharmaceutical composition as described herein comprises sodium citrate, trehalose, and polysorbate-20, and optionally, further comprises glycerol and/or methionine.

In some embodiments, the pharmaceutical composition as described herein comprises sorbitol, sodium citrate, and polysorbate-20, and optionally, further comprises albumin, glycerol, and/or methionine. In some embodiments, the pharmaceutical composition as described herein comprises sorbitol, histidine, and polysorbate-20, and optionally, further comprises albumin, glycerol, and/or methionine.

In some embodiments, the pharmaceutical compositions comprise a CD20-binding molecule, wherein the pharmaceutical composition comprises at least one pharmaceutically acceptable excipient or carrier. In some embodiments, the pharmaceutical composition comprises sorbitol, sodium citrate and polysorbate-20. In some embodiments, the pharmaceutical composition comprises about 5 to about 500 mM sorbitol; about 1 to about 50 mM sodium citrate, and/or about 0.01 to about 1% polysorbate 20. In some embodiments, the pH of the pharmaceutical compositions is about 4.5 to about 7.5, such as about 4.5, about 5.0, about 5.5, about 6.0, about 6.5, about 7.0 or about 7.5. In some embodiments, the pharmaceutical composition comprises 200 mM sorbitol, 20 mM sodium citrate and 0.1% (v/v) polysorbate-20; and wherein the pharmaceutical composition has a pH of about 5.5. In some embodiments, the pharmaceutical composition comprises a CD20-binding molecule comprising the sequence of SEQ ID NO: 54, wherein the pharmaceutical compositions comprises 200 mM sorbitol, 20 mM sodium citrate and 0.1% (v/v) polysorbate-20; and wherein the pharmaceutical composition has a pH of about 5.5.

In some embodiments, the pharmaceutical composition is provided as a concentrated solution, which is diluted before administration to a subject. The pharmaceutical composition may be diluted using standard diluents commonly used in the art, such as saline or water. In some embodiments, the concentrated form of the pharmaceutical composition comprises about 5 to about 500 mM sorbitol; about 1 to about 50 mM sodium citrate, and/or about 0.01 to about 1% polysorbate 20. In some embodiments, the concentrated form of the pharmaceutical composition comprises 200 mM sorbitol, 20 mM sodium citrate and 0.1% (v/v) polysorbate-20. In some embodiments, the concentrated form of the pharmaceutical composition comprises a CD20-binding molecule comprising the sequence of SEQ ID NO: 54, wherein the pharmaceutical compositions comprises 200 mM sorbitol, 20 mM sodium citrate and 0.1% (v/v) polysorbate-20; and wherein the pharmaceutical composition has a pH of about 5.5. In some embodiments, the concentrated form of the pharmaceutical composition is diluted 2-fold, 5-fold, 10-fold, 20-fold, 25-fold, 50-fold, 100-fold, 250-fold, 500-fold, 1,000-fold, 1,500-fold, 2,000-fold, 5,000-fold, 10,000-fold, or more before use.

In some embodiments, the diluted form of the pharmaceutical composition is administered to a subject. In some embodiments, the diluted form of the pharmaceutical composition comprises about 5 to about 500 mM sorbitol; about 1 to about 50 mM sodium citrate, and/or about 0.01 to about 1% polysorbate 20. In some embodiments, the diluted form of the pharmaceutical composition comprises 200 mM sorbitol, 20 mM sodium citrate and 0.1% (v/v) polysorbate-20. In some embodiments, the diluted form of the pharmaceutical composition comprises a CD20-binding molecule comprising the sequence of SEQ ID NO: 54, wherein the pharmaceutical compositions comprises 200 mM sorbitol, 20 mM sodium citrate and 0.1% (v/v) polysorbate-20; and wherein the pharmaceutical composition has a pH of about 5.5.

The formulations of the pharmaceutical compositions as described herein may conveniently be presented in unit dosage form and may be prepared by any of the methods well known in the art of pharmacy. In such form, the composition is divided into unit doses containing appropriate quantities of the active component. The unit dosage form can be a packaged preparation, the package containing discrete quantities of the preparations, for example, packeted tablets, capsules, and powders in vials or ampoules. The unit dosage form can also be a capsule, cachet, or tablet itself, or it can be the appropriate number of any of these packaged forms. It may be provided in single dose injectable form, for example in the form of a pen. Compositions may be formulated for any suitable route and means of administration. Subcutaneous or transdermal modes of administration may be particularly suitable for pharmaceutical compositions and therapeutic molecules described herein.

Therapeutic compositions are typically sterile and stable under the conditions of manufacture and storage. The composition may be formulated as a solution, microemulsion, liposome, or other ordered structure suitable to high drug concentration. The carrier may be a solvent or dispersion medium containing, for example, water, alcohol such as ethanol, polyol (e.g. glycerol, propylene glycol, and liquid polyethylene glycol), or any suitable mixtures. The proper fluidity may be maintained, for example, by the use of a coating such as lecithin, by the maintenance of the required particle size in the case of dispersion and by use of surfactants according to formulation chemistry well known in the art. In some embodiments, isotonic agents, e.g. sugars, polyalcohols such as mannitol, sorbitol, or sodium chloride may be desirable in the composition. Prolonged absorption of injectable compositions may be brought about by including in the composition an agent that delays absorption for example, monostearate salts and gelatin.

Solutions or suspensions used for intradermal or subcutaneous application typically include one or more of: a sterile diluent such as water for injection, saline solution, fixed oils, polyethylene glycols, glycerine, propylene glycol or other synthetic solvents; antibacterial agents such as benzyl alcohol or methyl parabens; antioxidants such as ascorbic acid or sodium bisulfite; chelating agents such as ethylenediaminetetraacetic acid; buffers such as acetates, citrates or phosphates; and tonicity adjusting agents such as, e.g., sodium chloride or dextrose. The pH can be adjusted with acids or bases, such as hydrochloric acid or sodium hydroxide, or buffers with citrate, phosphate, acetate and the like. Such preparations may be enclosed in ampoules, disposable syringes or multiple dose vials made of glass or plastic.

Sterile injectable solutions may be prepared by incorporating a CD20-binding molecule as described herein in the required amount in an appropriate solvent with one or a combination of ingredients described above, as required, followed by sterilization microfiltration. Dispersions may be prepared by incorporating the active compound into a sterile vehicle that contains a dispersion medium and other ingredients, such as those described above. In the case of sterile powders for the preparation of sterile injectable solutions, the methods of preparation are vacuum drying and freeze-drying (lyophilization) that yield a powder of the active ingredient in addition to any additional desired ingredient from a sterile-filtered solution thereof.

When a therapeutically effective amount of a CD20-binding molecule as described herein is designed to be administered by, e.g. intravenous, cutaneous or subcutaneous injection, the binding agent will be in the form of a pyrogen-free, parenterally acceptable aqueous solution. Methods for preparing parenterally acceptable protein solutions, taking into consideration appropriate pH, isotonicity, stability, and the like, are within the skill in the art. A preferred pharmaceutical composition for intravenous, cutaneous, or subcutaneous injection will contain, in addition to binding agents, an isotonic vehicle such as sodium chloride injection, Ringer's injection, dextrose injection, dextrose and sodium chloride injection, lactated Ringer's injection, or other vehicle as known in the art. A pharmaceutical composition as described herein may also contain stabilizers, preservatives, buffers, antioxidants, or other additives well known to those of skill in the art.

As described elsewhere herein, a CD20-binding molecule as described herein or composition thereof (e.g. pharmaceutical or diagnostic composition) may be prepared with carriers that will protect the CD20-binding molecule described herein against rapid release, such as a controlled release formulation, including implants, transdermal patches, and microencapsulated delivery systems. Biodegradable, biocompatible polymers can be used, such as ethylene vinyl acetate, polyanhydrides, polyglycolic acid, collagen, polyorthoesters, and polylactic acid. Many methods for the preparation of such formulations are patented or generally known to those skilled in the art (see e.g. Sustained and Controlled Release Drug Delivery Systems (Robinson J, ed., Marcel Dekker, Inc., NY, U.S., 1978).

In some embodiments, the composition as described herein (e.g. pharmaceutical or diagnostic compositions) may be formulated to ensure a desired distribution in vivo. For example, the blood-brain barrier excludes many large and/or hydrophilic compounds. To target a therapeutic molecule or composition as described herein to a particular in vivo location, it can be formulated, for example, in liposomes which may comprise one or more moieties that are selectively transported into specific cells or organs, thus enhancing targeted drug delivery. Illustrative targeting moieties include folate or biotin; mannosides; antibodies; surfactant protein A receptor; p120 catenin and the like.

Pharmaceutical compositions include parenteral formulations designed to be used as implants or particulate systems. Examples of implants are depot formulations composed of polymeric or hydrophobic components such as emulsions, ion exchange resins, and soluble salt solutions. Examples of particulate systems are microspheres, microparticles, nanocapsules, nanospheres, and nanoparticles. Controlled release formulations may be prepared using polymers sensitive to ions, such as, e.g. liposomes, polaxamer 407, and hydroxyapatite.

Pharmaceutical compositions as described herein may be produced using techniques known in the art such that the produced compositions comprise emulsions, liposomes, niosomes, polymeric nanoparticles, and/or solid lipid nanoparticles (SLNs) (see e.g. Lakshmi P et al., Venereal Leprol 73: 157-161 (2007); A Revolution in Dosage Form Design and Development, Recent Advances in Novel Drug Carrier Systems (Sezer A, ed., InTech, 2012)).

### VIII. Polynucleotides, Expression Vectors, and Host Cells Described Herein

Beyond the CD20-binding molecules as described herein and compositions thereof, the polynucleotides that encode such CD20-binding molecules, the proteinaceous components of such CD20-binding molecules, or functional portions thereof, are also encompassed within the scope as described herein. The term “polynucleotide” is equivalent to the term “nucleic acids,” each of which includes one or more of: polymers of deoxyribonucleic acids (DNAs), polymers of ribonucleic acids (RNAs), analogs of these DNAs or RNAs generated using nucleotide analogs, and derivatives, fragments and homologs thereof. The polynucleotide described herein may be single-, double-, or triple-stranded. Such polynucleotides are specifically disclosed to include all polynucleotides capable of encoding an illustrative protein, for example, taking into account the wobble known to be tolerated in the third position of RNA codons, yet encoding for the same amino acid as a different RNA codon (see Stothard P, Biotechniques 28: 1102-4 (2000)).

Also provided herein are polynucleotides which encode a CD20-binding molecule (e.g. a CD20-binding protein as described herein), or a component, fragment or derivative thereof. The polynucleotides may include, e.g., a nucleic acid sequence encoding a polypeptide at least 50%, 55%, 60%, 65%, 70%, 75%, 80%, 85%, 90%, 95%, 99% or more, identical to a polypeptide comprising one of the amino acid sequences of all or part of a CD20-binding molecule as described herein. Also described herein are polynucleotides comprising nucleotide sequences that hybridize under stringent conditions to a polynucleotide which encodes all or part of a CD20-binding molecule as described herein, or a fragment or derivative thereof, or the antisense or complement of any such sequence.

Derivatives or analogs of the polynucleotides (or CD20-binding proteins) described herein include, inter alia, polynucleotide (or polypeptide) molecules having regions that are substantially homologous to the polynucleotides or CD20-binding proteins described herein, e.g. by at least about 45%, 50%, 70%, 80%, 95%, 98%, or even 99% identity (with a preferred identity of 80-99%) over a polynucleotide or polypeptide sequence of the same size or when compared to an aligned sequence in which the alignment is done by a computer homology program known in the art. An illustrative program is the GAP program (Wisconsin Sequence Analysis Package, Version 8 for UNIX, Genetics Computer Group, University Research Park, Madison, Wis., U.S.) using the default settings, which uses the algorithm of Smith T, Waterman M, Adv Appl Math 2: 482-9 (1981). Also included are polynucleotides capable of hybridizing to the complement of a sequence encoding the CD20-binding proteins described herein under stringent conditions (see e.g. Ausubel F et al., Current Protocols in Molecular Biology (John Wiley & Sons, New York, N.Y., U.S., 1993)), and below. Stringent conditions are known to those skilled in the art and may be found, e.g., in Current Protocols in Molecular Biology (John Wiley & Sons, NY, U.S., Ch. Sec. 6.3.1-6.3.6 (1989)).

Also described are expression vectors that comprise the polynucleotides within the scope as described herein. The polynucleotides capable of encoding the CD20-binding proteins described herein may be inserted into known vectors, including bacterial plasmids, viral vectors and phage vectors, using material and methods well known in the art to produce expression vectors. Such expression vectors will include the polynucleotides necessary to support production of contemplated CD20-binding proteins described herein within any host cell of choice or cell-free expression systems (e.g., pTxb1 and pIVEX2.3). The specific polynucleotides comprising expression vectors for use with specific types of host cells or cell-free expression systems are well known to one of ordinary skill in the art, can be determined using routine experimentation, or may be purchased.

The term “expression vector,” as used herein, refers to a polynucleotide, linear or circular, comprising one or more expression units. The term “expression unit” denotes a polynucleotide segment encoding a polypeptide of interest and capable of providing expression of the nucleic acid segment in a host cell. An expression unit typically comprises a transcription promoter, an open reading frame encoding the polypeptide of interest, and a transcription terminator, all in operable configuration. An expression vector contains one or more expression units. Thus, in the context as described herein, an expression vector encoding a protein comprising a single polypeptide chain (e.g. a scFv genetically recombined with a Shiga toxin effector region) includes at least an expression unit for the single polypeptide chain, whereas a protein comprising, e.g. two or more polypeptide chains (e.g. one chain comprising a V_{L} domain and a second chain comprising a V_{H} domain linked to a toxin effector region) includes at least two expression units, one for each of the two polypeptide chains of the protein. For expression of multi-chain proteins described herein, an expression unit for each polypeptide chain may also be separately contained on different expression vectors (e.g. expression may be achieved with a single host cell into which expression vectors for each polypeptide chain has been introduced).

Expression vectors capable of directing transient or stable expression of polypeptides and proteins are well known in the art. The expression vectors generally include, but are not limited to, one or more of the following: a heterologous signal sequence or peptide, an origin of replication, one or more marker genes, an enhancer element, a promoter, and a transcription termination sequence, each of which is well known in the art. Optional regulatory control sequences, integration sequences, and useful markers that can be employed are known in the art.

The term “host cell” refers to a cell which can support the replication or expression of the expression vector. Host cells may be prokaryotic cells, such as E. coli or eukaryotic cells (e.g. yeast, insect, amphibian, bird, or mammalian cells). Creation and isolation of host cell lines comprising a polynucleotide described herein or capable of producing a CD20-binding protein described herein may be accomplished using standard techniques known in the art.

Molecules and compositions within the scope as described herein may comprise variants or derivatives of the CD20-binding molecules described herein that are produced by modifying the polynucleotide encoding a proteinaceous component of a CD20-binding molecule described herein and/or a CD20-binding protein described herein by altering one or more amino acids or deleting or inserting one or more amino acids that may render it more suitable to achieve desired properties, such as more optimal expression by a host cell.

### IX. Delivery Devices and Kits

In some embodiments, provided herein are a device comprising one or more compositions of matter as described herein, such as, e.g., a pharmaceutical composition, for delivery to a subject in need thereof. Thus, among certain embodiments as described herein are kits comprising a composition of matter as described herein, and optionally, instructions for use, additional reagent(s), and/or pharmaceutical delivery device(s). A delivery device comprising one or more proteinaceous compositions as described herein (e.g. a CD20-binding molecule as described herein) may be used to administer to a patient a composition of matter described herein by various delivery methods, including: intravenous, subcutaneous, intramuscular or intraperitoneal injection; oral administration; transdermal administration; pulmonary or transmucosal administration; administration by implant, osmotic pump, cartridge or micro pump; or by other means recognized by a person of skill in the art.

Kits may be useful for drug administration and/or diagnostic information gathering. A kit described herein may optionally comprise at least one additional reagent (e.g. standards, markers and the like). Kits typically include a label indicating the intended use of the contents of the kit. The kit may further comprise reagents and other tools for detecting a cell type (e.g. a tumor cell) in a sample or in a subject, or for diagnosing whether a patient belongs to a group that responds to a therapeutic strategy which makes use of a CD20-binding molecule, composition, or related method as described herein as described herein.

### X. Illustrative Methods for Using the Compositions Described Herein

Generally, it is an object of this disclosure to provide pharmacologically active agents, as well as compositions comprising the same, that can be used in the prevention and/or treatment of diseases, disorders, and conditions, such as certain cancers, tumors, growth abnormalities, immune disorders, or further pathological conditions mentioned herein. Accordingly, provided herein are methods of using the CD20-binding molecules of the present, solvates as described herein, salts as described herein, and compositions of any of the aforementioned, such as pharmaceutical compositions described herein, for the targeted killing of CD20 expressing cells, for delivering additional exogenous materials into certain CD20 expressing cells, for labeling of the interiors of certain CD20 expressing cells, for collecting diagnostic information, and for treating various diseases, disorders, and conditions as described herein.

In particular, it is an object described herein to provide such pharmacologically active agents, compositions, and/or methods that have certain advantages compared to the agents, compositions, and/or methods that are currently known in the art. Accordingly, provided herein are methods of using CD20-binding molecules, and compositions thereof, characterized by specified proteinaceous components. For example, any of the molecules shown in SEQ ID NOs: 1-304 may be specifically utilized as a component of the CD20-binding molecule or composition used in the following methods.

Additionally, provided herein is a method of rapidly inducing cellular internalization of a CD20-binding molecule into a CD20 expressing cell(s), the method comprising the step of contacting the cell(s) with a CD20-binding molecule as described herein or a pharmaceutical composition thereof. Similarly, provided herein is a method of internalizing a cell surface localized CD20 bound by a CD20-binding molecule in a patient, the method comprising the step of administering to the patient a CD20-binding molecule or pharmaceutical composition as described herein.

Also provided herein are methods of killing a cell comprising the step of contacting the cell, either in vitro or in vivo, with a CD20-binding molecule, and/or composition thereof. The CD20-binding molecule as described herein, and compositions thereof, can be used to kill a specific cell type upon contacting a cell or cells with one of the claimed compositions of matter. In some embodiments, a CD20-binding molecule as described herein and/or a composition thereof can be used to kill specific CD20+ cell types in a mixture of different cell types, such as mixtures comprising CD20+ cancer, tumor, hematologic, immune, and/or infected cells.

Also provided herein are methods of killing cell(s), the method comprising the step of contacting a cell(s), with a CD20-binding molecule as described herein, a CD20-binding molecule composition as described herein, a solvate as described herein, a salt as described herein, and/or a pharmaceutical composition as described herein; wherein the cell(s) is physically coupled with a CD20 having the extracellular part bound by the two or more binding regions of the CD20-binding molecule or a CD20-binding molecule of a composition as described herein. For certain embodiments, the step of contacting the cell(s) occurs in vitro. For certain other embodiments, the step of contacting the cell(s) occurs in vivo. In some embodiments, the cell(s) express at a cellular surface the CD20 which (1) have the extracellular part bound by the two or more CD20 binding regions of the CD20-binding molecule, (2) have a transmembrane domain, and (3) remain physically coupled to the cell. In some embodiments, the cell(s) is CD20 positive cells. For certain embodiments, the cell(s) is physically coupled with a significant amount of extracellular CD20 which (1) have the extracellular part bound by the two or more CD20 binding regions of the CD20-binding molecule. For certain embodiments, the cell(s) are descendants or members of a B-cell lineage. For certain embodiments, the cell(s) is a malignant B-cell, B-cell leukemia cell, B-cell lymphoma cell, B-cell myeloma cell, acute myeloid leukemia cell, acute non-lymphocytic leukemia cell, B-cell chronic lymphocytic leukemia cell, B-cell lymphoma cell, B-cell non-Hodgkin's lymphoma cell, B-cell precursor acute lymphoblastic leukemia cell, B-cell prolymphocytic leukemia cell, Burkitt's lymphoma cell, chronic lymphocytic leukemia cell, chronic myeloid leukemia cell, diffuse large B-cell lymphoma cell, follicular lymphoma cell, hairy cell leukemia cell, Hodgkin's lymphoma cell, immunoblastic large cell lymphoma cell, mantle cell lymphoma cell, melanoma cell, multiple myeloma cell, neoplastic plasma cell, nodular lymphocyte predominant Hodgkin's lymphoma cell, non-Hodgkin's lymphoma cell, plasmablastic lymphoma cell, plasma cell myeloma cell, precursor B-lymphoblastic lymphoma cell, small lymphocytic lymphoma cell, malignant T-cell, T-cell leukemia cell, T-cell lymphoma cell, T-cell large granular lymphocyte leukemia cell, T-cell prolymphocytic leukemia, healthy B-cell lineage cell, or healthy T-cell.

The CD20-binding molecule as described herein, and compositions thereof, have varied applications, including, e.g., uses in depleting unwanted CD20+ cell types from tissues either in vitro or in vivo, uses in modulating immune responses to treat graft-versus-host disease, and uses in purging transplantation tissues of unwanted CD20+ cell types. In some embodiments, a CD20-binding molecule as described herein and/or a composition thereof can be used to kill CD20+ cancer cells in a mixture of different cell types. In some embodiments, a CD20-binding molecule as described herein and/or a composition thereof can be used to kill specific CD20+ cell types in a mixture of different cell types, such as cells in pre-transplantation tissues. In some embodiments, a CD20-binding molecule as described herein and/or a composition thereof can be used to kill specific CD20+ cell types in a mixture of cell types, such as cells in pre-administration tissue material for therapeutic purposes.

In some embodiments, a CD20-binding molecule as described herein and/or a composition thereof, alone or in combination with other compounds or pharmaceutical compositions, can show potent cell-kill activity when administered to a population of cells in vitro or in vivo in a subject, such as, e.g., in a patient in need of treatment. By targeting the delivery of enzymatically active Shiga toxin regions using high-affinity binding regions to CD20, this potent cell-kill activity can be restricted to specifically and selectively kill certain cell types within an organism, such as certain CD20 positive cancer cells, neoplastic cells, malignant tumor cells, non-malignant tumor cells, and/or immune cells.

Additionally, provided herein is a method for delivering exogenous material to the inside of a cell(s) comprising contacting the cell(s), either in vitro or in vivo, with a CD20-binding molecule or pharmaceutical composition as described herein.

Further provided is a method for delivering exogenous material to the inside of a cell(s) in a patient, wherein the cell expresses CD20 on its surface, the method comprising the step of administering to the patient a CD20-binding molecule as described herein.

In humans, CD20 is expressed by normal, B-cell lineage cells within certain cell developmental stages as well as numerous mature B-cell neoplasms, such as in NHL and CLL. In addition, CD20 is expressed by mature T-cell and NK-cell neoplasms. CD20 is expressed by a subset of normal T-cells as well as malignant T-cells such as in T-cell lymphomas (TCLs), including mycosis fungoides (MF), natural killer cell lymphoma (NK-cell lymphoma), peripheral T-cell lymphomas (PTCLs), and cutaneous T-cell lymphomas. CD20 is expressed by malignant T-cells in T-cell large granular lymphocyte leukemia (T-LGLL).

Also provided herein is a method of killing a CD20+ cell in a patient in need thereof, the method comprising the step of administering to the patient at least one CD20-binding molecule as described herein or a composition thereof, such as, e.g., a pharmaceutical composition comprising a CD20-binding molecule as described herein.

Additionally, provided herein are methods of killing cells comprising the step of contacting the cell with a CD20-binding molecule described herein or a pharmaceutical composition described herein. In some embodiments, the step of contacting the cell(s) occurs in vitro. In certain other embodiments, the step of contacting the cell(s) occurs or in vivo.

In some embodiments, the CD20-binding molecule as described herein and/or a composition thereof is useful for killing malignant cells which express elevated levels of CD20 at a cellular surface. The CD20-binding molecule as described herein and/or a composition thereof is particularly useful for killing neoplastic cells which express elevated levels of CD20 at a cellular surface.

Certain embodiments of the CD20-binding molecule as described herein and/or a composition thereof can be used to kill a CD20-expressing cancer and/or tumor cell in a patient, such as, e.g. B-cell or T-cell cancers. The term “cancer cell” or “cancerous cell” refers to various neoplastic cells which grow and divide in an abnormally accelerated and/or unregulated fashion and will be clear to the skilled person. The term “tumor cell” includes both malignant and non-malignant cells (e.g. non-cancerous, benign tumor cells and non-cancerous “cancer” stem cells, tumor stem cells, pre-malignant cancer-initiating cells, tumor-initiating cells, or tumorigenic cells—all of which can give rise to daughter cells which become malignant tumor and/or cancer cells but are unable to metastasize on their own (see e.g. Martinez-Climent J et al., Haematologica 95: 293-302 (2010)). Generally, cancers and/or tumors can be defined as diseases, disorders, or conditions that are amenable to treatment and/or prevention. Neoplastic cells are often associated with one or more of the following: unregulated growth, lack of differentiation, local tissue invasion, angiogenesis, and metastasis. The cancers and tumors (either malignant or non-malignant) which are comprised of cancer cells and/or tumor cells which may benefit from methods and compositions described herein will be clear to the skilled person. The CD20-binding molecules and compositions described herein may be used to kill cancer stem cells, tumor stem cells, pre-malignant cancer-initiating cells, and tumor-initiating cells which commonly are slow dividing and resistant to cancer therapies like chemotherapy and radiation. For example, the following non-limiting examples of conditions involving cells with limited malignant potential may be diagnosed and/or treated using CD20-binding molecules as described herein: monoclonal B-cell lymphocytosis (MBL), localized follicular lymphoma (localized FL), gastric extranodal marginal zone (MALT) lymphomas, and intrafollicular neoplasia (Limpens J et al., Oncogene 6: 2271-6 (1991); Liu H et al., Lancet 357: 39-40 (2001); Richard P et al., J Clin Pathol 59: 995-6 (2006); Roulland S et. al.., J Exp Med 203: 2425-31 (2006); Marti G et al., Br J Haematol 139:701-8 (2007); Aqel N et al., Histopathology 52: 256-60 (2008); Rawstron A et al., N Engl J Med 359: 575-83 (2008)). Similarly, cancer initiating cells and/or cancer stem cells may be detected and/or treated using CD20-binding molecules described herein, such as, e.g., acute myeloid leukemia (AML) stem cells, B-cell non-Hodgkin's lymphoma (B-cell NHL) initiating cells, chronic myeloid leukemia (CML) stem cells, Hodgkin's lymphoma (HL or HD) stem-like cells, and mantle cell lymphoma (MCL) initiating cells (see e.g. Hope K et al., Nat Immunol 5: 738-43 (2004); Wang J, Dick J, Trends Cell Biol 15: 494-501 (2005); Ishikawa F et al., Nat Biotechnol 25: 1315-21 (2007); Jones R et al., Blood 113: 5920-6 (2009); Chen Z et al., Stem Cell Res 5: 212-225 (2010); Chomel J et al., Blood 118: 3657-60 (2011); Druker B, J Clin Invest 121: 396-409 (2011); Gerber J et al., Blood 119: 3571-7 (2012)).

Certain embodiments of the CD20-binding molecule as described herein and/or a composition thereof can be used to kill an immune cell (whether healthy or malignant) in a patient by targeting an extracellular part of CD20 found physically coupled with the immune cell. Certain embodiments of the CD20-binding molecules described herein, and compositions thereof, may be used to kill a healthy CD20+ immune cell(s) in a patient. CD20 is expressed by normal, B-cell lineage cells within certain cell developmental stages (van Meerten T et al., Clin Cancer Res 12: 4027-35 (2006)). CD20 is expressed by a subset of normal T-cells (Martin B et al., J Cutan Pathol 38: 663-9 (2011)).

It is within the scope as described herein to utilize the CD20-binding molecule as described herein and/or a composition thereof for the purposes of purging patient cell populations (e.g. bone marrow) of malignant, neoplastic, or otherwise unwanted B-cells and/or T-cells and then reinfusing the B-cell and/or T-cell depleted material into the patient.

It is within the scope as described herein to utilize the CD20-binding molecule as described herein and/or a composition thereof for the purposes of ex vivo depletion of B-cells and/or T-cells from isolated cell populations removed from a patient. In one non-limiting example, the CD20-binding molecule as described herein and/or a composition thereof may be used in a method for prophylaxis of organ and/or tissue transplant rejection wherein the donor organ or tissue is perfused prior to transplant with a cytotoxic CD20-binding molecule as described herein and/or a composition thereof in order to purge the organ of unwanted donor B-cells and/or T-cells.

It is also within the scope as described herein to utilize the CD20-binding molecule as described herein and/or a composition thereof for the purposes of depleting B-cells and/or T-cells from a donor cell population as a prophylaxis against graft-versus-host disease, and induction of tolerance, in a patient to undergo a bone marrow and or stem cell transplant.

It is within the scope as described herein to provide a bone marrow recipient for prophylaxis or treatment of host-versus-graft disease via the targeted cell-killing of host B-cells and/or T-cells using CD20-binding molecule as described herein and/or a composition thereof (see e.g. Sarantopoulos S et al., Biol Blood Marrow Transplant 21: 16-23 (2015)).

Additionally, provided herein is a method of treating a disease, disorder, or condition in a patient comprising the step of administering to a patient in need thereof a therapeutically effective amount of at least one of the CD20-binding molecule as described herein and/or composition thereof. Contemplated diseases, disorders, and conditions that can be treated using this method include cancers, malignant tumors, non-malignant tumors, growth abnormalities, and immune disorders. In some embodiments, the disease, disorder, or condition to be treated using this method described herein involves a cell(s) or cell type(s) which express CD20 on a cellular surface, such as, e.g., a cancer cell, a tumor cell, or an immune cell. A further embodiment is a method of treating a disease involving a cancer or tumor cell associated with the disease selected from the group consisting of: bone cancer, leukemia, lymphoma, melanoma, or myeloma. In some embodiments of this method, the disorder is an immune disorder associated with a disease selected from the group consisting of: amyloidosis, ankylosing spondylitis, asthma, Crohn's disease, diabetes, graft rejection, graft-vs.-host disease, Hashimoto's thyroiditis, hemolytic uremic syndrome, HIV-related diseases, lupus erythmatosis, multiple sclerosis, polyarteritis, psoriasis, psoriatic arthritis, rheumatoid arthritis, scleroderma, septic shock, Sjorgren's syndrome, ulcerative colitis, and vasculitis. Administration of a “therapeutically effective dosage” of a composition described herein may result in a decrease in severity of disease symptoms, an increase in frequency and duration of disease symptom-free periods, or a prevention of impairment or disability due to the disease affliction.

The therapeutically effective amount of a composition as described herein will depend on the route of administration, the type of mammal being treated, and the physical characteristics of the specific patient under consideration. These factors and their relationship to determining this amount are well known to skilled practitioners in the medical arts. This amount and the method of administration can be tailored to achieve optimal efficacy, and may depend on such factors as weight, diet, concurrent medication and other factors, well known to those skilled in the medical arts. The dosage sizes and dosing regimen most appropriate for human use may be guided by the results obtained and may be confirmed in properly designed clinical trials. An effective dosage and treatment protocol may be determined by conventional means, starting with a low dose in laboratory animals and then increasing the dosage while monitoring the effects, and systematically varying the dosage regimen as well. Numerous factors may be taken into consideration by a clinician when determining an optimal dosage for a given subject. Such considerations are known to the skilled person.

In some embodiments, the CD20-binding molecule is administered intravenously to the subject in an amount of about 1 μg/kg to about 100 μg/kg of the subject's body weight, wherein the CD20-binding molecule is administered once per week, twice per week, or three times per week for two or more consecutive weeks, such as four or five consecutive weeks. In some embodiments, the CD20-binding molecule is administered intravenously to the subject in an amount of about 30 μg/kg to about 100 μg/kg of the subject's body weight, wherein the CD20-binding molecule is administered once per week, twice per week, or three times per week for two or more consecutive weeks, such as four or five consecutive weeks. In some embodiments, the CD20-binding molecule is administered intravenously to the subject in an amount of about 15 μg/kg to about 50 μg/kg of the subject's body weight, wherein the CD20-binding molecule is administered once per week, twice per week, or three times per week for two or more consecutive weeks, such as four or five consecutive weeks. In some embodiments, the CD20-binding molecule is administered intravenously to the subject in an amount of about 1 μg/kg to about 10 μg/kg, about 10 μg/kg to about 25 μg/kg, about 25 μg/kg to about 50 μg/kg or about 50 μg/kg to about 750 μg/kg of the subject's body weight. In some embodiments, the CD20-binding molecule is administered intravenously to the subject in an amount of about 1 μg/kg, about 2 μg/kg, about 3 μg/kg, about 4 μg/kg, about 5 μg/kg, about 6 μg/kg, about 7 μg/kg, about 8 μg/kg, about 9 μg/kg, about 10 μg/kg, about 11 μg/kg, about 12 μg/kg, about 13 μg/kg, about 14 μg/kg, about 15 μg/kg, about 16 μg/kg, about 17 μg/kg, about 18 μg/kg, about 19 μg/kg, about 20 μg/kg, about 21 μg/kg, about 22 μg/kg, about 23 μg/kg, about 24 μg/kg, about 25 μg/kg, about 26 μg/kg, about 27 μg/kg, about 28 μg/kg, about 29 μg/kg, about 30 μg/kg, about 31 μg/kg, about 32 μg/kg, about 33 μg/kg, about 34 μg/kg, about 35 μg/kg, about 36 μg/kg, about 37 μg/kg, about 38 μg/kg, about 39 μg/kg, about 40 μg/kg, about 41 μg/kg, about 42 μg/kg, about 43 μg/kg, about 44 μg/kg, about 45 μg/kg, about 46 μg/kg, about 47 μg/kg, about 48 μg/kg, about 49 μg/kg, about 50 μg/kg, about 51 μg/kg, about 52 μg/kg, about 53 μg/kg, about 54 μg/kg, about 55 μg/kg, about 56 μg/kg, about 57 μg/kg, about 58 μg/kg, about 59 μg/kg, about 60 μg/kg, about 61 μg/kg, about 62 μg/kg, about 63 μg/kg, about 64 μg/kg, about 65 μg/kg, about 66 μg/kg, about 67 μg/kg, about 68 μg/kg, about 69 μg/kg, about 70 μg/kg, about 71 μg/kg, about 72 μg/kg, about 73 μg/kg, about 74 μg/kg, about 75 μg/kg, about 80 μg/kg, about 85 μg/kg, about 90 μg/kg, about 95 μg/kg, or about 100 μg/kg of the subject's body weight. In some embodiments, the CD20-binding molecule is administered once per week, twice per week, or three times per week for two or more consecutive weeks, such as four or five consecutive weeks. In some embodiments, the CD20-binding molecule is administered intravenously to the subject in an amount of about 10 μg/kg, about 25 μg/kg, about 50 μg/kg, or about 75 μg/kg of the subject's body weight, wherein the CD20-binding molecule is administered once, twice, or three times per week for two or more consecutive weeks, such as four or five consecutive weeks.

In some embodiments, the CD20-binding molecule is administered intravenously to the subject in an amount of about 1 μg/kg to about 100 μg/kg of the subject's body weight, wherein the CD20-binding molecule is administered once per week, twice per week, or three times per week for two or more weeks, such as five weeks with a dosing holiday for the entire third week. In some embodiments, the CD20-binding molecule is administered intravenously to the subject in an amount of about 30 μg/kg to about 100 μg/kg of the subject's body weight, wherein the CD20-binding molecule is administered once per week, twice per week, or three times per week for two or more weeks, such as five weeks with a dosing holiday for the entire third week. In some embodiments, the CD20-binding molecule is administered intravenously to the subject in an amount of about 15 μg/kg to about 50 μg/kg of the subject's body weight, wherein the CD20-binding molecule is administered once per week, twice per week, or three times per week for two or more weeks, such as five weeks with a dosing holiday for the entire third week. In some embodiments, the CD20-binding molecule is administered intravenously to the subject in an amount of about 1 μg/kg, about 2 μg/kg, about 3 μg/kg, about 4 μg/kg, about 5 μg/kg, about 6 μg/kg, about 7 μg/kg, about 8 μg/kg, about 9 μg/kg, about 10 μg/kg, about 11 μg/kg, about 12 μg/kg, about 13 μg/kg, about 14 μg/kg, about 15 μg/kg, about 16 μg/kg, about 17 μg/kg, about 18 μg/kg, about 19 μg/kg, about 20 μg/kg, about 21 μg/kg, about 22 μg/kg, about 23 μg/kg, about 24 μg/kg, about 25 μg/kg, about 26 μg/kg, about 27 μg/kg, about 28 μg/kg, about 29 μg/kg, about 30 μg/kg, about 31 μg/kg, about 32 μg/kg, about 33 μg/kg, about 34 μg/kg, about 35 μg/kg, about 36 μg/kg, about 37 μg/kg, about 38 μg/kg, about 39 μg/kg, about 40 μg/kg, about 41 μg/kg, about 42 μg/kg, about 43 μg/kg, about 44 μg/kg, about 45 μg/kg, about 46 μg/kg, about 47 μg/kg, about 48 μg/kg, about 49 μg/kg, about 50 μg/kg, about 51 μg/kg, about 52 μg/kg, about 53 μg/kg, about 54 μg/kg, about 55 μg/kg, about 56 μg/kg, about 57 μg/kg, about 58 μg/kg, about 59 μg/kg, about 60 μg/kg, about 61 μg/kg, about 62 μg/kg, about 63 μg/kg, about 64 μg/kg, about 65 μg/kg, about 66 μg/kg, about 67 μg/kg, about 68 μg/kg, about 69 μg/kg, about 70 μg/kg, about 71 μg/kg, about 72 μg/kg, about 73 μg/kg, about 74 μg/kg, about 75 μg/kg, about 80 μg/kg, about 85 μg/kg, about 90 μg/kg, about 95 μg/kg, or about 100 μg/kg of the subject's body weight, wherein the CD20-binding molecule is administered once per week, twice per week, or three times per week for two or more weeks, such as five weeks with a dosing holiday for the entire third week. In some embodiments, the CD20-binding molecule is administered intravenously to the subject in an amount of about 10 μg/kg, about 25 μg/kg, about 50 μg/kg, or about 75 μg/kg of the subject's body weight, wherein the CD20-binding molecule is administered once, twice, or three times per week for two or more weeks, such as five weeks with a dosing holiday for the entire third week.

In some embodiments, the CD20-binding molecules described herein may be administered to the same subject on multiple occasions. Intervals between single doses can be, for example, 1 to 4 days, weekly, monthly, every two or three months, every six months, or yearly. Intervals between administrations can also be irregular, based on regulating blood levels of the active compound or based on other markers, indications, or signs present in the subject.

An acceptable route of administration may refer to any administration pathway known in the art, including but not limited to aerosol, enteral, nasal, ophthalmic, oral, parenteral, rectal, vaginal, or transdermal (e.g. topical administration of a cream, gel or ointment, or by means of a transdermal patch). “Parenteral administration” is typically associated with injection at or in communication with the intended site of action, including infraorbital, infusion, intraarterial, intracapsular, intracardiac, intradermal, intramuscular, intraperitoneal, intrapulmonary, intraspinal, intrasternal, intrathecal, intrauterine, intravenous, subarachnoid, subcapsular, subcutaneous, transmucosal, or transtracheal administration.

In some embodiments of the methods described herein, the CD20-binding molecule is administered intravenously.

In some embodiments, a method for treating or slowing the progression of a disease, disorder, or condition comprises administering to a subject in need thereof an effective amount of a CD20-binding molecule. In some embodiments, the CD20-binding molecule comprises a polypeptide having the sequence of SEQ ID NO: 54. In some embodiments, the CD20-binding molecule is a homodimer comprising two identical polypeptides. In some embodiments, each identical polypeptide has the amino acid sequence of SEQ ID NO: 54; and wherein there is a disulfide bond at each cysteine residue at amino acid position 503 of SEQ ID NO: 54.

In some embodiments, the effective amount is administered in a single dose. For example, in some embodiments, the effective amount is a single dose of about 1 μg/kg, about 2 μg/kg, about 3 μg/kg, about 4 μg/kg, about 5 μg/kg, about 6 μg/kg, about 7 μg/kg, about 8 μg/kg, about 9 μg/kg, about 10 μg/kg, about 11 μg/kg, about 12 μg/kg, about 13 μg/kg, about 14 μg/kg, about 15 μg/kg, about 16 μg/kg, about 17 μg/kg, about 18 μg/kg, about 19 μg/kg, about 20 μg/kg, about 21 μg/kg, about 22 μg/kg, about 23 μg/kg, about 24 μg/kg, about 25 μg/kg, about 26 μg/kg, about 27 μg/kg, about 28 μg/kg, about 29 μg/kg, about 30 μg/kg, about 31 μg/kg, about 32 μg/kg, about 33 μg/kg, about 34 μg/kg, about 35 μg/kg, about 36 μg/kg, about 37 μg/kg, about 38 μg/kg, about 39 μg/kg, about 40 μg/kg, about 41 μg/kg, about 42 μg/kg, about 43 μg/kg, about 44 μg/kg, about 45 μg/kg, about 46 μg/kg, about 47 μg/kg, about 48 μg/kg, about 49 μg/kg, about 50 μg/kg, about 51 μg/kg, about 52 μg/kg, about 53 μg/kg, about 54 μg/kg, about 55 μg/kg, about 56 μg/kg, about 57 μg/kg, about 58 μg/kg, about 59 μg/kg, about 60 μg/kg, about 61 μg/kg, about 62 μg/kg, about 63 μg/kg, about 64 μg/kg, about 65 μg/kg, about 66 μg/kg, about 67 μg/kg, about 68 μg/kg, about 69 μg/kg, about 70 μg/kg, about 71 μg/kg, about 72 μg/kg, about 73 μg/kg, about 74 μg/kg, about 75 μg/kg, about 80 μg/kg, about 85 μg/kg, about 90 μg/kg, about 95 μg/kg, about 100 μg/kg, about 125 μg/kg, about 150 μg/kg, about 175 μg/kg, about 200 μg/kg, about 225 μg/kg, about 250 μg/kg, about 275 μg/kg, about 300 μg/kg, about 325 μg/kg, about 350 μg/kg, about 375 μg/kg, about 400 μg/kg, about 425 μg/kg, about 450 μg/kg, about 500 μg/kg, about 525 μg/kg, or about 550 μg/kg. In some embodiments, the effective amount is a single dose of about 10 μg/kg, about 25 μg/kg, about 50 μg/kg, or about 75 μg/kg.

In some embodiments, the amount of CD20-binding molecule administered to the subject in a single dose is about 0.1 mg to about 10 mg. In some embodiments, the amount of CD20-binding molecule administered to the subject in a single dose is about 0.5 mg to about 10 mg. In some embodiments, the amount of CD20-binding molecule administered to the subject in a single dose is about 0.1 mg, about 0.2 mg, about 0.3 mg, about 0.4 mg, about 0.5 mg, about 0.6 mg, about 0.7 mg, about 0.8 mg, about 0.9 mg, about 1.0 mg, about 1.1 mg, about 1.2 mg, about 1.3 mg, about 1.4 mg, about 1.5 mg, about 1.6 mg, about 1.7 mg, about 1.8 mg, about 1.9 mg, about 2.0 mg, about 2.1 mg, about 2.2 mg, about 2.3 mg, about 2.4 mg, about 2.5 mg, about 2.6 mg, about 2.7 mg, about 2.8 mg, about 2.9 mg, about 3.0 mg, about 3.1 mg, about 3.2 mg, about 3.3 mg, about 3.4 mg, about 3.5 mg, about 3.6 mg, about 3.7 mg, about 3.8 mg, about 3.9 mg, about 4.0 mg, about 4.1 mg, about 4.2 mg, about 4.3 mg, about 4.4 mg, about 4.5 mg, about 4.6 mg, about 4.7 mg, about 4.8 mg, about 4.9 mg, about 5.0 mg, about 5.1 mg, about 5.2 mg, about 5.3 mg, about 5.4 mg, about 5.5 mg, about 5.6 mg, about 5.7 mg, about 5.8 mg, about 5.9 mg, about 6.0 mg, about 6.5 mg, about 7.0 mg, about 7.5 mg, about 8.0 mg, about 8.5 mg, about 9.0 mg, about 9.5 mg, or about 10 mg.

In some embodiments, the amount of CD20-binding molecule administered to the subject in a single cycle (e.g., a 28-day cycle or 42-day cycle) is about 1 mg to about 100 mg. In some embodiments, the amount of CD20-binding molecule administered to the subject in a single cycle (e.g., a 28-day cycle or 42-day cycle) is about 1 mg to about 50 mg. In some embodiments, the amount of CD20-binding molecule administered to the subject in a single cycle (e.g., a 28-day cycle or 42-day cycle) is about 5 mg to about 100 mg. In some embodiments, the amount of the CD20-binding molecule administered to the subject in a single cycle (e.g., a 28-day cycle or 42-day cycle) is about 1 mg, about 2 mg, about 3 mg, about 4 mg, about 5 mg, about 6 mg, about 7 mg, about 8 mg, about 9 mg, about 10 mg, about 11 mg, about 12 mg, about 13 mg, about 14 mg, about 15 mg, about 16 mg, about 17 mg, about 18 mg, about 19 mg, about 20 mg, about 21 mg, about 22 mg, about 23 mg, about 24 mg, about 25 mg, about 26 mg, about 27 mg, about 28 mg, about 29 mg, about 30 mg, about 31 mg, about 32 mg, about 33 mg, about 34 mg, about 35 mg, about 36 mg, about 37 mg, about 38 mg, about 39 mg, about 40 mg, about 41 mg, about 42 mg, about 43 mg, about 44 mg, about 45 mg, about 46 mg, about 47 mg, about 48 mg, about 49 mg, about 50 mg, about 51 mg, about 52 mg, about 53 mg, about 54 mg, about 55 mg, about 56 mg, about 57 mg, about 58 mg, about 59 mg, about 60 mg, about 61 mg, about 62 mg, about 63 mg, about 64 mg, about 65 mg, about 66 mg, about 67 mg, about 68 mg, about 69 mg, about 70 mg, about 71 mg, about 72 mg, about 73 mg, about 74 mg, about 75 mg, about 76 mg, about 77 mg, about 78 mg, about 79 mg, about 80 mg, about 81 mg, about 82 mg, about 83 mg, about 84 mg, about 85 mg, about 86 mg, about 87 mg, about 88 mg, about 89 mg, about 90 mg, about 91 mg, about 92 mg, about 93 mg, about 94 mg, about 95 mg, about 96 mg, about 97 mg, about 98 mg, about 99 mg, or about 100 mg.

In some embodiments, the amount of CD20-binding molecule administered to the subject over one or more cycles is about 5 mg to about 1000 mg. In some embodiments, the amount of CD20-binding molecule administered to the subject over one or more cycles is about 5 mg to about 250 mg. In some embodiments, the amount of CD20-binding molecule administered to the subject over one or more cycles is about 5 mg, about 10 mg, about 15 mg, about 20 mg, about 25 mg, about 30 mg, about 35 mg, about 40 mg, about 45 mg, about 50 mg, about 55 mg, about 60 mg, about 65 mg, about 70 mg, about 75 mg, about 80 mg, about 85 mg, about 90 mg, about 95 mg, about 100 mg, about 105 mg, about 110 mg, about 115 mg, about 120 mg, about 125 mg, about 130 mg, about 135 mg, about 140 mg, about 145 mg, about 150 mg, about 155 mg, about 160 mg, about 165 mg, about 170 mg, about 175 mg, about 180 mg, about 185 mg, about 190 mg, about 195 mg, about 200 mg, about 205 mg, about 210 mg, about 215 mg, about 220 mg, about 225 mg, about 230 mg, about 235 mg, about 240 mg, about 245 mg, about 250 mg, about 260 mg, about 270 mg, about 280 mg, about 290 mg, about 300 mg, about 310 mg, about 320 mg, about 330 mg, about 340 mg, about 350 mg, about 360 mg, about 370 mg, about 380 mg, about 390 mg, about 400 mg, about 410 mg, about 420 mg, about 430 mg, about 440 mg, about 450 mg, about 460 mg, about 470 mg, about 480 mg, about 490 mg, about 500 mg, about 525 mg, about 550 mg, about 575 mg, about 600 mg, about 625 mg, about 650 mg, about 675 mg, about 700 mg, about 725 mg, about 750 mg, about 775 mg, about 800 mg, about 825 mg, about 850 mg, about 875 mg, about 900 mg, about 925 mg, about 950 mg, about 975 mg, or about 1000 mg.

In some embodiments, the effective amount is divided and administered over multiple doses, such that the total cumulative amount administered over all doses is the effective amount. For example, in some embodiments, the effective amount is divided and administered over multiple doses, wherein each dose is individually selected from about 10 μg/kg, about 25 μg/kg, about 50 μg/kg, or about 75 μg/kg of the subject's body weight. In some embodiments, the effective amount is about 100 μg/kg to about 2000 μg/kg of the subject's body weight (i.e., about 200 μg/kg to about 400 μg/kg, about 300 μg/kg to about 800 μg/kg, or about 500 μg/kg to about 1000 μg/kg of the subject's body weight), wherein the effective amount is divided and administered over multiple doses. In some embodiments, the effective amount is about 150 μg/kg, about 155 μg/kg, about 160 μg/kg about 165 μg/kg, about 170 μg/kg, about 175 μg/kg, about 180 μg/kg, about 185 μg/kg, about 190 μg/kg, about 195 μg/kg, about 200 μg/kg, about 205 μg/kg, about 210 μg/kg, about 215 μg/kg, about 220 μg/kg, about 225 μg/kg, about 230 μg/kg, about 235 μg/kg, about 240 μg/kg, about 245 μg/kg, about 250 μg/kg, about 255 μg/kg, about 260 μg/kg, about 265 μg/kg, about 270 μg/kg, about 275 μg/kg, about 280 μg/kg, about 285 μg/kg, about 290 μg/kg, about 295 μg/kg, about 300 μg/kg, about 305 μg/kg, about 310 μg/kg, about 315 μg/kg, about 320 μg/kg, about 325 μg/kg, about 330 μg/kg, about 335 μg/kg, about 340 μg/kg, about 345 μg/kg, about 350 μg/kg, about 355 μg/kg, about 360 μg/kg, about 365 μg/kg, about 370 μg/kg, about 375 μg/kg, about 380 μg/kg, about 385 μg/kg, about 390 μg/kg, about 395 μg/kg, about 400 μg/kg, about 405 μg/kg, about 410 μg/kg, about 415 μg/kg, about 420 μg/kg, about 425 μg/kg, about 430 μg/kg, about 435 μg/kg, about 440 μg/kg, about 445 μg/kg, about 450 μg/kg, about 455 μg/kg, about 460 μg/kg, about 465 μg/kg, about 470 μg/kg, about 475 μg/kg, about 480 μg/kg, about 485 μg/kg, about 490 μg/kg, about 495 μg/kg, about 500 μg/kg, about 505 μg/kg, about 510 μg/kg, about 515 μg/kg, about 520 μg/kg, about 525 μg/kg, about 530 μg/kg, about 535 μg/kg, about 540 μg/kg, about 545 μg/kg, or about 550 μg/kg of the subject's body weight, wherein the effective amount is divided and administered over multiple doses. In some embodiments, the effective amount is divided and administered over 2, 3, 4, 5, 6, 7, 8, 9, 10, 11, 12, 13, 14, 15, 16, 17, 18, 19, 20, 21, 22, 23, 24, 25, or more doses.

In some embodiments, a first dose and a second dose of a CD20-binding molecule are administered to a subject. In some embodiments, the second dose is higher than the first dose. For example, the second dose may be greater than the first dose by about 1.1-fold, about 1.2-fold, about 1.3-fold, about 1.4-fold, about 1.5-fold, about 1.6-fold, about 1.7-fold, about 1.8-fold, about 1.9-fold, about 2-fold, about 3-fold, about 5-fold, about 10-fold, about 25-fold, or about 50-fold higher than the first dose, or more. In some embodiments, the second dose is lower than the first dose. For example, the second dose may lower than the first dose by about 5%, about 10%, about 15%, about 20%, about 25%, about 30%, about 35%, about 40%, about 45%, about 50%, about 60%, about 70%, about 80%, about 90%, about 100%, about 200%, about 500%, or more.

In some embodiments, the dose of the CD20-binding molecule is increased during the course of treatment. In some embodiments, the dose of the CD20-binding molecule is increased once per week, twice per week, three times per week, four times per week, 5 times per week, once every 2 weeks, once every 3 weeks, once per month, once every 2 months, once every 3 months, once every 4 months, once every 5 months, once every 6 months, or once per year during the course of treatment. In some embodiments, the dose of the CD20-binding molecule is increased from about 10 μg/kg to about 20 μg/kg, about 25 μg/kg, about 30 μg/kg, about 35 μg/kg, about 40 μg/kg, about 45 μg/kg, about 50 μg/kg, about 55 μg/kg, about 60 μg/kg, about 65 μg/kg, about 70 μg/kg, about 75 μg/kg, about 80 μg/kg, about 85 μg/kg, about 90 μg/kg, about 95 μg/kg, about 100 μg/kg, about 125 μg/kg, about 150 μg/kg, about 175 μg/kg, about 200 μg/kg, about 225 μg/kg, or about 250 μg/kg of the subject's body weight. In some embodiments, the dose of the CD20-binding molecule is increased from about 25 μg/kg to about 30 μg/kg, about 35 μg/kg, about 40 μg/kg, about 45 μg/kg, about 50 μg/kg, about 55 μg/kg, about 60 μg/kg, about 65 μg/kg, about 70 μg/kg, about 75 μg/kg, about 80 μg/kg, about 85 μg/kg, about 90 μg/kg, about 95 μg/kg, about 100 μg/kg, about 125 μg/kg, about 150 μg/kg, about 175 μg/kg, about 200 μg/kg, about 225 μg/kg, about 250 μg/kg, about 275 μg/kg, or about 300 μg/kg of the subject's body weight. In some embodiments, the dose of the CD20-binding molecule is increased from about 50 μg/kg to about 55 μg/kg, about 60 μg/kg, about 65 μg/kg, about 70 μg/kg, about 75 μg/kg, about 80 μg/kg, about 85 μg/kg, about 90 μg/kg, about 95 μg/kg, about 100 μg/kg, about 125 μg/kg, about 150 μg/kg, about 175 μg/kg, about 200 μg/kg, about 225 μg/kg, about 250 μg/kg, about 275 μg/kg, about 300 μg/kg, about 325 μg/kg, about 350 μg/kg, about 375 μg/kg, or about 400 μg/kg of the subject's body weight. In some embodiments, the dose of the CD20-binding molecule is increased from about 75 μg/kg to about 80 μg/kg, about 85 μg/kg, about 90 μg/kg, about 95 μg/kg, about 100 μg/kg, about 125 μg/kg, about 150 μg/kg, about 175 μg/kg, about 200 μg/kg, about 225 μg/kg, about 250 μg/kg, about 275 μg/kg, about 300 μg/kg, about 325 μg/kg, about 350 μg/kg, about 375 μg/kg, about 400 μg/kg, about 425 μg/kg, about 450 μg/kg, about 475 μg/kg, or about 500 μg/kg of the subject's body weight.

In some embodiments, the dose of the CD20-binding molecule is increased during a 42-day cycle. In some embodiments, the dose of the CD20-binding molecule is increased during a first 28-day cycle following the 42-day cycle. In some embodiments, the dose of the CD20-binding molecule is increased during a second 28-day cycle following the first 28-day cycle and the 42-day cycle. In some embodiments, the dose of the CD20-binding molecule is increased during a third 28-day cycle following the first and second 28-day cycles and the 42-day cycle. In some embodiments, the dose of the CD20-binding molecule is increased during an at least one additional 28-day cycle following the first, second, and third 28-day cycles and the 42-day cycle.

In some embodiments, the dose of the CD20-binding molecule is increased during a first 28-day cycle. In some embodiments, the dose of the CD20-binding molecule is increased during a second 28-day cycle following the first 28-day cycle. In some embodiments, the dose of the CD20-binding molecule is increased during a third 28-day cycle following the first and second 28-day cycles. In some embodiments, the dose of the CD20-binding molecule is increased during an at least one additional 28-day cycle following the first, second, and third 28-day cycles.

In some embodiments, the dose of the CD20-binding molecule is decreased during the course of treatment. In some embodiments, the dose of the CD20-binding molecule is decreased once per week, twice per week, three times per week, four times per week, 5 times per week, once every 2 weeks, once every 3 weeks, once per month, once every 2 months, once every 3 months, once every 4 months, once every 5 months, once every 6 months, or once per year during the course of treatment.

In some embodiments, the dose of the CD20-binding molecule is decreased from about 10 μg/kg to about 5 μg/kg, about 2.5 μg/kg, about 1 μg/kg, about 0.5 μg/kg, about 0.1 μg/kg, about 0.05 μg/kg, or about 0.01 μg/kg of the subject's body weight. In some embodiments, the dose of the CD20-binding molecule is decreased from about 25 μg/kg to about 20 μg/kg, about 15 μg/kg, about 10 μg/kg, about 5 μg/kg, about 2.5 μg/kg, about 1 μg/kg, about 0.5 μg/kg, about 0.1 μg/kg, about 0.05 μg/kg, or about 0.01 μg/kg of the subject's body weight. In some embodiments, the dose of the CD20-binding molecule is decreased from about 50 μg/kg to about 45 μg/kg, about 40 μg/kg, about 35 μg/kg, about 30 μg/kg, about 25 μg/kg, about 20 μg/kg, about 15 μg/kg, about 10 μg/kg, about 5 μg/kg, about 1 μg/kg, about 0.5 μg/kg, about 0.1 μg/kg, about 0.05 μg/kg, or about 0.01 μg/kg of the subject's body weight. In some embodiments, the dose of the CD20-binding molecule is decreased from about 75 μg/kg to about 70 μg/kg, about 65 μg/kg, about 60 μg/kg, about 55 μg/kg, about 50 μg/kg, about 45 μg/kg, about 40 μg/kg, about 35 μg/kg, about 30 μg/kg, about 25 μg/kg, about 20 μg/kg, about 15 μg/kg, about 10 μg/kg, about 5 μg/kg, about 1 μg/kg, about 0.5 μg/kg, about 0.1 μg/kg, about 0.05 μg/kg, or about 0.01 μg/kg of the subject's body weight.

In some embodiments, the dose of the CD20-binding molecule is decreased during a 42-day cycle. In some embodiments, the dose of the CD20-binding molecule is decreased during a first 28-day cycle following the 42-day cycle. In some embodiments, the dose of the CD20-binding molecule is decreased during a second 28-day cycle following the first 28-day cycle and the 42-day cycle. In some embodiments, the dose of the CD20-binding molecule is decreased during a third 28-day cycle following the first and second 28-day cycles and the 42-day cycle. In some embodiments, the dose of the CD20-binding molecule is decreased during an at least one additional 28-day cycle following the first, second, and third 28-day cycles and the 42-day cycle.

In some embodiments, the dose of the CD20-binding molecule is decreased during a first 28-day cycle. In some embodiments, the dose of the CD20-binding molecule is decreased during a second 28-day cycle following the first 28-day cycle. In some embodiments, the dose of the CD20-binding molecule is decreased during a third 28-day cycle following the first and second 28-day cycles. In some embodiments, the dose of the CD20-binding molecule is decreased during an at least one additional 28-day cycle following the first, second, and third 28-day cycles.

In some embodiments, the CD20-binding molecule is administered once per week, twice per week, three times per week, four times per week, five times per week, six times per week, or seven times per week. In some embodiments, the CD20-binding molecule is administered once per week, wherein the dose of the CD20-binding molecule is in an amount of about 10 μg/kg, about 25 μg/kg, about 50 μg/kg, or about 75 μg/kg of the subject's body weight. In some embodiments, the CD20-binding molecule is administered twice per week, wherein each dose of the CD20-binding molecule is in an amount of about 10 μg/kg, about 25 μg/kg, about 50 μg/kg, or about 75 μg/kg of the subject's body weight. In some embodiments, the CD20-binding molecule is administered three times per week, wherein each dose of the CD20-binding molecule is in an amount of about 10 μg/kg, about 25 μg/kg, about 50 μg/kg, or about 75 μg/kg of the subject's body weight. In some embodiments, the CD20-binding molecule is administered four times per week, wherein each dose of the CD20-binding molecule is in an amount of about 10 μg/kg, about 25 μg/kg, about 50 μg/kg, or about 75 μg/kg of the subject's body weight. In some embodiments, the CD20-binding molecule is administered five times per week, wherein each dose of the CD20-binding molecule is in an amount of about 10 μg/kg, about 25 μg/kg, about 50 μg/kg, or about 75 μg/kg of the subject's body weight. In some embodiments, the CD20-binding molecule is administered six times per week, wherein each dose of the CD20-binding molecule is in an amount of about 10 μg/kg, about 25 μg/kg, about 50 μg/kg, or about 75 μg/kg of the subject's body weight. In some embodiments, the CD20-binding molecule is administered seven times per week, wherein each dose of the CD20-binding molecule is in an amount of about 10 μg/kg, about 25 μg/kg, about 50 μg/kg, or about 75 μg/kg of the subject's body weight.

In some embodiments, the step of administering the CD20-binding molecule comprises administering ten doses of the CD20-binding molecule on about 1, 3, 5, 8, 10, 12, 15, 22, 29 and 36 of a 42-day cycle; wherein each dose is in an amount of about 10 μg/kg, about 25 μg/kg, about 50 μg/kg, or about 75 μg/kg of the subject's body weight. In some embodiments, the method further comprises administering the CD20-binding molecule weekly during a first 28-day cycle following the 42-day cycle on days 1, 8, 15, and 22 of the first 28-day cycle. In some embodiments, the method further comprises administering the CD20-binding molecule weekly during a second 28-day cycle following the first 28-day cycle and the 42-day cycle, wherein the CD20 binding molecule is administered on days 1, 8, 15, and 22 of the second 28-day cycle. In some embodiments, the method further comprises administering the CD20-binding molecule weekly during a third 28-day cycle following the first and second 28-day cycle and the 42-day cycle, wherein the CD20 binding molecule is administered on days 1, 8, 15, and 22 of the third 28-day cycle. In some embodiments, the method further comprises administering the CD20-binding molecule weekly during a fourth 28-day cycle following the first, second, and third 28-day cycle and the 42-day cycle, wherein the CD20 binding molecule is administered on days 1, 8, 15, and 22 of the fourth 28-day cycle. In some embodiments, each dose administered during the first, second, third, and/or fourth 28-day cycle is in an amount of about 10 μg/kg, about 25 μg/kg, about 50 μg/kg, or about 75 μg/kg of the subject's body weight. As used herein, the numbered days of the cycle refer to days relative to day 1, i.e., the day in which the first dose of the CD20-binding molecule is administered in that cycle.

In some embodiments, the step of administering the CD20-binding molecule comprises administering six doses of the CD20-binding molecule on days 1, 3, 5, 8, 10, and 12 of a first 28-day cycle, wherein each dose is in an amount of about 10 μg/kg, about 20 μg/kg, about 25 μg/kg, about 50 μg/kg, or about 75 μg/kg of the subject's body weight. In some embodiments, the method further comprises administering the CD20-binding molecule weekly during a second 28-day cycle following the first 28-day cycle, wherein the CD20-binding molecule is administered on days 1, 8, 15, and 22 of the second 28-day cycle. In some embodiments, the method further comprises administering the CD20-binding molecule weekly during a third 28-day cycle following the first and second 28-day cycle, wherein the CD20-binding molecule is administered on days 1, 8, 15, and 22 of the third 28-day cycle. In some embodiments, the method further comprises administering the CD20-binding molecule weekly during a fourth 28-day cycle following the first, second, and third 28-day cycle, wherein the CD20-binding molecule is administered on days 1, 8, 15, and 22 of the fourth 28-day cycle. In some embodiments, the method further comprises administering the CD20-binding molecule weekly during a fifth 28-day cycle following the first, second, third, and fourth 28-day cycle, wherein the CD20-binding molecule is administered on days 1, 8, 15, and 22 of the fifth 28-day cycle. In some embodiments, the method further comprises administering the CD20-binding molecule weekly during a sixth 28-day cycle following the first, second, third, fourth, and fifth 28-day cycle, wherein the CD20-binding molecule is administered on days 1, 8, 15, and 22 of the sixth 28-day cycle.

In some embodiments, the step of administering the CD20-binding molecule comprises administering six doses of the CD20-binding molecule on days 1, 3, 5, 8, 10, and 12 of a first 28-day cycle, wherein each dose is in an amount of about 10 μg/kg of the subject's body weight. In some embodiments, the method further comprises administering the CD20-binding molecule weekly during a second 28-day cycle following the first 28-day cycle, wherein the CD20-binding molecule is administered on days 1, 8, 15, and 22 of the second 28-day cycle, wherein each dose is in an amount of about 10 μg/kg of the subject's body weight.

In some embodiments, the step of administering the CD20-binding molecule comprises administering six doses of the CD20-binding molecule on days 1, 3, 5, 8, 10, and 12 of a first 28-day cycle, wherein each dose is in an amount of about 25 μg/kg of the subject's body weight. In some embodiments, the method further comprises comprising administering the CD20-binding molecule weekly during a second 28-day cycle following the first 28-day cycle, wherein the CD20-binding molecule is administered on days 1, 8, 15, and 22 of the second 28-day cycle, wherein each dose is in an amount of about 25 μg/kg of the subject's body weight.

In some embodiments, the step of administering the CD20-binding molecule comprises administering six doses of the CD20-binding molecule on days 1, 3, 5, 8, 10, and 12 of a first 28-day cycle, wherein each dose is in an amount of about 20 μg/kg of the subject's body weight. In some embodiments, the method further comprises administering the CD20-binding molecule weekly during a second 28-day cycle following the first 28-day cycle, wherein the CD20-binding molecule is administered on days 1, 8, 15, and 22 of the second 28-day cycle, wherein each dose is in an amount of about 20 μg/kg of the subject's body weight.

In some embodiments, the step of administering the CD20-binding molecule comprises administering four doses of the CD20-binding molecule on days 1, 5, 8, and 12 of a first 28-day cycle, wherein each dose is in an amount of about 25 μg/kg of the subject's body weight. In some embodiments, the method further comprises administering the CD20-binding molecule during a second 28-day cycle following the first 28-day cycle, wherein the CD20-binding molecule is administered on days 1, 5, 8, and 12 of the second 28-day cycle, wherein each dose is in an amount of about 25 μg/kg of the subject's body weight. In some embodiments, the method further comprises administering the CD20-binding molecule during a third 28-day cycle following the first and second 28-day cycle, wherein the CD20-binding molecule is administered on days 1, 8, 15, and 22 of the third 28-day cycle, wherein each dose is in an amount of about 25 μg/kg of the subject's body weight.

In some embodiments, the step of administering the CD20-binding molecule comprises administering four doses of the CD20-binding molecule on days 1, 5, 8, and 12 of a first 28-day cycle, wherein each dose is in an amount of about 50 μg/kg of the subject's body weight. In some embodiments, the method further comprises administering the CD20-binding molecule during a second 28-day cycle following the first 28-day cycle, wherein the CD20-binding molecule is administered on days 1, 5, 8, and 12 of the second 28-day cycle, wherein each dose is in an amount of about 50 μg/kg of the subject's body weight. In some embodiments, the method further comprises administering the CD20-binding molecule during a third 28-day cycle following the first and second 28-day cycle, wherein the CD20-binding molecule is administered on days 1, 8, 15, and 22 of the third 28-day cycle, wherein each dose is in an amount of about 50 μg/kg of the subject's body weight.

In some embodiments, each dose of the CD20-binding molecule is an intravenous infusion that is administered over about 30 to about 75 minutes, such as about 31, about 32, about 33, about 34, about 35, about 36, about 37, about 38, about 39, about 40, about 41, about 42, about 43, about 44, about 45, about 46, about 47, about 48, about 49, about 50, about 51, about 52, about 53, about 54, about 55, about 56, about 57, about 58, about 59, about 60, about 61, about 62, about 63, about 64, about 65, about 66, about 67, about 68, about 69, about 70, about 71, about 72, about 73, about 74, or about 75 minutes. In some embodiments, each dose of the CD20-binding molecule is an intravenous infusion that is administered over about 60 minutes.

In some embodiments, the step of administering the CD20-binding molecule comprises administering six doses of the CD20-binding molecule over 12 days of a 21-day cycle, each dose being an intravenous infusion that is administered over about 1 hour, wherein each dose is in an amount of about 10 μg/kg, about 25 μg/kg, about 50 μg/kg, or about 75 μg/kg of the subject's body weight. In some embodiments, administering a subsequent dose of the CD20-binding molecules occurs at least about 24 hours administering a prior dose. In some embodiments, the administering of the CD20-binding molecule takes occurs on a Monday, Wednesday and Friday of two successive weeks in a 21-day cycle. In some embodiments, the methods may further comprise repeating the 21-day cycle of administering the CD20-binding molecule. In some embodiments, the methods may further comprise repeating the 21-day cycle of administering the CD20-binding molecule once, twice, three times, four times, five times, six times, seven times, or more than seven times.

In some embodiments, after the completion of the one or more 21-day cycles of administering the CD20-binding molecule, the methods further comprise administering the CD20-binding molecule weekly following the 21-day cycle(s). In some embodiments, the methods further comprise administering the CD20-binding molecule weekly following the 21-day cycle(s) for 2, 3, 4, 5, 6, 7, 8, 9, 10, 11, or more than 11 weeks. In some embodiments, the methods further comprise administering the CD20-binding molecule weekly during a 28-day cycle following the 21-day cycle(s).

In some embodiments, the step of administering the CD20-binding molecule comprises administering ten doses of the CD20-binding molecule, each dose being an intravenous infusion that is administered over about 1 hour on days 1, 3, 5, 8, 10, 12, 15, 22, 29 and 36 of a 42-day cycle, wherein each dose is in an amount of about 10 μg/kg, about 25 μg/kg or about 50 μg/kg of the subject's body weight. In some embodiments, the methods may further comprise repeating the 42-day cycle of administering the CD20-binding molecule once, twice, three times, four times, five times, six times, seven times, or more than seven times.

In some embodiments, after the completion of the one or more 42-day cycles of administering the CD20-binding molecule, the methods further comprise administering the CD20-binding molecule weekly following the 42-day cycle(s). In some embodiments, the methods further comprise administering the CD20-binding molecule weekly following the 42-day cycle(s) for 2, 3, 4, 5, 6, 7, 8, 9, 10, 11, or more than 11 weeks. In some embodiments, the methods further comprise administering the CD20-binding molecule weekly during a 28-day cycle following the 42-day cycle(s).

In some embodiments, a method for treating or slowing the progression of a disease, disorder, or condition that comprises administering to a subject in need thereof an effective amount of a CD20-binding molecule further comprises administering to the subject one or more additional therapeutic agents. Thus, in some embodiments, a method for treating or slowing the progression of a disease, disorder, or condition, the method comprising administering to a subject in need thereof an effective amount of: (i) a CD20-binding molecule; and (ii) one or more additional therapeutic agents; wherein the CD20-binding molecule comprises a polypeptide having the sequence of SEQ ID NO: 54.

In some embodiments, the one or more additional therapeutic agents are administered on the same day as the CD20-binding molecule. In some embodiments, the one or more additional therapeutic agents are administered at least one day after the CD20-binding molecule is administered.

In some embodiments wherein the CD20-binding molecule is administered on days 1, 3, 5, 8, 10, 12, 15, 22, 29 and 36 of a 42 day cycle, the one or more additional therapeutic agents are administered on day 16 and day 30 of the 42-day cycle. In some embodiments wherein the CD20 binding molecule is administered on days 1, 3, 5, 8, 10, and 12 of a 28-day cycle, the one or more additional therapeutic agents are administered daily on days 1-21 of that 28-day cycle.

In some embodiments wherein the CD20-binding molecule is administered on days 1, 8, 15, and 22 of a 28-day cycle, the one or more additional therapeutic agents are administered on day 2 and day 16 of the 28-day cycle. In some embodiments wherein the CD20 binding molecule is administered on days 1, 8, 15, and 22 of a 28-day cycle, the one or more additional therapeutic agents are administered daily on days 1-21 of that 28-day cycle.

In some embodiments, the one or more additional therapeutic agents comprise oxaliplatin and gemcitabine. Thus, in some embodiments, the methods comprise administering (a) the CD20-binding molecule as described herein and (b) (i) gemcitabine or a pharmaceutically acceptable salt thereof or (ii) oxaliplatin. In some embodiments, the methods comprise administering (a) the CD20-binding molecule as described herein and (b) (i) gemcitabine or a pharmaceutically acceptable salt thereof and (ii) oxaliplatin. In some embodiments, the gemcitabine (or pharmaceutically acceptable salt thereof) and oxaliplatin are a GEMOX regimen known in the art. In some embodiments, the gemcitabine and the oxaliplatin are administered in separate compositions. In some embodiments, the gemcitabine and the oxaliplatin are adminstered as a fixed-dose combination (FDC).

In some embodiments, the gemcitabine is administered at about 1,000 mg/m² as an intravenous infusion over about 30 minutes (e.g., about 15 to about 45 minutes). In some embodiments, the oxaliplatin is administered at about 100 mg/m² as an intravenous infusion over about 2 hours (e.g., about 1 hour to about 3 hours). In some embodiments, the oxaliplatin and the gemcitabine are administered on the same day, and wherein the oxaliplatin is administered after administration of the gemcitabine on that day. In some embodiments, the oxaliplatin and the gemcitabine are administered on the same day, and wherein the oxaliplatin is administered before administration of the gemcitabine on that day. In some embodiments, the oxaliplatin is infusion is initiated about 1 hour after the infusion of the gemcitabine concludes (e.g., about 30 minutes to about 2 hours after the infusion of the gemcitabine concludes).

In some embodiments, the methods further comprise administering to the subject gemcitabine or a pharmaceutically acceptable salt thereof at a dose of about 1,000 mg/m² on days 16 and 30 of a 42-day cycle; wherein each dose of gemcitabine or pharmaceutically acceptable salt thereof is administered as an intravenous infusion over about 30 minutes. In some embodiments, the methods further comprise administering to the subject gemcitabine or a pharmaceutically acceptable salt thereof at a dose of about 1,000 mg/m² on days 2 and 16 of the 28-day cycle; wherein each dose of gemcitabine or pharmaceutically acceptable salt thereof is administered as an intravenous infusion over about 30 minutes. In some embodiments, a first 42-day cycle is followed by one or more 28-day cycles. In some embodiments, each subsequent 28-day cycle includes administering to the subject gemcitabine or a pharmaceutically acceptable salt thereof at a dose of about 1,000 mg/m² on days 2 and 16 of the 28-day cycle; wherein each dose of gemcitabine or pharmaceutically acceptable salt thereof is administered as an intravenous infusion over about 30 minutes. In some embodiments, gemcitabine is not administered in the same week as the CD20 binding molecule.

In some embodiments, the methods further comprise administering to the subject gemcitabine or a pharmaceutically acceptable salt thereof at a dose of about 1,000 mg/m² on days 2 and 16 of one or more 28-day cycles (cycle 2 and on following a first cycle of 42-days); wherein each dose of gemcitabine or pharmaceutically acceptable salt thereof is administered as an intravenous infusion over about 30 minutes. In some further embodiments, gemcitabine is not administered in the same week as the CD20 binding molecule.

In some embodiments, the methods further comprises administering to the subject oxaliplatin at a dose of about 100 mg/m² on days 16 and 30 of the 42-day cycle; wherein each dose of oxaliplatin is administered as an intravenous infusion over about 2 hours; and wherein each dose of oxaliplatin is administered after the start of each infusion of gemcitabine or a pharmaceutically acceptable salt thereof. In some embodiments, the methods further comprise administering to the subject oxaliplatin at a dose of about 100 mg/m² on days 16 and 30 of the 42-day cycle; wherein each dose of oxaliplatin is administered as an intravenous infusion over about 2 hours; and wherein each dose of oxaliplatin or a pharmaceutically acceptable salt thereof is administered about one hour after the start of each infusion of gemcitabine or a pharmaceutically acceptable salt thereof. In some embodiments, the methods further comprise administering to the subject oxaliplatin at a dose of about 100 mg/m² on days 2 and 16 of each 28-day cycle; wherein each dose of oxaliplatin is administered as an intravenous infusion over about 2 hours; and wherein each dose of oxaliplatin is administered about one hour after the start of each infusion of gemcitabine or a pharmaceutically acceptable salt thereof. In some embodiments, oxaliplatin is not administered in the same week as the CD20 binding molecule.

In some embodiments, after the completion of the one or more 21-day cycles of administering the CD20-binding molecule as described herein, the methods further comprise administering the CD20-binding molecule once every other week following the 21-day cycle(s) and administering gemcitabine or a pharmaceutically acceptable salt thereof and oxaliplatin on alternate weeks from the administering of the CD20-binding molecule.

In some embodiments, the one or more additional therapeutic agents comprise an immunomodulatory drug. Thus, in some embodiments, the methods further comprise administering to the subject an immunomodulatory drug. In some embodiments, the immunomodulatory drug is administered orally. In some embodiments, the immunomodulatory drug is adminstered at a dose of about 10 mg to about 100 mg daily, such as about 20 mg. In some embodiments, the immunomodulatory drug is administered orally at a dose of about 20 mg on each of days 1 and 21 of the 28-day cycle.

In some embodiments, the immunomodulatory drug is lenalidomide or a pharmaceutically acceptable salt thereof. In some embodiments, the lenalidomide is administered at a dose of about 20 milligram per day. In some embodiments, the lenalidomide is administered orally. In some embodiments, the lenalidomide is administered orally at a dose of about 20 mg on each of days 1 and 21 of a 28-day cycle. In some embodiments, the lenalidomide is administered orally at a dose of about 20 mg on each of days 1 and 21 of a 28-day cycle, wherein the CD20-binding molecule is administered on days 1, 5, 8, and 12 of that cycle or days 1, 8, 15, and 22 of that cycle.

In some embodiments, the one or more additional therapeutic agents is a Bruton's tyrosine kinase (BTK) inhibitor. In some embodiments, the methods comprise administering to the subject lenalidomide or a pharmaceutically acceptable salt thereof and a BTK inhibitor. In some embodiments, the methods comprise administering to the subject gemcitabine and/or oxaliplatin, and a BTK inhibitor. Exemplary BTK inhibitors include, but are not limited to, ibrutinib, zanubrutinib, and acalabrutinib.

In some embodiments, the one or more additional therapeutic agents is an inhibitor of phosphoinositide 3-kinase (PI3K). In some embodiments, the methods comprise administering to the subject lenalidomide or a pharmaceutically acceptable salt thereof and a PI3K inhibitor. In some embodiments, the methods comprise administering to the subject gemcitabine and/or oxaliplatin, and a PI3K inhibitor. Exemplary PI3K inhibitors include, but are not limited to, alpelisib, idelalisib, copanlisib, and duvelisib.

In some embodiments, the methods comprise administering the CD20-binding molecule as described herein and an immunomodulatory drug. In some embodiments, the immunomodulatory drug is an mTOR inhibitor, rapalog, and/or an ATP-competitive mTOR kinase inhibitor. In some embodiments, the methods further comprise administering to the subject everolimus, temsirolimus, ridaforolimus, sirolimus (rapamycin), or a derivative thereof. In some embodiments, the methods further comprise administering to the subject the immunomodulatory drug orally at a dose of about 0.1 to 5 mg/kg. In some embodiments, the methods further comprise administering to the subject an immunomodulatory drug on any one of or all of days 1 through 14 of the first cycle. In some embodiments, the methods further comprise administering to the subject the immunomodulatory drug orally using a loading dose of about 1 to 15 mg/kg on day 1 and one or more maintenance doses of about 0.1 to 5 mg/kg throughout days 2 to 14. In some embodiments, the CD20-binding protein is administered by an intravenous infusion that is administered over about 1 hour on about days 3, 5, 7, 10, 12, and 14 of a 24-day cycle.

In some embodiments, the dosage range for administration of the pharmaceutical composition may generally be from about 0.0001 to 1 milligram per kilogram (mg/kg), and more usually 0.005 to 0.5 mg/kg, of the subject's body weight. Illustrative dosages may be 0.01 mg/kg body weight, 0.015 mg/kg body weight, 0.05 mg/kg body weight, 0.085 mg/kg body weight or 0.1 mg/kg body weight or within the range of 0.01-0.1 mg/kg. An illustrative treatment regime is a once or twice daily administration, or a once or twice weekly administration, once every two weeks, once every three weeks, once every four weeks, once a month, once every two or three months or once every three to 6 months. Dosages may be selected and readjusted by the skilled health care professional as required to maximize therapeutic benefit for a particular patient.

For intravenous administration of a pharmaceutical composition described herein, the dosage range may generally be from about 0.01 to 500 micrograms (μg) per kilogram (kg) of the subject's body weight (μg/kg), and more, usually 0.5 to 75 μg/kg. Illustrative dosages may be 1 μg/kg body weight, 5 μg/kg body weight, 10 μg/kg body weight, 20 μg/kg body weight, 25 μg/kg body weight, 35 μg/kg body weight, or 50 μg/kg body weight or within the range of 1 to 50 μg/kg. An illustrative treatment regime is a once or twice daily administration, or a once or twice weekly administration, once every two weeks, once every three weeks, once every four weeks, once a month, once every two or three months or once every three to 6 months.

A CD20-binding molecule as described herein and/or a composition thereof may be administered via one or more routes of administration, using one or more of a variety of methods known in the art. As will be appreciated by the skilled worker, the route and/or mode of administration will vary depending upon the desired results. Routes of administration for CD20-binding molecule compositions, pharmaceutical compositions, and diagnostic compositions as described herein include, e.g. intravenous, intramuscular, intradermal, intraperitoneal, subcutaneous, spinal, or other parenteral routes of administration, for example by injection or infusion. In other embodiments, a CD20-binding molecule as described herein and/or a composition thereof may be administered by a non-parenteral route, such as a topical, epidermal or mucosal route of administration, for example, intranasally, orally, vaginally, rectally, sublingually, or topically.

For certain embodiments of the methods as described herein, the administering step involves a parenteral administration of a CD20-binding molecule as described herein and/or a composition thereof (e.g. a pharmaceutical composition as described herein). In some embodiments, the administering step involves an intravenous administration of a CD20-binding molecule as described herein and/or a composition thereof (e.g. a pharmaceutical composition as described herein).

For certain embodiments of the methods as described herein, the method comprises the step of diluting a pharmaceutical composition as described herein in an aqueous solution, sterile water, dextrose solution, dextrose monohydrate, hydrous dextrose, saline solution, and/or sodium chloride solution prior to the administering step.

Therapeutic CD20-binding molecule as described herein and/or a composition thereof may be administered with one or more of a variety of medical devices known in the art. For example, in one embodiment, a pharmaceutical composition described herein may be administered with a needleless hypodermic injection device. Examples of well-known implants and modules useful in the methods disclosed herein are in the art, including e.g., implantable micro-infusion pumps for controlled rate delivery; devices for administering through the skin; infusion pumps for delivery at a precise infusion rate; variable flow implantable infusion devices for continuous drug delivery; and osmotic drug delivery systems. These and other such implants, delivery systems, and modules are known to those skilled in the art.

A CD20-binding molecule as described herein and/or a composition thereof may be administered alone or in combination with one or more other therapeutic or diagnostic agents. A combination therapy may include a CD20-binding molecule as described herein and/or a pharmaceutical composition thereof combined with at least one other therapeutic agent selected based on the particular patient, disease or condition to be treated. Examples of other such agents include, inter alia, a cytotoxic, anti-cancer or chemotherapeutic agent, an anti-inflammatory or anti-proliferative agent, an antimicrobial or antiviral agent, growth factors, cytokines, an analgesic, a therapeutically active small molecule or polypeptide, a single chain antibody, a classical antibody or fragment thereof, or a nucleic acid molecule which modulates one or more signaling pathways, and similar modulating therapeutics which may complement or otherwise be beneficial in a therapeutic or prophylactic treatment regimen.

Treatment of a patient with a CD20-binding molecule as described herein and/or a composition thereof preferably leads to cell death of targeted CD20+ cells and/or the inhibition of growth of targeted CD20+ cells. As such, certain CD20-binding molecules as described herein, and pharmaceutical compositions comprising them, will be useful in methods for treating a variety of pathological disorders in which killing or depleting CD20+ target cells may be beneficial, such as, inter alia, cancers, tumors, immune disorders, and growth abnormalities involving CD20+ cells. Also provided herein are methods for suppressing cell proliferation, and treating cell disorders, including neoplasia, overactive B-cells, and overactive T-cells.

CD20 is expressed by cells involved in a variety of malignancies, such as, e.g., hematologic diseases, rheumatic diseases, hematologic cancers, leukemias, lymphomas, melanomas, myelomas, B-cell lymphomas, B-cell non-Hodgkins lymphomas (B-cell NHL), Burkitt's lymphomas (BL), B-cell chronic lymphocytic leukemias (B-cell CLL), chronic lymphocytic leukemias (CLL), diffuse large B-cell lymphomas (DLBCL or DLBL), follicular lymphomas (FL), hairy cell leukemias (HCL), Hodgkins lymphomas (HD), immunoblastic large cell lymphomas, mantle cell lymphomas (MCL), melanomas, non-Hodgkins lymphomas (NHL), precursor B-lymphoblastic lymphomas (B-LBL), small lymphocytic lymphoma (SLL), and T-cell lymphomas (TCL), amyloidosis, ankylosing spondylitis, asthma, Crohn's disease, diabetes, graft rejection, graft-versus-host disease, Hashimoto's thyroiditis, hemolytic uremic syndrome, HIV-related diseases, lupus erythematosus, multiple sclerosis, polyarteritis nodosa, polyarthritis, psoriasis, psoriatic arthritis, rheumatoid arthritis, scleroderma, septic shock, Sjorgren's syndrome, ulcerative colitis, and/or vasculitis.

In some embodiments CD20-binding molecules as described herein and/or a composition thereof can be used to treat or prevent cancers, tumors (malignant and non-malignant), growth abnormalities, and immune disorders. In a further aspect, the above ex vivo method can be combined with the above in vivo method to provide methods of treating or preventing rejection in bone marrow transplant recipients, and for achieving immunological tolerance.

In some embodiments, the CD20-binding molecules described herein are used to treat cancer and, in some embodiments, the cancer is a hematologic cancer or a solid tumor. In some embodiments, the cancer large B-cell lymphoma, such as large B-cell lymphoma that is relapsed or refractory to at least one additional treatment (i.e., treatment with an anti-cancer drug). In some embodiments, the cancer is diffuse large B-cell lymphoma, such as diffuse large B-cell lymphoma that is relapsed or refractory to at least one additional treatment (i.e., treatment with an anti-cancer drug). In some embodiments, the cancer is non-Hodgkin's B-cell lymphoma, such as non-Hodgkin's B-cell lymphoma that is relapsed or refractory to at least one additional treatment (i.e., treatment with an anti-cancer drug). In some embodiments, the cancer is mantle cell lymphoma, such as mantle cell lymphoma that is relapsed or refractory to at least one additional treatment (i.e., treatment with an anti-cancer drug). In some embodiments, the cancer is follicular lymphoma, such as follicular lymphoma that is relapsed/refractory to at least one additional therapy.

Also provided herein are methods for treating or slowing the progression of a disease, disorder, or condition, comprising administering to a subject in need thereof an effective amount of (i) a CD20-binding molecule and (ii) an additional therapeutic agent, wherein: the CD20-binding molecule comprises: (a) a CD20-binding region, wherein the CD20-binding region is capable of specifically binding an extracellular portion of a CD20 protein, and (b) a Shiga toxin effector region.

In some embodiments, the CD20-binding molecule is a multivalent CD20-binding molecule. In some embodiments, the CD20-binding molecule comprises two CD20-binding regions, wherein each CD20-binding region is capable of specifically binding an extracellular portion of a CD20 protein.

In some embodiments of the methods described herein, the additional therapeutic agent is a first additional therapeutic agent, and the method further comprises administering a second additional therapeutic agent. In some embodiments, the first additional therapeutic agent and the second additional therapeutic agent are present in the same composition. In some embodiments, the first additional therapeutic agent is present in a first composition and the second additional therapeutic agent is present in a second composition.

In some embodiments, the additional therapeutic agent is a chemotherapeutic agent. In some embodiments, the chemotherapeutic agent is a nucleoside analog or a platinum-based chemotherapeutic agent. In some embodiments, the nucleoside analog is gemcitabine, capecitabine, cladribine, clofarabine, cytarabine, decitabine, fludarabine or troxacitabine, or a pharmaceutically acceptable salt thereof. In some embodiments, the platinum-based chemotherapeutic agent is oxaliplatin, carboplatin, cisplatin or nedaplatin.

In some embodiments, the first additional therapeutic agent or the second additional therapeutic agent is a nucleoside analog or a platinum-based chemotherapeutic agent. In some embodiments, the first additional therapeutic agent is gemcitabine or a pharmaceutically acceptable salt thereof and the second additional therapeutic agent is oxaliplatin or a pharmaceutically acceptable salt thereof. In some embodiments, a CD20-binding molecule is administered in combination with a gemcitabine and oxaliplatin (GEMOX) regimen.

In some embodiments, the additional therapeutic agent is an immunomodulatory drug (IMiD). In some embodiments, the immunomodulatory drug is lenalidomide, apremilast, pomalidomide or thalidomide, or a pharmaceutically acceptable salt thereof. In some embodiments, the immunomodulatory drug is an mTOR inhibitor, rapalog, and/or an ATP-competitive mTOR kinase inhibitor; such as everolimus, temsirolimus, ridaforolimus, or sirolimus, or a pharmaceutically acceptable salt thereof.

In some embodiments, the additional therapeutic agent is a phosphoinositide 3-kinase (PI3K) inhibitor. In some embodiments, the PI3K inhibitor is alpelisib, idelalisib, copanlisib, or duvelisib, or a pharmaceutically acceptable salt thereof.

In some embodiments, the additional therapeutic agent is a Bruton's tyrosine kinase (BTK) inhibitor. In some embodiments, the BTK inhibitor is acalabrutinib, evobrutinib, ibrutinib, spebrutinib or zanubrutinib, or a pharmaceutically acceptable salt thereof.

In some embodiments, the first additional therapeutic agent is an immunomodulatory drug and the second additional therapeutic agent is a BTK inhibitor. In some embodiments, the first additional therapeutic agent is lenalidomide or a pharmaceutically acceptable salt thereof and the second additional therapeutic agent is a BTK inhibitor. In some embodiments, the first additional therapeutic agent is pomalidomide or a pharmaceutically acceptable salt thereof and the second additional therapeutic agent is a BTK inhibitor.

In some embodiments, the additional therapeutic agent is an mTOR inhibitor, rapalog, and/or an ATP-competitive mTOR kinase inhibitor; and optionally wherein the rapalog is everolimus, temsirolimus, ridaforolimus, or sirolimus, or a derivative of any of the aforementioned.

In some embodiments of the methods described herein, the CD20-binding molecule comprises or consists essentially of a polypeptide having the amino acid sequence of any one of SEQ ID NOs: 47-175 and 249-304. In some embodiments, the CD20-binding molecule is multimeric and comprises or consists essentially of: (a) two polypeptides, each polypeptide having the amino acid sequence of any one of SEQ ID NOs: 47-175 and 249-304; and (b) a cysteine disulfide bond linking the two polypeptides, wherein the cysteine disulfide bond is between a cysteine residue in each of the two polypeptides located at amino acid position 242, 482, 483, 484, 490, 491, 492, 493, 494, 495, 499, 500, 501, 502, 503, 504, 505, 510, 511, 512, 513, or 521 of the amino acid sequence of the polypeptide. In some embodiments, each polypeptide comprises an amino-terminal methionine residue.

In some embodiments, the CD20-binding molecule is a homodimer and consists essentially of (a) the two polypeptides, wherein the two polypeptides are identical; and (b) the cysteine disulfide bond.

In some embodiments, the two polypeptides are identical, and each polypeptide has the amino acid sequence of SEQ ID NO: 49; and the disulfide bond is between each cysteine residue at amino acid position 490 in SEQ ID NO:49. In some embodiments, the two polypeptides are identical, and each identical polypeptide has the amino acid sequence of any one of SEQ ID NO:50, SEQ ID NO:61, SEQ ID NO:73, SEQ ID NO:96, SEQ ID NO:101, and SEQ ID NO:102; and the disulfide bond is between each cysteine residue at amino acid position 501 of the amino acid sequence of each identical polypeptide. In some embodiments, the two polypeptides are identical, and each identical polypeptide has the amino acid sequence of any one of SEQ ID NO:53, SEQ ID NO:63, SEQ ID NO:66, SEQ ID NO:75, SEQ ID NO:83, SEQ ID NO:89, and SEQ ID NO:95; and the disulfide bond is between each cysteine residue at amino acid position 512 of the amino acid sequence of each identical polypeptide. In some embodiments, the two polypeptides are identical, and each identical polypeptide has the amino acid sequence of SEQ ID NO:54; and the disulfide bond is between each cysteine residue at amino acid position 503 of SEQ ID NO:54. In some embodiments, the two polypeptides are identical, and each identical polypeptide has the amino acid sequence of any one of SEQ ID NO:55, SEQ ID NO:64, SEQ ID NO:67, SEQ ID NO:76, SEQ ID NO:90, SEQ ID NO:92, SEQ ID NO:93, SEQ ID NO:97, and SEQ ID NO:98; and the disulfide bond is between each cysteine residue at amino acid position 502 of the amino acid sequence of each identical polypeptide. In some embodiments, the two polypeptides are identical, and each identical polypeptide has the amino acid sequence of any one of SEQ ID NO:56, SEQ ID NO:68, SEQ ID NO:91, SEQ ID NO:99, SEQ ID NO:103, and SEQ ID NO:104; and the disulfide bond is between each cysteine residue at amino acid position 492 of the amino acid sequence of each identical polypeptide. In some embodiments, the two polypeptides are identical, and each identical polypeptide has the amino acid sequence of any one of SEQ ID NO:57, SEQ ID NO:69, SEQ ID NO:78, SEQ ID NO:82, SEQ ID NO:84, SEQ ID NO:87, SEQ ID NO:88, SEQ ID NO:94, SEQ ID NO:110, SEQ ID NO:111, or SEQ ID NO:115; and the disulfide bond is between each cysteine residue at amino acid position 503 of the amino acid sequence of each identical polypeptide. In some embodiments, the two polypeptides are identical, and each identical polypeptide has the amino acid sequence of any one of SEQ ID NO:58, SEQ ID NO:70, and SEQ ID NO:81; and the disulfide bond is between each cysteine residue at amino acid position 493 of the amino acid sequence of each identical polypeptide. In some embodiments, the two polypeptides are identical, and each identical polypeptide has the amino acid sequence of any one of SEQ ID NOs: 249-304; and the disulfide bond is between each cysteine residue at amino acid position 242 of the amino acid sequence of each identical polypeptide.

In some embodiments of the methods described herein, the CD20-binding molecule is present as a mixture of monomers and dimers (e.g., homodimers). In some embodiments, the composition comprises CD20-binding molecule dimers that are substantially free of CD20-binding molecule monomers. “Substantially free” may refer to a mixture comprising less than about 5%, less than about 7.5% or less than about 10% of monomer molecules.

In some embodiments of the methods described herein, the CD20-binding molecule is present in a pharmaceutical composition, and the pharmaceutical composition comprises a pharmaceutically acceptable excipient or carrier. In some embodiments, the pharmaceutically acceptable excipient is acetate, alcohol, alpha-tocopherol, aluminum monostearate, ascorbic acid, ascorbyl palmitate, benzyl alcohol, butylated hydroxyanisole, butylated hydroxytoluene, chlorobutanol, citrate, cysteine hydrochloride, dextrose, ethanol, ethylenediaminetetraacetic acid, ethyloleate, gelatin, glycerine, glycerol, lactic acid, lecithin, mannitol, methyl parabens, monostearate salt, organic ester, paraben, phenol phosphate, phosphoric acid, polyalcohol, polyethylene glycol, polyol, propylene glycol, propylgallate, sodium bisulfate, sodium bisulfite, sodium chloride, sodium metabisulfite, sodium sulfite, sorbic acid, sorbitol, sugar, tartaric acid, or vegetable oil.

In some embodiments, the methods described herein are useful to treat a disease, disorder, or condition such as cancer, an abnormal growth condition or an immune disorder. In some embodiments, the cancer is a hematologic cancer or a solid tumor. In some embodiments, the disease is relapsed diffuse large B-cell lymphoma, refractory diffuse large B-cell lymphoma, relapsed/refractory diffuse large B-cell lymphoma or non-Hodgkin's B-cell lymphoma.

Certain embodiments described herein are below, numbered 1-49.

1. A method for treating or slowing the progression of a disease, disorder, or condition, comprising administering to a subject in need thereof an effective amount of (i) a CD20-binding molecule and (ii) an additional therapeutic agent, wherein: the CD20-binding molecule comprises: (a) a CD20-binding region, wherein the CD20-binding region is capable of specifically binding an extracellular portion of a CD20 protein, and (b) a Shiga toxin effector polypeptide.
2. The method of embodiment 1, wherein the CD20-binding molecule is a CD20-binding molecule.
3. The method of embodiment 1 or 2, wherein the CD20-binding molecule comprises two CD20-binding regions, wherein each CD20-binding region is capable of specifically binding an extracellular portion of a CD20 protein.
4. The method of any one of embodiments 1-3, wherein the additional therapeutic agent is a first additional therapeutic agent, and the method further comprises administering a second additional therapeutic agent.
5. The method of any one of embodiments 1-3, wherein the additional therapeutic agent is a chemotherapeutic agent.
6. The method of embodiment 5, wherein the chemotherapeutic agent is a nucleoside analog or a platinum-based chemotherapeutic agent.
7. The method of embodiment 4, wherein the first additional therapeutic agent or the second additional therapeutic agent is a nucleoside analog or a platinum-based chemotherapeutic agent.
8. The method of embodiment 6 or 7, wherein the nucleoside analog is gemcitabine, capecitabine, cladribine, clofarabine, cytarabine, decitabine, fludarabine, or troxacitabine, or a pharmaceutically acceptable salt thereof.
9. The method of embodiment 6 or 7, wherein the platinum-based chemotherapeutic agent is oxaliplatin, carboplatin, cisplatin, or nedaplatin.
10. The method of embodiment 4 or 7, wherein the first additional therapeutic agent and the second additional therapeutic agent are present in the same composition.
11. The method of embodiment 4 or 7, wherein the first additional therapeutic agent is present in a first composition and the second additional therapeutic agent is present in a second composition.
12. The method of any one of embodiments 4, 7, 10 and 11, wherein the first additional therapeutic agent is gemcitabine or a pharmaceutically acceptable salt thereof and wherein the second additional therapeutic agent is oxaliplatin or a pharmaceutically acceptable salt thereof.
13. The method of any one of embodiments 1-3, wherein the additional therapeutic agent is an immunomodulatory drug.
14. The method of embodiment 13, wherein the immunomodulatory drug is lenalidomide, apremilast, pomalidomide, or thalidomide, or a pharmaceutically acceptable salt thereof.
15. The method of embodiment 13, wherein the immunomodulatory drug is an mTOR inhibitor, rapalog, and/or an ATP-competitive mTOR kinase inhibitor; and optionally wherein the rapalog is everolimus, temsirolimus, ridaforolimus, or sirolimus or a derivative of any of the aforementioned.
16. The method of any one of embodiments 1-3, wherein the additional therapeutic agent is a Bruton's tyrosine kinase (BTK) inhibitor.
17. The method of embodiment 16, wherein the BTK inhibitor is acalabrutinib, evobrutinib, ibrutinib, spebrutinib or zanubrutinib, or a pharmaceutically acceptable salt thereof.
18. The method of any one of embodiments 4, 7, 10 and 11, wherein the first additional therapeutic agent is an immunomodulatory drug and wherein the second additional therapeutic agent is a BTK inhibitor. The method of any one of embodiments 4, 7, 10 and 11, wherein the first additional therapeutic agent is lenalidomide or pomalidomide and wherein the second additional therapeutic agent is a BTK inhibitor.
19. The method of any one of embodiments 1-18, wherein the CD20-binding molecule comprises, consists essentially of, or consists of a polypeptide having the amino acid sequence of any one of SEQ ID NOs: 47-175 and 249-304.
20. The method of any one of embodiments 1-18, wherein the CD20-binding molecule is multimeric and comprises, consists essentially of, or consists of: (a) two polypeptides, each polypeptide having the amino acid sequence of any one of SEQ ID NOs: 47-175 and 249-304; and (b) a cysteine disulfide bond linking the two polypeptides, wherein the cysteine disulfide bond is between a cysteine residue in each of the two polypeptides located at amino acid position 242, 482, 483, 484, 490, 491, 492, 493, 494, 495, 499, 500, 501, 502, 503, 504, 505, 510, 511, 512, 513, or 521 of the amino acid sequence of the polypeptide.
21. The method of embodiment 20, wherein each polypeptide comprises an amino-terminal methionine residue.
22. The method of embodiment 20 or 21, wherein the CD20-binding molecule is a homodimer and consists of or consists essentially of (a) the two polypeptides, wherein the two polypeptides are identical; and (b) the cysteine disulfide bond.
23. The method of embodiment 22, wherein each identical polypeptide has the amino acid sequence of SEQ ID NO:49; and wherein the disulfide bond is between each cysteine residue at amino acid position 490 in SEQ ID NO:49.
24. The method of embodiment 22, wherein each identical polypeptide has the amino acid sequence of any one of SEQ ID NO:50, SEQ ID NO:61, SEQ ID NO:73, SEQ ID NO:96, SEQ ID NO:101, and SEQ ID NO:102; and wherein the disulfide bond is between each cysteine residue at amino acid position 501 of the amino acid sequence of each identical polypeptide.
25. The method of embodiment 22, wherein each identical polypeptide has the amino acid sequence of any one of SEQ ID NO:53, SEQ ID NO:63, SEQ ID NO:66, SEQ ID NO:75, SEQ ID NO:83, SEQ ID NO:89, and SEQ ID NO:95; and wherein the disulfide bond is between each cysteine residue at amino acid position 512 of the amino acid sequence of each identical polypeptide.
26. The method of embodiment 22, wherein each identical polypeptide has the amino acid sequence of SEQ ID NO:54; and wherein the disulfide bond is between each cysteine residue at amino acid position 503 of SEQ ID NO:54.
27. The method of embodiment 22, wherein each identical polypeptide has the amino acid sequence of any one of SEQ ID NO:55, SEQ ID NO:64, SEQ ID NO:67, SEQ ID NO:76, SEQ ID NO:90, SEQ ID NO:92, SEQ ID NO:93, SEQ ID NO:97, and SEQ ID NO:98; and wherein the disulfide bond is between each cysteine residue at amino acid position 502 of the amino acid sequence of each identical polypeptide.
28. The method of embodiment 22, wherein each identical polypeptide has the amino acid sequence of any one of SEQ ID NO:56, SEQ ID NO:68, SEQ ID NO:91, SEQ ID NO:99, SEQ ID NO:103, and SEQ ID NO:104; and wherein the disulfide bond is between each cysteine residue at amino acid position 492 of the amino acid sequence of each identical polypeptide.
29. The method of embodiment 22, wherein each identical polypeptide has the amino acid sequence of any one of SEQ ID NO:57, SEQ ID NO:69, SEQ ID NO:78, SEQ ID NO:82, SEQ ID NO:84, SEQ ID NO:87, SEQ ID NO:88, SEQ ID NO:94, SEQ ID NO:110, SEQ ID NO:111, or SEQ ID NO:115; and wherein the disulfide bond is between each cysteine residue at amino acid position 503 of the amino acid sequence of each identical polypeptide.
30. The method of embodiment 22, wherein each identical polypeptide has the amino acid sequence of any one of SEQ ID NO:58, SEQ ID NO:70, and SEQ ID NO:81; and wherein the disulfide bond is between each cysteine residue at amino acid position 493 of the amino acid sequence of each identical polypeptide.
31. The method of embodiment 22, wherein each identical polypeptide has the amino acid sequence of any one of SEQ ID NOs: 249-304; and wherein the disulfide bond is between each cysteine residue at amino acid position 242 of the amino acid sequence of each identical polypeptide.
32. The method of any one of embodiments 1-31, wherein the CD20-binding molecule is present in a pharmaceutical composition, and wherein the pharmaceutical composition comprises at least one pharmaceutically acceptable excipient or carrier.
33. The method of embodiment 32, wherein the pharmaceutically acceptable excipient is acetate, alcohol, alpha-tocopherol, aluminum monostearate, ascorbic acid, ascorbyl palmitate, benzyl alcohol, butylated hydroxyanisole, butylated hydroxytoluene, chlorobutanol, citrate, cysteine hydrochloride, dextrose, ethanol, ethylenediaminetetraacetic acid, ethyloleate, gelatin, glycerine, glycerol, lactic acid, lecithin, mannitol, methyl parabens, monostearate salt, organic ester, paraben, phenol phosphate, phosphoric acid, polyalcohol, polyethylene glycol, polyol, propylene glycol, propylgallate, sodium bisulfate, sodium bisulfite, sodium chloride, sodium metabisulfite, sodium sulfite, sorbic acid, sorbitol, sugar, tartaric acid, or vegetable oil.
34. The method of any one of embodiments 1-33, wherein the disease, disorder, or condition is a cancer, an abnormal growth condition or an immune disorder.
35. The method of embodiment 34, wherein the disease, disorder or condition is cancer, and the cancer is a hematologic cancer or a solid tumor.
356. The method of any one of embodiments 1-35, wherein the disease, disorder, or condition is leukemia, lymphoma, melanoma, myeloma, acute myeloid leukemia, acute non-lymphocytic leukemia, B-cell chronic lymphocytic leukemia, B-cell lymphoma, B-cell non-Hodgkin's lymphoma, B-cell precursor acute lymphoblastic leukemia, B-cell prolymphocytic leukemia, Burkitt's lymphoma, chronic lymphocytic leukemia, chronic myeloid leukemia, diffuse large B-cell lymphoma, follicular lymphoma, hairy cell leukemia, Hodgkin's lymphoma, immunoblastic large cell lymphoma, mantle cell lymphoma, multiple myeloma, nodular lymphocyte predominant Hodgkin's lymphoma, non-Hodgkin's lymphoma, plasmablastic lymphoma, plasma cell neoplasma, plasma cell myeloma, precursor B-lymphoblastic lymphoma, small lymphocytic lymphoma, T-cell large granular lymphocyte leukemia, T-cell lymphoma, T-cell prolymphocytic leukemia, Waldenström's macroglobulinemia, amyloidosis, ankylosing spondylitis, asthma, Crohn's disease, diabetes, graft rejection, graft-versus-host disease, Graves' disease, Graves' ophthalmopathy, Hashimoto's thyroiditis, hemolytic uremic syndrome, HIV-related disease, lupus erythematosus, multiple sclerosis, neuromyelitis optica spectrum disorders, N-methyl D-aspartate receptor encephalitis, opsoclonus myoclonus syndrome, paroxysmal nocturnal hemoglobinuria, polyarteritis nodosa, polyarthritis, psoriasis, psoriatic arthritis, rheumatoid arthritis, scleritis, scleroderma, septic shock, Sjorgren's syndrome, ulcerative colitis, or vasculitis.
37. The method of any one of embodiments 1-35, wherein the disease is relapsed diffuse large B-cell lymphoma, refractory diffuse large B-cell lymphoma, relapsed/refractory diffuse large B-cell lymphoma or non-Hodgkin's B-cell lymphoma.
38. The method of any one of embodiments 1-37, wherein the administering of the CD20-binding molecule is administering intravenously.
39. The method of embodiment 38, wherein the administering of the CD20-binding molecule comprises administering six doses of the CD20-binding molecule, each dose being an intravenous infusion that is administered over about 1 hour, for 6 doses over 12 days of a 21-day cycle, wherein each dose is in an amount of about 10 μg/kg, about 25 μg/kg or about 50 μg/kg of the subject's body weight.
40. The method of embodiment 39, further comprising repeating the 21-day cycle of administering the CD20-binding molecule.
41. The method of embodiment 39 or 40, further comprising administering the CD20-binding molecule weekly following the 21-day cycle.
42. The method of embodiment 38, wherein the administering of the CD20-binding molecule comprises administering ten doses of the CD20-binding molecule, each dose being an intravenous infusion that is administered over about 1 hour on days 1, 3, 5, 8, 10, 12, 15, 22, 29 and 36 of a 42-day cycle; wherein each dose is in an amount of about 10 μg/kg, about 25 μg/kg, about 50 μg/kg, or about 75 μg/kg of the subject's body weight.
43. The method of embodiment 42, further comprising administering the CD20-binding molecule weekly during a 28-day cycle following the 42-day cycle.
44. The method of embodiment 42, further comprising administering to the subject gemcitabine or a pharmaceutically acceptable salt thereof at a dose of about 1,000 mg/m² on days 16 and 30 of the 42-day cycle; wherein each dose of gemcitabine or pharmaceutically acceptable salt thereof is administered as an intravenous infusion over about 30 minutes.
45. The method of embodiment 43, further comprising administering to the subject gemcitabine or a pharmaceutically acceptable salt thereof at a dose of about 1,000 mg/m² on days 2 and 16 of the 28-day cycle; wherein each dose of gemcitabine or pharmaceutically acceptable salt thereof is administered as an intravenous infusion over about 30 minutes.
46. The method of embodiment 42 or 44, further comprising administering to the subject oxaliplatin or a pharmaceutically acceptable salt thereof at a dose of about 100 mg/m² on days 16 and 30 of the 42-day cycle; wherein each dose of oxaliplatin or a pharmaceutically acceptable salt thereof is administered as an intravenous infusion over about 2 hours; and wherein each dose of oxaliplatin or a pharmaceutically acceptable salt thereof is administered about one hour after the start of each infusion of gemcitabine or a pharmaceutically acceptable salt thereof.
47. The method of embodiment 43 or 45, further comprising administering to the subject oxaliplatin or a pharmaceutically acceptable salt thereof at a dose of about 100 mg/m² on days 2 and 16 of each 28-day cycle; wherein each dose of oxaliplatin or a pharmaceutically acceptable salt thereof is administered as an intravenous infusion over about 2 hours; and wherein each dose of oxaliplatin or a pharmaceutically acceptable salt thereof is administered about one hour after the start of each infusion of gemcitabine or a pharmaceutically acceptable salt thereof.
48. The method of embodiment 39, further comprising administering to the subject an immunomodulatory drug orally at a dose of about 20 mg on each of days 1 and 21 of the 21-day cycle.
49. The method of embodiment 48, wherein the immunomodulatory drug is lenalidomide or a pharmaceutically acceptable salt thereof.

In some embodiments, provided herein are methods for treating a malignancy, a neoplasm or another blood cell associated cancer in a mammalian subject, such as a human, the method comprising the step of administering to a subject in need thereof an effective amount of a CD20-binding molecule as described herein and/or a composition thereof.

The CD20-binding molecule as described herein and/or a composition thereof have varied applications, including, e.g., uses in removing unwanted B-cells and/or T-cells, uses in modulating immune responses to treat graft-versus-host disease, uses as antiviral agents, uses as antimicrobial agents, and uses in purging transplantation tissues of unwanted cell types. The CD20-binding molecule as described herein and/or a composition thereof are commonly anti-neoplastic agents—meaning they are capable of treating and/or preventing the development, maturation, or spread of neoplastic or malignant cells by inhibiting the growth and/or causing the death of CD20+ cancer, neoplastic, or tumor cells.

In some embodiments, a CD20-binding molecule as described herein and/or a composition thereof is useful to treat a B-cell-, plasma cell-, T-cell- or antibody-mediated disease or disorder, such as for example hematologic diseases, rheumatic diseases, hematologic cancers, leukemias, lymphomas, melanomas, myelomas, B-cell lymphomas, B-cell non-Hodgkins lymphomas (B-cell NHL), Burkitt's lymphomas (BL), B-cell chronic lymphocytic leukemias (B-cell CLL), chronic lymphocytic leukemias (CLL), diffuse large B-cell lymphomas (DLBCL or DLBL), follicular lymphomas (FL), hairy cell leukemias (HCL), Hodgkins lymphomas (HD), immunoblastic large cell lymphomas, mantle cell lymphomas (MCL), melanomas, non-Hodgkins lymphomas (NHL), precursor B-lymphoblastic lymphomas (B-LBL), small lymphocytic lymphoma (SLL), and T-cell lymphomas (TCL), amyloidosis, ankylosing spondylitis, asthma, Crohn's disease, diabetes, graft rejection, graft-versus-host disease, Hashimoto's thyroiditis, hemolytic uremic syndrome, HIV-related diseases, lupus erythematosus, multiple sclerosis, polyarteritis nodosa, polyarthritis, psoriasis, psoriatic arthritis, rheumatoid arthritis, scleroderma, septic shock, Sjorgren's syndrome, ulcerative colitis, and/or vasculitis.

It is within the scope of this disclosure to provide a prophylaxis or treatment for diseases or conditions mediated by B-cells and/or T-cells by administering the CD20-binding molecule as described herein and/or a composition thereof, to a patient for the purpose of killing B-cells and/or T-cells in the patient. This usage is compatible with preparing or conditioning a patient for bone marrow transplantation, stem cell transplantation, tissue transplantation, or organ transplantation, regardless of the source of the transplanted material, e.g., human or non-human sources.

It is within the scope as described herein to provide a bone marrow recipient for prophylaxis or treatment of host-versus-graft disease via the targeted CD20+ cell-killing of host T-cells using a CD20-binding molecule as described herein and/or a composition thereof.

The CD20-binding molecule as described herein and/or a composition thereof may be utilized in a method of treating cancer comprising administering to a patient, in need thereof, a therapeutically effective amount of the protein composition or a pharmaceutical composition as described herein. For certain embodiments of the methods as described herein, the condition, disease, or disorder being treated is related to hematologic diseases, rheumatic diseases, hematologic cancers, leukemias, lymphomas, melanomas, myelomas, B-cell lymphomas, B-cell non-Hodgkins lymphomas (B-cell NHL), Burkitt's lymphomas (BL), B-cell chronic lymphocytic leukemias (B-cell CLL), chronic lymphocytic leukemias (CLL), diffuse large B-cell lymphomas (DLBCL or DLBL), follicular lymphomas (FL), hairy cell leukemias (HCL), Hodgkins lymphomas (HD), immunoblastic large cell lymphomas, mantle cell lymphomas (MCL), melanomas, non-Hodgkins lymphomas (NHL), precursor B-lymphoblastic lymphomas (B-LBL), small lymphocytic lymphoma (SLL), and T-cell lymphomas (TCL), amyloidosis, ankylosing spondylitis, asthma, Crohn's disease, diabetes, graft rejection, graft-versus-host disease, Hashimoto's thyroiditis, hemolytic uremic syndrome, HIV-related diseases, lupus erythematosus, multiple sclerosis, polyarteritis nodosa, polyarthritis, psoriasis, psoriatic arthritis, rheumatoid arthritis, scleroderma, septic shock, Sjorgren's syndrome, ulcerative colitis, and/or vasculitis.

Non-limiting examples of subtypes of hematologic cancers (e.g. leukemias, lymphomas, and myelomas) that may be treated with the CD20-binding molecules and compositions as described herein include acute myeloid leukemias (acute myelogenous leukemia or AML), acute non-lymphocytic leukemias, B-cell lymphomas, B-cell non-Hodgkin's lymphomas (B-cell NHL), B-cell acute lymphoblastic leukemias (B-ALL or BCP-ALL), B-cell prolymphocytic leukemias (B-PLL), B-lymphoblastic lymphomas (B-LBL), Burkitt's lymphomas (BL), atypical Burkitt's lymphomas (atypical BL), chronic lymphocytic leukemias (CLL), chronic myeloid leukemias (CML), cutaneous B-cell lymphomas (CBCL), diffuse large B-cell lymphomas (DLBCL or DLBL), follicular lymphomas (FL), hairy cell leukemias (HCL), heavy chain diseases, Hodgkin's lymphomas (HL or HD), immunoblastic large cell lymphomas, granulomatosis (LG or LYG), lymphoplasmacytic lymphomas, mantle cell lymphomas (MCL), marginal zone lymphomas (MZL), multiple myelomas (MM), nodular lymphocyte predominant Hodgkin's lymphomas (NLPHL), non-Hodgkin's lymphomas (NHL), plasmablastic lymphomas (PBL), plasmablastic lymphomas associated with multicentric Castleman disease, plasma cell neoplasmas, plasma cell myelomas, primary effusion lymphomas (PEL), small lymphocytic lymphomas (SLL), T-cell large granular lymphocyte leukemias (T-LGLL), T-cell lymphomas (TCL), peripheral T-cell lymphomas (PTCL), T-cell prolymphocytic leukemias (T-PLL), mycosis fungiodes (MF), and Waldenström's macroglobulinemias (WM).

The CD20-binding molecule as described herein and/or a composition thereof may be utilized in a method of treating an immune disorder comprising administering to a patient, in need thereof, a therapeutically effective amount of the CD20-binding molecule as described herein and/or a composition thereof. For certain embodiments of the methods as described herein, the immune disorder is related to an inflammation associated with a disease such as amyloidosis, ankylosing spondylitis, asthma, Crohn's disease, diabetes, graft rejection, graft-versus-host disease, Graves' disease, Graves' ophthalmopathy, Hashimoto's thyroiditis, heavy chain disease, hemolytic uremic syndrome, HIV-related diseases, lupus erythematosus, multiple sclerosis, neuromyelitis optica spectrum disorders, N-methyl D-aspartate (NMDA) receptor encephalitis, opsoclonus myoclonus syndrome (OMS), paroxysmal nocturnal hemoglobinuria, polyarteritis nodosa, polyarthritis, psoriasis, psoriatic arthritis, rheumatoid arthritis, scleritis, scleroderma, septic shock, Sjorgren's syndrome, ulcerative colitis, or vasculitis.

Among certain embodiments as described herein is using the CD20-binding molecule as described herein and/or a composition thereof or a solvate or salt as described herein as a component of a pharmaceutical composition or medicament for the treatment or prevention of a cancer, tumor, immune disorder, and/or growth abnormality involving a CD20+ cell. For example, immune disorders presenting on the skin of a patient may be treated with such a medicament in efforts to reduce inflammation. In another example, skin tumors may be treated with such a medicament in efforts to reduce tumor size or eliminate the tumor completely.

For certain cancers, depletion and/or inhibition of B-cells generally may improve disease outcomes, such as, e.g. by depleting cancer escape promoting regulatory B-cells (see e.g. Olkhanud P et al., Cancer Res 69: 5996-6004 (2009); Olkhanud P et al., Cancer Res 71: 3505-15 (2011)).

Among certain embodiments as described herein are methods of inducing cellular internalization of a CD20-binding molecule into a cell(s) and/or internalizing a cell surface localized CD20 bound by a CD20-binding molecule as described herein, the method comprising the step of contacting the cell(s) with a CD20-binding molecule as described herein, a CD20-binding molecule composition as described herein, a solvate as described herein, a salt as described herein, a pharmaceutical composition as described herein, and/or a diagnostic composition as described herein. For certain embodiments of this inducing internalization method, the cell(s) is physically coupled with CD20, which have the extracellular part bound by two or more CD20 binding regions of the CD20-binding molecule. In some embodiments of the inducing cellular internalization method, the step of contacting the cell(s) occurs in vitro. For certain other embodiments, the step of contacting the cell(s) occurs in vivo, such as, e.g., within a patient. In some embodiments of the inducing cellular internalization method, the cellular internalization of the CD20-binding molecule occurs in about five hours, four hours, three hours, two hours, one hour, thirty minutes, or less at a physiological temperature appropriate for the cell and/or at about 37 degrees Celsius. In some embodiments, the cell expresses at a cellular surface the CD20 which (1) have the extracellular part bound by the two or more CD20 binding regions of the CD20-binding molecule, (2) have a transmembrane domain, and (3) remain physically coupled to the cell. In some embodiments, the cell is a CD20 positive cell. In some embodiments, the cell is physically coupled with a significant amount of extracellular CD20 which (1) have the extracellular part bound by the two or more CD20 binding regions of the CD20-binding molecule. In some embodiments, the cell is a descendant or member of a B-cell lineage. In some embodiments, the cell is a malignant B-cell, B-cell leukemia cell, B-cell lymphoma cell, B-cell myeloma cell, acute myeloid leukemia cell, acute non-lymphocytic leukemia cell, B-cell chronic lymphocytic leukemia cell, B-cell lymphoma cell, B-cell non-Hodgkin's lymphoma cell, B-cell precursor acute lymphoblastic leukemia cell, B-cell prolymphocytic leukemia cell, Burkitt's lymphoma cell, chronic lymphocytic leukemia cell, chronic myeloid leukemia cell, diffuse large B-cell lymphoma cell, follicular lymphoma cell, hairy cell leukemia cell, Hodgkin's lymphoma cell, immunoblastic large cell lymphoma cell, mantle cell lymphoma cell, melanoma cell, multiple myeloma cell, neoplastic plasma cell, nodular lymphocyte predominant Hodgkin's lymphoma cell, non-Hodgkin's lymphoma cell, plasmablastic lymphoma cell, plasma cell myeloma cell, precursor B-lymphoblastic lymphoma cell, small lymphocytic lymphoma cell, malignant T-cell, T-cell leukemia cell, T-cell lymphoma cell, T-cell large granular lymphocyte leukemia cell, T-cell prolymphocytic leukemia, healthy B-cell lineage cell, or healthy T-cell.

In some embodiments, the methods as described herein provide a method of inducing cellular internalization of a cell surface localized CD20 bound by a CD20-binding molecule in a patient, the method comprising the step of administering to the patient a CD20-binding molecule as described herein, a solvate as described herein, a salt as described herein, a CD20-binding molecule composition as described herein, pharmaceutical composition as described herein, and/or a diagnostic composition as described herein.

Additionally, provided herein are methods for delivering an exogenous material to the inside of a cell, the method comprising the step of contacting the cell(s), either in vitro or in vivo, with a CD20-binding molecule as described herein which comprises an additional exogenous material, a CD20-binding molecule composition as described herein comprising a CD20-binding molecule as described herein which comprises an additional exogenous material, a solvate as described herein comprising a CD20-binding molecule as described herein which comprises an additional exogenous material, a salt as described herein comprising a CD20-binding molecule as described herein which comprises an additional exogenous material, a pharmaceutical composition as described herein comprising a CD20-binding molecule as described herein which comprises an additional exogenous material, and/or a diagnostic composition as described herein comprising a CD20-binding molecule as described herein which comprises an additional exogenous material. In some embodiments, the cell is physically coupled with CD20 which have the extracellular part bound by two or more CD20 binding regions of the CD20-binding molecule. In some embodiments, the cell expresses at a cellular surface the CD20 which (1) have the extracellular part bound by the two or more CD20 binding regions of the CD20-binding molecule, (2) have a transmembrane domain, and (3) remain physically coupled to the cell. In some embodiments, the cell is a CD20 positive cell. For certain embodiments, the cell is physically coupled with a significant amount of extracellular CD20 which (1) have the extracellular part bound by the two or more CD20 binding regions of the CD20-binding molecule. For certain embodiments, the cell is a descendant or member of a B-cell lineage. For certain embodiments, the cell is a malignant B-cell, B-cell leukemia cell, B-cell lymphoma cell, B-cell myeloma cell, acute myeloid leukemia cell, acute non-lymphocytic leukemia cell, B-cell chronic lymphocytic leukemia cell, B-cell lymphoma cell, B-cell non-Hodgkin's lymphoma cell, B-cell precursor acute lymphoblastic leukemia cell, B-cell prolymphocytic leukemia cell, Burkitt's lymphoma cell, chronic lymphocytic leukemia cell, chronic myeloid leukemia cell, diffuse large B-cell lymphoma cell, follicular lymphoma cell, hairy cell leukemia cell, Hodgkin's lymphoma cell, immunoblastic large cell lymphoma cell, mantle cell lymphoma cell, melanoma cell, multiple myeloma cell, neoplastic plasma cell, nodular lymphocyte predominant Hodgkin's lymphoma cell, non-Hodgkin's lymphoma cell, plasmablastic lymphoma cell, plasma cell myeloma cell, precursor B-lymphoblastic lymphoma cell, small lymphocytic lymphoma cell, malignant T-cell, T-cell leukemia cell, T-cell lymphoma cell, T-cell large granular lymphocyte leukemia cell, T-cell prolymphocytic leukemia, healthy B-cell lineage cell, or healthy T-cell.

For certain embodiments, a method of delivering an exogenous material (e.g., a detection promoting agent for collecting diagnostic information) to the inside of a cell in a patient is provided, the method comprising the step of administering to the patient a CD20-binding molecule as described herein which comprises an additional exogenous material, a CD20-binding molecule composition as described herein comprising a CD20-binding molecule as described herein which comprises an additional exogenous material, a solvate as described herein comprising a CD20-binding molecule as described herein which comprises an additional exogenous material, a salt as described herein comprising a CD20-binding molecule as described herein which comprises an additional exogenous material, a pharmaceutical composition as described herein comprising a CD20-binding molecule as described herein which comprises an additional exogenous material, and/or a diagnostic composition as described herein comprising a CD20-binding molecule as described herein which comprises an additional exogenous material.

Also provided herein are various embodiments of CD20-binding molecules, and compositions thereof, wherein each CD20-binding molecule comprises 1) two or more CD20 binding regions, such as a binding region derived from an immunoglobulin, and 2) at least one Shiga toxin A Subunit effector polypeptide region derived from the A Subunit of at least one member of the Shiga toxin family. Each CD20 binding region of these multivalent CD20-binding molecules as described herein is capable of specifically binding an extracellular part of a CD20, such as, e.g., a part of a CD20 exposed to the extracellular environment when CD20 is expressed at a cellular surface by a cell and remains physically coupled to the cell.

The linking of multiple CD20 binding regions with one or more Shiga toxin A Subunit-derived polypeptides enables the engineering of CD20-targeting molecules that can promote rapid cellular internalization of cell-surface CD20 and thus efficiently enter the interiors of CD20-expressing cells. Therefore, certain multivalent CD20-binding molecules as described herein, and compositions thereof, are useful to selectively deliver cargo(s) to a CD20-expressing cell type(s) in the presence of one or more other cell types based on its CD20-targeting and cellular internalization activity(ies), such as, e.g., a cargo having a desired, intracellular function. In addition, certain CD20-binding molecules as described herein, and compositions thereof, are useful to selectively kill a CD20-expressing cell in the presence of one or more other cell types based on its CD20-targeting activity and cellular internalization activity(ies), such as, e.g., by delivering into the interior of the targeted, CD20-expressing cell a component of the CD20-binding molecule which is cytotoxic at an intracellular location. For example, certain CD20-binding molecules as described herein may be potently cytotoxic to CD20-expressing cells via their abilities to efficiently deliver into the interior of a CD20-expressing cell a catalytically active, Shiga toxin effector polypeptide(s) that is able to effectively route to the cytosol.

In some embodiments, the multivalent CD20-binding molecule as described herein comprises (1) two or more CD20 binding regions, each capable of specifically binding an extracellular part of a CD20 molecule; and (2) one or more Shiga toxin effector polypeptides derived from the amino acid sequence of the A Subunit of at least one member of the Shiga toxin family. In some embodiments, the multivalent CD20-binding molecule as described herein does not comprise an immunoglobulin Fc region or any immunoglobulin domain required for an extracellular mechanism(s) of cell killing other than a domain(s) required for antigen binding. In some embodiments, the multivalent CD20-binding molecule does not comprise any immunoglobulin domains other than 1) one or more heavy chain variable domains, (2) one or more heavy chain variable domains and one or more light chain variable domains, (3) six or more CDRs, and/or (4) one or more single-chain variable fragments. In some embodiments, the CD20-binding molecule as described herein comprises only two, Shiga toxin effector polypeptides.

In some embodiments, the multivalent CD20-binding molecule as described herein comprises (1) two or more CD20 binding regions, each capable of specifically binding an extracellular part of a CD20 molecule; and (2) one or more Shiga toxin effector regions, each comprising a polypeptide derived from the amino acid sequence of the A Subunit of at least one member of the Shiga toxin family. In some embodiments, the multivalent CD20-binding molecule as described herein comprises (1) two or more CD20 binding regions, each capable on its own of specifically binding an extracellular part of a CD20 molecule; and (2) one or more Shiga toxin effector regions, each comprising a polypeptide derived from the amino acid sequence of the A Subunit of at least one member of the Shiga toxin family. In some embodiments, the multivalent CD20-binding molecule as described herein does not comprise an immunoglobulin Fc region or any immunoglobulin domain required for an extracellular mechanism(s) of cell killing other than a domain(s) required for antigen binding. In some embodiments, the multivalent CD20-binding molecule does not comprise any immunoglobulin domains other than 1) one or more heavy chain variable domains, (2) one or more heavy chain variable domains and one or more light chain variable domains, (3) six or more CDRs, and/or (4) one or more single-chain variable fragments. In some embodiments, the CD20-binding molecule as described herein comprises only two, Shiga toxin effector polypeptides.

For certain embodiments of the multivalent CD20-binding molecule as described herein, upon administration of the multivalent CD20-binding molecule to a cell physically coupled with CD20, which have the extracellular part bound by two or more CD20 binding regions of the multivalent CD20-binding molecule, results in one or more of the following: (1) internalizing the multivalent CD20-binding molecule inside the cell, (2) subcellular routing of a Shiga toxin effector polypeptide of the multivalent CD20-binding molecule to the cell's cytosol, (3) disrupting the cell's ribosome function, and (4) killing of the cell. In some embodiments, the internalizing occurs in about five hours, four hours, three hours, two hours, one hour, thirty minutes, or less at a physiological temperature appropriate for the cell and/or at about 37 degrees Celsius. In some embodiments, the multivalent CD20-binding molecule induces cellular internalization of a molecular complex comprising the multivalent CD20-binding molecule bound to CD20. In some embodiments, the cell expresses at a cellular surface the CD20 which (1) have the extracellular part bound by the two or more CD20 binding regions of the multivalent CD20-binding molecule, (2) have a transmembrane domain, and (3) remain physically coupled to the cell. In some embodiments, the cell is a CD20 positive cell. For certain embodiments, the cell is physically coupled with a significant amount of extracellular CD20 which (1) have the extracellular part bound by the two or more CD20 binding regions of the multivalent CD20-binding molecule. For certain embodiments, the cell is a descendant or member of a B-cell lineage. For certain embodiments, the cell is a malignant B-cell, B-cell leukemia cell, B-cell lymphoma cell, B-cell myeloma cell, acute myeloid leukemia cell, acute non-lymphocytic leukemia cell, B-cell chronic lymphocytic leukemia cell, B-cell lymphoma cell, B-cell non-Hodgkin's lymphoma cell, B-cell precursor acute lymphoblastic leukemia cell, B-cell prolymphocytic leukemia cell, Burkitt's lymphoma cell, chronic lymphocytic leukemia cell, chronic myeloid leukemia cell, diffuse large B-cell lymphoma cell, follicular lymphoma cell, hairy cell leukemia cell, Hodgkin's lymphoma cell, immunoblastic large cell lymphoma cell, mantle cell lymphoma cell, melanoma cell, multiple myeloma cell, neoplastic plasma cell, nodular lymphocyte predominant Hodgkin's lymphoma cell, non-Hodgkin's lymphoma cell, plasmablastic lymphoma cell, plasma cell myeloma cell, precursor B-lymphoblastic lymphoma cell, small lymphocytic lymphoma cell, malignant T-cell, T-cell leukemia cell, T-cell lymphoma cell, T-cell large granular lymphocyte leukemia cell, T-cell prolymphocytic leukemia, healthy B-cell lineage cell, or healthy T-cell.

For certain embodiments of the multivalent CD20-binding molecule as described herein, upon administration of the multivalent CD20-binding molecule to a cell physically coupled with CD20, which have the extracellular part bound by two or more CD20 binding regions of the multivalent CD20-binding molecule, results in one or more of the following: (1) internalizing the multivalent CD20-binding molecule inside the cell, (2) subcellular routing of a Shiga toxin effector region of the multivalent CD20-binding molecule to the cell's cytosol, (3) disrupting the cell's ribosome function, and (4) killing of the cell. In some embodiments, the internalizing occurs in about five hours, four hours, three hours, two hours, one hour, thirty minutes, or less at a physiological temperature appropriate for the cell and/or at about 37 degrees Celsius. In some embodiments, the CD20-binding molecule induces cellular internalization of a molecular complex comprising the CD20-binding molecule bound to CD20. In some embodiments, the cell expresses at a cellular surface the CD20 which (1) have the extracellular part bound by the two or more CD20 binding regions of the CD20-binding molecule, (2) have a transmembrane domain, and (3) remain physically coupled to the cell. In some embodiments, the cell is a CD20 positive cell. For certain embodiments, the cell is physically coupled with a significant amount of extracellular CD20 which (1) have the extracellular part bound by the two or more CD20 binding regions of the CD20-binding molecule. For certain embodiments, the cell is a descendant or member of a B-cell lineage. For certain embodiments, the cell is a malignant B-cell, B-cell leukemia cell, B-cell lymphoma cell, B-cell myeloma cell, acute myeloid leukemia cell, acute non-lymphocytic leukemia cell, B-cell chronic lymphocytic leukemia cell, B-cell lymphoma cell, B-cell non-Hodgkin's lymphoma cell, B-cell precursor acute lymphoblastic leukemia cell, B-cell prolymphocytic leukemia cell, Burkitt's lymphoma cell, chronic lymphocytic leukemia cell, chronic myeloid leukemia cell, diffuse large B-cell lymphoma cell, follicular lymphoma cell, hairy cell leukemia cell, Hodgkin's lymphoma cell, immunoblastic large cell lymphoma cell, mantle cell lymphoma cell, melanoma cell, multiple myeloma cell, neoplastic plasma cell, nodular lymphocyte predominant Hodgkin's lymphoma cell, non-Hodgkin's lymphoma cell, plasmablastic lymphoma cell, plasma cell myeloma cell, precursor B-lymphoblastic lymphoma cell, small lymphocytic lymphoma cell, malignant T-cell, T-cell leukemia cell, T-cell lymphoma cell, T-cell large granular lymphocyte leukemia cell, T-cell prolymphocytic leukemia, healthy B-cell lineage cell, or healthy T-cell.

For certain embodiments of the CD20-binding molecule as described herein, upon administration of the CD20-binding molecule to a plurality of cells physically coupled with CD20, which have the extracellular part bound by two or more CD20 binding regions of the CD20-binding molecule, results in one or more of the following activities: (1) internalizing the CD20-binding molecule inside the cell, (2) subcellular routing of a Shiga toxin effector polypeptide of the CD20-binding molecule to the cell's cytosol, (3) disrupting the cell's ribosome function, and (4) killing of the cell. In some embodiments, the CD20-binding molecule induces cellular internalization of a molecular complex comprising the CD20-binding molecule bound to CD20. In some embodiments, upon administration of the CD20-binding molecule to a plurality of cells physically coupled with CD20, which have the extracellular part bound by two or more CD20 binding regions of the multivalent CD20-binding molecule, at a concentration of multivalent CD20-binding molecule equivalent to five or thirty-eight percent to fifty percent cell-surface occupancy, the majority of the multivalent CD20-binding molecule internalizes into the plurality of cells in about five hours, four hours, three hours, two hours, one hour, thirty minutes, or less at a physiological temperature appropriate for the cell and/or at about 37 degrees Celsius. In some embodiments, members of the plurality of cells express at a cellular surface the CD20 which (1) have the extracellular part bound by the two or more CD20 binding regions of the multivalent CD20-binding molecule, (2) have a transmembrane domain, and (3) remain physically coupled to the cell. In some embodiments, members of the plurality of cells are CD20 positive cells. For certain embodiments, the members of the plurality of cells are physically coupled with a significant amount of extracellular CD20 which (1) have the extracellular part bound by the two or more CD20 binding regions of the multivalent CD20-binding molecule. For certain embodiments, members of the plurality of cells are descendants or members of a B-cell lineage. For certain embodiments, members of the plurality of cells are s malignant B-cell, B-cell leukemia cell, B-cell lymphoma cell, B-cell myeloma cell, acute myeloid leukemia cell, acute non-lymphocytic leukemia cell, B-cell chronic lymphocytic leukemia cell, B-cell lymphoma cell, B-cell non-Hodgkin's lymphoma cell, B-cell precursor acute lymphoblastic leukemia cell, B-cell prolymphocytic leukemia cell, Burkitt's lymphoma cell, chronic lymphocytic leukemia cell, chronic myeloid leukemia cell, diffuse large B-cell lymphoma cell, follicular lymphoma cell, hairy cell leukemia cell, Hodgkin's lymphoma cell, immunoblastic large cell lymphoma cell, mantle cell lymphoma cell, melanoma cell, multiple myeloma cell, neoplastic plasma cell, nodular lymphocyte predominant Hodgkin's lymphoma cell, non-Hodgkin's lymphoma cell, plasmablastic lymphoma cell, plasma cell myeloma cell, precursor B-lymphoblastic lymphoma cell, small lymphocytic lymphoma cell, malignant T-cell, T-cell leukemia cell, T-cell lymphoma cell, T-cell large granular lymphocyte leukemia cell, T-cell prolymphocytic leukemia, healthy B-cell lineage cell, or healthy T-cell members.

For certain embodiments of the multivalent CD20-binding molecule as described herein, upon administration of the multivalent CD20-binding molecule to a plurality of cells physically coupled with CD20, which have the extracellular part bound by two or more CD20 binding regions of the multivalent CD20-binding molecule, results in one or more of the following activities: (1) internalizing the multivalent CD20-binding molecule inside the cell, (2) subcellular routing of a Shiga toxin effector region of the multivalent CD20-binding molecule to the cell's cytosol, (3) disrupting the cell's ribosome function, and (4) killing of the cell. In some embodiments, the multivalent CD20-binding molecule induces cellular internalization of a molecular complex comprising the multivalent CD20-binding molecule bound to CD20. In some embodiments, upon administration of the multivalent CD20-binding molecule to a plurality of cells physically coupled with CD20, which have the extracellular part bound by two or more CD20 binding regions of the multivalent CD20-binding molecule, at a concentration of multivalent CD20-binding molecule equivalent to five or thirty-eight percent to fifty percent cell-surface occupancy, the majority of the multivalent CD20-binding molecule internalizes into the plurality of cells in about five hours, four hours, three hours, two hours, one hour, thirty minutes, or less at a physiological temperature appropriate for the cell and/or at about 37 degrees Celsius. In some embodiments, members of the plurality of cells express at a cellular surface the CD20 which (1) have the extracellular part bound by the two or more CD20 binding regions of the multivalent CD20-binding molecule, (2) have a transmembrane domain, and (3) remain physically coupled to the cell. In some embodiments, members of the plurality of cells are CD20 positive cells. For certain embodiments, the members of the plurality of cells are physically coupled with a significant amount of extracellular CD20 which (1) have the extracellular part bound by the two or more CD20 binding regions of the CD20-binding molecule. For certain embodiments, members of the plurality of cells are descendants or members of a B-cell lineage. For certain embodiments, members of the plurality of cells are malignant B-cell, B-cell leukemia cell, B-cell lymphoma cell, B-cell myeloma cell, acute myeloid leukemia cell, acute non-lymphocytic leukemia cell, B-cell chronic lymphocytic leukemia cell, B-cell lymphoma cell, B-cell non-Hodgkin's lymphoma cell, B-cell precursor acute lymphoblastic leukemia cell, B-cell prolymphocytic leukemia cell, Burkitt's lymphoma cell, chronic lymphocytic leukemia cell, chronic myeloid leukemia cell, diffuse large B-cell lymphoma cell, follicular lymphoma cell, hairy cell leukemia cell, Hodgkin's lymphoma cell, immunoblastic large cell lymphoma cell, mantle cell lymphoma cell, melanoma cell, multiple myeloma cell, neoplastic plasma cell, nodular lymphocyte predominant Hodgkin's lymphoma cell, non-Hodgkin's lymphoma cell, plasmablastic lymphoma cell, plasma cell myeloma cell, precursor B-lymphoblastic lymphoma cell, small lymphocytic lymphoma cell, malignant T-cell, T-cell leukemia cell, T-cell lymphoma cell, T-cell large granular lymphocyte leukemia cell, T-cell prolymphocytic leukemia, healthy B-cell lineage cell, or healthy T-cell members.

For certain embodiments of the multivalent CD20-binding molecule as described herein, administration of the multivalent CD20-binding molecule to a CD20-expressing cell, the multivalent CD20-binding molecule is capable of causing death of the cell, i.e. killing the cell. For certain embodiments of the multivalent CD20-binding molecule as described herein, upon administration of the multivalent CD20-binding molecule to a CD20-expressing cell expressing CD20 having the extracellular part bound by two or more CD20 binding regions of the multivalent CD20-binding molecule, the multivalent CD20-binding molecule is capable of causing death of the cell. In some embodiments, the cell express at a cellular surface the CD20 which (1) have the extracellular part bound by the two or more CD20 binding regions of the multivalent CD20-binding molecule, (2) have a transmembrane domain, and (3) remain physically coupled to the cell. In some embodiments, the cell is a CD20 positive cell. In some embodiments, the cell is physically coupled with a significant amount of extracellular CD20 which (1) have the extracellular part bound by the two or more CD20 binding regions of the multivalent CD20-binding molecule. This cell killing activity may or may not depend on the catalytic activity of one or more Shiga toxin effector regions (e.g. Shiga toxin effector polypeptides) of the multivalent CD20-binding molecule.

For certain embodiments of the multivalent CD20-binding molecule as described herein, upon administration of the multivalent CD20-binding molecule to a first population of cells physically coupled to CD20, and a second population of cells, a cytotoxic effect of the multivalent CD20-binding molecule to members of said first population of cells relative to members of said second population of cells is at least 3-fold greater. In some embodiments, members of the first population of cells express at a cellular surface the CD20 which (1) have the extracellular part bound by the two or more CD20 binding regions of the multivalent CD20-binding molecule, (2) have a transmembrane domain, and (3) remain physically coupled to the cell. In some embodiments, members of the first population of cells are CD20 positive cells. For certain embodiments, the members of the first population of cells are physically coupled with a significant amount of extracellular CD20 which (1) have the extracellular part bound by the two or more CD20 binding regions of the multivalent CD20-binding molecule. For certain embodiments, members of the first population of cells over-express, at a cellular surface, CD20 which have the extracellular part bound by the two or more CD20 binding regions of the multivalent CD20-binding molecule. For certain embodiments, members of the first population of cells over-express CD20 which (1) have the extracellular part bound by the two or more CD20 binding regions of the multivalent CD20-binding molecule, (2) have a transmembrane domain, and (3) remain physically coupled to the cell. For certain embodiments, members of the first population of cells are descendants or members of a B-cell lineage. For certain embodiments, members of the first population of cells are malignant B-cell, B-cell leukemia cell, B-cell lymphoma cell, B-cell myeloma cell, acute myeloid leukemia cell, acute non-lymphocytic leukemia cell, B-cell chronic lymphocytic leukemia cell, B-cell lymphoma cell, B-cell non-Hodgkin's lymphoma cell, B-cell precursor acute lymphoblastic leukemia cell, B-cell prolymphocytic leukemia cell, Burkitt's lymphoma cell, chronic lymphocytic leukemia cell, chronic myeloid leukemia cell, diffuse large B-cell lymphoma cell, follicular lymphoma cell, hairy cell leukemia cell, Hodgkin's lymphoma cell, immunoblastic large cell lymphoma cell, mantle cell lymphoma cell, melanoma cell, multiple myeloma cell, neoplastic plasma cell, nodular lymphocyte predominant Hodgkin's lymphoma cell, non-Hodgkin's lymphoma cell, plasmablastic lymphoma cell, plasma cell myeloma cell, precursor B-lymphoblastic lymphoma cell, small lymphocytic lymphoma cell, malignant T-cell, T-cell leukemia cell, T-cell lymphoma cell, T-cell large granular lymphocyte leukemia cell, T-cell prolymphocytic leukemia, healthy B-cell lineage cell, or healthy T-cell members. For certain embodiments, the members of the second population of cells are not physically coupled with extracellular CD20 and/or are CD20 negative. For certain embodiments, the members of the second population of cells are not physically coupled with extracellular CD20 which (1) have the extracellular part bound by the two or more CD20 binding regions of the multivalent CD20-binding molecule. In some embodiments, members of the second population of cells express at a cellular surface the CD20 which (1) have the extracellular part bound by the two or more CD20 binding regions of the multivalent CD20-binding molecule, (2) have a transmembrane domain, and (3) remain physically coupled to the cell. For certain embodiments, the members of the second population of cells are not physically coupled with a significant amount of extracellular CD20 which (1) have the extracellular part bound by the two or more CD20 binding regions of the multivalent CD20-binding molecule. For certain embodiments of the multivalent CD20-binding molecule as described herein, upon administration of the multivalent CD20-binding molecule to a first population of cells whose members are CD20 positive, and a second population of cells whose members are not CD20 positive, a cytotoxic effect of the multivalent CD20-binding molecule to members of said first population of cells relative to members of said second population of cells is at least 3-fold greater.

In some embodiments, the multivalent CD20-binding molecule as described herein comprises two or more proteinaceous components (e.g. protein subunits), wherein each proteinaceous component comprises (1) at least one CD20 binding region capable of specifically binding an extracellular part of a CD20 molecule, and, optionally, (2) one or more Shiga toxin effector polypeptides, each comprising a polypeptide derived from the amino acid sequence of the A Subunit of at least one member of the Shiga toxin family. In some embodiments, each proteinaceous component comprises (1) only one CD20 binding region capable of specifically binding an extracellular part of a CD20 molecule, and (2) only one Shiga toxin effector polypeptide. In some embodiments, the multivalent CD20-binding molecule as described herein comprises exactly two proteinaceous components. In some embodiments, the multivalent CD20-binding molecule as described herein comprises two or more components, wherein at least one component is associated with the multivalent CD20-binding molecule through one or more non-covalent interactions. In some embodiments, at least one of the components is proteinaceous. In some embodiments, the multivalent CD20-binding molecule as described herein comprises two or more proteinaceous components associated with each other, either directly or indirectly, through one or more non-covalent interactions. In some embodiments, each proteinaceous component comprises (1) at least one CD20 binding region capable of specifically binding an extracellular part of a CD20, and (2) a Shiga toxin effector polypeptide.

In some embodiments, the multivalent CD20-binding molecule as described herein comprises two or more proteinaceous components (e.g. protein subunits), wherein each proteinaceous component comprises (1) at least one CD20 binding region capable of specifically binding an extracellular part of a CD20 molecule, and, optionally, (2) one or more Shiga toxin effector regions, each comprising a polypeptide derived from the amino acid sequence of the A Subunit of at least one member of the Shiga toxin family. In some embodiments, each proteinaceous component comprises (1) only one CD20 binding region capable of specifically binding an extracellular part of a CD20 molecule, and (2) only one Shiga toxin effector region. In some embodiments, the multivalent CD20-binding molecule as described herein comprises exactly two proteinaceous components. In some embodiments, the multivalent CD20-binding molecule as described herein comprises two or more components, wherein at least one component is associated with the multivalent CD20-binding molecule through one or more non-covalent interactions. In some embodiments, at least one of the components is proteinaceous. In some embodiments, the multivalent CD20-binding molecule as described herein comprises two or more proteinaceous components associated with each other, either directly or indirectly, through one or more non-covalent interactions. In some embodiments, each proteinaceous component comprises (1) at least one CD20 binding region capable of specifically binding an extracellular part of a CD20, and (2) a Shiga toxin effector region.

In some embodiments, the multivalent CD20-binding molecule as described herein comprises at least one CD20 binding region comprising an immunoglobulin-type binding region. In some embodiments, the multivalent CD20-binding molecule as described herein comprises the CD20 binding region comprising an immunoglobulin-type binding region comprising a polypeptide, the polypeptide being an autonomous V_{H} domain, single-domain antibody fragment (sdAb), nanobody, heavy chain-antibody domain derived from a camelid (V_{H}H or V_{H} domain fragment), heavy-chain antibody domain derived from a cartilaginous fish (V_{H}H or V_{H} domain fragment), immunoglobulin new antigen receptor (IgNAR), V_{NAR} fragment, single-chain variable fragment (scFv), antibody variable fragment (Fv), complementary determining region 3 fragment (CDR3), constrained FR3-CDR3-FR4 polypeptide (FR3-CDR3-FR4), Fd fragment, small modular immunopharmaceutical (SMIP) domain, antigen-binding fragment (Fab), Armadillo repeat polypeptide (ArmRP), fibronectin-derived 10^{th} fibronectin type III domain (10Fn3), tenascin type III domain (TNfn3), ankyrin repeat motif domain, low-density-lipoprotein-receptor-derived A-domain (LDLR-A), lipocalin (anticalin), Kunitz domain, Protein-A-derived Z domain, gamma-B crystalline-derived domain, ubiquitin-derived domain, Sac7d-derived polypeptide (affitin), Fyn-derived SH2 domain, miniprotein, C-type lectin-like domain scaffold or engineered antibody mimic, or any genetically manipulated counterpart of any of the foregoing which retains binding functionality.

In some embodiments, the multivalent CD20-binding molecule as described herein does not comprise an immunoglobulin Fc region or Fc region effector which retains an Fc region function, such as, e.g., involving extracellular signaling to immune system factors, cells, and/or tissues. Non-limiting examples of Fc region functions include activating T-cells, stimulating the release of inflammatory mediators such as cytokines like TNF-alpha, initiating complement dependent cytotoxicity (CDC), antibody-dependent cytotoxicity (ADCC), and phagocytosis of the cell bound extracellularly by the molecule comprising the Fc region.

In some embodiments, the multivalent CD20-binding molecule as described herein does not comprise any immunoglobulin heavy chain constant region, immunoglobulin light chain constant region, immunoglobulin C_{L} domain, immunoglobulin C_{H}1 domain, immunoglobulin C_{H}2 domain, and/or immunoglobulin C_{H}3 domain. In some embodiments, the multivalent CD20-binding molecule does not comprise any immunoglobulin domains other than the immunoglobulin domains selected from (1) CDR, (2) ABR, and/or (3) any immunoglobulin domain present in an autonomous V_{H} domain, single-domain antibody domains (sdAb), heavy-chain antibody domain fragment (V_{H}H fragments or V_{H} domain fragment), and single-chain variable fragment (scFv). In some embodiments, the multivalent CD20-binding molecule as described herein does not comprise any immunoglobulin domain or any polypeptide derived from an immunoglobulin.

In some embodiments, the multivalent CD20-binding molecule as described herein comprises at least one Shiga toxin effector polypeptide comprising, consisting essentially of, or consisting of the following polypeptide: (a) amino acids 75 to 251 of SEQ ID NO:1, SEQ ID NO:2, or SEQ ID NO:3; (b) amino acids 1 to 241 of SEQ ID NO:1, SEQ ID NO:2, or SEQ ID NO:3; (c) amino acids 1 to 251 of SEQ ID NO:1, SEQ ID NO:2, or SEQ ID NO:3; or (d) amino acids 1 to 261 of SEQ ID NO:1, SEQ ID NO:2, or SEQ ID NO:3.

In some embodiments, the multivalent CD20-binding molecule as described herein comprises at least one Shiga toxin effector region comprising, consisting essentially of, or consisting of the following polypeptide: (a) amino acids 75 to 251 of SEQ ID NO:1, SEQ ID NO:2, or SEQ ID NO:3; (b) amino acids 1 to 241 of SEQ ID NO:1, SEQ ID NO:2, or SEQ ID NO:3; (c) amino acids 1 to 251 of SEQ ID NO:1, SEQ ID NO:2, or SEQ ID NO:3; or (d) amino acids 1 to 261 of SEQ ID NO:1, SEQ ID NO:2, or SEQ ID NO:3.

In some embodiments, the multivalent CD20-binding molecule is monomeric. In some embodiments, the multivalent CD20-binding molecule is multimeric, e.g. the molecule comprises at least two, independent, polypeptide chains (e.g. protein subunits) associated either directly or indirectly to form a single molecule. In some embodiments, the multimeric CD20-binding molecule as described herein comprises two or more protein components, e.g., two or more individual CD20-binding proteins. In some embodiments, the multimeric CD20-binding molecule as described herein comprises two or more protein components (e.g. subunits) which are associated through one or more non-covalent interactions.

In some embodiments, the multimeric CD20-binding molecule as described herein comprises two or more proteins components (e.g. subunits) which are associated through one or more covalent interactions. In some embodiments, the multivalent CD20-binding molecule as described herein comprises two or more protein components, e.g., two or more individual CD20-binding proteins, which are associated through one or more covalent interactions, wherein at a least one covalent interaction is a disulfide bond between at least two of the protein components (e.g., between different protein subunits).

In some embodiments, the multivalent CD20-binding molecule as described herein comprises the molecule shown in any one of SEQ ID NOs: 1-304.

In some embodiments, the multivalent CD20-binding molecule as described herein comprises a CD20 binding region which comprises the immunoglobulin-type binding region comprising the following polypeptide(s): (a) a heavy chain variable (V_{H}) domain comprising (i) a HCDR1 comprising, consisting essentially of, or consisting of the amino acid sequence as shown in SEQ ID NO:5, SEQ ID NO:11, SEQ ID NO:17, SEQ ID NO:23, SEQ ID NO:29, or SEQ ID NO:35; (ii) a HCDR2 comprising, consisting essentially of, or consisting of the amino acid sequence as shown in SEQ ID NO:6, SEQ ID NO:12, SEQ ID NO:18, SEQ ID NO:24, SEQ ID NO:30, or SEQ ID NO:36; and/or (iii) a HCDR3 comprising, consisting essentially of, or consisting of the amino acid sequence as shown in SEQ ID NO:7, SEQ ID NO:13, SEQ ID NO:19, SEQ ID NO:25, SEQ ID NO:31, or SEQ ID NO:37; and/or (b) a light chain variable (V_{L}) domain comprising (i) a LCDR1 comprising, consisting essentially of, or consisting of the amino acid sequence as shown in SEQ ID NO:8, SEQ ID NO:14, SEQ ID NO:20, SEQ ID NO:26, SEQ ID NO:32, or SEQ ID NO:38; (ii) a LCDR2 comprising, consisting essentially of, or consisting of the amino acid sequence as shown in SEQ ID NO:9, SEQ ID NO:15, SEQ ID NO:21, SEQ ID NO:27, SEQ ID NO:33, or SEQ ID NO:39; and/or (iii) a LCDR3 comprising, consisting essentially of, or consisting of the amino acid sequence as shown in SEQ ID NO:10, SEQ ID NO:16, SEQ ID NO:22, SEQ ID NO:28, SEQ ID NO:34, or SEQ ID NO:40.

In some embodiments, a multivalent CD20-binding molecule as described herein comprises a CD20 binding region comprising, consisting essentially of, or consisting of amino acids 1-232, 1-233, 1-234, 1-235, 1-236, 1-242, 1-243, 1-244, 1-245, 1-246, 1-252, 1-253, 1-254, 1-255, or 1-256 of any one of SEQ ID NOs: 47-119 and 176-248.

In some embodiments, a CD20-binding molecule comprises the protein shown in any one of SEQ ID NOs: 47-304, and optionally, the protein further comprises an amino-terminal methionine residue. In some embodiments, a CD20-binding molecule is multivalent and comprises, consists essentially of, or consists of two proteins, each protein selected from any one of the polypeptides shown in SEQ ID NOs: 47-304, and optionally, each protein further comprises an amino-terminal methionine residue. In some embodiments, the protein is any one of the proteins having the sequence of SEQ ID NOs: 47-175 and further comprises a disulfide bond involving a cysteine residue at position 242, 482, 483, 484, 490, 491, 492, 493, 494, 495, 499, 500, 501, 502, 503, 504, 505, 510, 511, 512, 513 or 521.

In some embodiments, a CD20-binding molecule is multimeric and comprises, consists essentially of, or consists of: (a) two polypeptides selected from the polypeptides shown in SEQ ID NOs: 47-175 and 249-304, which each optionally may further comprise an amino-terminal methionine residue, and (b) a cysteine disulfide bond linking the two polypeptides, wherein the cysteine disulfide bond involves a cysteine residue in each of the two polypeptides located at amino acid position 242, 482, 483, 484, 490, 491, 492, 493, 494, 495, 499, 500, 501, 502, 503, 504, 505, 510, 511, 512, 513, or 521. In some embodiments, a multimeric CD20-binding molecule as described herein is a homodimer and consists essentially of: (a) two identical polypeptides, the polypeptides selected from one of the polypeptides shown in SEQ ID NOs: 47-175 and 249-304, and (b) a cysteine disulfide bond linking the two identical polypeptides, wherein the cysteine disulfide bond involves a cysteine residue in each of the two identical polypeptides located at amino acid position 242, 482, 483, 484, 490, 491, 492, 493, 494, 495, 499, 500, 501, 502, 503, 504, 505, 510, 511, 512, 513, or 521. In some embodiments, a multimeric CD20-binding molecule is a homodimer, wherein the two identical polypeptides are both the polypeptide shown in SEQ ID NO:49, and the amino acid position of the cysteine residue is 490. In some embodiments, a multivalent CD20-binding molecule is a homodimer, wherein the two identical polypeptides are both selected from the polypeptide shown in SEQ ID NO:50, SEQ ID NO:61, SEQ ID NO:73, SEQ ID NO:96, SEQ ID NO:101, or SEQ ID NO:102; and the amino acid position of the cysteine residue is 501. In some embodiments, a multivalent CD20-binding molecule is a homodimer, wherein the two identical polypeptides are both selected from the polypeptide shown in SEQ ID NO:53, SEQ ID NO:63, SEQ ID NO:66, SEQ ID NO:75, SEQ ID NO:83, SEQ ID NO:89, or SEQ ID NO:95; and the amino acid position of the cysteine residue is 512. In some embodiments, a CD20-binding molecule is a homodimer, wherein the two identical polypeptides are both the polypeptide shown in SEQ ID NO:54, and the amino acid position of the cysteine residue is 503. In some embodiments, a CD20-binding molecule is a homodimer, wherein the two identical polypeptides are both selected from the polypeptide shown in SEQ ID NO:55, SEQ ID NO:64, SEQ ID NO:67, SEQ ID NO:76, SEQ ID NO:90, SEQ ID NO:92, SEQ ID NO:93, SEQ ID NO:97, or SEQ ID NO:98; and the amino acid position of the cysteine residue is 502. In some embodiments, CD20-binding molecule is a homodimer, wherein the two identical polypeptides are both selected from the polypeptide shown in SEQ ID NO:56, SEQ ID NO:68, SEQ ID NO:91, SEQ ID NO:99, SEQ ID NO:103, or SEQ ID NO:104; and the amino acid position of the cysteine residue is 492. In some embodiments, a CD20-binding molecule is a homodimer, wherein the two identical polypeptides are both selected from the polypeptide shown in SEQ ID NO:54, SEQ ID NO:57, SEQ ID NO:69, SEQ ID NO:78, SEQ ID NO:82, SEQ ID NO:84, SEQ ID NO:87, SEQ ID NO:88, SEQ ID NO:94, SEQ ID NO:110, SEQ ID NO:111, or SEQ ID NO:115; and the amino acid position of the cysteine residue is 503. In some embodiments, a CD20-binding molecule is a homodimer, wherein the two identical polypeptides are both selected from the polypeptide shown in SEQ ID NO:58, SEQ ID NO:70, or SEQ ID NO:81; and the amino acid position of the cysteine residue is 493. In some embodiments, a CD20-binding molecule is a homodimer, wherein the two identical polypeptides are both selected from only the polypeptides shown in SEQ ID NOs: 249-304, and the amino acid position of the cysteine residue is 242.

In some embodiments, a CD20-binding molecule is monospecific for CD20-binding. In some embodiments, all the CD20 binding regions present in the CD20-binding molecule bind, on their own, the same extracellular part of the same CD20 (e.g. monoparatopic). In some embodiments, all the CD20 binding regions present in the CD20-binding molecule bind, on their own, the same extracellular CD20 epitope with equivalent specificities.

In some embodiments, the multivalent CD20-binding molecule comprises one or more monovalent CD20-binding molecule components; and whereby upon administration of the multivalent CD20-binding molecule as described herein to a population of cells physically coupled with CD20, which have the extracellular part bound by two or more CD20 binding regions of the multivalent CD20-binding molecule, the multivalent CD20-binding molecule exhibits a cytotoxic effect to the population of cells which is greater than a cytotoxic effect resulting from administration of an equivalent amount, mass, or molarity of any one of the monovalent CD20-binding molecule components of the multivalent CD20-binding molecule to a population of the same CD20 positive cells under same conditions by a factor of 1.33, 1.5, 1.75, 2, 3, 5, 7.5, 10, 20, 100, or greater than the change in CD20-binding valence between the monovalent CD20-binding component and the multivalent CD20-binding molecule. In some embodiments, members of the population of cells express at a cellular surface the CD20 which (1) have the extracellular part bound by the two or more CD20 binding regions of the multivalent CD20-binding molecule, (2) have a transmembrane domain, and (3) remain physically coupled to the cell. In some embodiments, members of the population of cells are CD20 positive cells. For certain embodiments, the members of the population of cells are physically coupled with a significant amount of extracellular CD20 which (1) have the extracellular part bound by the two or more CD20 binding regions of the multivalent CD20-binding molecule. For certain embodiments, members of the population of cells are descendants or members of a B-cell lineage. For certain embodiments, members of the population of cells are malignant B-cell, B-cell leukemia cell, B-cell lymphoma cell, B-cell myeloma cell, acute myeloid leukemia cell, acute non-lymphocytic leukemia cell, B-cell chronic lymphocytic leukemia cell, B-cell lymphoma cell, B-cell non-Hodgkin's lymphoma cell, B-cell precursor acute lymphoblastic leukemia cell, B-cell prolymphocytic leukemia cell, Burkitt's lymphoma cell, chronic lymphocytic leukemia cell, chronic myeloid leukemia cell, diffuse large B-cell lymphoma cell, follicular lymphoma cell, hairy cell leukemia cell, Hodgkin's lymphoma cell, immunoblastic large cell lymphoma cell, mantle cell lymphoma cell, melanoma cell, multiple myeloma cell, neoplastic plasma cell, nodular lymphocyte predominant Hodgkin's lymphoma cell, non-Hodgkin's lymphoma cell, plasmablastic lymphoma cell, plasma cell myeloma cell, precursor B-lymphoblastic lymphoma cell, small lymphocytic lymphoma cell, malignant T-cell, T-cell leukemia cell, T-cell lymphoma cell, T-cell large granular lymphocyte leukemia cell, T-cell prolymphocytic leukemia, healthy B-cell lineage cell, or healthy T-cell members. In some embodiments, the multivalent CD20-binding molecule as described herein comprises one or more monovalent CD20-binding molecule components; and whereby upon administration of the multivalent CD20-binding molecule as described herein to a population of CD20 positive cells expressing CD20 which have the extracellular part bound by two or more CD20 binding regions of the multivalent CD20-binding molecule, the multivalent CD20-binding molecule exhibits a cytotoxic effect which is greater than a cytotoxic effect resulting from administration of an equivalent amount, mass, or molarity of any one of the monovalent CD20-binding molecule components of the multivalent CD20-binding molecule to a population of the same CD20 positive cells under same conditions by a factor of 1.33, 1.5, 1.75, 2, 3, 5, 7.5, 10, 20, 100, or greater than the change in CD20-binding valence between the monovalent CD20-binding component and the multivalent CD20-binding molecule.

In some embodiments, the multivalent CD20-binding molecule as described herein comprises one or more monovalent CD20-binding molecule components; and whereby upon administration of the multivalent CD20-binding molecule as described herein to a population of cells physically coupled with CD20, which have the extracellular part bound by two or more CD20 binding regions of the multivalent CD20-binding molecule, the multivalent CD20-binding molecule exhibits a cytotoxic effect which is greater than a cytotoxic effect resulting from administration of an equivalent amount, mass, or molarity of any one of the monovalent CD20-binding molecule components of the multivalent CD20-binding molecule to a population of the same CD20 positive cells under same conditions by a factor of 1.33, 1.5, 1.75, 2, 3, 5, 7.5, 10, 20, 100, or greater than the change in equilibrium binding constants (K_{D}) between the multivalent CD20-binding molecule and the monovalent CD20-binding component for binding to CD20 or CD20-expressing cell. In some embodiments, members of the population of cells express at a cellular surface the CD20 which (1) have the extracellular part bound by the two or more CD20 binding regions of the multivalent CD20-binding molecule, (2) have a transmembrane domain, and (3) remain physically coupled to the cell. In some embodiments, members of the population of cells are CD20 positive cells. For certain embodiments, the members of the population of cells are physically coupled with a significant amount of extracellular CD20 which (1) have the extracellular part bound by the two or more CD20 binding regions of the multivalent CD20-binding molecule. For certain embodiments, members of the population of cells are descendants or members of a B-cell lineage. For certain embodiments, members of the population of cells are malignant B-cell, B-cell leukemia cell, B-cell lymphoma cell, B-cell myeloma cell, acute myeloid leukemia cell, acute non-lymphocytic leukemia cell, B-cell chronic lymphocytic leukemia cell, B-cell lymphoma cell, B-cell non-Hodgkin's lymphoma cell, B-cell precursor acute lymphoblastic leukemia cell, B-cell prolymphocytic leukemia cell, Burkitt's lymphoma cell, chronic lymphocytic leukemia cell, chronic myeloid leukemia cell, diffuse large B-cell lymphoma cell, follicular lymphoma cell, hairy cell leukemia cell, Hodgkin's lymphoma cell, immunoblastic large cell lymphoma cell, mantle cell lymphoma cell, melanoma cell, multiple myeloma cell, neoplastic plasma cell, nodular lymphocyte predominant Hodgkin's lymphoma cell, non-Hodgkin's lymphoma cell, plasmablastic lymphoma cell, plasma cell myeloma cell, precursor B-lymphoblastic lymphoma cell, small lymphocytic lymphoma cell, malignant T-cell, T-cell leukemia cell, T-cell lymphoma cell, T-cell large granular lymphocyte leukemia cell, T-cell prolymphocytic leukemia, healthy B-cell lineage cell, or healthy T-cell members. In some embodiments, the multivalent CD20-binding molecule as described herein comprises one or more monovalent CD20-binding molecule components; and whereby upon administration of the multivalent CD20-binding molecule as described herein to a population of CD20 positive cells expressing CD20 which have the extracellular part bound by two or more CD20 binding regions of the multivalent CD20-binding molecule, the multivalent CD20-binding molecule exhibits a cytotoxic effect which is greater than a cytotoxic effect resulting from administration of an equivalent amount, mass, or molarity of any one of the monovalent CD20-binding molecule components of the multivalent CD20-binding molecule to a population of the same CD20 positive cells under same conditions by a factor of 1.33, 1.5, 1.75, 2, 3, 5, 7.5, 10, 20, 100, or greater than the change in equilibrium binding constants (K_{D}) between the multivalent CD20-binding molecule and the monovalent CD20-binding component for binding to CD20 or CD20-expressing cell.

In some embodiments, the multivalent CD20-binding molecule as described herein comprises one or more monovalent CD20-binding molecule components; and whereby upon administration of the multivalent CD20-binding molecule as described herein to a population of cells physically coupled with CD20, which have the extracellular part bound by two or more CD20 binding regions of the multivalent CD20-binding molecule, the multivalent CD20-binding molecule exhibits a cytotoxic effect which is greater than a cytotoxic effect resulting from administration of an equivalent amount, mass, or molarity of any one of the monovalent CD20-binding molecule components of the multivalent CD20-binding molecule to a population of the same CD20 positive cells under same conditions by a factor of 1.33, 1.5, 1.75, 2, 3, 5, 7.5, 10, 20, 100, or greater than the change in affinity constant (1/K_{D}) between the multivalent CD20-binding molecule and the monovalent CD20-binding component for binding to CD20 or CD20-expressing cell. In some embodiments, members of the population of cells express at a cellular surface the CD20 which (1) have the extracellular part bound by the two or more CD20 binding regions of the multivalent CD20-binding molecule, (2) have a transmembrane domain, and (3) remain physically coupled to the cell. In some embodiments, members of the population of cells are CD20 positive cells. For certain embodiments, the members of the population of cells are physically coupled with a significant amount of extracellular CD20 which (1) have the extracellular part bound by the two or more CD20 binding regions of the multivalent CD20-binding molecule. For certain embodiments, members of the population of cells are descendants or members of a B-cell lineage. For certain embodiments, members of the population of cells are malignant B-cell, B-cell leukemia cell, B-cell lymphoma cell, B-cell myeloma cell, acute myeloid leukemia cell, acute non-lymphocytic leukemia cell, B-cell chronic lymphocytic leukemia cell, B-cell lymphoma cell, B-cell non-Hodgkin's lymphoma cell, B-cell precursor acute lymphoblastic leukemia cell, B-cell prolymphocytic leukemia cell, Burkitt's lymphoma cell, chronic lymphocytic leukemia cell, chronic myeloid leukemia cell, diffuse large B-cell lymphoma cell, follicular lymphoma cell, hairy cell leukemia cell, Hodgkin's lymphoma cell, immunoblastic large cell lymphoma cell, mantle cell lymphoma cell, melanoma cell, multiple myeloma cell, neoplastic plasma cell, nodular lymphocyte predominant Hodgkin's lymphoma cell, non-Hodgkin's lymphoma cell, plasmablastic lymphoma cell, plasma cell myeloma cell, precursor B-lymphoblastic lymphoma cell, small lymphocytic lymphoma cell, malignant T-cell, T-cell leukemia cell, T-cell lymphoma cell, T-cell large granular lymphocyte leukemia cell, T-cell prolymphocytic leukemia, healthy B-cell lineage cell, or healthy T-cell members. In some embodiments, the multivalent CD20-binding molecule as described herein comprises one or more monovalent CD20-binding molecule components; and whereby upon administration of the multivalent CD20-binding molecule as described herein to a population of CD20 positive cells expressing CD20 which have the extracellular part bound by two or more CD20 binding regions of the multivalent CD20-binding molecule, the multivalent CD20-binding molecule exhibits a cytotoxic effect which is greater than a cytotoxic effect resulting from administration of an equivalent amount, mass, or molarity of any one of the monovalent CD20-binding molecule components of the multivalent CD20-binding molecule to a population of the same CD20 positive cells under same conditions by a factor of 1.33, 1.5, 1.75, 2, 3, 5, 7.5, 10, 20, 100, or greater than the change in affinity constant (1/K_{D}) between the multivalent CD20-binding molecule and the monovalent CD20-binding component for binding to CD20 or CD20-expressing cell.

In some embodiments of the multivalent CD20-binding molecule as described herein, one or more polypeptide components of the CD20-binding molecule comprises a carboxy-terminal endoplasmic reticulum retention/retrieval signal motif of a member of the KDEL family. In some embodiments, the carboxy-terminal endoplasmic reticulum retention/retrieval signal motif has the sequence of: KDEL (SEQ ID NO:305), HDEF (SEQ ID NO:306), HDEL (SEQ ID NO:307), RDEF (SEQ ID NO:308), RDEL (SEQ ID NO:309), WDEL (SEQ ID NO:310), YDEL (SEQ ID NO:311), HEEF (SEQ ID NO:312), HEEL (SEQ ID NO:313), KEEL (SEQ ID NO:314), REEL (SEQ ID NO:315), KAEL (SEQ ID NO:316), KCEL (SEQ ID NO:317), KFEL (SEQ ID NO:318), KGEL (SEQ ID NO:319), KHEL (SEQ ID NO:320), KLEL (SEQ ID NO:321), KNEL (SEQ ID NO:322), KQEL (SEQ ID NO:323), KREL (SEQ ID NO:324), KSEL (SEQ ID NO:325), KVEL (SEQ ID NO:326), KWEL (SEQ ID NO:327), KYEL (SEQ ID NO:328), KEDL (SEQ ID NO:329), KIEL (SEQ ID NO:330), DKEL (SEQ ID NO:331), FDEL (SEQ ID NO:332), KDEF (SEQ ID NO:333), KKEL (SEQ ID NO:334), HADL (SEQ ID NO:335), HAEL (SEQ ID NO:336), HIEL (SEQ ID NO:337), HNEL (SEQ ID NO:338), HTEL (SEQ ID NO:339), KTEL (SEQ ID NO:340), HVEL (SEQ ID NO:341), NDEL (SEQ ID NO:342), QDEL (SEQ ID NO:343), REDL (SEQ ID NO:344), RNEL (SEQ ID NO:345), RTDL (SEQ ID NO:346), RTEL (SEQ ID NO:347), SDEL (SEQ ID NO:348), TDEL (SEQ ID NO:349), SKEL (SEQ ID NO:350), STEL (SEQ ID NO:351), or EDEL (SEQ ID NO:352).

In some embodiments of the multivalent CD20-binding molecule as described herein, one or more Shiga toxin effector polypeptides comprises a mutation relative to a naturally occurring A Subunit of a member of the Shiga toxin family that changes the enzymatic activity of the Shiga toxin effector polypeptide, the mutation selected from at least one amino acid residue deletion, insertion, or substitution, such as, e.g., A231E, R75A, Y77S, Y114S, E167D, R170A, R176K and/or W203A in SEQ ID NO:1, SEQ ID NO:2, SEQ ID NO:3, or SEQ ID NO:4. In some embodiments, the mutation is selected from at least one amino acid residue deletion, insertion, or substitution that reduces or eliminates catalytic activity but retains at least one other Shiga toxin effector function, such as, e.g., inducing cellular internalization and/or directing subcellular routing. In some embodiments, the mutation reduces or eliminates cytotoxicity of the Shiga toxin effecter polypeptide.

In some embodiments of the multivalent CD20-binding molecule as described herein, one or more Shiga toxin effector regions comprises a mutation relative to a naturally occurring A Subunit of a member of the Shiga toxin family that changes the enzymatic activity of the Shiga toxin effector region, the mutation selected from at least one amino acid residue deletion, insertion, or substitution, such as, e.g., A231E, R75A, Y77S, Y114S, E167D, R170A, R176K and/or W203A in SEQ ID NO:1, SEQ ID NO:2, SEQ ID NO:3, or SEQ ID NO:4. In some embodiments, the mutation is selected from at least one amino acid residue deletion, insertion, or substitution that reduces or eliminates catalytic activity but retains at least one other Shiga toxin effector function, such as, e.g., inducing cellular internalization and/or directing subcellular routing. In some embodiments, the mutation reduces or eliminates cytotoxicity of the Shiga toxin effecter region.

For certain embodiments of the multivalent CD20-binding molecule, the multivalent CD20-binding molecule may be utilized for the delivery of additional exogenous material into a cell. In some embodiments, the multivalent CD20-binding molecule as described herein comprises an additional exogenous material. For certain embodiments of the multivalent CD20-binding molecule as described herein, which comprises an additional exogenous material; whereby upon administration of the multivalent CD20-binding molecule to one or more cells physically coupled with CD20, which have the extracellular part bound by the two or more CD20 binding regions of the multivalent CD20-binding molecule, the multivalent CD20-binding molecule internalizes into the one or more cells in about five hours, four hours, three hours, two hours, one hour, thirty minutes, or less at a physiological temperature appropriate for the cell and/or at about 37 degrees Celsius. In some embodiments, the one or more cell(s) expresses at a cellular surface the CD20 which (1) have the extracellular part bound by the two or more CD20 binding regions of the multivalent CD20-binding molecule, (2) have a transmembrane domain, and (3) remain physically coupled to the cell. In some embodiments, one or more cell(s) is a CD20 positive cell. For certain embodiments, one or more cell(s) is physically coupled with a significant amount of extracellular CD20 which (1) have the extracellular part bound by the two or more CD20 binding regions of the multivalent CD20-binding molecule. For certain embodiments, one or more cell(s) is a descendant or member of a B-cell lineage. For certain embodiments, one or more cell(s) is a malignant B-cell, B-cell leukemia cell, B-cell lymphoma cell, B-cell myeloma cell, acute myeloid leukemia cell, acute non-lymphocytic leukemia cell, B-cell chronic lymphocytic leukemia cell, B-cell lymphoma cell, B-cell non-Hodgkin's lymphoma cell, B-cell precursor acute lymphoblastic leukemia cell, B-cell prolymphocytic leukemia cell, Burkitt's lymphoma cell, chronic lymphocytic leukemia cell, chronic myeloid leukemia cell, diffuse large B-cell lymphoma cell, follicular lymphoma cell, hairy cell leukemia cell, Hodgkin's lymphoma cell, immunoblastic large cell lymphoma cell, mantle cell lymphoma cell, melanoma cell, multiple myeloma cell, neoplastic plasma cell, nodular lymphocyte predominant Hodgkin's lymphoma cell, non-Hodgkin's lymphoma cell, plasmablastic lymphoma cell, plasma cell myeloma cell, precursor B-lymphoblastic lymphoma cell, small lymphocytic lymphoma cell, malignant T-cell, T-cell leukemia cell, T-cell lymphoma cell, T-cell large granular lymphocyte leukemia cell, T-cell prolymphocytic leukemia, healthy B-cell lineage cell, or healthy T-cell.

For certain embodiments of the multivalent CD20-binding molecule as described herein, which comprises an additional exogenous material; whereby upon administration of the multivalent CD20-binding molecule to one or more cells physically coupled with CD20, which have the extracellular part bound by the two or more CD20 binding regions of the multivalent CD20-binding molecule, the multivalent CD20-binding molecule internalizes into the one or more cells and delivers the additional exogenous material into the interior of the cell in about five hours, four hours, three hours, two hours, one hour, thirty minutes, or less at a physiological temperature appropriate for the cell and/or at about 37 degrees Celsius. In some embodiments, the one or more cell(s) expresses at a cellular surface the CD20 which (1) have the extracellular part bound by the two or more CD20 binding regions of the multivalent CD20-binding molecule, (2) have a transmembrane domain, and (3) remain physically coupled to the cell. In some embodiments, one or more cell(s) is a CD20 positive cell. For certain embodiments, one or more cell(s) is physically coupled with a significant amount of extracellular CD20 which (1) have the extracellular part bound by the two or more CD20 binding regions of the multivalent CD20-binding molecule. For certain embodiments, one or more cell(s) is a descendant or member of a B-cell lineage. For certain embodiments, one or more cell(s) is a malignant B-cell, B-cell leukemia cell, B-cell lymphoma cell, B-cell myeloma cell, acute myeloid leukemia cell, acute non-lymphocytic leukemia cell, B-cell chronic lymphocytic leukemia cell, B-cell lymphoma cell, B-cell non-Hodgkin's lymphoma cell, B-cell precursor acute lymphoblastic leukemia cell, B-cell prolymphocytic leukemia cell, Burkitt's lymphoma cell, chronic lymphocytic leukemia cell, chronic myeloid leukemia cell, diffuse large B-cell lymphoma cell, follicular lymphoma cell, hairy cell leukemia cell, Hodgkin's lymphoma cell, immunoblastic large cell lymphoma cell, mantle cell lymphoma cell, melanoma cell, multiple myeloma cell, neoplastic plasma cell, nodular lymphocyte predominant Hodgkin's lymphoma cell, non-Hodgkin's lymphoma cell, plasmablastic lymphoma cell, plasma cell myeloma cell, precursor B-lymphoblastic lymphoma cell, small lymphocytic lymphoma cell, malignant T-cell, T-cell leukemia cell, T-cell lymphoma cell, T-cell large granular lymphocyte leukemia cell, T-cell prolymphocytic leukemia, healthy B-cell lineage cell, or healthy T-cell.

For certain embodiments of the multivalent CD20-binding molecule as described herein, which comprises an additional exogenous material; whereby upon administration of the multivalent CD20-binding molecule to a plurality of cells physically coupled with CD20, which have the extracellular part bound by the two or more CD20 binding regions of the multivalent CD20-binding molecule, at a concentration of multivalent CD20-binding molecule equivalent to five or thirty-eight percent to fifty percent cell-surface occupancy, the majority of the multivalent CD20-binding molecule internalizes into the plurality of cells in about five hours, four hours, three hours, two hours, one hour, thirty minutes, or less at a physiological temperature appropriate for the cell and/or at about 37 degrees Celsius. In some embodiments, members of the plurality of cells express at a cellular surface the CD20 which (1) have the extracellular part bound by the two or more CD20 binding regions of the multivalent CD20-binding molecule, (2) have a transmembrane domain, and (3) remain physically coupled to the cell. In some embodiments, members of the plurality of cells are CD20 positive cells. For certain embodiments, the members of the plurality of cells are physically coupled with a significant amount of extracellular CD20 which (1) have the extracellular part bound by the two or more CD20 binding regions of the multivalent CD20-binding molecule. For certain embodiments, members of the plurality of cells are descendants or members of a B-cell lineage. For certain embodiments, members of the plurality of cells are malignant B-cell, B-cell leukemia cell, B-cell lymphoma cell, B-cell myeloma cell, acute myeloid leukemia cell, acute non-lymphocytic leukemia cell, B-cell chronic lymphocytic leukemia cell, B-cell lymphoma cell, B-cell non-Hodgkin's lymphoma cell, B-cell precursor acute lymphoblastic leukemia cell, B-cell prolymphocytic leukemia cell, Burkitt's lymphoma cell, chronic lymphocytic leukemia cell, chronic myeloid leukemia cell, diffuse large B-cell lymphoma cell, follicular lymphoma cell, hairy cell leukemia cell, Hodgkin's lymphoma cell, immunoblastic large cell lymphoma cell, mantle cell lymphoma cell, melanoma cell, multiple myeloma cell, neoplastic plasma cell, nodular lymphocyte predominant Hodgkin's lymphoma cell, non-Hodgkin's lymphoma cell, plasmablastic lymphoma cell, plasma cell myeloma cell, precursor B-lymphoblastic lymphoma cell, small lymphocytic lymphoma cell, malignant T-cell, T-cell leukemia cell, T-cell lymphoma cell, T-cell large granular lymphocyte leukemia cell, T-cell prolymphocytic leukemia, healthy B-cell lineage cell, or healthy T-cell members.

For certain embodiments of the multivalent CD20-binding molecule as described herein, which comprises an additional exogenous material; whereby upon administration of the multivalent CD20-binding molecule to one or more cells physically coupled with CD20, which have the extracellular part bound by the two or more CD20 binding regions of the multivalent CD20-binding molecule, the multivalent CD20-binding molecule internalizes into the one or more cells and delivers the additional exogenous material into the interior of the cell in about five hours, four hours, three hours, two hours, one hour, thirty minutes, or less at a physiological temperature appropriate for the cell and/or at about 37 degrees Celsius. In some embodiments, the one or more cell(s) expresses at a cellular surface the CD20 which (1) have the extracellular part bound by the two or more CD20 binding regions of the multivalent CD20-binding molecule, (2) have a transmembrane domain, and (3) remain physically coupled to the cell. In some embodiments, one or more cell(s) is a CD20 positive cell. For certain embodiments, one or more cell(s) is physically coupled with a significant amount of extracellular CD20 which (1) have the extracellular part bound by the two or more CD20 binding regions of the multivalent CD20-binding molecule. For certain embodiments, one or more cell(s) is a descendant or member of a B-cell lineage. For certain embodiments, one or more cell(s) is a malignant B-cell, B-cell leukemia cell, B-cell lymphoma cell, B-cell myeloma cell, acute myeloid leukemia cell, acute non-lymphocytic leukemia cell, B-cell chronic lymphocytic leukemia cell, B-cell lymphoma cell, B-cell non-Hodgkin's lymphoma cell, B-cell precursor acute lymphoblastic leukemia cell, B-cell prolymphocytic leukemia cell, Burkitt's lymphoma cell, chronic lymphocytic leukemia cell, chronic myeloid leukemia cell, diffuse large B-cell lymphoma cell, follicular lymphoma cell, hairy cell leukemia cell, Hodgkin's lymphoma cell, immunoblastic large cell lymphoma cell, mantle cell lymphoma cell, melanoma cell, multiple myeloma cell, neoplastic plasma cell, nodular lymphocyte predominant Hodgkin's lymphoma cell, non-Hodgkin's lymphoma cell, plasmablastic lymphoma cell, plasma cell myeloma cell, precursor B-lymphoblastic lymphoma cell, small lymphocytic lymphoma cell, malignant T-cell, T-cell leukemia cell, T-cell lymphoma cell, T-cell large granular lymphocyte leukemia cell, T-cell prolymphocytic leukemia, healthy B-cell lineage cell, or healthy T-cell.

For certain embodiments of the multivalent CD20-binding molecule as described herein, which comprises an additional exogenous material; whereby upon administration of the multivalent CD20-binding molecule to a plurality of cells physically coupled with CD20, which have the extracellular part bound by the two or more CD20 binding regions of the multivalent CD20-binding molecule, at a concentration of multivalent CD20-binding molecule equivalent to five or thirty-eight percent to fifty percent cell-surface occupancy, the majority of the multivalent CD20-binding molecule internalizes into the plurality of cells and delivers the additional exogenous material into the interiors of the cells in about five hours, four hours, three hours, two hours, one hour, thirty minutes, or less at a physiological temperature appropriate for the cell and/or at about 37 degrees Celsius. In some embodiments, members of the plurality of cells express at a cellular surface the CD20 which (1) have the extracellular part bound by the two or more CD20 binding regions of the multivalent CD20-binding molecule, (2) have a transmembrane domain, and (3) remain physically coupled to the cell. In some embodiments, members of the plurality of cells are CD20 positive cells. For certain embodiments, the members of the plurality of cells are physically coupled with a significant amount of extracellular CD20 which (1) have the extracellular part bound by the two or more CD20 binding regions of the multivalent CD20-binding molecule. For certain embodiments, members of the plurality of cells are descendants or members of a B-cell lineage. For certain embodiments, members of the plurality of cells are malignant B-cell, B-cell leukemia cell, B-cell lymphoma cell, B-cell myeloma cell, acute myeloid leukemia cell, acute non-lymphocytic leukemia cell, B-cell chronic lymphocytic leukemia cell, B-cell lymphoma cell, B-cell non-Hodgkin's lymphoma cell, B-cell precursor acute lymphoblastic leukemia cell, B-cell prolymphocytic leukemia cell, Burkitt's lymphoma cell, chronic lymphocytic leukemia cell, chronic myeloid leukemia cell, diffuse large B-cell lymphoma cell, follicular lymphoma cell, hairy cell leukemia cell, Hodgkin's lymphoma cell, immunoblastic large cell lymphoma cell, mantle cell lymphoma cell, melanoma cell, multiple myeloma cell, neoplastic plasma cell, nodular lymphocyte predominant Hodgkin's lymphoma cell, non-Hodgkin's lymphoma cell, plasmablastic lymphoma cell, plasma cell myeloma cell, precursor B-lymphoblastic lymphoma cell, small lymphocytic lymphoma cell, malignant T-cell, T-cell leukemia cell, T-cell lymphoma cell, T-cell large granular lymphocyte leukemia cell, T-cell prolymphocytic leukemia, healthy B-cell lineage cell, or healthy T-cell members.

The embodiments of the CD20-binding molecules as described herein for the delivery of additional exogenous material into a cell each comprise (1) one or more CD20 binding regions capable of specifically binding an extracellular part of a CD20, (2) a Shiga toxin effector polypeptide, and (3) an additional exogenous material. In some embodiments, the CD20-binding molecule as described herein comprises an additional exogenous material such as a cytotoxic agent, detection promoting agent, peptide, polypeptide, protein, or polynucleotide. In some embodiments, the additional exogenous material is the protein comprising an enzyme. In certain other embodiments, the additional exogenous material is the polynucleotide which functions as a small inhibiting RNA (siRNA) or microRNA (miRNA). In some embodiments, the additional exogenous material is the peptide which is an antigen, such as, e.g., from a pathogen. In some embodiments, the antigen comprises, consisting essentially of, or consisting of SEQ ID NO:46. In some embodiments, the antigen is derived from a bacterial protein, protein mutated in cancer, protein aberrantly expressed in cancer, T-cell complementary determining region polypeptide, and/or viral protein. In some embodiments, the cytotoxic agent is a chemotherapeutic agent, cytotoxic antibiotic, alkylating agent, antimetabolite, topoisomerase inhibitor, or tubulin inhibitor.

Also provided herein are compositions comprising a multivalent CD20-binding molecule (CD20-binding molecule compositions) as described herein, such as, e.g., compositions enriched for a multivalent CD20-binding molecule as described herein and/or compositions with relatively large proportions of multivalent CD20-binding molecule relative to monovalent CD20-binding molecules. In some embodiments, the multivalent CD20-binding molecule composition as described herein comprises a multivalent CD20-binding molecule as described herein, wherein the composition comprises a ratio of monovalent CD20-binding molecule concentration to total CD20-binding molecule concentration of less than one to three; and wherein each monovalent CD20-binding molecule comprises only one CD20 binding region capable of specifically binding an extracellular part of a CD20 and comprises at least one Shiga toxin effector polypeptide. In some embodiments, the multivalent CD20-binding molecule composition comprises the ratio of monovalent CD20-binding molecule concentration to total CD20-binding protein concentration of less than the ratio selected from the following: 1:5, 1:6, 1:7, 1:8, 1:9, 1:10, and 1:11. In some embodiments, the multivalent CD20-binding molecule composition as described herein comprises a ratio of multivalent CD20-binding molecule concentration to total CD20-binding molecule concentration of more than two to three.

In some embodiments, the multivalent CD20-binding molecule composition as described herein comprises a ratio of relatively large valence, CD20-binding molecule concentration to total CD20-binding molecule concentration of less than the ratio selected from the following: 1:4, 1:7, 1:11, 1:21, 1:41, 1:71, 1:111, and 1:161; wherein each relatively large valence, CD20-binding molecule comprises three or more CD20 binding regions capable of specifically binding an extracellular part of a CD20 and comprises at least one Shiga toxin effector polypeptide.

In some embodiments, the multivalent CD20-binding molecule composition as described herein comprises a ratio of bivalent CD20-binding molecule concentration to total CD20-binding molecule concentration of more than a ratio selected from the following: 1:2, 2:3, 3:4, 4:5, 5:6, 7:8, 8:9, 9:10, 10:11, 11:12, 12:13, 13:14, and 14:15; wherein each bivalent CD20-binding molecule comprises (1) only two CD20 binding regions capable of specifically binding an extracellular part of a CD20 and (2) one or more Shiga toxin effector polypeptides.

For certain embodiments of the multivalent CD20-binding molecule composition as described herein, upon administration of the multivalent CD20-binding molecule composition to a first population of cells physically coupled to CD20, and a second population of cells, a cytotoxic effect of the multivalent CD20-binding molecule composition to members of said first population of cells relative to members of said second population of cells is at least 3-fold greater. In some embodiments, members of the first population of cells express at a cellular surface the CD20 which (1) have the extracellular part bound by the two or more CD20 binding regions of a multivalent CD20-binding molecule of the multivalent CD20-binding molecule composition, (2) have a transmembrane domain, and (3) remain physically coupled to the cell. In some embodiments, members of the first population of cells are CD20 positive cells. For certain embodiments, the members of the first population of cells are physically coupled with a significant amount of extracellular CD20 which (1) have the extracellular part bound by the two or more CD20 binding regions of a multivalent CD20-binding molecule of the multivalent CD20-binding molecule composition. For certain embodiments, members of the first population of cells over-express, at a cellular surface, CD20 which have the extracellular part bound by the two or more CD20 binding regions of a multivalent CD20-binding molecule of the multivalent CD20-binding molecule composition. For certain embodiments, members of the first population of cells over-express CD20 which (1) have the extracellular part bound by the two or more CD20 binding regions of a multivalent CD20-binding molecule of the multivalent CD20-binding molecule composition, (2) have a transmembrane domain, and (3) remain physically coupled to the cell. For certain embodiments, members of the first population of cells are descendants or members of a B-cell lineage. For certain embodiments, members of the first population of cells are malignant B-cell, B-cell leukemia cell, B-cell lymphoma cell, B-cell myeloma cell, acute myeloid leukemia cell, acute non-lymphocytic leukemia cell, B-cell chronic lymphocytic leukemia cell, B-cell lymphoma cell, B-cell non-Hodgkin's lymphoma cell, B-cell precursor acute lymphoblastic leukemia cell, B-cell prolymphocytic leukemia cell, Burkitt's lymphoma cell, chronic lymphocytic leukemia cell, chronic myeloid leukemia cell, diffuse large B-cell lymphoma cell, follicular lymphoma cell, hairy cell leukemia cell, Hodgkin's lymphoma cell, immunoblastic large cell lymphoma cell, mantle cell lymphoma cell, melanoma cell, multiple myeloma cell, neoplastic plasma cell, nodular lymphocyte predominant Hodgkin's lymphoma cell, non-Hodgkin's lymphoma cell, plasmablastic lymphoma cell, plasma cell myeloma cell, precursor B-lymphoblastic lymphoma cell, small lymphocytic lymphoma cell, malignant T-cell, T-cell leukemia cell, T-cell lymphoma cell, T-cell large granular lymphocyte leukemia cell, T-cell prolymphocytic leukemia, healthy B-cell lineage cell, or healthy T-cell members. For certain embodiments, the members of the second population of cells are not physically coupled with extracellular CD20 and/or are CD20 negative. For certain embodiments, the members of the second population of cells are not physically coupled with extracellular CD20 which (1) have the extracellular part bound by the two or more CD20 binding regions of the multivalent CD20-binding molecule. In some embodiments, members of the second population of cells express at a cellular surface the CD20 which (1) have the extracellular part bound by the two or more CD20 binding regions of a multivalent CD20-binding molecule of the multivalent CD20-binding molecule composition, (2) have a transmembrane domain, and (3) remain physically coupled to the cell. For certain embodiments, the members of the second population of cells are not physically coupled with a significant amount of extracellular CD20 which (1) have the extracellular part bound by the two or more CD20 binding regions of any multivalent CD20-binding molecule of the multivalent CD20-binding molecule composition. For certain embodiments of the multivalent CD20-binding molecule composition as described herein, upon administration of the multivalent CD20-binding molecule composition to a first population of cells whose members are CD20 positive, and a second population of cells whose members are not CD20 positive, a cytotoxic effect of the multivalent CD20-binding molecule composition to members of said first population of cells relative to members of said second population of cells is at least 3-fold greater.

In some embodiments, the multivalent CD20-binding molecule composition as described herein comprises a multivalent CD20-binding molecule having one or more monovalent CD20-binding molecule components; and whereby upon administration of the multivalent CD20-binding molecule composition as described herein to a population of cells physically coupled with CD20, which have the extracellular part bound by two or more CD20 binding regions of the multivalent CD20-binding molecule, the multivalent CD20-binding molecule composition exhibits a cytotoxic effect which is greater than a cytotoxic effect resulting from administration of an equivalent amount, mass, or molarity of any one of the monovalent CD20-binding molecule components to a population of the same CD20 positive cells under same conditions by a factor of 1.33, 1.5, 1.75, 2, 3, 5, 7.5, 10, 20, 100, or greater than the change in CD20-binding valence between the monovalent CD20-binding component and the multivalent CD20-binding molecule. In some embodiments, members of the population of cells express at a cellular surface the CD20 which (1) have the extracellular part bound by the two or more CD20 binding regions of the multivalent CD20-binding molecule, (2) have a transmembrane domain, and (3) remain physically coupled to the cell. In some embodiments, members of the population of cells are CD20 positive cells. For certain embodiments, the members of the population of cells are physically coupled with a significant amount of extracellular CD20 which (1) have the extracellular part bound by the two or more CD20 binding regions of the multivalent CD20-binding molecule. For certain embodiments, members of the population of cells are descendants or members of a B-cell lineage. For certain embodiments, members of the population of cells are malignant B-cell, B-cell leukemia cell, B-cell lymphoma cell, B-cell myeloma cell, acute myeloid leukemia cell, acute non-lymphocytic leukemia cell, B-cell chronic lymphocytic leukemia cell, B-cell lymphoma cell, B-cell non-Hodgkin's lymphoma cell, B-cell precursor acute lymphoblastic leukemia cell, B-cell prolymphocytic leukemia cell, Burkitt's lymphoma cell, chronic lymphocytic leukemia cell, chronic myeloid leukemia cell, diffuse large B-cell lymphoma cell, follicular lymphoma cell, hairy cell leukemia cell, Hodgkin's lymphoma cell, immunoblastic large cell lymphoma cell, mantle cell lymphoma cell, melanoma cell, multiple myeloma cell, neoplastic plasma cell, nodular lymphocyte predominant Hodgkin's lymphoma cell, non-Hodgkin's lymphoma cell, plasmablastic lymphoma cell, plasma cell myeloma cell, precursor B-lymphoblastic lymphoma cell, small lymphocytic lymphoma cell, malignant T-cell, T-cell leukemia cell, T-cell lymphoma cell, T-cell large granular lymphocyte leukemia cell, T-cell prolymphocytic leukemia, healthy B-cell lineage cell, or healthy T-cell members. In some embodiments, the multivalent CD20-binding molecule composition as described herein comprises a multivalent CD20-binding molecule having one or more monovalent CD20-binding molecule components; and whereby upon administration of the multivalent CD20-binding molecule composition as described herein to a population of CD20 positive cells expressing CD20 which have the extracellular part bound by two or more CD20 binding regions of the multivalent CD20-binding molecule, the multivalent CD20-binding molecule composition exhibits a cytotoxic effect which is greater than a cytotoxic effect resulting from administration of an equivalent amount, mass, or molarity of any one of the monovalent CD20-binding molecule components to a population of the same CD20 positive cells under same conditions by a factor of 1.33, 1.5, 1.75, 2, 3, 5, 7.5, 10, 20, 100, or greater than the change in CD20-binding valence between the monovalent CD20-binding component and the multivalent CD20-binding molecule.

For certain embodiments of the multivalent CD20-binding molecule composition as described herein, which comprises a multivalent CD20-binding molecule having an additional exogenous material; whereby upon administration of the multivalent CD20-binding molecule composition to a plurality of cells physically coupled with CD20, which have the extracellular part bound by the two or more CD20 binding regions of the multivalent CD20-binding molecule, at a concentration of multivalent CD20-binding molecule equivalent to five or thirty-eight percent to fifty percent cell-surface occupancy, the majority of the multivalent CD20-binding molecule internalizes into the plurality of cells in about five hours, four hours, three hours, two hours, one hour, thirty minutes, or less at a physiological temperature appropriate for the cell and/or at about 37 degrees Celsius. In some embodiments, members of the plurality of cells express at a cellular surface the CD20 which (1) have the extracellular part bound by the two or more CD20 binding regions of the multivalent CD20-binding molecule, (2) have a transmembrane domain, and (3) remain physically coupled to the cell. In some embodiments, members of the plurality of cells are CD20 positive cells. For certain embodiments, the members of the plurality of cells are physically coupled with a significant amount of extracellular CD20 which (1) have the extracellular part bound by the two or more CD20 binding regions of the multivalent CD20-binding molecule. For certain embodiments, members of the plurality of cells are descendants or members of a B-cell lineage. For certain embodiments, members of the plurality of cells are malignant B-cell, B-cell leukemia cell, B-cell lymphoma cell, B-cell myeloma cell, acute myeloid leukemia cell, acute non-lymphocytic leukemia cell, B-cell chronic lymphocytic leukemia cell, B-cell lymphoma cell, B-cell non-Hodgkin's lymphoma cell, B-cell precursor acute lymphoblastic leukemia cell, B-cell prolymphocytic leukemia cell, Burkitt's lymphoma cell, chronic lymphocytic leukemia cell, chronic myeloid leukemia cell, diffuse large B-cell lymphoma cell, follicular lymphoma cell, hairy cell leukemia cell, Hodgkin's lymphoma cell, immunoblastic large cell lymphoma cell, mantle cell lymphoma cell, melanoma cell, multiple myeloma cell, neoplastic plasma cell, nodular lymphocyte predominant Hodgkin's lymphoma cell, non-Hodgkin's lymphoma cell, plasmablastic lymphoma cell, plasma cell myeloma cell, precursor B-lymphoblastic lymphoma cell, small lymphocytic lymphoma cell, malignant T-cell, T-cell leukemia cell, T-cell lymphoma cell, T-cell large granular lymphocyte leukemia cell, T-cell prolymphocytic leukemia, healthy B-cell lineage cell, or healthy T-cell members.

For certain embodiments of the multivalent CD20-binding molecule composition as described herein, which comprises a multivalent CD20-binding molecule having an additional exogenous material; whereby upon administration of the multivalent CD20-binding molecule composition to one or more cells physically coupled with CD20, which have the extracellular part bound by the two or more CD20 binding regions of the multivalent CD20-binding molecule, the multivalent CD20-binding molecule internalizes into the one or more cells and delivers the additional exogenous material into the interior of the cell in about five hours, four hours, three hours, two hours, one hour, thirty minutes, or less at a physiological temperature appropriate for the cell and/or at about 37 degrees Celsius. In some embodiments, the one or more cell(s) expresses at a cellular surface the CD20 which (1) have the extracellular part bound by the two or more CD20 binding regions of the multivalent CD20-binding molecule, (2) have a transmembrane domain, and (3) remain physically coupled to the cell. In some embodiments, one or more cell(s) is a CD20 positive cell. For certain embodiments, one or more cell(s) is physically coupled with a significant amount of extracellular CD20 which (1) have the extracellular part bound by the two or more CD20 binding regions of the multivalent CD20-binding molecule. For certain embodiments, one or more cell(s) is a descendant or member of a B-cell lineage. For certain embodiments, one or more cell(s) is a malignant B-cell, B-cell leukemia cell, B-cell lymphoma cell, B-cell myeloma cell, acute myeloid leukemia cell, acute non-lymphocytic leukemia cell, B-cell chronic lymphocytic leukemia cell, B-cell lymphoma cell, B-cell non-Hodgkin's lymphoma cell, B-cell precursor acute lymphoblastic leukemia cell, B-cell prolymphocytic leukemia cell, Burkitt's lymphoma cell, chronic lymphocytic leukemia cell, chronic myeloid leukemia cell, diffuse large B-cell lymphoma cell, follicular lymphoma cell, hairy cell leukemia cell, Hodgkin's lymphoma cell, immunoblastic large cell lymphoma cell, mantle cell lymphoma cell, melanoma cell, multiple myeloma cell, neoplastic plasma cell, nodular lymphocyte predominant Hodgkin's lymphoma cell, non-Hodgkin's lymphoma cell, plasmablastic lymphoma cell, plasma cell myeloma cell, precursor B-lymphoblastic lymphoma cell, small lymphocytic lymphoma cell, malignant T-cell, T-cell leukemia cell, T-cell lymphoma cell, T-cell large granular lymphocyte leukemia cell, T-cell prolymphocytic leukemia, healthy B-cell lineage cell, or healthy T-cell.

For certain embodiments of the multivalent CD20-binding molecule composition as described herein, which comprises a multivalent CD20-binding molecule having an additional exogenous material; whereby upon administration of the multivalent CD20-binding molecule composition to a plurality of cells physically coupled with CD20, which have the extracellular part bound by the two or more CD20 binding regions of the multivalent CD20-binding molecule, at a concentration of multivalent CD20-binding molecule equivalent to five or thirty-eight percent to fifty percent cell-surface occupancy, the majority of the multivalent CD20-binding molecule internalizes into the plurality of cells and delivers the additional exogenous material into the interiors of the cells in about five hours, four hours, three hours, two hours, one hour, thirty minutes, or less at a physiological temperature appropriate for the cell and/or at about 37 degrees Celsius. In some embodiments, members of the plurality of cells express at a cellular surface the CD20 which (1) have the extracellular part bound by the two or more CD20 binding regions of the multivalent CD20-binding molecule, (2) have a transmembrane domain, and (3) remain physically coupled to the cell. In some embodiments, members of the plurality of cells are CD20 positive cells. For certain embodiments, the members of the plurality of cells are physically coupled with a significant amount of extracellular CD20 which (1) have the extracellular part bound by the two or more CD20 binding regions of the multivalent CD20-binding molecule. For certain embodiments, members of the plurality of cells are descendants or members of a B-cell lineage. For certain embodiments, members of the plurality of cells are malignant B-cell, B-cell leukemia cell, B-cell lymphoma cell, B-cell myeloma cell, acute myeloid leukemia cell, acute non-lymphocytic leukemia cell, B-cell chronic lymphocytic leukemia cell, B-cell lymphoma cell, B-cell non-Hodgkin's lymphoma cell, B-cell precursor acute lymphoblastic leukemia cell, B-cell prolymphocytic leukemia cell, Burkitt's lymphoma cell, chronic lymphocytic leukemia cell, chronic myeloid leukemia cell, diffuse large B-cell lymphoma cell, follicular lymphoma cell, hairy cell leukemia cell, Hodgkin's lymphoma cell, immunoblastic large cell lymphoma cell, mantle cell lymphoma cell, melanoma cell, multiple myeloma cell, neoplastic plasma cell, nodular lymphocyte predominant Hodgkin's lymphoma cell, non-Hodgkin's lymphoma cell, plasmablastic lymphoma cell, plasma cell myeloma cell, precursor B-lymphoblastic lymphoma cell, small lymphocytic lymphoma cell, malignant T-cell, T-cell leukemia cell, T-cell lymphoma cell, T-cell large granular lymphocyte leukemia cell, T-cell prolymphocytic leukemia, healthy B-cell lineage cell, or healthy T-cell members.

Also provided herein are solvates, hydrates, salts, and/or powders comprising a multivalent CD20-binding molecule and/or multivalent CD20-binding molecule composition as described herein. For certain embodiments, the solvate as described herein comprises one or more multivalent CD20-binding molecules as described herein and/or a multivalent CD20-binding molecule composition as described herein. For certain embodiments, the salt as described herein comprises one or more multivalent CD20-binding molecules as described herein and/or a multivalent CD20-binding molecule composition as described herein.

Also provided herein are pharmaceutical compositions comprising a multivalent CD20-binding molecule as described herein and/or a multivalent CD20-binding molecule composition as described herein, and comprising at least one pharmaceutically acceptable excipient or carrier; and the use of such a multivalent CD20-binding molecule or a composition comprising it in making such pharmaceutical compositions and in methods as described herein as further described herein. Certain embodiments as described herein are pharmaceutical compositions comprising any multivalent CD20 binding molecule as described herein (e.g. a multivalent CD20-binding protein as described herein) and at least one pharmaceutically acceptable excipient or carrier.

Also provided herein are pharmaceutical compositions comprising a multivalent CD20-binding molecule as described herein, a multivalent CD20-binding molecule composition as described herein, a solvate as described herein, and/or a salt as described herein; and comprising at least one pharmaceutically acceptable excipient, carrier, buffer, isotonic agent, surfactant, antioxidant, and/or metal-chelating agent.

Certain embodiments as described herein are pharmaceutical compositions comprising any multivalent CD20 binding molecule as described herein (e.g. a multivalent CD20-binding protein as described herein) and at least one pharmaceutically acceptable carrier, solvent, vehicle, sterile aqueous solution, buffer, powder, sterile powder, surfactant, antioxidant, chelating agent, antimicrobial agent, preservative, isotonic agent, dispersion medium, coating, adjuvant, wetting agent, emulsifying agent, dispersing agent, adsorption delaying agent, stabilizer, and/or additive.

In some embodiments CD20-binding molecule composition, the solvate, the salt, or the pharmaceutical composition as described herein comprises: acetate, alcohol, alpha-tocopherol, aluminum monostearate, ascorbic acid, ascorbyl palmitate, benzyl alcohol, butylated hydroxyanisole, butylated hydroxytoluene, chlorobutanol, citrate, cysteine hydrochloride, dextrose, ethanol, ethylenediaminetetraacetic acid, ethyloleate, gelatin, glycerine, glycerol, lactic acid, lecithin, mannitol, methyl parabens, monostearate salt, organic ester, paraben, phenol phosphate, phosphoric acid, polyalcohol, polyethylene glycol, polyol, propylene glycol, propylgallate, Ringer's solution, saline, sodium bisulfate, sodium bisulfite, sodium chloride, sodium metabisulfite, sodium sulfite, sorbic acid, sorbitol, sugar, tartaric acid, vegetable oil, and/or water.

Certain embodiments as described herein are pharmaceutical compositions comprising any multivalent CD20 binding molecule as described herein (e.g. a multivalent CD20-binding protein as described herein) and at least one pharmaceutically acceptable excipient or carrier. In some embodiments, the excipient is acetate, alcohol, alpha-tocopherol, aluminum monostearate, ascorbic acid, ascorbyl palmitate, benzyl alcohol, butylated hydroxyanisole, butylated hydroxytoluene, chlorobutanol, citrate, cysteine hydrochloride, dextrose, ethanol, ethylenediaminetetraacetic acid, ethyloleate, gelatin, glycerine, glycerol, lactic acid, lecithin, mannitol, methyl parabens, monostearate salt, organic ester, paraben, phenol phosphate, phosphoric acid, polyalcohol, polyethylene glycol, polyol, propylene glycol, propylgallate, sodium bisulfate, sodium bisulfite, sodium chloride, sodium metabisulfite, sodium sulfite, sorbic acid, sorbitol, sugar, tartaric acid, and/or vegetable oil.

Among certain embodiments as described herein is a diagnostic composition comprising a multivalent CD20-binding molecule as described herein that further comprises a detection promoting agent for the collection of information about a cell, cell type, tissue, organ, disease, disorder, condition, subject, and/or patient.

In some embodiments, the solvate, salt, pharmaceutical composition, and/or diagnostic composition as described herein comprises a ratio of monovalent CD20-binding molecule concentration to total CD20-binding molecule concentration of less than one to three; and wherein each monovalent CD20-binding molecule comprises only one CD20 binding region capable of specifically binding an extracellular part of a CD20 and comprises at least one Shiga toxin effector polypeptide. In some embodiments, the solvate, salt, pharmaceutical composition, and/or diagnostic composition comprises the ratio of monovalent CD20-binding molecule concentration to total CD20-binding protein concentration of less than the ratio selected from the following: 1:5, 1:6, 1:7, 1:8, 1:9, 1:10, and 1:11. In some embodiments, the solvate, salt, pharmaceutical composition, and/or diagnostic composition as described herein comprises a ratio of multivalent CD20-binding molecule concentration to total CD20-binding molecule concentration of more than two to three.

In some embodiments, the solvate, salt, pharmaceutical composition, and/or diagnostic composition as described herein comprises a ratio of relatively large valence, CD20-binding molecule concentration to total CD20-binding molecule concentration of less than the ratio selected from the following: 1:4, 1:7, 1:11, 1:21, 1:41, 1:71, 1:111, and 1:161; wherein each relatively large valence, CD20-binding molecule comprises three or more CD20 binding regions capable of specifically binding an extracellular part of a CD20 and comprises at least one Shiga toxin effector polypeptide.

In some embodiments, the solvate, salt, pharmaceutical composition, and/or diagnostic composition comprises a ratio of bivalent CD20-binding molecule concentration to total CD20-binding molecule concentration of more than a ratio selected from the following: 1:2, 2:3, 3:4, 4:5, 5:6, 7:8, 8:9, 9:10, 10:11, 11:12, 12:13, 13:14, and 14:15; wherein each bivalent CD20-binding molecule comprises (1) only two CD20 binding regions capable of specifically binding an extracellular part of a CD20 and (2) one or more Shiga toxin effector polypeptides.

Beyond the multivalent CD20-binding molecules as described herein, and compositions thereof, polynucleotides capable of encoding a multivalent CD20-binding molecule as described herein (e.g. a multivalent CD20-binding protein), or polypeptide component thereof, are within the scope as described herein, as well as expression vectors which comprise a polynucleotide described herein and host cells comprising an expression vector as described herein. Host cells comprising an expression vector as described herein may be used, e.g., in methods for producing a multivalent CD20-binding molecule as described herein or a polypeptide component or fragment thereof by recombinant expression. Similarly, host cells comprising an expression vector as described herein may be used, e.g., in methods for producing a multivalent CD20-binding molecule composition as described herein, or a polypeptide component thereof.

Also encompassed herein is any composition of matter as described herein which is immobilized on a solid substrate. Such arrangements of the compositions of matter as described herein may be utilized, e.g., in methods of screening molecules as described herein.

Also provided herein are methods of killing cell(s) comprising the step of contacting a cell(s) with a multivalent CD20-binding molecule as described herein, a multivalent CD20-binding molecule composition as described herein, a solvate as described herein, a salt as described herein, and/or a pharmaceutical composition as described herein. For certain embodiments, the step of contacting the cell(s) occurs in vitro. For certain other embodiments, the step of contacting the cell(s) occurs in vivo. For certain embodiments of the cell-killing methods as described herein, the method is capable of selectively killing cell(s) and/or cell types preferentially over other cell(s) and/or cell types when contacting a mixture of cells comprising different cells which differ with respect to the cell-surface presence and/or expression level of a CD20 bound by the two or more CD20 binding regions of the multivalent CD20-binding molecule and/or the multivalent CD20-binding molecule of the composition as described herein (e.g., a multivalent CD20-binding molecule composition and/or a pharmaceutical composition as described herein).

Also provided herein is a method of inducing cellular internalization of a multivalent CD20-binding molecule into a cell(s) physically coupled with CD20, which have the extracellular part bound by two or more CD20 binding regions of the multivalent CD20-binding molecule, the method comprising the step of contacting the cell(s) with a multivalent CD20-binding molecule as described herein, a multivalent CD20-binding molecule composition as described herein, a solvate as described herein, a salt as described herein, a pharmaceutical composition as described herein, and/or a diagnostic composition as described herein. In some embodiments of the inducing cellular internalization method, the step of contacting the cell(s) occurs in vitro. For certain other embodiments, the step of contacting the cell(s) occurs in vivo, such as, e.g., within a patient. In some embodiments of the inducing cellular internalization method, the cellular internalization of the multivalent CD20-binding molecule occurs in about five hours, four hours, three hours, two hours, one hour, thirty minutes, or less at a physiological temperature appropriate for the cell and/or at about 37 degrees Celsius. In some embodiments, the cell expresses at a cellular surface the CD20 which (1) have the extracellular part bound by the two or more CD20 binding regions of the multivalent CD20-binding molecule, (2) have a transmembrane domain, and (3) remain physically coupled to the cell. In some embodiments, the cell is a CD20 positive cell. For certain embodiments, the cell is physically coupled with a significant amount of extracellular CD20 which (1) have the extracellular part bound by the two or more CD20 binding regions of the multivalent CD20-binding molecule. For certain embodiments, the cell is a descendant or member of a B-cell lineage. For certain embodiments, the cell is a malignant B-cell, B-cell leukemia cell, B-cell lymphoma cell, B-cell myeloma cell, acute myeloid leukemia cell, acute non-lymphocytic leukemia cell, B-cell chronic lymphocytic leukemia cell, B-cell lymphoma cell, B-cell non-Hodgkin's lymphoma cell, B-cell precursor acute lymphoblastic leukemia cell, B-cell prolymphocytic leukemia cell, Burkitt's lymphoma cell, chronic lymphocytic leukemia cell, chronic myeloid leukemia cell, diffuse large B-cell lymphoma cell, follicular lymphoma cell, hairy cell leukemia cell, Hodgkin's lymphoma cell, immunoblastic large cell lymphoma cell, mantle cell lymphoma cell, melanoma cell, multiple myeloma cell, neoplastic plasma cell, nodular lymphocyte predominant Hodgkin's lymphoma cell, non-Hodgkin's lymphoma cell, plasmablastic lymphoma cell, plasma cell myeloma cell, precursor B-lymphoblastic lymphoma cell, small lymphocytic lymphoma cell, malignant T-cell, T-cell leukemia cell, T-cell lymphoma cell, T-cell large granular lymphocyte leukemia cell, T-cell prolymphocytic leukemia, healthy B-cell lineage cell, or healthy T-cell.

Also provided herein is a method of inducing cellular internalization of a multivalent CD20-binding molecule into a plurality of cells physically coupled with CD20, which have the extracellular part bound by two or more CD20 binding regions of the multivalent CD20-binding molecule, the method comprising the step of contacting the plurality of cells with a multivalent CD20-binding molecule as described herein, a multivalent CD20-binding molecule composition as described herein, a solvate as described herein, a salt as described herein, a pharmaceutical composition as described herein, and/or a diagnostic composition as described herein. In some embodiments of the inducing cellular internalization method, the step of contacting the cell(s) occurs in vitro. For certain other embodiments, the step of contacting the cell(s) occurs in vivo, such as, e.g., within a patient. In some embodiments of the inducing cellular internalization method, the cellular internalization of the multivalent CD20-binding molecule occurs in about five hours, four hours, three hours, two hours, one hour, thirty minutes, or less at a physiological temperature appropriate for the cell and/or at about 37 degrees Celsius. In some embodiments, members of the plurality of cells express at a cellular surface the CD20 which (1) have the extracellular part bound by the two or more CD20 binding regions of the multivalent CD20-binding molecule, (2) have a transmembrane domain, and (3) remain physically coupled to the cell. In some embodiments, members of the plurality of cells are CD20 positive cells. For certain embodiments, the members of the plurality of cells are physically coupled with a significant amount of extracellular CD20 which (1) have the extracellular part bound by the two or more CD20 binding regions of the multivalent CD20-binding molecule. For certain embodiments, members of the plurality of cells are descendants or members of a B-cell lineage. For certain embodiments, members of the plurality of cells are malignant B-cell, B-cell leukemia cell, B-cell lymphoma cell, B-cell myeloma cell, acute myeloid leukemia cell, acute non-lymphocytic leukemia cell, B-cell chronic lymphocytic leukemia cell, B-cell lymphoma cell, B-cell non-Hodgkin's lymphoma cell, B-cell precursor acute lymphoblastic leukemia cell, B-cell prolymphocytic leukemia cell, Burkitt's lymphoma cell, chronic lymphocytic leukemia cell, chronic myeloid leukemia cell, diffuse large B-cell lymphoma cell, follicular lymphoma cell, hairy cell leukemia cell, Hodgkin's lymphoma cell, immunoblastic large cell lymphoma cell, mantle cell lymphoma cell, melanoma cell, multiple myeloma cell, neoplastic plasma cell, nodular lymphocyte predominant Hodgkin's lymphoma cell, non-Hodgkin's lymphoma cell, plasmablastic lymphoma cell, plasma cell myeloma cell, precursor B-lymphoblastic lymphoma cell, small lymphocytic lymphoma cell, malignant T-cell, T-cell leukemia cell, T-cell lymphoma cell, T-cell large granular lymphocyte leukemia cell, T-cell prolymphocytic leukemia, healthy B-cell lineage cell, or healthy T-cell members.

Also provided herein is a method of internalizing a cell surface localized CD20 bound by a multivalent CD20-binding molecule as described herein, the method comprising the step of contacting a cell(s) having cell surface localized CD20, which have the extracellular part bound by two or more CD20 binding regions of the multivalent CD20-binding molecule, with a multivalent CD20-binding molecule as described herein, a multivalent CD20-binding molecule composition as described herein, pharmaceutical composition as described herein, and/or a diagnostic composition as described herein. In some embodiments of the method of internalizing cell surface localized CD20, the step of contacting the cell(s) occurs in vitro. For certain other embodiments, the step of contacting the cell(s) occurs in vivo, such as, e.g., within a patient. In some embodiments of the of the method of internalizing cell surface localized CD20, the internalization of cell surface localized CD20 occurs in about five hours, four hours, three hours, two hours, one hour, thirty minutes, or less at a physiological temperature appropriate for the cell and/or at about 37 degrees Celsius. In some embodiments, the cell expresses at a cellular surface the CD20 which (1) have the extracellular part bound by the two or more CD20 binding regions of the multivalent CD20-binding molecule, (2) have a transmembrane domain, and (3) remain physically coupled to the cell. In some embodiments, the cell is a CD20 positive cell. For certain embodiments, the cell is physically coupled with a significant amount of extracellular CD20 which (1) have the extracellular part bound by the two or more CD20 binding regions of the multivalent CD20-binding molecule. For certain embodiments, the cell is a descendant or member of a B-cell lineage. For certain embodiments, the cell is a malignant B-cell, B-cell leukemia cell, B-cell lymphoma cell, B-cell myeloma cell, acute myeloid leukemia cell, acute non-lymphocytic leukemia cell, B-cell chronic lymphocytic leukemia cell, B-cell lymphoma cell, B-cell non-Hodgkin's lymphoma cell, B-cell precursor acute lymphoblastic leukemia cell, B-cell prolymphocytic leukemia cell, Burkitt's lymphoma cell, chronic lymphocytic leukemia cell, chronic myeloid leukemia cell, diffuse large B-cell lymphoma cell, follicular lymphoma cell, hairy cell leukemia cell, Hodgkin's lymphoma cell, immunoblastic large cell lymphoma cell, mantle cell lymphoma cell, melanoma cell, multiple myeloma cell, neoplastic plasma cell, nodular lymphocyte predominant Hodgkin's lymphoma cell, non-Hodgkin's lymphoma cell, plasmablastic lymphoma cell, plasma cell myeloma cell, precursor B-lymphoblastic lymphoma cell, small lymphocytic lymphoma cell, malignant T-cell, T-cell leukemia cell, T-cell lymphoma cell, T-cell large granular lymphocyte leukemia cell, T-cell prolymphocytic leukemia, healthy B-cell lineage cell, or healthy T-cell.

Also provided herein is a method of internalizing a cell surface localized CD20 bound by a multivalent CD20-binding molecule, the method comprising the step of contacting a plurality of cells having cell surface localized CD20, which have the extracellular part bound by two or more CD20 binding regions of the multivalent CD20-binding molecule, with a multivalent CD20-binding molecule as described herein, a multivalent CD20-binding molecule composition as described herein, pharmaceutical composition as described herein, and/or a diagnostic composition as described herein. In some embodiments of the method of internalizing cell surface localized CD20, the step of contacting the plurality of cells occurs in vitro. For certain other embodiments, the step of contacting the plurality of cells occurs in vivo, such as, e.g., within a patient. In some embodiments of the of the method of internalizing cell surface localized CD20, the internalization of cell surface localized CD20 occurs in a majority of the cells of the plurality of cells in about five hours, four hours, three hours, two hours, one hour, thirty minutes, or less at a physiological temperature appropriate for the cell and/or at about 37 degrees Celsius. In some embodiments, members of the plurality of cells express at a cellular surface the CD20 which (1) have the extracellular part bound by the two or more CD20 binding regions of the multivalent CD20-binding molecule, (2) have a transmembrane domain, and (3) remain physically coupled to the cell. In some embodiments, members of the plurality of cells are CD20 positive cells. For certain embodiments, the members of the plurality of cells are physically coupled with a significant amount of extracellular CD20 which (1) have the extracellular part bound by the two or more CD20 binding regions of the multivalent CD20-binding molecule. For certain embodiments, members of the plurality of cells are descendants or members of a B-cell lineage. For certain embodiments, members of the plurality of cells are malignant B-cell, B-cell leukemia cell, B-cell lymphoma cell, B-cell myeloma cell, acute myeloid leukemia cell, acute non-lymphocytic leukemia cell, B-cell chronic lymphocytic leukemia cell, B-cell lymphoma cell, B-cell non-Hodgkin's lymphoma cell, B-cell precursor acute lymphoblastic leukemia cell, B-cell prolymphocytic leukemia cell, Burkitt's lymphoma cell, chronic lymphocytic leukemia cell, chronic myeloid leukemia cell, diffuse large B-cell lymphoma cell, follicular lymphoma cell, hairy cell leukemia cell, Hodgkin's lymphoma cell, immunoblastic large cell lymphoma cell, mantle cell lymphoma cell, melanoma cell, multiple myeloma cell, neoplastic plasma cell, nodular lymphocyte predominant Hodgkin's lymphoma cell, non-Hodgkin's lymphoma cell, plasmablastic lymphoma cell, plasma cell myeloma cell, precursor B-lymphoblastic lymphoma cell, small lymphocytic lymphoma cell, malignant T-cell, T-cell leukemia cell, T-cell lymphoma cell, T-cell large granular lymphocyte leukemia cell, T-cell prolymphocytic leukemia, healthy B-cell lineage cell, or healthy T-cell members.

In some embodiments, provided herein is a method of inducing cellular internalization of a cell surface localized CD20 bound by a multivalent CD20-binding molecule in a subject, the method comprising the step of administering to the subject a multivalent CD20-binding molecule as described herein, a multivalent CD20-binding molecule composition as described herein, pharmaceutical composition as described herein, and/or a diagnostic composition as described herein.

Additionally, provided herein is a method for delivering an exogenous material to the inside of a cell, the method comprising the step of contacting the cell(s), either in vitro or in vivo, with a multivalent CD20-binding molecule as described herein which comprises an additional exogenous material, a multivalent CD20-binding molecule composition as described herein comprising a multivalent CD20-binding molecule as described herein which comprises an additional exogenous material, a pharmaceutical composition as described herein comprising a multivalent CD20-binding molecule as described herein which comprises an additional exogenous material, and/or a diagnostic composition as described herein comprising a multivalent CD20-binding molecule as described herein which comprises an additional exogenous material. In some embodiments, the cell is physically coupled with CD20 which have the extracellular part bound by two or more CD20 binding regions of the multivalent CD20-binding molecule. In some embodiments, the cell expresses at a cellular surface the CD20 which (1) have the extracellular part bound by the two or more CD20 binding regions of the multivalent CD20-binding molecule, (2) have a transmembrane domain, and (3) remain physically coupled to the cell. In some embodiments, the cell is a CD20 positive cell. For certain embodiments, the cell is physically coupled with a significant amount of extracellular CD20 which (1) have the extracellular part bound by the two or more CD20 binding regions of the multivalent CD20-binding molecule. For certain embodiments, the cell is a descendant or member of a B-cell lineage. For certain embodiments, the cell is a malignant B-cell, B-cell leukemia cell, B-cell lymphoma cell, B-cell myeloma cell, acute myeloid leukemia cell, acute non-lymphocytic leukemia cell, B-cell chronic lymphocytic leukemia cell, B-cell lymphoma cell, B-cell non-Hodgkin's lymphoma cell, B-cell precursor acute lymphoblastic leukemia cell, B-cell prolymphocytic leukemia cell, Burkitt's lymphoma cell, chronic lymphocytic leukemia cell, chronic myeloid leukemia cell, diffuse large B-cell lymphoma cell, follicular lymphoma cell, hairy cell leukemia cell, Hodgkin's lymphoma cell, immunoblastic large cell lymphoma cell, mantle cell lymphoma cell, melanoma cell, multiple myeloma cell, neoplastic plasma cell, nodular lymphocyte predominant Hodgkin's lymphoma cell, non-Hodgkin's lymphoma cell, plasmablastic lymphoma cell, plasma cell myeloma cell, precursor B-lymphoblastic lymphoma cell, small lymphocytic lymphoma cell, malignant T-cell, T-cell leukemia cell, T-cell lymphoma cell, T-cell large granular lymphocyte leukemia cell, T-cell prolymphocytic leukemia, healthy B-cell lineage cell, or healthy T-cell.

In some embodiments, a method of delivering an exogenous material to the inside of a cell comprises the step of administering to a subject a multivalent CD20-binding molecule as described herein which comprises an additional exogenous material, a multivalent CD20-binding molecule composition as described herein comprising a multivalent CD20-binding molecule as described herein which comprises an additional exogenous material, a pharmaceutical composition as described herein comprising a multivalent CD20-binding molecule as described herein which comprises an additional exogenous material, and/or a diagnostic composition as described herein comprising a multivalent CD20-binding molecule as described herein which comprises an additional exogenous material. In some embodiments, the cell is physically coupled with CD20 which have the extracellular part bound by two or more CD20 binding regions of the multivalent CD20-binding molecule. In some embodiments, the cell expresses at a cellular surface the CD20 which (1) have the extracellular part bound by the two or more CD20 binding regions of the multivalent CD20-binding molecule, (2) have a transmembrane domain, and (3) remain physically coupled to the cell. In some embodiments, the cell is a CD20 positive cell. For certain embodiments, the cell is physically coupled with a significant amount of extracellular CD20 which (1) have the extracellular part bound by the two or more CD20 binding regions of the multivalent CD20-binding molecule. For certain embodiments, the cell is a descendant or member of a B-cell lineage. For certain embodiments, the cell is a malignant B-cell, B-cell leukemia cell, B-cell lymphoma cell, B-cell myeloma cell, acute myeloid leukemia cell, acute non-lymphocytic leukemia cell, B-cell chronic lymphocytic leukemia cell, B-cell lymphoma cell, B-cell non-Hodgkin's lymphoma cell, B-cell precursor acute lymphoblastic leukemia cell, B-cell prolymphocytic leukemia cell, Burkitt's lymphoma cell, chronic lymphocytic leukemia cell, chronic myeloid leukemia cell, diffuse large B-cell lymphoma cell, follicular lymphoma cell, hairy cell leukemia cell, Hodgkin's lymphoma cell, immunoblastic large cell lymphoma cell, mantle cell lymphoma cell, melanoma cell, multiple myeloma cell, neoplastic plasma cell, nodular lymphocyte predominant Hodgkin's lymphoma cell, non-Hodgkin's lymphoma cell, plasmablastic lymphoma cell, plasma cell myeloma cell, precursor B-lymphoblastic lymphoma cell, small lymphocytic lymphoma cell, malignant T-cell, T-cell leukemia cell, T-cell lymphoma cell, T-cell large granular lymphocyte leukemia cell, T-cell prolymphocytic leukemia, healthy B-cell lineage cell, or healthy T-cell.

In some embodiments, the cell, cells, and population of cells referred to as (1) “cell”; (2) “cell physically coupled with CD20”; (3) “cell expressing, at a cellular surface, CD20”; (4) “CD20 positive cell”; (5) “plurality of cells”; (6) “plurality of cells physically coupled with CD20”; (7) “population of cells”; (8) “population of CD20 positive cells”; or (9) “one or more cells” are a cell, cells, or population of cells that (a) is physically coupled with extracellular CD20; (b) expresses at a cellular surface the CD20 which (i) have the extracellular part bound by the two or more CD20 binding regions of the CD20-binding molecule, (ii) have a transmembrane domain, and (iii) remain physically coupled to the cell(s); (c) is a CD20 positive; (d) is physically coupled with a significant amount of extracellular CD20 which have the extracellular part bound by the two or more CD20 binding regions of the CD20-binding molecule; (e) is a descendant or member of a B-cell lineage; (f) is cultured in a laboratory setting, is a member of an immortalized cell line, is a member of a laboratory cell strain, and/or is a member of an established human cell line; and/or (g) is one or more of the following: malignant B-cell, B-cell leukemia cell, B-cell lymphoma cell, B-cell myeloma cell, acute myeloid leukemia cell, acute non-lymphocytic leukemia cell, B-cell chronic lymphocytic leukemia cell, B-cell lymphoma cell, B-cell non-Hodgkin's lymphoma cell, B-cell precursor acute lymphoblastic leukemia cell, B-cell prolymphocytic leukemia cell, Burkitt's lymphoma cell, chronic lymphocytic leukemia cell, chronic myeloid leukemia cell, diffuse large B-cell lymphoma cell, follicular lymphoma cell, hairy cell leukemia cell, Hodgkin's lymphoma cell, immunoblastic large cell lymphoma cell, mantle cell lymphoma cell, melanoma cell, multiple myeloma cell, neoplastic plasma cell, nodular lymphocyte predominant Hodgkin's lymphoma cell, non-Hodgkin's lymphoma cell, plasmablastic lymphoma cell, plasma cell myeloma cell, precursor B-lymphoblastic lymphoma cell, small lymphocytic lymphoma cell, malignant T-cell, T-cell leukemia cell, T-cell lymphoma cell, T-cell large granular lymphocyte leukemia cell, T-cell prolymphocytic leukemia, healthy B-cell lineage cell, and/or healthy T-cell, including a laboratory culture of any of the aforementioned.

The use of any composition as described herein for the diagnosis, prognosis, and/or characterization of a disease, disorder, and/or condition is within the scope as described herein. Among certain embodiments as described herein is the use of one or more compositions of matter as described herein (e.g. a pharmaceutical composition as described herein) in the treatment or prevention of a cancer, tumor, abnormal growth condition, and/or immune disorder. Among certain embodiments as described herein is the use of one or more compositions of matter described herein (e.g. a solvate, salt, or pharmaceutical composition as described herein) in the manufacture of a medicament for the treatment or prevention of a cancer, tumor, abnormal growth condition, and/or immune disorder. Among certain embodiments as described herein is a cytotoxic protein or a pharmaceutical composition comprising said protein for use in the treatment or prevention of a cancer, tumor, or immune disorder.

Further provided herein are methods of treating diseases, disorders, and/or conditions in subjects, the methods comprising the step of administering to a subject in need thereof a therapeutically effective amount of a CD20-binding molecule as described herein, a CD20-binding molecule composition as described herein, a solvate as described herein, a salt as described herein, and/or a pharmaceutical composition as described herein. For certain embodiments of these treatment methods described herein, the disease, disorder, or condition to be treated using a method described herein involves a cell, cancer cell, tumor cell, and/or immune cell which express CD20 at a cellular surface. For certain embodiments of these treatment methods described herein, the disease, disorder, or condition to be treated using a method described herein is a cancer, tumor, abnormal growth condition, and/or immune disorder. For certain embodiments of these treatment methods described herein, the disease to be treated is hematologic cancer, leukemia, lymphoma, melanoma, or myeloma. For certain embodiments of these treatment methods described herein, the immune disorder to be treated is amyloidosis, ankylosing spondylitis, asthma, Crohn's disease, diabetes, graft rejection, graft-versus-host disease, Graves' disease, Graves' ophthalmopathy, Hashimoto's thyroiditis, hemolytic uremic syndrome, HIV-related diseases, lupus erythematosus, multiple sclerosis, neuromyelitis optica spectrum disorders, N-methyl D-aspartate (NMDA) receptor encephalitis, opsoclonus myoclonus syndrome (OMS), paroxysmal nocturnal hemoglobinuria, polyarteritis nodosa, polyarthritis, psoriasis, psoriatic arthritis, rheumatoid arthritis, scleritis, scleroderma, septic shock, Sjorgren's syndrome, ulcerative colitis, and vasculitis. For certain embodiments of these treatment methods as described herein, the cancer to be treated is acute myeloid leukemia (acute myelogenous leukemia or AML), acute non-lymphocytic leukemia, B-cell chronic lymphocytic leukemia (B-cell CLL), B-cell lymphoma, B-cell non-Hodgkin's lymphoma (B-cell NHL), B-cell precursor acute lymphoblastic leukemia (BCP-ALL or B-ALL), B-cell prolymphocytic leukemia (B-PLL), Burkitt's lymphoma (BL), chronic lymphocytic leukemia (CLL), chronic myeloid leukemia (CML), diffuse large B-cell lymphoma (DLBCL or DLBL), follicular lymphoma (FL), hairy cell leukemia (HCL), Hodgkin's lymphoma (HL or HD), immunoblastic large cell lymphoma, mantle cell lymphoma (MCL), multiple myeloma (MM), nodular lymphocyte predominant Hodgkin's lymphoma (NLPHL), non-Hodgkin's lymphoma (NHL), plasmablastic lymphoma, plasma cell neoplasma, plasma cell myeloma, precursor B-lymphoblastic lymphoma (B-LBL), small lymphocytic lymphoma (SLL), T-cell large granular lymphocyte leukemia (T-LGLL), T-cell lymphoma (TCL), T-cell prolymphocytic leukemia (T-PLL), or Waldenström's macroglobulinemia (WM).

Among certain embodiments as described herein is a method of producing a CD20-binding molecule as described herein and/or CD20-binding molecule composition, the method comprising the step of purifying a CD20-binding molecule or protein component thereof using an affinity purification step, such as, e.g., based on a chitin binding interaction. In some embodiments, the affinity purification step uses a chitin binding interaction. In some embodiments, the purifying step of the method involves the molecule comprising, consisting essentially of, or consisting of any one of the molecules shown in SEQ ID NOs: 4-304.

Among certain embodiments as described herein is a method of producing a CD20-binding molecule as described herein and/or CD20-binding molecule composition as described herein, the method comprising the step of oxidizing a CD20-binding molecule and/or a composition comprising a CD20-binding molecule. In some embodiments, the oxidizing step of the method uses a metal oxide or metal carboxylate as a catalyst. In some embodiments, the oxidizing step of the method uses copper sulfate as a catalyst. For certain embodiments, the oxidizing step of the method involves the CD20-binding molecule comprising, consisting essentially of, or consisting of any one of the molecules shown in SEQ ID NOs: 47-175 and 249-304.

Among certain embodiments as described herein is a method of using a CD20-binding protein described herein comprising a detection promoting agent for the collection of information useful in the diagnosis, prognosis, or characterization of a disease, disorder, or condition. Among certain embodiments as described herein is a method of detecting a cell using a CD20-binding protein and/or diagnostic composition described herein, the method comprising the steps of contacting a cell with the CD20-binding protein and/or diagnostic composition described herein and detecting the presence of the CD20-binding molecule and/or diagnostic composition. For certain embodiments, the step of contacting the cell(s) occurs in vitro and/or ex vivo. For certain embodiments, the step of contacting the cell(s) occurs in vivo. For certain embodiments, the step of detecting the cell(s) occurs in vitro and/or ex vivo. For certain embodiments, the step of detecting the cell(s) occurs in vivo.

The present invention is further illustrated by the following non-limiting illustrative examples of cytotoxic CD20-binding molecules and compositions comprising selectively cytotoxic CD20-binding molecules comprising Shiga toxin effector regions derived from A Subunits of members of the Shiga toxin family and at least one CD20 binding region capable of binding extracellular parts of CD20 physically coupled to specific, CD20-expressing cell types.

### EXAMPLES

The following Examples describe different CD20-binding molecules comprising Shiga toxin A Subunit effector polypeptides. Illustrative, CD20-binding molecules as described herein 1) bound to CD20 expressed at the surface of target cell types, such as, e.g., human lymphoma cells or healthy primate B-cells, 2) entered target cells and effectively routed catalytically active, Shiga toxin effector polypeptide to the cytosol of target cells resulting in the death of these CD20-expressing cells.

The illustrative CD20-binding molecule compositions as described herein, which were enriched with high-proportions of multivalent CD20-binding molecule(s) relative to monovalent CD20-binding molecule(s), showed greater cytotoxic activity compared to a protein composition predominantly composed of a monovalent CD20-binding protein, which was a component of the illustrative CD20-binding molecules as described herein shown in Example 1. The improved cytotoxic effects of illustrative CD20-binding molecule compositions as described herein could not be accounted for by predicted increases in cytotoxicity resulting from increases in the CD20-binding valences of the CD20-binding molecule variants as compared to the monovalent CD20-binding molecule.

Throughout the Examples, the term “CD20-binding protein” is used to refer to a Shiga toxin A Subunit derived, immunotoxin comprising one or more recombinant fusion polypeptides, which each comprise 1) one or more immunoglobulin-type CD20 binding regions capable of binding an extracellular part of a CD20 with high affinity, and 2) one or more Shiga toxin effector polypeptides. Certain CD20-binding proteins as described herein shown in the Examples below were multimeric, such as, e.g., a homodimer consisting essentially of two, identical, monovalent CD20-binding proteins which are coupled together.

### Example 1. Illustrative, Multivalent CD20-Binding Proteins as Described Herein and Enriched Compositions Thereof

Illustrative, multivalent CD20-binding molecules as described herein were created by linking multiple, CD20-binding, single-chain, variable fragment (scFv) polypeptides with multiple, Shiga toxin A Subunit effector polypeptides using reagents and techniques known to the skilled worker. In this Example, the illustrative, multivalent CD20-binding molecules as described herein were multivalent CD20-binding proteins which each comprised 1) two or more single-chain, variable fragment (scFv), binding regions capable of binding an extracellular CD20 with high affinity linked with 2) two or more Shiga toxin A Subunit derived toxin effector regions.

Multivalent CD20-binding proteins were designed, produced, and purified using techniques known to the skilled worker to create protein compositions where the predominant protein(s) in the composition were multivalent CD20-binding proteins as described herein. For example, the illustrative compositions (αCD20-scFv::SLT-1A)₂ and (αCD20-scFv::SLT-1A)₂₊ₙ were predominantly composed of proteins that were multivalent CD20-binding proteins as described herein. As shown below, illustrative, multivalent, CD20-binding protein compositions as described herein were capable, via the activity of their multivalent CD20-binding protein constituent(s), of selectively killing cells that express CD20 on their surface by internalizing, routing a toxin effector region to the cytosol, and inactivating ribosomes.

However, the protein composition αCD20-scFv::SLT-1A, which was predominantly composed of monovalent CD20-binding protein representing a component of the multivalent CD20-binding proteins of this Example, did not exhibit potent, CD20-targeted cytotoxicity over a wide-range of protein concentrations. The monovalent CD20-binding protein composition αCD20-scFv::SLT-1A was unexpectedly found to be inactive at concentrations with similar total molecule binding levels to CD20-expressing cells as concentrations of illustrative, multivalent CD20-binding protein compositions as described herein at which these illustrative multivalent CD20-binding protein compositions exhibited potent targeted-cytotoxicity to CD20-expressing cells. This was surprising because the monovalent CD20-binding protein 1) had the same CD20 binding region and Shiga toxin effector region as the illustrative, multivalent CD20-binding proteins and 2) exhibited a similar catalytic activity in vitro as the illustrative, multivalent CD20-binding proteins.

### A. Construction, Production, and Purification of Cytotoxic, Multivalent, CD20-Binding Proteins to Produce the Composition (αCD20-scFv::SLT-1A)ₙ

In this Example, the Shiga toxin effector region was derived from the A Subunit of Shiga-like toxin 1 (SLT-1A). A polynucleotide was obtained that encoded amino acids 1-251 of SLT-1A (Cheung M et al., Mol Cancer 9: 28 (2010)). Immunoglobulin-type binding regions comprising single-chain variable fragments (scFv) αCD20-scFv were derived from a monoclonal antibody, developed to bind human CD20, such that a single-chain variable fragment was created with the two immunoglobulin variable regions (V_{L} and V_{H}) separated by a linker known in the art. The immunoglobulin-type binding region and the Shiga toxin effector region were cloned in frame to form a genetically fused protein.

A polynucleotide encoding the Shiga toxin effector region comprising the polypeptide shown in SEQ ID NO:4 (corresponding to amino acids 1-251 of SEQ ID NO:1) was cloned in frame with a polynucleotide encoding an immunoglobulin-type binding region αCD20-scFv. In certain experiments, the full-length coding sequence of the subunit of the multivalent cytotoxic proteins of this Example included a polynucleotide encoding a myc tag or Strep-Tag® II to facilitate detection and/or purification. A polynucleotide encoding an αCD20-scFv::SLT-1A fusion protein subunit (SEQ ID NO:54) of the multivalent CD20-binding protein(s) of the illustrative protein composition “(αCD20-scFv::SLT-1A)ₙ” were synthesized using services from DNA 2.0, Inc. (Menlo Park, Calif., U.S.) or cloned using standard techniques.

CD20-binding proteins comprising the constituents of the protein composition (αCD20-scFv::SLT-1A)ₙ were produced by protein expression from the polynucleotide template encoding the protein αCD20-scFv::SLT-1A (SEQ ID NO:54) using a bacterial system or cell-free expression system known in the art. Protein purification was accomplished using standard techniques known in the art, including capto-L and chitin affinity chromatography. For certain purifications, multivalent CD20-binding proteins were produced in bacteria and purified with the IMPACT™ (Intein Mediated Purification with an Affinity Chitin-binding Tag) system (New England Biolabs, Ipswich, Mass., U.S.). Chitin affinity purification was performed according to the manufacturer's instructions except in certain purifications, a protein L column chromatography step was performed and then the intein was cleaved. Then uncleaved material was removed using chromatography through a chitin resin in flow-through mode.

After purification, the (αCD20-scFv::SLT-1A)ₙ protein composition was oxidized using copper sulfate as a catalyst. Then, the oxidized (αCD20-scFv::SLT-1A)ₙ composition was purified into three different CD20-binding protein compositions using hydroxyapatite (hydroxylapatite) chromatography. Hydroxyapatite chromatography was used to separate different monomeric and multimeric forms of the CD20-binding proteins of the (αCD20-scFv::SLT-1A)ₙ composition into different chromatographic fractions and then certain fractions were pooled—one protein pool named “αCD20-scFv::SLT-1A composition” predominantly comprised the monovalent CD20-binding protein αCD20-scFv::SLT-1A (n=1); another protein pool named “(αCD20-scFv::SLT-1A)2 composition” predominantly comprised monospecific, bivalent CD20-binding protein (n=2) and represents an illustrative, multivalent CD20-binding molecule composition as described herein; and a third protein pool predominantly comprised monospecific, multivalent CD20-binding proteins of various sizes (n=2, 3, 4, 5, 6, etc.) with only a minority of the size of a bivalent CD20-binding protein. The third protein pool was further purified to reduce the amounts of monovalent CD20-binding protein αCD20-scFv::SLT-1A and bivalent CD20-binding protein using a second hydroxyapatite chromatography purification step followed by a size exclusion chromatography step and chromatographic fraction collection. Certain fractions comprising molecular sizes estimated to be large than bivalent CD20-binding protein were pooled to create the illustrative, multivalent CD20-binding molecule composition as described herein named “(αCD20-scFv::SLT-1A)ₙ₊₂ composition” (i.e. n+2≥3). The three protein pools (CD20-binding protein compositions) were individually concentrated.

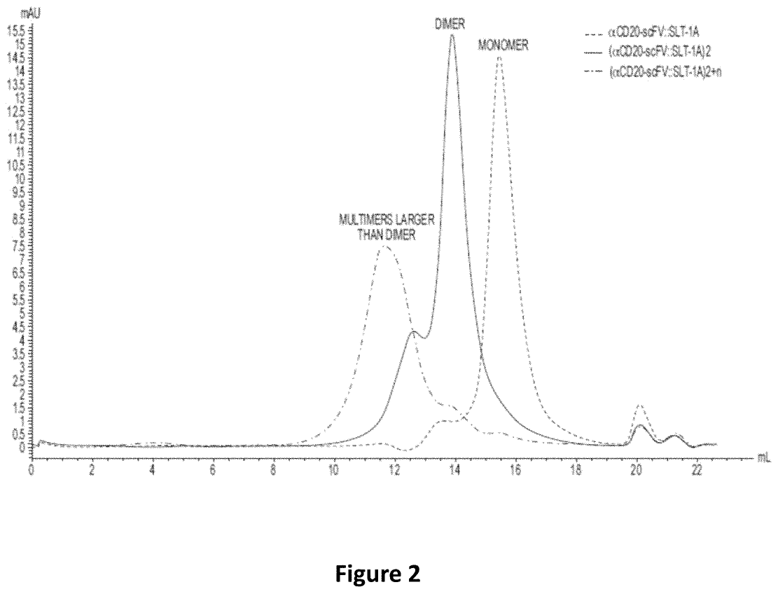

The protein makeup of each of the three CD20-binding protein compositions (1) αCD20-scFv::SLT-1A, (2) (αCD20-scFv::SLT-1A)2, and (3) (αCD20-scFv::SLT-1A)ₙ₊₂ was analyzed by size exclusion chromatography (SEC) using a Superdex 200 30/300 column (GE Healthcare, Little Chalfont, Buckinghamshire, U.K.) with a 24 mL bed volume (FIG. 2; Table 3). For the SEC chromatographic analysis, each sample was loaded and then at least 24 mL of buffer was flowed through the column while an ultraviolet (uv) light detector monitored the absorbance at 280 nanometers (nm) of the eluted materials as reported in milli-absorbance units (mAU) (FIG. 2; Table 3). Generally, smaller-sized molecules are retarded when flowing through matrices used for size exclusion chromatography as compared to larger-sized molecules and, therefore, smaller-sized molecules exhibit longer, SEC retention times than larger-sized molecules. Thus, the measuring of SEC retention times can provide an estimate of the relative proportion(s) of differently-sized molecular constituents within in a composition, and, thus, the purity of a composition with regard to molecular constituents of certain sizes.

TABLE 3
Size Exclusion Chromatographic Analysis of Purified Compositions
Having Different Forms of αCD20-scFv::SLT-1A: Relative
proportions of different monomeric and multimeric
αCD20-scFv::SLT-1A structures

| | Monomer | Dimer | MultimersD |
|---|---|---|---|
| αCD20-scFv::SLT-1A pool | 95.0% | 5.0% | 0.0% |
| (αCD20-scFv::SLT-1A)₂ pool | 0.0% | 79.0% | 21.0% |
| (αCD20-scFv::SLT-1A)₂₊ₙ pool | 3.0% | 9.0% | 88.0% |
| average retention (mL) | 15.5 | 13.8 | 12.1 |

*“Multimers > Dimer” refers to a class of multivalent CD20-binding protein multimers of sizes larger than a dimer

Table 3 shows the relative proportions of three different classes of CD20-binding protein present in the protein compositions as differentiated by size: 1) αCD20-scFv::SLT-1A class, (2) (αCD20-scFv::SLT-1A)₂ class, and (3) (αCD20-scFv::SLT-1A)ₙ₊₂ class. The αCD20-scFv::SLT-1A composition comprised 95 percent monomeric CD20-binding protein of the total protein present, and each of these monomeric CD20-binding proteins was monovalent for CD20 binding. The (αCD20-scFv::SLT-1A)₂ composition did not comprise any measurable amount of monomeric CD20-binding protein instead comprising 100 percent multivalent CD20-binding protein of the total protein present. For the (αCD20-scFv::SLT-1A)₂ composition, 79 percent of the protein present was dimeric and 21 percent of the protein present was the size of a multimeric form(s) greater than the size of any dimeric form. Finally, the analysis showed that the (αCD20-scFv::SLT-1A)₂₊ₙ composition comprised mostly multivalent CD20-binding protein of molecular sizes greater than the size of a dimeric form (88 percent of the total protein was of a size greater than the size of a dimer), but this composition also comprised a minor proportion of dimeric form(s) of multivalent CD20-binding protein and an even smaller proportion of the monomeric CD20-binding protein. The dimeric form(s) of the CD20-binding protein present in these three compositions represent illustrative, multivalent CD20-binding protein(s) as described herein that are both bivalent and multimeric.

The three CD20-binding protein compositions (1) αCD20-scFv::SLT-1A, (2) (αCD20-scFv::SLT-1A)₂, and (3) (αCD20-scFv::SLT-1A)ₙ₊₂ were also analyzed by SDS polyacrylamide gel electrophoresis (SDS-PAGE). A sample from each of the three purified protein compositions was subjected to reducing conditions of 42 millimolar (mM) dithiolthreitol (DTT) and denatured at 95° C. for 5 minutes to investigate the presence of reducible covalent bonds, such as, e.g. cysteine disulfide bonds, linking proteinaceous components of the multivalent CD20-binding proteins present in the compositions. Samples were diluted with 3×SDS Blue Loading Buffer (187.5 mM Tris-HCl (pH 6.8), 6% mass/volume percentage (w/v) sodium dodecyl sulfate (SDS), 30% glycerol and 0.03% (w/v) bromophenol blue, Catalog #B7703S, New England BioLabs, Inc., Ipswich, Mass., U.S.) or 3×SDS Reducing Blue Loading Buffer (187.5 mM Tris-HCl (pH 6.8), 6% (w/v) SDS, 30% glycerol, 0.03% (w/v) bromophenol blue, and 125 mM DTT, Catalog #B7703 S, New England BioLabs, Inc., Ipswich, Mass., U.S.) to a final composition of 1× buffer and mixed well. The samples were heated at 95° C. for 5 minutes and then 5 micrograms (μg) of protein sample per well was loaded into wells of a 4-20% SDS polyacrylamide gel and subjected to electrophoresis.

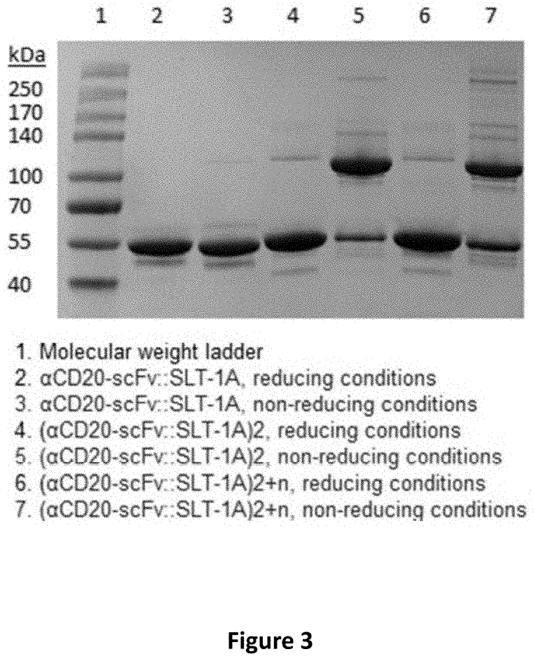

Samples of both reduced and non-reduced purified protein pools were analyzed in denaturing conditions by SDS-PAGE (FIG. 3). FIG. 3 shows images of a Coomassie-stained, 4-20% SDS-PAGE gel (Lonza, Basel, CH) with the lanes of the gel numbered and the figure legend indicating which sample was loaded into each lane by the same respective numbering. Multimeric forms of the multivalent CD20-binding proteins present in a sample whose subunits are associated only from non-covalent interactions were expected to dissociate into their component monovalent proteins in this denaturing gel analysis regardless of redox state due to the nature of the SDS-PAGE technique performed. In contrast, multimeric forms of the multivalent CD20-binding proteins present in a sample that result from reducible covalent bonds, such as, e.g., cysteine disulfide bridge-dependent forms, might be observed to dissociate into proteinaceous components in reduced samples but not in unreduced samples. However, in both situations incomplete, disulfide bond reduction and/or protein denaturation could permit the persistence of multimeric structures.

The electrophoresis analysis showed that the majority proteinaceous species in the αCD20-scFv::SLT-1A composition migrated at a molecular mass of about 55 kDa (FIG. 3), which was the approximate size expected for SEQ ID NO:54, which has an anti-CD20 scFv fused to the Shiga toxin A Subunit effector polypeptide SLT-1A 1-251. The size of this species was unchanged between non-reducing and reducing conditions (FIG. 3). These results were consistent with the majority protein species in the αCD20-scFv::SLT-1A composition as being the monomeric, monovalent CD20-binding protein component of the illustrative, multivalent CD20-binding protein compositions of this Example.

The electrophoresis analysis showed that the majority proteinaceous species in the (αCD20-scFv::SLT-1A)₂ composition migrated at a molecular mass of about 110 kDa (FIG. 3, lane 5), which was the approximate size expected for a dimeric form consisting of exactly two of the monomeric, monovalent CD20-binding proteins of the αCD20-scFv::SLT-1A composition. There was also a minority proteinaceous species present in the (αCD20-scFv::SLT-1A)₂ composition which migrated at a molecular mass of about 55 kDa under non-reducing conditions and might represent the monomeric, monovalent CD20-binding protein component αCD20-scFv::SLT-1A after the denaturation of a dimeric form(s) that result from one or more non-covalent association(s) but not any covalent linkage(s), such as, e.g., a cysteine disulfide bond. The size of the vast majority of this majority species changed to about 55 kDa under reducing conditions (FIG. 3, lane 5), which was consistent with the existence, under non-reducing conditions, of one or more disulfide bonds linking two, monomeric, monovalent CD20-binding protein molecules together in a dimeric form(s) for the majority protein species present in the (αCD20-scFv::SLT-1A)₂ composition.

The electrophoresis analysis showed that the majority proteinaceous species in the (αCD20-scFv::SLT-1A)₂+. composition migrated at a molecular mass of about 55 kDa or 110 kDa (FIG. 3, lane 7), which was the approximate size expected for either the monomeric, monovalent CD20-binding protein component αCD20-scFv::SLT-1A or a dimeric form(s) consisting of exactly two monovalent CD20-binding protein components. The size of the vast majority of the dimeric protein species changed to about 55 kDa under reducing conditions (FIG. 3, lane 6), which was consistent with the existence, under non-reducing conditions, of one or more disulfide bonds linking two, monovalent CD20-binding protein molecules together in a dimeric form(s) in the (αCD20-scFv::SLT-1A)₂₊ₙ composition.

The comparison of the reduced and non-reduced samples electrophoretically separated through SD S-PAGE gels under denaturing showed that the compositions (αCD20-scFv::SLT-1A)₂ and (αCD20-scFv::SLT-1A)ₙ₊₂ included both covalent and non-covalent multimers (FIG. 3, lanes 4-7). These illustrative compositions of multivalent CD20-binding proteins as described herein, (αCD20-scFv::SLT-1A)₂ and (αCD20-scFv::SLT-1A)ₙ₊₂ comprise multivalent CD20-binding proteins which have covalently linked, protein subunits and/or non-covalent linked protein subunits.

### B. Determining the Cell-Binding Characteristics of Multivalent CD20-Binding Proteins Present in Illustrative Compositions

The binding characteristics of the multivalent CD20-binding protein compositions (αCD20-scFv::SLT-1A)₂ and (αCD20-scFv::SLT-1A)ₙ₊₂ to human tumor-derived, cell lines were studied using a fluorescence-based, flow-cytometry assay. The protein compositions (αCD20-scFv::SLT-1A)₂ and (αCD20-scFv::SLT-1A)ₙ₊₂, which were produced as described above, were analyzed for the ability of their multimeric, multivalent CD20-binding proteins having Shiga toxin effector regions to bind to human tumor-derived, cell lines that express human CD20 at a cellular surface.

Samples containing CD20 positive (CD20+) Raji cells or CD20 negative (CD20-) U266 cells were suspended in 1×PBS containing one percent bovine serum albumin (BSA) (Calbiochem, San Diego, Calif., U.S.), hereinafter referred to as “1×PBS+1% BSA” and incubated for one hour at 4 degrees Celsius (° C.) with 100 microliters (μL) of various dilutions of the multivalent CD20-binding protein compositions to be assayed. After the one-hour incubation, samples comprising a mixture of cells and a multivalent CD20-binding protein composition were washed twice with 1×PBS+1% BSA. Then the samples were incubated for one hour at 4° C. with 100 μL of 1×PBS+1% BSA solution comprising a murine monoclonal antibody anti-SLT-1A (BEI NR-867 BEI Resources, Manassas, Va., U.S.; cross reactive with Shiga toxin and Shiga-like toxin 1 A subunits) at an antibody concentration larger than the total protein concentration present in each sample. The samples were washed with 1×PBS+1% BSA and then incubated in the same manner with an anti-mouse IgG secondary antibody conjugated with FITC at an antibody concentration larger than the total protein concentration present in each sample. Then the samples were washed twice with 1×PBS+1% BSA, resuspended in 200 μL of 1×PBS, and subjected to fluorescence-based, flow cytometry in order to measure protein binding to the cells.

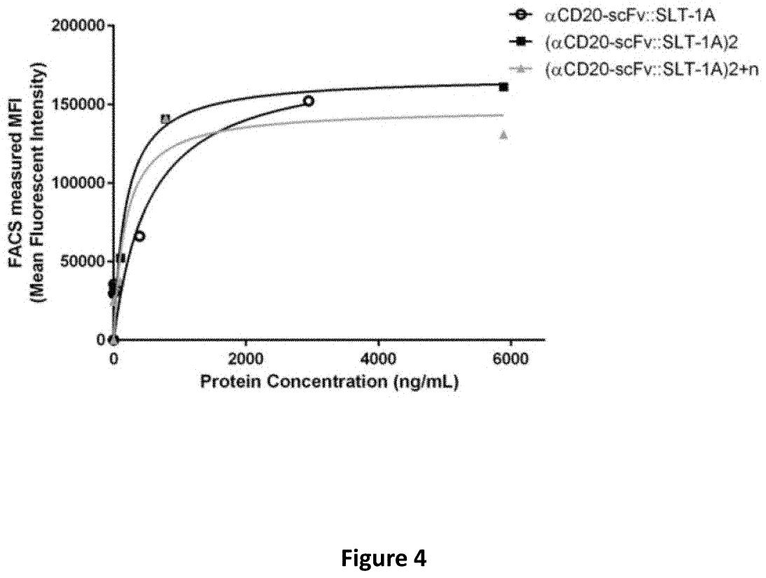

The maximum specific binding (Borax) and equilibrium binding constants (K_{D}) of αCD20-scFv::SLT-1A, (αCD20-scFv::SLT-1A)₂ and (αCD20-scFv::SLT-1A)ₙ₊₂ samples to human tumor-derived, cell lines were determined as follows. The mean fluorescence intensity (MFI) data from the fluorescence-based, flow cytometry for all the samples was obtained by gating the data using a cell sample incubated only with the secondary antibody as a negative control. Graphs were plotted of the MFI data versus “concentration of protein” using Prism software (GraphPad Software, San Diego, Calif., U.S.) (FIG. 4). Using the Prism software function of one-site binding [Y=Bₘₐₓ*X/(K_{D}+X)] under the heading binding-saturation, the Bₘₐₓ and K_{D} were calculated using baseline corrected data. Light absorbance (Abs) values were corrected for background by subtracting the Abs values measured for wells containing only PBS. Bₘₐₓ is the maximum specific binding reported in MFI. K_{D} is the equilibrium binding constant, reported in nanograms per milliliter (ng/mL). The K_{D} and Bₘₐₓ values for the compositions (αCD20-scFv::SLT-1A)₂ and (αCD20-scFv::SLT-1A)ₙ₊₂ are reported in Table 4 and shown in FIG. 4.

TABLE 4
Binding of Illustrative, Multivalent αCD20-scFv::SLT-1A Compositions
described herein to CD20+ Raji Cells as Compared to a Monovalent
CD20-Binding Protein Composition

| Protein Composition | Bₘₐₓ (MFI) | K_{D} (ng/mL) |
|---|---|---|
| (αCD20-scFv::SLT-1A)₂ dimer | 167,728 | 180.2 |
| (αCD20-scFv::SLT-1A)ₙ₊₂ | 147,366 | 176.9 |
| αCD20-scFv::SLT-1A monomer | 178,118 | 544.1 |

The Bₘₐₓ for (CD20-scFv::SLT-1A)₂ binding to CD20⁺ Raji cells was measured to be about 170,000 MFI with a K_{D} of about 180 ng/mL (Table 4; FIG. 4). The Bₘₐₓ for (CD20-scFv::SLT-1A)ₙ₊₂ binding to CD20⁺ Raji cells was measured to be about 150,000 MFI with a K_{D} of about 180 ng/mL (Table 4; FIG. 4). Thus, illustrative protein compositions as described herein (αCD20-scFv::SLT-1A)₂ and (αCD20-scFv::SLT-1A)ₙ₊₂ (which were predominantly composed of multimeric, multivalent CD20-binding proteins) both exhibited high-affinity binding to human CD20-expressing human cells expressing CD20 at a cell surface (e.g. CD20⁺ human cells). It is unknown whether any multimeric form of CD20-scFv::SLT-1A present in either the (αCD20-scFv::SLT-1A)₂ or (αCD20-scFv::SLT-1A)ₙ₊₂ compositions is capable of simultaneously binding two, different, CD20 target biomolecules present at the cell surface of a single, CD20-expressing cell.

C. Determining the Half-Maximal Inhibitory Concentration (IC₅₀) of the Proteins (αCD20-scFv::SLT-1A)₂ and (αCD20-scFv::SLT-1A)ₙ₊₂ to Eukaryotic Ribosomes In Vitro

The ribosome inactivation capabilities of the (αCD20-scFv::SLT-1A)₂ and (αCD20-scFv::SLT-1A)ₙ₊₂ protein compositions were determined in a cell-free, in vitro protein translation assay using the TNT® Quick Coupled Transcription/Translation Kit (L1170 Promega, Madison, Wis., U.S.). The kit includes Luciferase T7 Control DNA and TNT® Quick Master Mix. The ribosome activity reaction was prepared according to the manufacturer's instructions to create “TNT” reaction mixtures. The concentrations of CD20-binding protein present in the samples were calculated based on the molarity of the SLT-1A components (see below). A series of 10-fold dilutions of the CD20-binding protein compositions to be analyzed was prepared in an appropriate buffer, and a series of identical TNT reaction mixture components was created for each sample dilution.

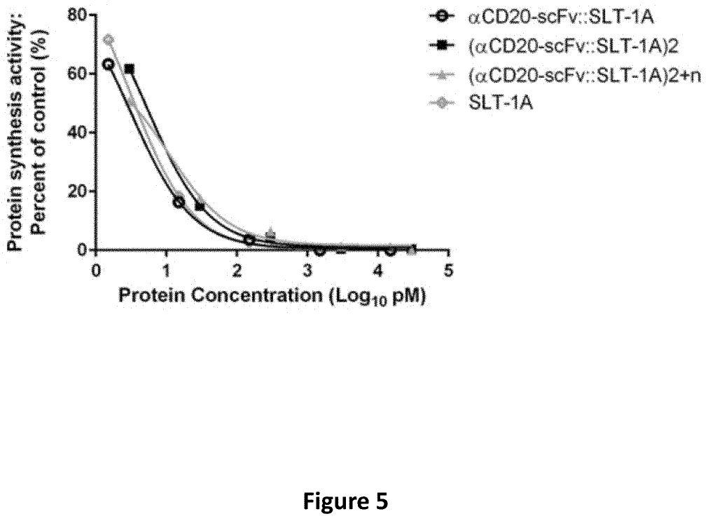

Each sample in the dilution series was combined with each of the TNT reaction mixtures along with the Luciferase T7 Control DNA. The test samples were incubated for 1.5 hours at 30° C. After the incubation, Luciferase Assay Reagent (E1483 Promega, Madison, Wis., U.S.) was added to all test samples, and the amount of luciferase protein translation was measured by luminescence according to the manufacturer's instructions. The level of translational inhibition was determined by non-linear regression analysis of log-transformed molar concentrations of the total protein, estimated based on the normalized molar concentration of Shiga toxin protein versus relative luminescence units. Using statistical software (GraphPad Prism, San Diego, Calif., U.S.), the half maximal inhibitory concentration (IC50) in picomolar (pM) value was calculated for each CD20-binding protein composition tested (FIG. 5; Table 5).

TABLE 5
Ribosome Inactivation Analysis: Representative half-maximal inhibitory
concentrations (IC₅₀) of multivalent CD20-binding proteins described
herein as compared to a monovalent CD20-binding protein

| Protein | IC₅₀ (pM) |
|---|---|
| (αCD20-scFv::SLT-1A)₂ | 5.29 |
| (αCD20-scFv::SLT-1A)ₙ₊₂ | 10.74 |
| αCD20-scFv::SLT-1A monomer | 3.14 |
| SLT-1 A (1-251) only positive control | 3.15 |

The inhibitory effects of the illustrative, multivalent CD20-binding protein compositions (CD20-scFv::SLT-1A)₂ and (CD20-scFv::SLT-1A)ₙ₊₂ on cell-free protein synthesis were strong (FIG. 5; Table 5). Dose-dependence experiments determined that the IC50 values of the multivalent CD20-binding molecules present in (CD20-scFv::SLT-1A)₂ and (CD20-scFv::SLT-1A)ₙ₊₂ to protein synthesis in this cell-free assay were about 5.3 pM and 11 pM, respectively (FIG. 5; Table 5).

D. Determining the Half-Maximal Cytotoxic Concentrations (CD₅₀) of the Multivalent CD20-Binding Proteins (αCD20-scFv::SLT-1A)₂ and (αCD20-scFv::SLT-1A)ₙ₊₂ Using a CD20+ Cell-Kill Assay

Dose dependence experiments were used to determine the CD₅₀ values of the illustrative CD20-binding protein compositions as described herein (αCD20-scFv::SLT-1A)₂ and (αCD20-scFv::SLT-1A)ₙ₊₂. The cytotoxicity characteristics of the (αCD20-scFv::SLT-1A)₂ and (αCD20-scFv::SLT-1A)ₙ₊₂ compositions were determined by the following CD20+ cell-kill assay. This assay determines the capacity of a protein sample to kill cells expressing at a cellular surface the CD20 target biomolecule of the multivalent CD20-binding protein's binding region(s). CD20+ Raji cells and CD20+ST486 cells were plated (7.5×10³ cells per well) in 20 μL cell culture medium in 384-well plates. The multivalent CD20-binding protein compositions to be tested were diluted 10-fold in a 1×PBS, and 5 μL of the dilutions were added to the CD20+ and CD20− cell samples in the 384-well plates. Control wells containing only cell culture medium were used for baseline correction. The cell samples were incubated with protein samples or just buffer for three days at 37° C. and in an atmosphere of 5% carbon dioxide (CO₂). The total cell survival or percent viability was determined using a luminescent readout using the CellTiter-Glo® Luminescent Cell Viability Assay (G7573 Promega Madison, Wis., U.S.) according to the manufacturer's instructions. The Percent Viability of experimental wells was calculated using the following equation: (Test RLU−Average Media RLU)/(Average Cells RLU−Average Media RLU)*100. Log polypeptide concentration versus Percent Viability was plotted in Prism (GraphPad Prism, San Diego, Calif., U.S.) and log (inhibitor) vs. response (3 parameter) analysis were used to determine the half-maximal cytotoxic concentration (CD₅₀) value for the multivalent CD20-binding protein compositions (αCD20-scFv::SLT-1A)₂ and (αCD20-scFv::SLT-1A)ₙ₊₂ to CD20+ cells.

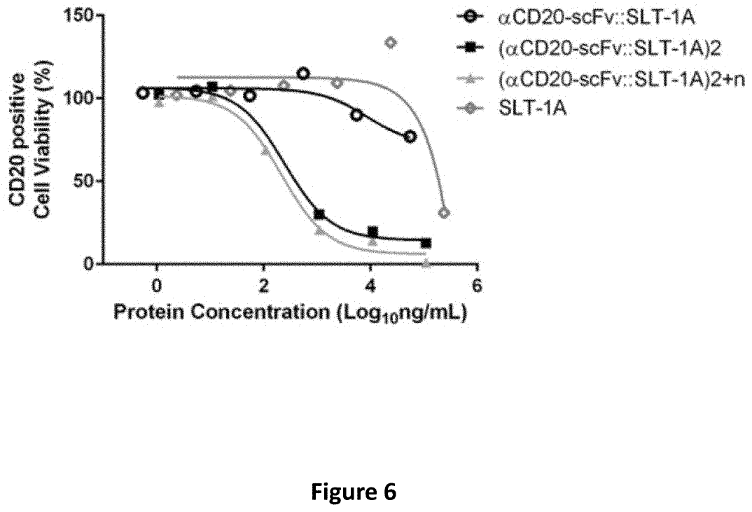

The CD₅₀ value of (αCD20-scFv::SLT-1A)₂ composition to CD20⁺ Raji cells was 250 ng/mL (Table 6; FIG. 6). The CD₅₀ value of the composition (αCD20-scFv::SLT-1A)ₙ₊₂ to CD20 Raji cells was about 220 ng/mL (Table 6; FIG. 6). In contrast, the CD₅₀ value of the monomeric, monovalent CD20-binding protein composition αCD20-scFv::SLT-1A was much higher (i.e. less potent) such that at the tested concentrations a CD₅₀ could not be accurately determined from the shape of the curve (Table 6, “NC” denotes not calculable; FIG. 6). For the protein concentrations and cell densities tested in this assay, it was estimated that at certain concentrations of the proteins tested, the available cell-surface CD20 present could be saturated by CD20-binding protein (see, Muller P, Brennan F, Clin Pharmacol Ther 85: 247-58 (2009), for an illustrative “RO model” used to estimate occupancy).

TABLE 6
Cytotoxicity: Representative half-maximal cytotoxic concentrations
(CD₅₀) for illustrative, multivalent CD20-binding protein
compositions as described herein to CD20+ Raji Cells

| Protein | CD₅₀ (ng/mL) |
|---|---|
| (αCD20-scFv::SLT-1A)₂ | 249.0 |
| (αCD20-scFv::SLT-1A)ₙ₊₂ | 217.7 |
| αCD20-scFv::SLT-1A | NC* |

*“NC” (not calculable) indicates that an accurate CD₅₀ could not be calculated based on the shape of the curve.

Using the same cell-kill assay, the (αCD20-scFv::SLT-1A)₂ protein composition was shown in other experiments to be nontoxic to CD20 negative cell lines, such as, e.g., BC-1, U266, and H929 cells, when tested at similar cell densities and CD20-binding protein concentrations, which included protein concentrations as high as 40,000 ng/mL. Also using the same cell-kill assay, both the SLT-1A (1-251) component alone and the monomeric, monovalent CD20-binding protein composition αCD20-scFv::SLT-1A did not exhibit specific cytotoxicity toward CD20+ Raji cells at protein concentrations as large as 24,000 ng/mL.

The cytotoxicity measurements of the monovalent CD20-binding protein composition αCD20-scFv::SLT-1A showed that αCD20-scFv::SLT-1A exhibited no greater cytotoxicity towards CD20⁺ Raji cells at the tested cell density and protein concentrations compared with the cytotoxicity of an SLT-1A (1-251) “only” negative control sample, which lacked any cell-targeting moiety like a cell-surface receptor binding region. Thus, the monovalent CD20-binding protein composition αCD20-scFv::SLT-1A exhibited only non-specific cytotoxicity regardless of cell-surface marker expression. In conclusion, the monovalent CD20-binding protein αCD20-scFv::SLT-1A was incapable of killing CD20+ cells at the protein concentrations tested; whereas, the illustrative, multivalent CD20-binding protein compositions as described herein (αCD20-scFv::SLT-1A)₂ and (αCD20-scFv::SLT-1A)ₙ₊₂ showed potent, cell-targeted cytotoxicity specifically to CD20-expressing cells.

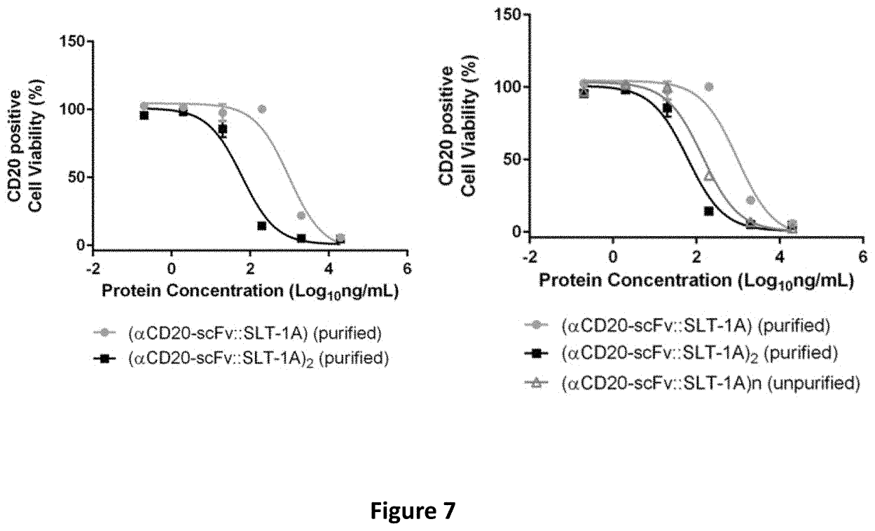

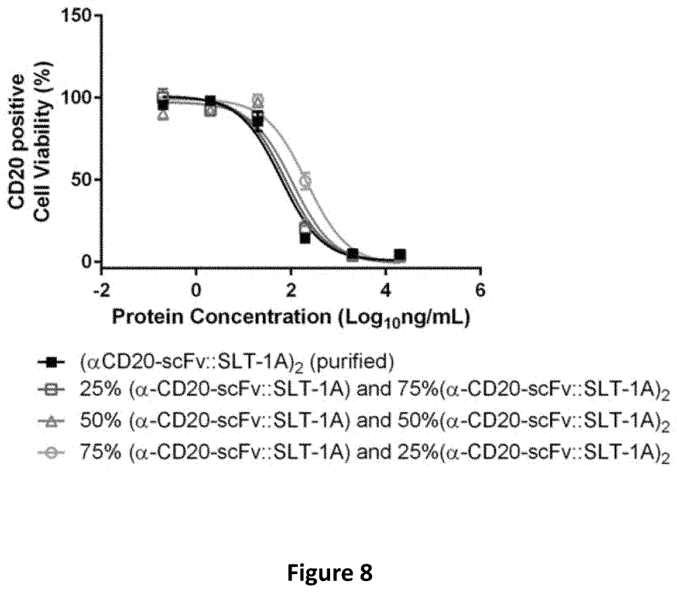

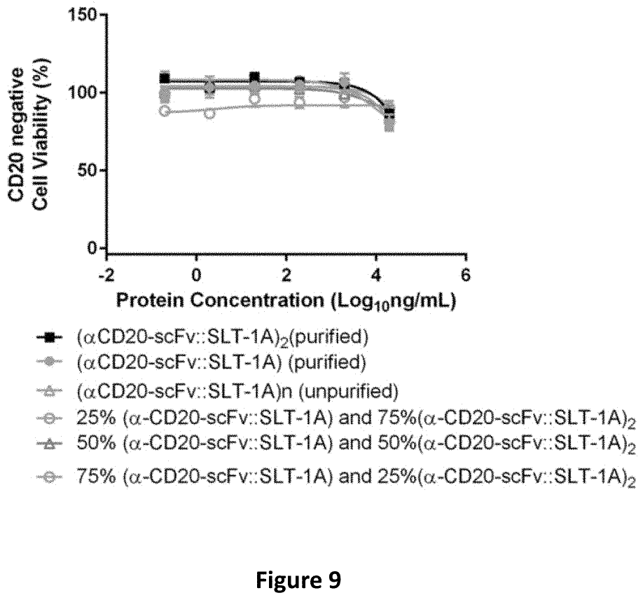

To further investigate these unexpected results, the protein compositions αCD20-scFv::SLT-1A and (αCD20-scFv::SLT-1A)₂ were mixed together to form new compositions to test the cytotoxic potency of their constituent CD20-binding proteins as a function of the ratio of CD20-binding protein constituents. The (αCD20-scFv::SLT-1A)₂ composition comprised 100% multivalent CD20-binding protein of the total protein present and 79% of that multivalent CD20-binding protein was bivalent CD20-binding protein (see Table 3, supra). The αCD20-scFv::SLT-1A composition comprised 95% monovalent CD20-binding protein of the total protein present (see Table 3, supra). Increasingly larger samples of the multivalent CD20-binding molecule composition (αCD20-scFv::SLT-1A)₂ were added to samples of the monovalent CD20-binding protein composition αCD20-scFv::SLT-1A to create a series of mixed samples with total protein concentration ratios of 1:3, 1:1, and 3:1 of the (αCD20-scFv::SLT-1A)₂ composition to the αCD20-scFv::SLT-1A composition. Samples of the fixed-ratio, mixed samples, along with samples of the original, unmixed αCD20-scFv::SLT-1A and (αCD20-scFv::SLT-1A)₂ compositions, were tested using the CD20+ cell-kill assay as described above to determine each sample's CD₅₀ value to CD20-expressing cells (ST486). The results are shown in FIG. 7, FIG. 8, and Table 7, along with the results for the unmixed αCD20-scFv::SLT-1A and (αCD20-scFv::SLT-1A)₂ compositions. In addition, none of these samples exhibited cytotoxicity toward CD20 negative cells using this assay at the concentrations tested (FIG. 9).

TABLE 7
Cytotoxicity: Representative half-maximal cytotoxic concentrations (CD₅₀)
for the multivalent (αCD20-scFv::SLT-1A)₂ composition diluted with
increasingly more of the monovalent αCD20-scFv::SLT-1A composition

| Protein | CD₅₀ (ng/mL) |
|---|---|
| 1:0 (αCD20-scFv::SLT-1A)₂ | 61.5 |
| 3:1 (αCD20-scFv::SLT-1A)₂ to αCD20-scFv::SLT-1A | 74.9 |
| 1:1 (αCD20-scFv::SLT-1A)₂ to αCD20-scFv::SLT-1A | 108.0 |
| 1:3 (αCD20-scFv::SLT-1A)₂ to αCD20-scFv::SLT-1A | 213.0 |
| 0:1 αCD20-scFv::SLT-1A | 967.0 |
| unpurified (αCD20-scFv::SLT-1A)₂ | 142.0 |

FIG. 7 shows the CD20+ cell-kill assay results for the illustrative, multivalent CD20-binding molecule composition as described herein (αCD20-scFv::SLT-1A)₂, the monovalent CD20-binding protein composition αCD20-scFv::SLT-1A and the “unpurified” protein composition (αCD20-scFv::SLT-1A)ₙ described above before any copper sulfate oxidation step.

FIG. 8 shows the CD20+ cell-kill assay results for the fixed-ratio mixtures of a 1:3 protein concentration ratio of (αCD20-scFv::SLT-1A)₂ to αCD20-scFv::SLT-1A, a 1:1 protein concentration ratio of (αCD20-scFv::SLT-1A)₂ to αCD20-scFv::SLT-1A, and a 3:1 protein concentration ratio of (αCD20-scFv::SLT-1A)₂ to αCD20-scFv::SLT-1A, along with the original, multivalent CD20-binding protein composition (αCD20-scFv::SLT-1A)₂.

Table 7 reports the CD₅₀ values for samples of the purified (αCD20-scFv::SLT-1A)₂ composition, the mixture of a 1:3 protein concentration ratio of (αCD20-scFv::SLT-1A)₂ to αCD20-scFv::SLT-1A, the mixture of a 1:1 protein concentration ratio of (αCD20-scFv::SLT-1A)₂ to αCD20-scFv::SLT-1A, the mixture of a 3:1 protein concentration ratio of (αCD20-scFv::SLT-1A)₂ to αCD20-scFv::SLT-1A, and “unpurified” (αCD20-scFv::SLT-1A)ₙ. Table 7 shows the cytotoxicity of the purified, multivalent CD20-binding molecule composition (αCD20-scFv::SLT-1A)₂ to CD20-expressing cells was about 16 times greater than the cytotoxicity of the purified, monovalent CD20-binding protein composition αCD20-scFv::SLT-1A to CD20-expressing cells. Table 7 shows the (αCD20-scFv::SLT-1A)₂ composition was about 2.3 times more cytotoxic than the “unpurified” (αCD20-scFv::SLT-1A)ₙ composition from which the (αCD20-scFv::SLT-1A)₂ composition was purified.

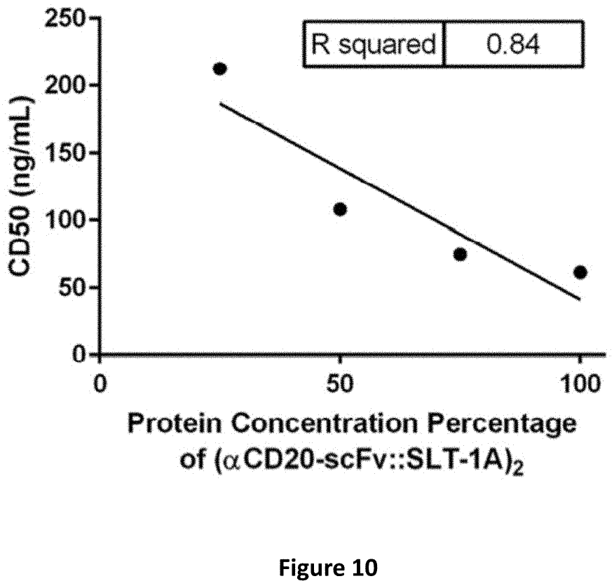

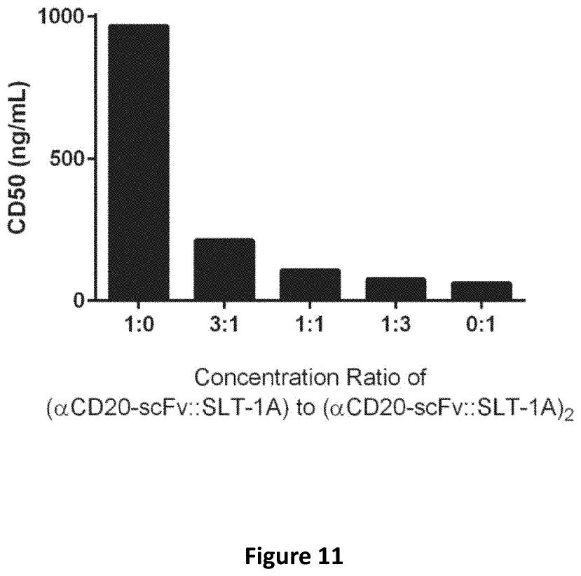

The CD₅₀ values in Table 7 were graphed as a function of the percentage of (αCD20-scFv::SLT-1A)₂ composition protein of the total protein in the sample tested, and a straight line was fitted to the data points using a simple linear regression, statistical model (FIG. 10). The coefficient of determination (“R squared”) of the line fit was 0.8424. In FIG. 11, the CD₅₀ values represented in Table 7 were graphed as a function of the percentage of (αCD20-scFv::SLT-1A)₂ composition protein of the total protein in the sample.

The results reported in Table 7, FIG. 10, and FIG. 11 show that as the multivalent CD20-binding protein composition (αCD20-scFv::SLT-1A)₂ was diluted with the monovalent CD20-binding protein composition αCD20-scFv::SLT-1A, the cytotoxicity of the mixed CD20-binding protein samples was reduced, such as, e.g., by 4-fold or greater, as assayed by CD₅₀ values measured with the CD20+ cell-kill assay. FIGS. 10 and 11 show that as the relative protein concentration of the multivalent CD20-binding protein composition (αCD20-scFv::SLT-1A)₂ increases over the total CD20-binding protein concentration, the cytotoxic potency of the mixture to CD20+ cells increased (represented by lower CD₅₀ values). The cytotoxicity of the (αCD20-scFv::SLT-1A)₂ composition to CD20+ cells was diluted in linear manner by the addition of more and more of the monovalent CD20-binding protein composition αCD20-scFv::SLT-1A (see FIGS. 8, 10, and 11). Thus, for achieving more maximal cytotoxicity of a multivalent CD20-binding protein composition as described herein, the amount of any monovalent CD20-binding protein (e.g., αCD20-scFv::SLT-1A or (αCD20-scFv::SLT-1A)ₙ where n=1) or the relative proportion monovalent CD20-binding protein to total CD20-binding protein should be minimized or eliminated because a monovalent CD20-binding protein represents a cytotoxicity-lowering constituent of the composition, such as, e.g., by functioning as a non-cytotoxic impurity or a constituent having significantly lower cytotoxicity than a multivalent CD20-binding molecule comprising it as a component.

Considering that the in vitro, ribosome inhibitory activities of both monovalent and multivalent variants of the CD20-binding protein compositions were similar (see Table 5), it was expected that changes in CD20-expressing cell binding caused by differences in CD20-binding valency would account for any differences in cytotoxic potency between monovalent and multivalent CD20-binding protein. However, the lack of cytotoxic potency of the monovalent CD20-binding protein composition αCD20-scFv::SLT-1A as compared to the (αCD20-scFv::SLT-1A)₂ composition cannot be explained merely by the difference in CD20+ cell-binding between the αCD20-scFv::SLT-1A composition and the (αCD20-scFv::SLT-1A)₂ composition (see Table 4 and FIG. 4).

Using the same cell-kill assay, the fixed-ratio mixtures of CD20-binding protein compositions were analyzed for cytotoxicity to CD20 negative H929 cells (FIG. 9). The CD₅₀ values of the mixture of a 1:3 protein concentration ratio of (αCD20-scFv::SLT-1A)₂ to αCD20-scFv::SLT-1A, the mixture of a 1:1 protein concentration ratio of (αCD20-scFv::SLT-1A)₂ to αCD20-scFv::SLT-1A, and the mixture of a 3:1 protein concentration ratio of (αCD20-scFv::SLT-1A)₂ to αCD20-scFv::SLT-1A to target negative cells were not calculable because of the shape of the curve produced by the dilution series. Thus, the fixed-ratio mixtures were not cytotoxicity to CD20 negative cells at the concentrations tested; whereas, each ratio tested showed some levels of targeted cytotoxicity to CD20+ cells (Table 7; FIG. 9).

In addition, CD20+ cell-kill assays were performed as described above to test the cytotoxicity of protein compositions comprising purified, catalytically inactive, bivalent CD20-binding protein. Protein compositions, which comprised multivalent CD20-binding protein, were produced as described above for the purification and production of the composition (αCD20-scFv::SLT-1A)₂. The Shiga toxin effector polypeptide regions of the CD20-binding proteins of these compositions were catalytically inactive or catalytically impaired due to the presence of the mutation(s) E167D, Y77S, or Y77S/E167D in their Shiga toxin effector regions. The multivalent CD20-binding proteins of these compositions comprised the monovalent CD20-binding protein αCD20-scFv::SLT-1A (SEQ ID NO:54) with at least one of the mutations noted above for studying the role of catalytic activity. The catalytically inactive variants of the multivalent CD20-binding proteins were not cytotoxic at the concentrations tested. Without being bound by theory, the requirement for a catalytically active, Shiga toxin effector region for cell-killing by multivalent CD20-binding molecules of this Example showed the mechanism of cytotoxicity of certain, multivalent CD20-binding molecule, and compositions thereof, 1) is Shiga toxin effector region dependent, 2) requires the Shiga toxin activity of ribosome inactivation within the target cell, and 3) does not involve any other cytotoxic effect of the multivalent CD20-binding molecule independent of Shiga toxin effector catalytic activity (i.e., no other cytotoxic effect of the multivalent CD20-binding molecule (e.g., a Shiga toxin effector region-independent, extracellular, cytotoxic effect) was observed in the assays of this Example at the protein concentrations tested in the absence of Shiga toxin effector region catalytic activity.

### E. Determining Relative Proportions of Multivalent CD20-Binding Molecules in Illustrative Compositions

Chromatographic and/or electrophoretic methods known to the skilled worker were used to determine 1) relative protein concentration ratios of different CD20-binding proteins within illustrative compositions as described herein.

In this Example, SEC analyses were used to analyze the amount of proteinaceous species of different sizes present in illustrative compositions as described herein, e.g., the ratios and/or percentages between monovalent, bivalent, and higher-order multivalent CD20-binding species were calculated.

SEC analyses were performed using the following assays. Samples were loaded onto a fast protein liquid chromatography (FPLC) column and buffer was flowed through the column while the absorbance at 280 nm of the eluted materials was recorded in mAU. Using the same column and setup, molecules of known sizes and migration characteristics (standards) were analyzed in order to calibrate which retention times corresponded to which protein sizes and/or protein sizes were predicted from the amino acid composition of each monovalent CD20-binding protein monomer present in the composition to be analyzed. Chromatographic data collected from commercial size standards were used to create calibration curves to help estimate sizes of molecular species in samples and focus analyses on specific retention time ranges. In certain instances of high purity, the total protein quantity for a given composition was estimated using the absorbance measurement at 280 nm from a SEC analysis, the predicted molecular weight of the majority molecular species present, and the extinction coefficient of that majority species.

In addition, SDS-PAGE and/or capillary gel electrophoretic analyses of both reduced and non-reduced samples was used to verify the sizes, estimate the relative quantities, and detect any disulfide bond, multimeric associations present in the molecular species of a given size or peak (see e.g. FIG. 3). For example, the size of a molecule in a SEC peak can be estimated based on non-reducing SDS-PAGE analysis of chromatographic fractions collected at retention times within that peak's retention duration.

In this Example, all the CD20-binding proteins in the illustrative compositions as described herein consisted essentially of monovalent CD20-binding protein and/or a multimeric form(s) of a monovalent CD20-binding protein(s). Thus, peaks and bands corresponding to species: 1) the same size of the monovalent CD20-binding protein were composed of monovalent CD20-binding molecule, 2) twice as large as the monovalent CD20-binding protein were composed of bivalent CD20-binding molecule(s), 3) three times as large as the monovalent CD20-binding protein were composed of trivalent CD20-binding molecule(s), and so forth.

Some SEC analyses involved assays performed with an ÄKTA system (GE Healthcare, Little Chalfont, Buckinghamshire, U.K.). UNICORN™ control software (GE Healthcare, Little Chalfont, Buckinghamshire, U.K.) was used to calculate the protein concentration percentages of different sized-species using the software's peak integration functions, which included baseline calculation, determination of the start and end of peaks, the retention time and the area under the curve. The identity of the CD20-binding protein species represented by a peak in the 280 nm trace from a chromatographic analysis were manually assigned based on the expected retention time determined using calibration standards (e.g. a gel filtration standard like GE Healthcare Life Science's product 28-4038-42 Gel Filtration HMW Calibration Kit).

Other SEC analyses involved a similar assay performed with a Waters system (Waters Corp., Milford, Mass., U.S.). SEC analysis was performed using a Waters Alliance HPLC system running Water Empower 2 software (Waters Corp., Milford, Mass., U.S.). The HPLC system included an analytical TSKgel G3000SW_{XL} size-exclusion column and a TSKgel Guard SW_{XL} size-exclusion guard column (Tosoh Bioscience LLC, King of Prussia, Pa., U.S.). Prior to use, the columns were equilibrated with mobile phase (20 mM sodium phosphate, 500 mM sodium chloride, pH 7.4) for at least thirty minutes at a flow rate of 0.5 mL per minute (mL/min). A blank sample of 100 μL of mobile phase was run through the columns to check the system was clean and working properly. Then composition samples were analyzed, including samples of known protein size standards (e.g. a commercially available, gel filtration standard like GE Healthcare Life Science's product 28-4038-42 Gel Filtration BMW Calibration Kit). For each composition sample, 50 μg of protein was injected and an isocratic pump was run for thirty minutes at a flow-rate of 0.5 mL per minute though the columns at a temperature of 22° C.

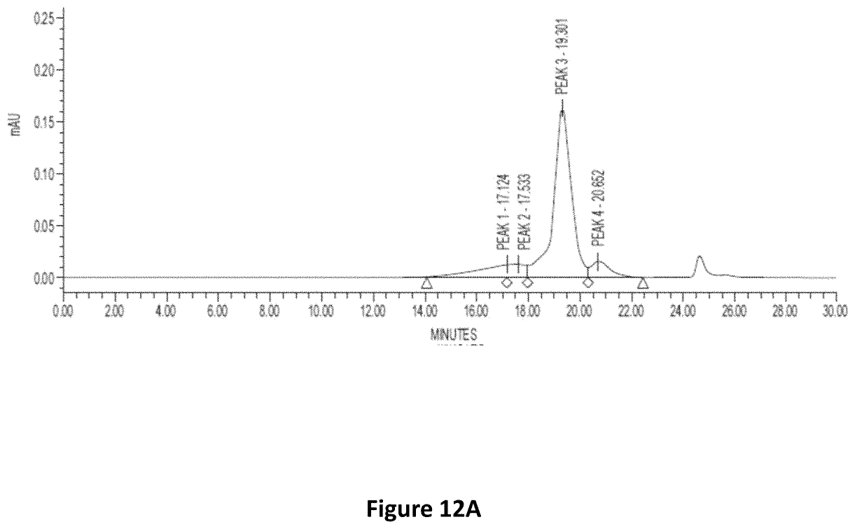

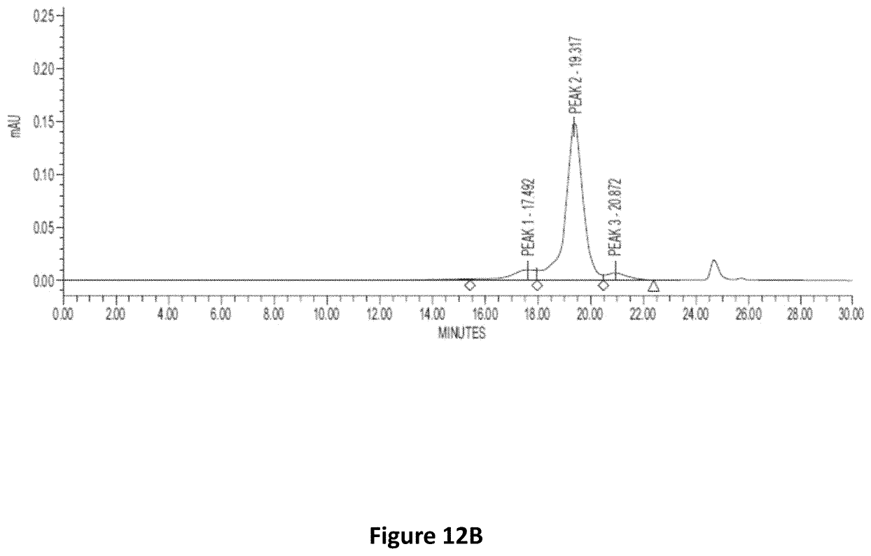

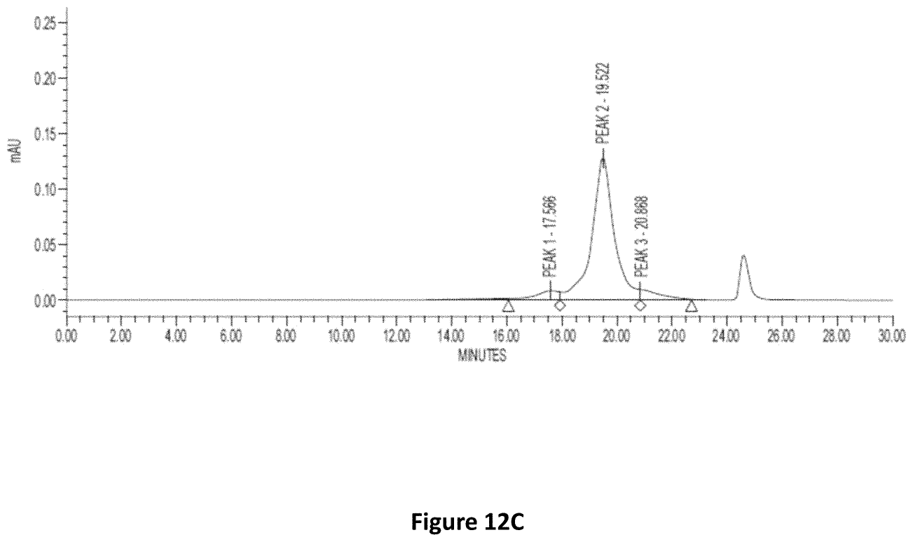

The percentages of multivalent CD20-binding molecule present in three, different, illustrative compositions as described herein were analyzed by SEC using the Waters system (see FIG. 12, panel A, panel B, and panel C), and the respective results of these analyses are shown in Tables 6-8. In this Example for the Waters system, a retention time of (1) about 19 to 20 minutes represented a molecular size equivalent to dimeric, bivalent CD20-binding protein (αCD20-scFv::SLT-1A)₂; (2) about 20 to 22 minutes represented a molecular size equivalent to the monomeric, monovalent CD20-binding protein αCD20-scFv::SLT-1A; (3) about 17 to 17.5 minutes represented a molecular size equivalent to multimeric CD20-binding proteins which were trivalent or tetravalent; and 4) about 17 minutes represented a molecular size equivalent to multimeric, hexavalent CD20-binding protein. The percent purity of the dimeric, bivalent CD20-binding protein (αCD20-scFv::SLT-1A)₂ was calculated from its peak area divided by the total peak area of all peaks from 14 to 27 minutes. The percentage of each peak to all peaks (Percent Area of Total) was determined using the sum of all peak areas as the denominator as shown in the following formula: (area under the curve of the peak of interest)/(sum of all areas under all peaks)×100.

TABLE 8
SEC Analysis of Illustrative, Multivalent CD20-Binding Molecule
Composition #1

| Peak | Peak Retention | Peak Height | | Percent Area |
|---|---|---|---|---|
| Number | Time (minutes) | (AU) | Peak Area | of Total (%) |
| #1 | 17.12 | 11,120 | 870,189 | 8.79 |
| #2 | 17.55 | 11,947 | 546,273 | 5.52 |
| #3 | 19.30 | 162,857 | 7,713,993 | 77.88 |
| #4 | 20.65 | 14,999 | 774,762 | 7.82 |

TABLE 9
SEC Analysis of Illustrative, Multivalent CD20-Binding Molecule
Composition #2

| Peak | Peak Retention | Peak Height | | Percent Area |
|---|---|---|---|---|
| Number | Time (minutes) | (AU) | Peak Area | of Total (%) |
| #1 | 17.49 | 9,138 | 597,819 | 7.51 |
| #2 | 19.32 | 148,854 | 7,003,742 | 87.95 |
| #3 | 20.87 | 6,418 | 361,658 | 4.54 |

TABLE 10
SEC Analysis of Illustrative, Multivalent CD20-Binding Molecule
Composition #3

| Peak | Peak Retention | Peak Height | | Percent Area |
|---|---|---|---|---|
| Number | Time (minutes) | (AU) | Peak Area | of Total (%) |
| #1 | 17.57 | 7,148 | 381,654 | 4.74 |
| #2 | 19.52 | 128,928 | 7,264,891 | 90.26 |
| #3 | 20.87 | 8,268 | 402,467 | 5.00 |

The results of the Percent Area of Total calculations shown in Tables 8-10 are based on the SEC profile data shown in FIG. 12A-12C. Table 8 shows the results for one, illustrative, multivalent CD20-binding molecule composition as described herein having a bivalent CD20-binding protein percentage of total protein of approximately 78%, as well as comprising about 8% monovalent CD20-binding protein and 14% relatively large valence, CD20-binding protein of the total protein. Table 9 shows the results for a second, illustrative, multivalent CD20-binding molecule composition as described herein having a bivalent CD20-binding protein percentage of total protein approximately 88%, as well as comprising about 4.5% monovalent CD20-binding protein and 7.5% relatively large valence, CD20-binding protein of the total protein. Table 10 shows the results for an illustrative, multivalent CD20-binding molecule composition as described herein having a bivalent CD20-binding protein percentage of total protein of approximately 90%, as well as comprising about 5% monovalent CD20-binding protein and 5% relatively large valence, CD20-binding protein of the total protein.

One illustrative, multivalent CD20-binding molecule composition as described herein (“multivalent CD20-binding molecule composition #1”) was analyzed 59 different times over an eighteen-month period using the SEC-HPLC, Waters system assay described above. The peak areas and total peak area were determined using the software analysis as described above with the minimum retention time set around 14 minutes (near the exclusion limit where molecules are too large to have any significant probability of penetrating the fractionation gel) and the maximum retention time set around 22-27 minutes, depending on calibration measurements of gel filtration standard markers and the multivalent CD20-binding molecule composition's solvent, which is near when molecules of sizes smaller than polypeptides flow off the column. The resulting empirical measurements produced a data set (n=59) describing peak #3 (bivalent CD20-binding protein as described herein) area to total peak (total protein) area (Percent Area of Total) with a mean of 77.40 (%), a median of 77.72 (%), a mode of 76.10 (%), a standard deviation of 1.533, and a relative standard deviation of 1.982. An illustrative, individual analysis of the illustrative, multivalent CD20-binding molecule composition #1 is shown in Table 8 and FIG. 12 A.

F. Determining the In Vivo Effects of Multivalent CD20-Binding Molecule Compositions (αCD20-scFv::SLT-1A)₂ and (αCD20-scFv::SLT-1A)ₙ₊₂ Using Animal Models

Using methods known to the skilled worker, animal models are used to determine the in vivo effects of the illustrative compositions (αCD20-scFv::SLT-1A)₂ and (αCD20-scFv::SLT-1A)ₙ₊₂ on CD20+ neoplastic and/or immune cells (see e.g. WO 2014/164680). Various mice strains are used to test the effects of the multivalent CD20-binding molecules as described herein, and compositions thereof, after intravenous administration on xenograft tumors in mice resulting from the injection into those mice of human neoplastic cells which express CD20 on at least one of their cell surfaces. Non-human primates are used to test the effects of the multivalent CD20-binding molecule compositions on CD20+B-cell populations after intravenous administration.

### Summary

Surprisingly, multivalent CD20-binding molecules as described herein, which each comprise cell-targeting, CD20 binding regions and Shiga toxin A subunit effector polypeptide regions, exhibit an unexpected improvement in CD20-expressing cell-kill activity compared to their monovalent protein component.

Given their similar ribosome inactivation activities, it was expected that differences in cytotoxic potencies between monovalent and multivalent variants would be predominantly if not completely explained by differences in the variants' abilities to binding CD20-expressing cells. The difference in K_{D} values for binding CD20-expressing cells between the bivalent CD20-binding molecule composition (CD20-scFv::SLT-1A)₂ of this Example and the monovalent CD20-binding protein composition αCD20-scFv::SLT-1A was about 3-fold with the bivalent CD20-binding molecule composition exhibiting the lower K_{D} value or about a three times greater binding affinity (Table 4; FIG. 4). Thus, if cytotoxic potency of CD20-binding molecule was directly related to the K_{D} of cell binding, then the cytotoxicity of the monovalent CD20-binding protein composition was predicted to be at most 3-fold less cytotoxic to CD20+ cells than the illustrative, bivalent CD20-binding molecule composition—meaning the expected CD₅₀ value of the monovalent CD20-binding protein should be no more than about three times the CD₅₀ value of the illustrative, bivalent CD20-binding molecule composition.

However, it was discovered instead that the monovalent CD20-binding protein composition did not exhibit a cytotoxicity within ten-fold of the cytotoxicity of compositions of multivalent CD20-binding molecules having that same monovalent CD20-binding protein as its only component. Surprisingly, the difference in cytotoxicity was qualitatively increased as by the assay described above, and this cytotoxic difference, while over ten-fold, has yet to be accurately quantified. Without being bound by theory, the increased cytotoxicity of the multivalent CD20-binding protein compositions of this Example might be caused by a qualitative change in the ability of multivalent CD20-binding molecules compared to monovalent CD20-binding molecules to do one or more of the following: 1) internalize into CD20-expressing cells, such as, e.g., with relatively great efficiency; 2) intracellular route to subcellular compartment(s) favorable for effectuating Shiga toxin effector polypeptide mediated cytotoxicity, such as, e.g., with relatively great efficiency; and/or 3) delivery of Shiga toxin effector polypeptides to the cytosol of cell in which the multivalent CD20-binding molecule is present, such as, e.g., with relatively great efficiency.

### Example 2. CD20-Binding Molecules Derived from Shiga Toxins and Various Immunoglobulin-Type Binding Regions, and Compositions Thereof

In this Example, illustrative compositions are created with multivalent CD20-binding proteins derived from Shiga toxin. A Shiga toxin effector region is derived from the A subunit of Shiga-like Toxin 1 (SLT-1A) (SEQ ID NO:1), Shiga toxin (StxA) (SEQ ID NO:2), and/or Shiga-like Toxin 2 (SLT-2A) (SEQ ID NO:3) or chosen from a Shiga toxin effector known in the art (see e.g., WO 2005/092917, WO 2007/033497, US 2013/196928, WO 2014/164680, WO 2014/164693, WO 2015/113005, WO 2015/113007, WO 2015/138435, WO 2015/138452, US 2015/259428, WO 2015/191764, and US 2016/0177284, each of which is incorporated herein by reference in its entirety). An immunoglobulin-type binding region is derived from the CD20-binding molecule chosen from Table 11 and which binds an extracellular part of CD20. The illustrative, multivalent CD20-binding molecules of this Example are created using techniques known in the art and/or as described in the previous Example. In addition, illustrative compositions enriched for these illustrative, multivalent CD20-binding molecules relative to monovalent CD20-binding molecule(s) are created using techniques known in the art and/or as described in the previous Example such that the compositions have a concentration ratio of monovalent CD20-binding molecule to total CD20-binding molecule concentration of less than one to three. The illustrative, multivalent CD20-binding molecules, and compositions thereof, of this Example are tested as described in the previous Example and/or using assays known to the skilled worker.

TABLE 11
Illustrative CD20 Binding Domains
Source of CD20 Binding Domain

| | |
|---|---|
| monoclonal antibody 1F5 and derivatives | See e.g. Golay J et al., J Immunol |
| such as, e.g., humanized variants and | 135: 3795-801 (1985); Press O et |
| immunoglobulin-derived binding domains | al., Blood 69: 584-91 (1987) |
| like scFvs | |
| monoclonal antibody 1H4 and derivatives | See e.g. Haisma H et al., Blood |
| such as, e.g., humanized variants and | 92: 184-90 (1998) |
| immunoglobulin-derived binding domains | |
| like scFvs | |
| monoclonal antibody 1K1791 and derivatives | See e.g. Nishida M et al., Intl J |
| such as, e.g., humanized variants and | Oncol 32: 1263-74 (2008) |
| immunoglobulin-derived binding domains | |
| like scFvs | |
| monoclonal antibody 2B8, Leu16, Leuδ, and | See e.g. Reff M et al., Blood 83: |
| derivatives such as, e.g., humanized variants | 435-45 (1994); Maloney D et al., |
| and immunoglobulin-derived binding | Blood 84: 2457-66 (1994); WO |
| domains like scFvs | 2005016969 |
| monoclonal antibody 2F2 and derivatives | See e.g. Teeling J et al., Blood |
| such as, e.g., humanized variants and | 104: 1793-800 (2004) |
| immunoglobulin-derived binding domains | |
| like scFvs | |
| monoclonal antibody 2H7 and derivatives | See e.g. Liu A et at., Proc Natl |
| such as, e.g., humanized variants and | Acad Sci 84: 3439-43 (1987); |
| immunoglobulin-derived binding domains | Polyak M et al., Blood 99: 3256- |
| like scFvs | 62 (2002); Nickerson-Nutter C et |
| | al., Rheumatology 50: 1033-44 |
| | (2011) |
| monoclonal antibody 7D8 and derivatives | See e.g. Teeling J et al., Blood |
| such as, e.g., humanized variants and | 104: 1793-800 (2004) |
| immunoglobulin-derived binding domains | |
| like scFvs | |
| monoclonal antibody 8E4 and derivatives | Wu L et al., Cancer Lett 292: |
| such as, e.g., humanized variants and | 208-14 (2010) |
| immunoglobulin-derived binding domains | |
| like scFvs | |
| monoclonal antibody 11B8 and derivatives | See e.g. Boross P et al., |
| such as, e.g., humanized variants and | Haematologica 96: 1822-30 |
| immunoglobulin-derived binding domains | (2011) |
| like scFvs | |
| monoclonal antibody AME-133v, | See e.g. Robak T, Robak E, |
| LY2469298, and derivatives such as, e.g., | BioDrugs 25: 13-25 (2011) |
| humanized variants and immunoglobulin- | |
| derived binding domains like scFvs | |
| antibodies recognizing the phosphor-CD20 | See e.g. Golay J et al., J Immunol |
| antigen B1, B-1y1 and derivatives such as, | 135: 3795-801 (1985); Tedder T |
| e.g., humanized variants and | et al., Eur J Immunol 16: 881-7 |
| immunoglobulin-detived binding domains | (1986); Cardarelli P et al., |
| like scFvs | Cancer Immunol Immunother 51: |
| | 15-24 (2002); U.S. Pat. No. |
| | 5,843,398 |
| monoclonal antibody B9E9 and derivatives | See e.g. Schultz J et al., Cancer |
| such as, e.g., humanized variants and | Res 60: 6663-9 (2000) |
| immunoglobulin-derived binding domains | |
| like scFvs | |
| BM-ca and derivatives such as, e.g., | See e.g. Kobayashi H et al., |
| humanized variants and immunoglobulin- | Cancer Med 2: 130-43 (2013 |
| derived binding domains like scFvs ( | |
| monoclonal antibody C2B8 and derivatives | See e.g. Reff M et al., Blood 83: |
| such as, e.g., humanized variants and | 435-45 (1994) |
| immunoglobulin-derived binding domains | |
| like scFvs | |
| monoclonal antibody CKI and derivatives | See e.g. Hooijberg E et al., |
| such as, e.g., humanized variants and | Cancer Res 55: 840-6 (1995); |
| immunoglobulin-derived binding domains | Hooijberg E et al., Hybridoma |
| like scFvs | 15: 23-31 (1996) |
| GA101, RO5072759, and derivatives such as, | See e.g. Mössner E et al., Blood |
| e.g., humanized variants and | 115: 4393-402 (2010); Alduaij W |
| immunoglobulin-derived binding domains | et al., Blood 117: 4519-29 |
| like scFvs | (2011); Robak T, Robak E, |
| | BioDrugs 25: 13-25 (2011); |
| | Salles Get al., Blood 119: 5126- |
| | 32 (2012) |
| monoclonal antibody LT20 and derivatives | See e.g. de Boer O et al., PLoS |
| such as, e.g., humanized variants and | One 2: e779 (2007) |
| immunoglobulin-derived binding domains | |
| like scFvs | |
| ibritumomab and derivatives such as, e.g., | See e.g. Wiseman G et al., Clin |
| humanized variants and immunoglobulin- | Cancer Res 5: 3281s-3286s |
| derived binding domains like scFvs | (1999); Cang S et al., J Hematol |
| | Oncol 5: 64 (2012) |
| monoclonal antibodies HB20-1-25, MB20-1- | See e.g. WO2005000901 |
| 18 and derivatives such as, e.g., humanized | |
| variants and immunoglobulin-derived binding | |
| domains like scFvs | |
| obinutuzumab and derivatives such as, e.g., | See e.g. Mössner E et al., Blood |
| humanized variants and immunoglobulin- | 115: 4393-402 (2010); Robak T, |
| derived binding domains like scFvs | Robak E, BioDrugs 25: 13-25 |
| | (2011); Salles G et al., Blood |
| | 119: 5126-32 (2012); Golay J et |
| | al., Blood 122: 3482-91 (2013) |
| ocaratuzumab and derivatives such as, e.g., | Cang S et al., J Hematol Oncol 5: |
| humanized variants and immunoglobulin- | 64 (2012) |
| derived binding domains like scFvs | |
| ocrelizumab, PRO70769, and derivatives such | See e.g. Morschhauser F et al., |
| as, e.g., humanized variants and | Ann Oncol 21: 1870-6 (2010); |
| immunoglobulin-derived binding domains | Cang S et al., J Hematol Oncol 5: |
| like scFvs | 64 (2012) |
| ofatumumab and derivatives such as, e.g., | See e.g. Hagenbeek A et al., |
| immunoglobulin-derived binding domains | Blood 111: 5486-95 (2008); |
| like scFvs | Cang S et al., J Hematol Oncol 5: |
| | 64 (2012) |
| monoclonal antibodies OUBM1-OUBM8 | See e.g. Uchiyama S et al., |
| | Cancer Sci 101: 201-9 (2010) |
| monoclonal antibody PRO131921 and | See e.g. Robak T, Robak E, |
| derivatives such as, e.g., humanized variants | BioDrugs 25: 13-25 (2011); |
| and immunoglobulin-derived binding | Cang S et al., J Hematol Oncol 5: |
| domains like scFvs | 64 (2012) |
| rituximab and derivatives such as, e.g., | See e.g. Reff M et al., Blood 83: |
| humanized variants and immunoglobulin- | 435-45 (1994); Anderson D et |
| derived binding domains like scFvs | al., Biochem Soc Trans 25: 705-8 |
| | (1997); Golay J et al., Blood 122: |
| | 3482-91 (2013); Kinder M et al., |
| | J Biol Chem 288: 3084-54 |
| | (2013); Zhang H et al., Cell |
| | Physiol Biochem 32: 645-54 |
| | (2013); Ahmadzadeh V et al., |
| | Protein Expr Purif 102: 45-41 |
| | (2014) |
| antibody TGLA and derivatives such as, e.g., | See e.g. Lv M et al., Cancer Lett |
| humanized variants and immunoglobulin- | 294: 66-73 (2010) |
| derived binding domains like scFvs | |
| tositumomab and derivatives such as, e.g., | See e.g. Cheson B, Curr Opin |
| humanized variants and immunoglobulin- | Investig Drugs 3: 165-70 (2002) |
| derived binding domains like scFvs | |
| TRU-015 and derivatives such as, e.g., | See e.g. Burge D et al., Clin Ther |
| humanized variants, scFv variants, and CDRs | 30: 1806-16 (2008); Robak T, |
| | Robak E, BioDrugs 25: 13-25 |
| | (2011)) |
| ublituximab and derivatives such as, e.g., | See e.g. Abdelwahed R et al., |
| humanized variants and immunoglobulin- | Invest Ophthalmol Vis Sci 54: |
| derived binding domains like scFvs | 3657-65 (2013); Garff-Tavernier |
| | M et al., Leukemia 28: 230-3 |
| | (2014) |
| veltuzumab, IMMU-106, hA20, and | See e.g. Morschhauser F et al., J |
| derivatives such as, e.g., humanized variants | Clin Oncol 27: 3346-53 (2009); |
| and immunoglobulin-derived binding | Cang S et al., J Hematol Oncol 5: |
| domains like scFvs | 64 (2012); Ellbrecht C et al., |
| | JAMA Dermatol 1939 (2014) |
| CD20 binding scFv(s) and derivatives such as, | See e.g. Geng S et al., Cell Mol |
| e.g., HL23, scFv-1, scFv-3, scFv-5, and scFv- | Immunol 3: 439-43 (2006); |
| 8 | Olafesn T et al., Protein Eng Des |
| | Sel 23: 243-9 (2010); Fang H et |
| | al., Sci China Life Sci 54: 255-62 |
| | (2011) |
| various CD20 binding antibodies, antigen | See e.g. Lim S et al., |
| binding portions thereof, and derivatives such | Haematologica 95: 135-43 |
| as, e.g., humanized variants and | (2010); U.S. Pat. No. 4,861,579; U.S. Pat. No. |
| immunoglobulin-derived binding domains | 5,500,362; U.S. Pat. No. 5,595,721; U.S. Pat. No. |
| like scFvs | 5,677,180; U.S. Pat. No. 5,721,108; U.S. Pat. No. |
| | 5,736,137; U.S. Pat. No. 5,776,456; U.S. Pat. No. |
| | 5,843,398; U.S. Pat. No. 5,849,898; U.S. Pat. No. |
| | 6,015,542; U.S. Pat. No. 6,090,365; U.S. Pat. No. |
| | 6,120,767; U.S. Pat. No. 6,171,586; U.S. Pat. No. |
| | 6,194,551; U.S. Pat. No. 6,224,866; U.S. Pat. No. |
| | 6,242,195; U.S. Pat. No. 6,287,537; U.S. Pat. No. |
| | 6,306,393; U.S. Pat. No. 6,368,596; U.S. Pat. No. |
| | 6,399,061; U.S. Pat. No. 6,410,391; U.S. Pat. No. |
| | 6,455,043; U.S. Pat. No. 6,528,624; U.S. Pat. No. |
| | 6,538,124; U.S. Pat. No. 6,565,827; U.S. Pat. No. |
| | 6,652,852; U.S. Pat. No. 6,682,734; U.S. Pat. No. |
| | 7,879,984; U.S. Pat. No. 8,101,179; U.S. Pat. No. |
| | 8,153,125; U.S. Pat. No. 8,337,844; |
| | WO95/03770; WO98/58964; |
| | WO99/22764; WO00/09160; |
| | WO00/27428; WO00/27433; |
| | WO00/42072; WO00/44788; |
| | WO00/67795; WO00/67796; |
| | WO00/76542; WO01/03734; |
| | WO01/10460; WO01/10461; |
| | WO01/10462; WO01/13945; |
| | WO01/72333; WO01/80884; |
| | WO01/97858; WO02/060955; |
| | WO02/079255; WO02/096948; |
| | WO02/102312; WO03/002607; |
| | WO03/061694; |
| | WO2004/032828; |
| | WO2005/000901; |
| | WO2006/106959; |
| | WO2009031230; |
| | WO2014076292 |
| CD20 binding, fibronectin domain FN3_{CD20} | See e.g. Natarajan A et al., Clin |
| based on the fibronectin-derived 10^{th} | Cance Res 19: 6820-9 (2013); |
| fibronectin type III domain as an alternative | |
| scaffold to antibody binding domains | |
| monoclonal antibodies which bind to various | U.S. 2011/0091483; U.S. |
| mammalian CD20 antigens | 12/0941,583; |
| | PCT/US2010/055826; |
| | EP20140151932; |
| | PCT/GB2012/052532; U.S. |
| | 13/048,135; EP20140151932; |
| | PCT/GB2012/052532; U.S. |
| | 13/048,135; |
| | PCT/US2006/046034 |
| nucleic acids which can be used to generate | U.S. Pat. No. 8,097,713; U.S. 12/0965956 |
| anti-CD20 antibodies, antigen binding | |
| fragments, and derivatives thereof | |

### Example 3. Formulation of a CD20-Binding Molecule

Molecule 001, an illustrative CD20-binding molecule, is a homodimer of two polypeptides, each polypeptide having the amino acid sequence of SEQ ID NO:54, wherein the two polypeptides are covalently joined by a disulfide bond at position 503. A pharmaceutical composition of Molecule 001 is formulated in an aqueous sodium citrate buffer and contains the following: Molecule 001 (0.5 mg/mL), sodium citrate (United States Pharmacopeia (USP), 4.5 mg/mL), citric acid (USP, 1 mg/mL), sorbitol (National Formulary (NF), 36.4 mg/mL), polysorbate 20 (NF, 0.1% v/v), and sodium hydroxide (USP) and hydrochloric acid (NF), as needed, to adjust the pH to between 5.3 to 5.7.

The pharmaceutical composition of Molecule 001 is shipped in a frozen form to clinical sites and stored in frozen form at −20±5° C. in a freezer. Once thawed, Molecule 001 should not be re-frozen. The stability of Molecule 001 is about 60 months at −20° C., about 31 days at 2 to 8° C., and about 24 hours at room temperature.

For each patient, the daily dose of the pharmaceutical composition is calculated based on body weight. A 100 mL infusion bag with 5% dextrose in water for injection (D5W) or normal saline (NS) is prepared by using a syringe to withdraw and remove the amount of D5W or NS from the bag that is equivalent to a calculated single dose drug product volume. This eliminated volume of D5W or NS is then restored by the calculated volume of the Molecule 001 pharmaceutical composition such that the total volume in the infusion bag is again 100 mL. The pharmaceutical composition of Molecule 001 is provided in Table 12 below.

TABLE 12
Pharmaceutical Composition of 1 mL of Molecule 001

| Ingredient | Amount | | Function | Specification |
|---|---|---|---|---|
| Molecule 001 | 0.50 | mg | Active Ingredient | As described |
| | | | | herein |
| Sodium Citrate | 4.5 | mg | Buffering agent, | USP |
| | | | conjugated base | |
| Citric Acid | 1.0 | mg | Buffering agent, | USP |
| | | | acid | |
| Sorbitol | 36.4 | mg | Cryogenic | NF |
| | | | protectant | |
| Polysorbate 20 | 0.001 | mL | Surfactant | NF |
| | | | stabilizer | |
| Water for Injection | q.s. to 1 | mL | Solvent | WFI (USP) |

| | | | |
|---|---|---|---|
| Sodium Chloride | As needed to | Base, adjust pH | USP |
| | adjust to pH 5.3 to | | |
| | 5.7 | | |
| Hydrochloric Acid | As needed to | Acid, adjust pH | NF |
| | adjust to pH 5.3 to | | |
| | 5.7 | | |

USP = United States Pharmacopeia
NF = National Formulary
q.s. = as much as is sufficient

### Example 4. Combination Therapy Using CD20 Binding Protein and Sirolimus (Rapamycin)

The pharmaceutical composition of Molecule 001 described in Example 3 was used in this Example for in vivo studies in animals. Animal studies were used to study the effects of co-administering sirolimus and Molecule 001. The studies showed that administration of sirolimus prolonged the serum exposure of Molecule 001, increased B-cell depletion, and decreased the levels of anti-Molecule 001 antibodies.

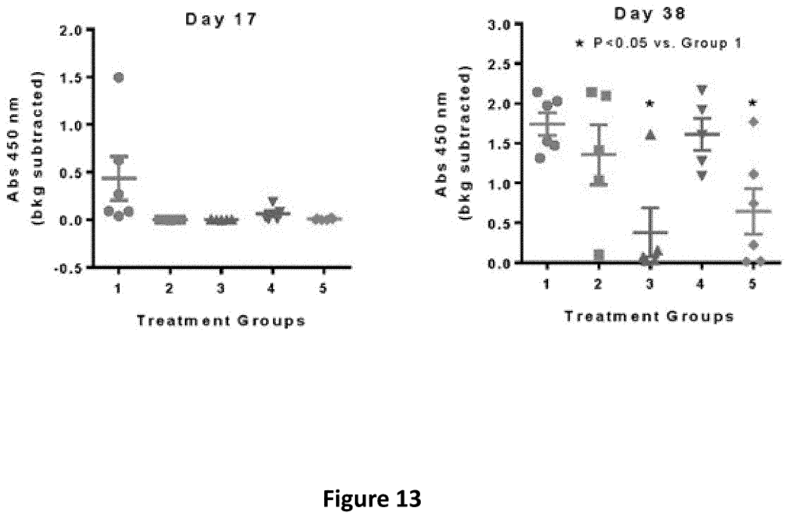

BALB/c mice were treated with Molecule 001 and sirolimus as follows. The mice in treatment groups 1 to 5 all received 0.25 mg/kg Molecule 001 on Study Days 3, 5, 7, 10, 12, 14, 24, 26, 28, 31, 33, and 35. The mice in groups 2-5 received sirolimus in addition to Molecule 001 while the mice in Group 1 received a vehicle only control for sirolimus. The mice in group 2 orally received 0.1 mg of sirolimus on each Study Day from Day 1 to 14. The mice in group 3 orally received 0.3 mg sirolimus on Day 1 and also orally received 0.1 mg of sirolimus on each Study Day from Day 2 to 14. The mice in group 4 orally received 0.3 mg sirolimus on Day 1 and also orally received 0.1 mg sirolimus on each day from Day 2 to 7. The mice in group 5 orally received 0.3 mg sirolimus on Day 1 and Day 22 and also orally received 0.1 mg sirolimus on each day from Day 2 to 14 and from Day 23 to 35. The levels of anti-Molecule 001 antibodies were measured in the serum of the mice in all treatment groups using an ELISA assay, and certain results are reported in Table 13 and FIG. 13. In FIG. 13, treatment group 1 data is shown in blue circles, group 2 is shown in red squares, group 3 is shown in green triangles pointing up, group 4 is shown in purple triangles pointing down, and group 5 is shown in orange diamonds.

TABLE 13
Anti-Molecule 001 IgG Levels as a Percentage of Vehicle-Only Control

| Treatment | Study Day | Study Day | Study Day | Study Day |
|---|---|---|---|---|
| group | 17 | 24 | 31 | 38 |
| #1 | 100% | 100% | 100% | 100% |
| #2 | 0% | 2% | 135% | 78% |
| #3 | 0% | 3% | 23% | 22% |
| #4 | 15% | 43% | 166% | 93% |
| #5 | 2% | 7% | 11% | 37% |

This mouse study showed that anti-Molecule 001 antibody levels were often reduced during cycle 1 (e.g. at Day 17 or Day 24) and cycle 2 (e.g. at Day 38) when sirolimus was administered (loading and maintenance) during the first cycle of Molecule 001 administration (see FIG. 13).

Non-human primate studies revealed that anti-Molecule 001 antibody levels were reduced at Days 10 and 17. Cynomolgus monkeys were intravenously administered Molecule 001 at doses between 40 to 80 μg/kg on Days 3, 5, 6, 10, 12, 14, 24, 26, 28, 31, 33, and 35, and sirolimus was orally administered at 3 mg/kg on Day 1 and at 1 mg/kg on Days 2-14. Peripheral B-cell levels were monitored during the study. Anti-Molecule 001 antibody levels were analyzed starting on Day 10. In non-human primates, coadministration of sirolimus with Molecule 001 reduced anti-Molecule 001 antibody levels and allows for longer serum exposure and prolonged B-cell depletion.

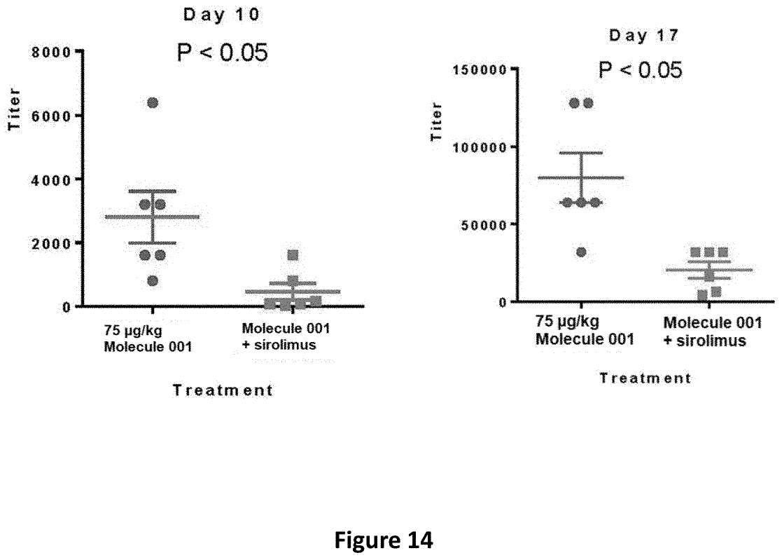

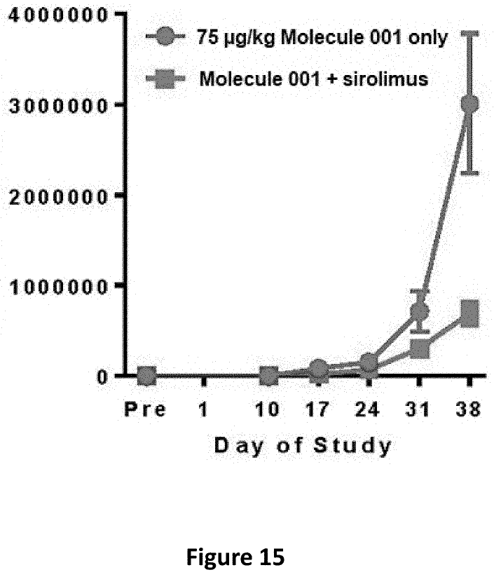

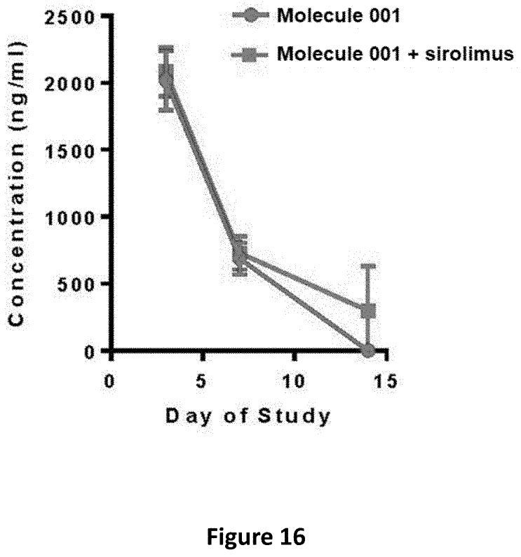

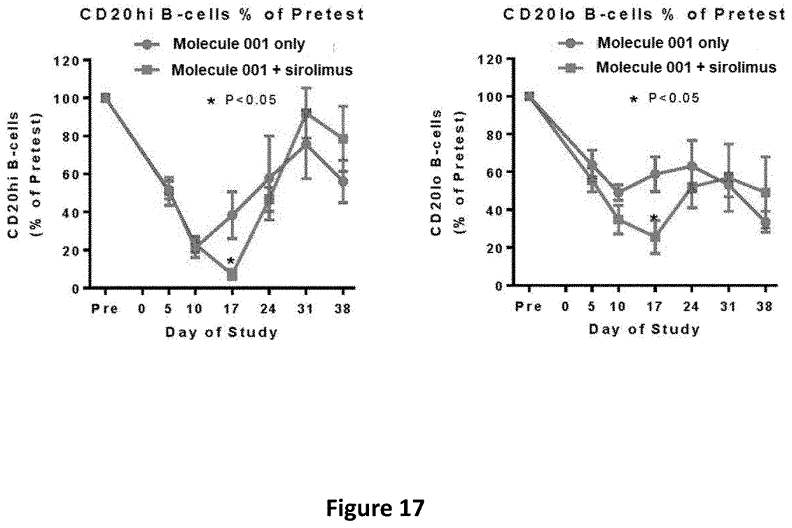

A study using cynomolgus macaques was conducted to investigate effects of the combination of Molecule 001 and sirolimus. Two groups were used with all the animals in both groups intravenously receiving 0.075 mg/kg of Molecule 001 on Study Days 3, 5, 7, 10, 12, 14, 24, 26, 28, 31, 33, and 35. The cynomolgus macaques in the treatment group orally received 3 mg/kg of sirolimus on Study Day 1 and 1 mg/kg of sirolimus on each of Study Days from Day 2 to Day 14. The animals in the other group only received Molecule 001. The levels of anti-Molecule 001 antibodies in the serum of the macaques were measured using an MSD bridging assay, and certain results are reported in FIG. 14-15. The population levels of peripheral B-cells (e.g. high CD20 expressing cells and low CD20 expressing cells) were measured along with certain aspects of pharmacokinetics (FIGS. 16-17).

FIG. 14 shows the anti-Molecule 001 antibody titer level plotted on Day 10 or Day 17 for the two primate treatment groups. FIG. 17 shows the percentage of high or low CD20 expressing B-cells relative to pretest values plotted by day of study for the two primate treatment groups.

This non-human primate study showed that coadministration of sirolimus with Molecule 001 may significantly reduce anti-Molecule 001 IgG responses after three or six doses of Molecule 001 (see e.g. FIGS. 14-15). The pharmacokinetic study showed that serum levels of Molecule 001 observed on Day 14 were often higher in primates co-administered sirolimus (see e.g. FIG. 16). Sirolimus treated primates demonstrated extended peripheral B-cell level depletion (nadir on Day 17 versus Day 10) and greater decreases (−93% vs −79%) in B-cells compared to Group 1 (see e.g. FIG. 17).

### Example 5: Combination of Molecule 001 with Chemotherapy Results in Synergistic Cytotoxicity In Vitro

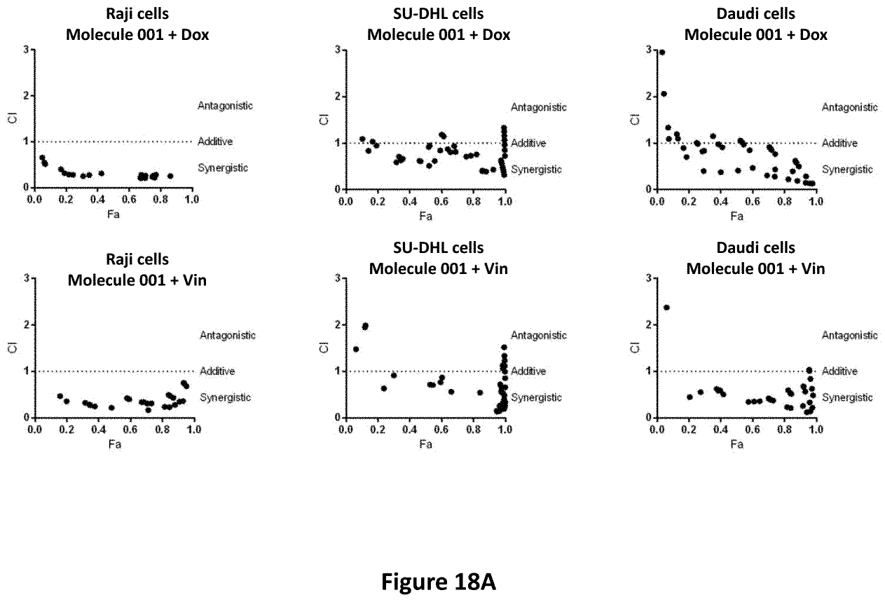

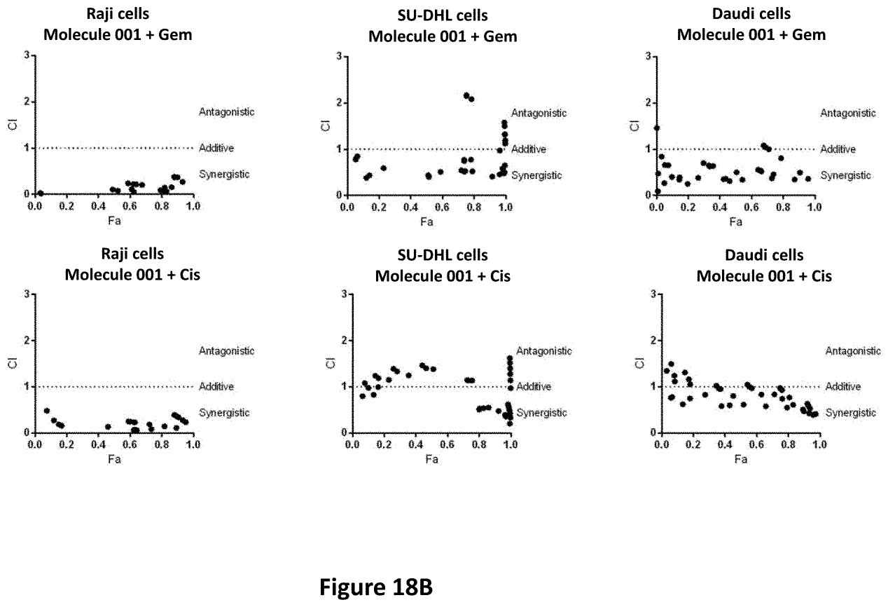

Molecule 001 was tested in combination with chemotherapy drugs in vitro. Specifically, cells from various immortalized cancer lines were incubated at 37° C. in a 5% CO₂ atmosphere with Molecule 001, a chemotherapeutic agent, or a combination of the two. Cell viability was measured after a three day incubation. Results are shown in FIG. 18A-18B and summarized in Table 14 below.

TABLE 14
In vitro responses to Molecule 001 in combination with chemotherapy

| | Chemotherapy | | | | |
|---|---|---|---|---|---|
| | Doxorubicin | Vincristine | Gemcitabine | Bendamustine | |
| Cell line | (Dox) | (Vin) | (Gem) | (Ben) | Cisplatin (Cis) |
| Raji | Synergistic | Synergistic | Synergistic | Not Reported | Synergistic |
| SU-DHL- | Predominantly | Predominantly | Predominantly | Synergistic | Mixed |
| 4 | Synergistic | Synergistic | Synergistic | | |
| Daudi | Mixed | Predominantly | Predominantly | Synergistic | Mixed |
| | | Synergistic | Synergistic | | |
| HBL-1 | Synergistic | Predominantly | Predominantly | Synergistic | Predominantly |
| | | Synergistic | Synergistic | | Synergistic |

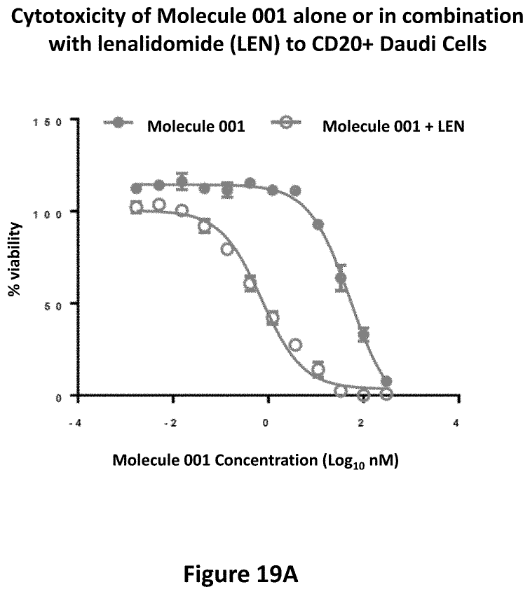

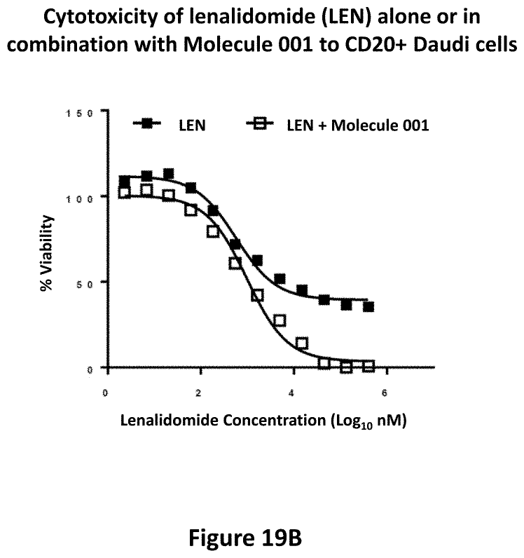

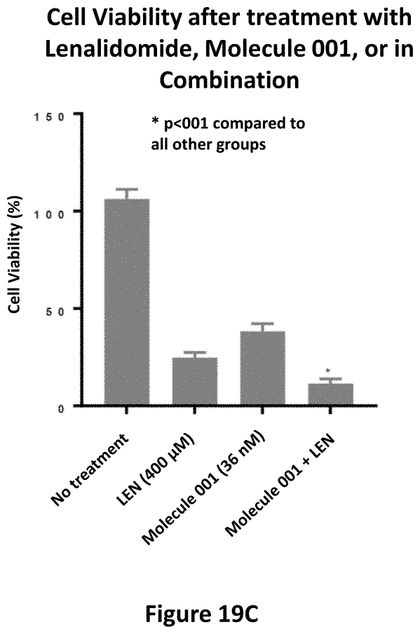

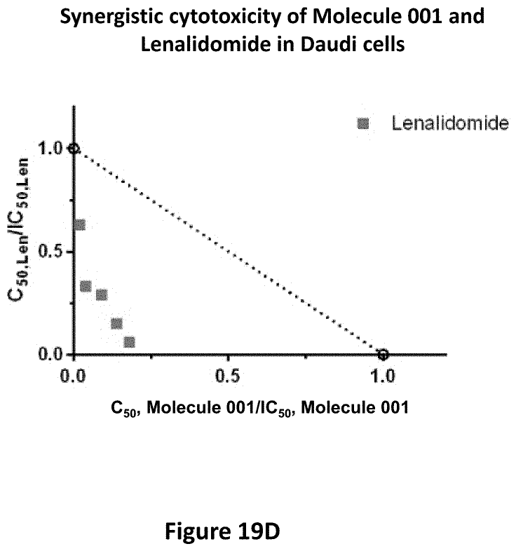

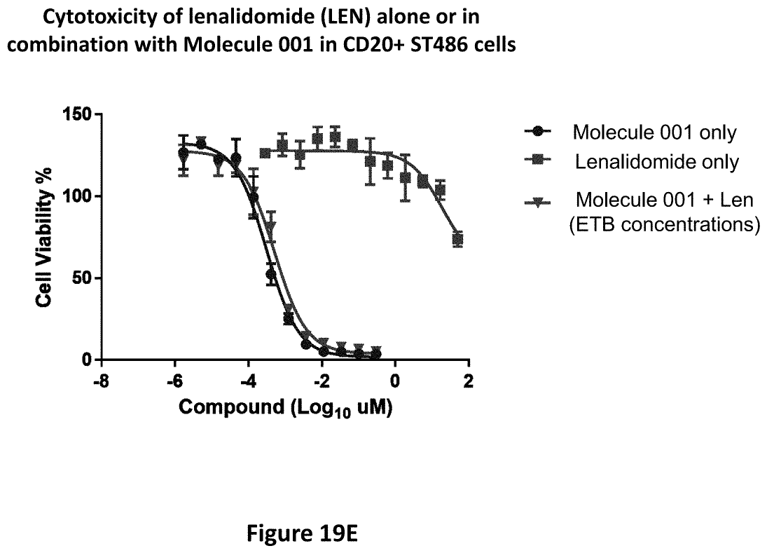

The cytotoxicity of Molecule 001 in combination with lenalidomide was also tested in vitro, in lymphoma (Daudi) cells. The cells were incubated with LEN or vehicle for 21 (FIG. 19A) or 48 hours (FIG. 19B). Following this, Molecule 001 was added and viability was measured after an additional 3 days, using the Promega® Cell Titer-Gb® assay (FIG. 19C). FIG. 19D is an isobologram depicting the synergistic toxicity of Molecule 001 on Daudi cells pre-treated with lenalidomide for 24 hours.

The cytotoxicity of Molecule 001 in combination with lenalidomide was also tested in Burkitt's lymphoma (ST-486) cells. The cells were incubated with Molecule 001 only, lenalidomide only, or a combination of Molecule 001 and lenalidomide. Cell viability was measured 72 hours after treatment using the Promega® Cell TiterGlo® assay (FIG. 19E). The concentrations of lenalidomide used in this assay had little effect on cell viability and had no effect on the activity of Molecule 001.

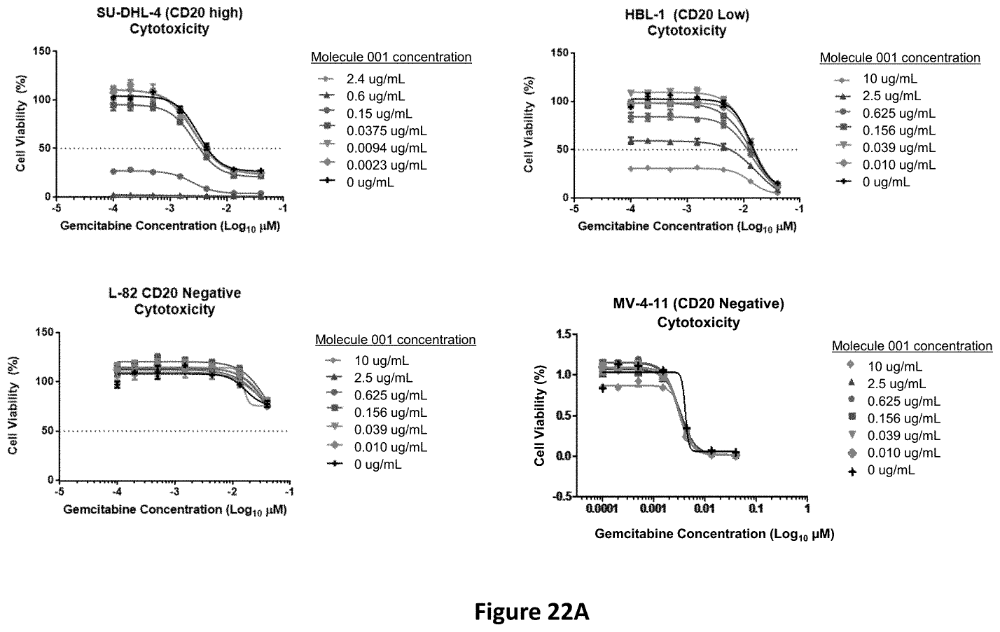

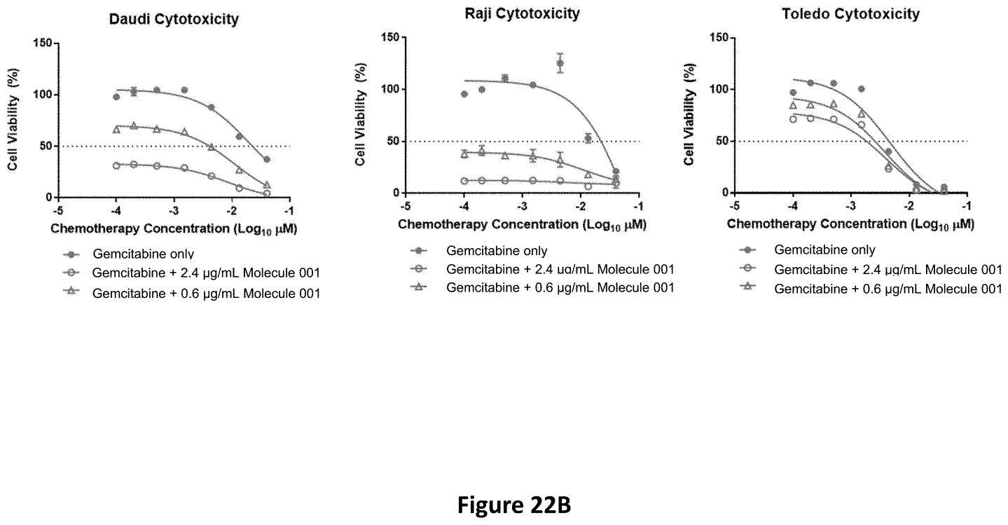

The cytotoxicity of Molecule 001 in combination with gemcitabine was also tested in CD20-positive (SU-DHL-4, Daudi, Raji, HBL-1, and Toledo) and CD20-negative (L-82 and MV-4-11) cell lines. The cells were incubated with gemcitabine alone or in combination with different concentrations of Molecule 001. Cell viability was measured 72 hours after treatment using the Promega® Cell Titer-Gb® assay (FIG. 22A and FIG. 22B).

Taken together, these data show that the combination of Molecule 001 and chemotherapy demonstrate synergistic cellular cytotoxicity, and that the combination of Molecule 001 and LEN demonstrate significantly greater potency against lymphoma cells than either agent alone.

### Example 6. A Phase 2a Open-Label Study of Molecule 001 in Combination with Lenalidomide (LEN) in Subjects with Relapsed or Refractory B-Cell Non-Hodgkin Lymphoma (NHL)

Molecule 001 is being tested in a phase 2a study in combination with LEN in adult patients with histologically confirmed, relapsed or refractory CD20+B-cell NHL. The primary objective of the study is to determine safety and tolerability, including the maximum tolerated dose (MTD), of Molecule 001 in combination with LEN. Secondary objectives include determining pharmacokinetics (PK), pharmacodynamics (PD), immunogenicity, and tumor response.

Key inclusion criteria for the study include:

1) Subjects must have received at least one approved therapy for NHL and have measurable disease by Lugano Criteria. Specifically, subjects should have bi-dimensionally measurable disease by Lugano Classification for NHL (>1.5 cm LDi for lymph noted; >1.0 cm LDi for extra nodal disease).

2) Subjects who have progressed following CAR T-cell therapy, autologous or allogeneic stem cell transplant are eligible. Subjects who underwent stem cell transplant (SCT)>100 days for autologous SCT or >180 days for allogeneic SCT before study drug administration and exhibited a full hematological recovery prior to relapse are eligible.

3) Serum rituximab level must be negative (<500 ng/mL) at screening, because rituximab competes with Molecule 001 for binding to CD20.

4) Adequate bone marrow function (ANC≥1,000/mm³, platelet count ≥50,000/mm^{3′} hemoglobin ≥8 g/dL), adequate hepatic function (total bilirubin ≤1.5×ULN, AST≤3×ULN, ALT≤3×ULN); adequate coagulation (INR or PT≤1.5×ULN, PTT≤1.5 ULN); adequate serum albumin ≥3.0 g/dL). Subjects must also have adequate kidney function (creatinine clearance (CLcr) to be ≥50 mL/min either measured or assessed by using the Cockcroft-Gault formula).

Key exclusion criteria for the study include:

1) Subjects which have received anti-CD20 monoclonal antibody therapy within the following periods before the start of treatment: Rituximab: 84 days; if a subject had received rituximab within 37 weeks before the start of treatment, then a serum rituximab level must be negative (<500 ng/mL) at screening; 2) Obinutuzumab: 184 days; 3) Ofatumumab: 88 days.

Other exclusion criteria include:

2) Current evidence of acute or chronic graft-versus-host disease.

3) History or current evidence of neoplastic disease that is histologically distinct from NHL, except cervical carcinoma in situ, superficial noninvasive bladder tumors, curatively treated Stage I-II non-melanoma skin cancer. Subjects with prior, curatively treated cancer >2 years ago before the start of treatment can be enrolled.

4) Current evidence of new or growing brain or spinal metastases during screening. However, subjects with known brain or spinal metastases may be eligible under some circumstances.

5) Current evidence of Common Terminology Criteria for Adverse Events (CTCAE) Grade >1 toxicity (before the start of treatment, except for hair loss, and those Grade 2 toxicities listed as permitted in other eligibility criteria).

6) Current evidence of incomplete recovery from surgery or radiotherapy before the start of treatment, or planned surgery or radiotherapy at any time during the study until the EoT Visit, except minor elective interventions deemed acceptable by the investigator.

7) Current evidence of significant (CTCAE Grade ≥2) infection or wound within 4 weeks before the start of treatment. Subjects with Grade 2 infection that has stabilized or improved with oral anti-infectives before the start of treatment may be eligible at the sponsor's discretion

8) Current evidence of significant cardiovascular disease including, but not limited to the following conditions:

a. Unstable angina (symptoms of angina at rest) or new-onset angina within ≤3 months before the start of treatment.

b. Arterial thrombosis or pulmonary embolism within ≤3 months before the start of treatment.

c. Myocardial infarction or stroke within ≤3 months before the start of treatment.

d. Pericarditis (any CTCAE grade), pericardial effusion (CTCAE Grade ≥2), non malignant pleural effusion (CTCAE Grade ≥2) or malignant pleural effusion (CTCAE Grade ≥3).

e. Congestive heart failure New York Heart Association (NYHA) Class III or IV at screening or left ventricular ejection fraction (LVEF)<45%, assessed by Echo or multiple-gated acquisition (MUGA) scan within 1 month before starting study treatment. Echo or MUGA scan performed within 6 months before screening and at least 28 days after the last cancer therapy is acceptable provided the subject has not received any potential cardiotoxic agents since then.

f. Cardiac arrhythmia requiring anti-arrhythmic therapy at Screening. Subjects receiving digoxin, calcium channel blockers, or beta-adrenergic blockers are eligible at the investigator's discretion after consultation with medical monitor if the dose has been stable for ≥2 weeks before the start of treatment with Molecule 001. Subjects with sinus arrhythmia and infrequent premature ventricular contractions are eligible at the investigator's discretion.

9) QT interval corrected according to Fridericia's formula (QTcF)>480 ms, determined as the average from three QTcF values on the triplicate electrocardiogram (ECG) obtained at Screening.

10) Current evidence of uncontrolled HIV, HBV or HCV at screening. Serology testing is not required if seronegativity is documented in the medical history and if there are no clinical signs suggestive of HIV or hepatitis infections, or suspected exposure. The following exceptions apply for subjects with positive viral serology:

a. Subjects with HIV and an undetectable viral load and CD4+ T-cells counts ≥350 cells/microliter may be enrolled, but must be taking appropriate opportunistic infection prophylaxis, if clinically relevant.

b. Subjects with positive HBV serology are eligible if they have an undetectable viral load and the subject will receive antiviral prophylaxis for potential HBV reactivation-per institutional guidelines.

c. Subjects with positive HCV serology are eligible if quantitative PCR for plasma HCV RNA is below the lower limit of detection. Concurrent antiviral HCV treatment per institutional guidelines is allowed.

11) Women who are pregnant or breastfeeding.

12) History or current evidence of hypersensitivity to any of the study drugs, or of current hypersensitivity requiring systemic steroids at doses >20 mg/day prednisone equivalent.

13) History or current evidence of any other medical or psychiatric condition or addictive disorder, or laboratory abnormality that, in the opinion of the investigator, may increase the risks associated with study participation, or require treatments that may interfere with the conduct of the study or the interpretation of study results.

14) Prior treatment with Molecule 001.

15) Received therapy for NHL (except the anti-CD20 Mab therapies listed above and radioimmunoconjugates) within 4 weeks before the start of treatment.

16) Any investigational drug treatment from 4 weeks or 5 half-lives of the agent before the start of treatment, whichever is longer, until the EoT Visit.

17) Received radiotherapy to tumor lesions that would be chosen as target lesions (measurable disease) within 4 weeks before the start of treatment, unless the lesion exhibited objective progression between the radiotherapy and the screening according to the Lugano Classification for NHL. Palliative radiotherapy to non-target lesions may be permitted at the investigator's discretion after consultation with the medical monitor.

18) Received any live vaccines within 4 weeks before of the start of treatment, unless the investigator believes the benefits outweigh the risks, after approval with the sponsor.

19) Require use of systemic immune modulators during study treatment. Systemic immune modulators include but are not limited to systemic corticosteroids at doses >20 mg/day or prednisone equivalent, cyclosporine and tacrolimus.

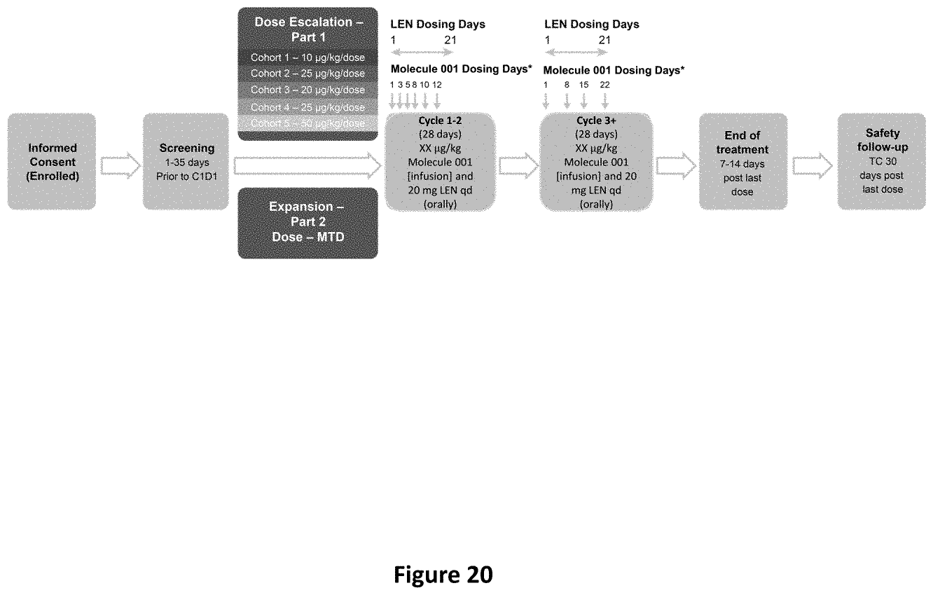

A study design schema is shown in FIG. 20. The study is being conducted in 2 parts. Part 1 includes Molecule 001 dose escalation over 5 dose cohorts according to a modified 3+3 design and will include up to 24 subjects with CD20+ NHL. The MTD for Molecule 001 will be defined as the highest dose of Molecule 001 that can be given in combination with LEN so that no more than 1 of 6 subjects experiences a dose-limiting toxicity (DLT) in Part 1 of this study. At least 6 subjects must be treated at the MTD dose level of Molecule 001 in combination with LEN and complete Cycle 1, or experience a DLT. All subtypes of B-cell NHL may be considered for Part 1. Only histologically confirmed documented DLBCL (including mixed histology) may be considered for Part 1.

Part 2 is designed to assess the safety and tolerability of Molecule 001 plus LEN in the MTD Expansion Cohort, where the dose declared as MTD of Molecule 001 in part 1 will be given in combination with LEN in up to 40 subjects with CD20+ relapsed or refractory diffuse large B-cell lymphoma. In addition, the PK, PD, immunogenicity and tumor response of Molecule 001 plust LEN will be evaluated in Part 2.

In the first two dose cohorts (10 and 25 μg/kg/dose of Part 1, subjects received Molecule 001 IV infusions over 1 hour three times weekly for 2 weeks (days 1, 3, 5, 8, 10, and 12), and LEN (20 mg daily) on days 1-21 of each 28-day treatment cycle for Cycles 1 and 2. In subsequent cycles, Molecule 001 was administered once weekly (days 1, 8, 15, and 22) with continued LEN dosing on Days 1-21.

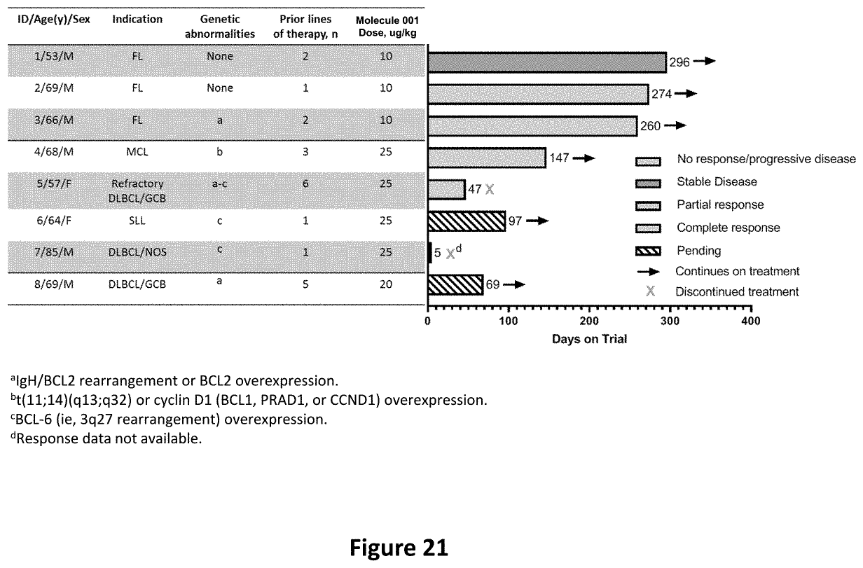

The analysis included 6 subjects (Cohort 1 & 2, both n=3), diagnosed as follows: follicular lymphoma (n=3), mantle cell lymphoma, small lymphocytic lymphoma & DLBCL (each n=1). Median age was 64.5 y (range, 52-69); subjects received a median of 2 (1-6) prior lines of therapy. All subjects had ≥1 treatment-emergent adverse event (TEAE) & 44 TEAEs occurred (Cohort 1, n=12; Cohort 2, n=32); the most common were peripheral edema, fatigue & myalgia (both n=3), hypoalbuminemia, hypokalemia, muscular weakness (each n=2), all grade (G) 1-2 except 2 G3 (fatigue, hypokalemia). Six treatment-related AEs occurred in Cohort 1, 20 in Cohort 2; no deaths occurred. One subject in Cohort 2 experienced 3 treatment-related serious AEs that led to hospitalization: G4 neutropenia, G3 anemia & G2 capillary leak syndrome (CLS). Two dose-limiting toxicities (DLTs) occurred in 2 subjects treated in Cohort 2 (both G2 CLS). Four subjects were radiologically assessed (Cohort 1, n=3; Cohort 2, n=1). In Cohort 1, the best responses were 1 complete response (CR, 1 prior treatment), 1 partial response & 1 stable disease. In Cohort 2, 1 subject (3 prior treatments) had a CR. This data is summarized in FIG. 21.

Because of 2 dose-limiting toxicities (grade 2 capillary leak syndrome) in Cohort 2, the dose in Cohort 23 was reduced from 50 to 20 μg/kg/dose. The protocol was amended so that in future Cohorts 4 and 5 (20 and 50 μg/kg/dose), Molecule 001 will be dosed biweekly for 2 weeks for Cycles 1 and 2 and then once weekly for subsequent cycles. The administered dose of Molecule 001 will be capped at 6000 μg/dose.

### Example 7. A Phase 2a Open-Label Study of Molecule 001 in Combination with Gemcitabine and Oxaliplatin (GemOx) in Subjects with Relapsed or Refractory B-Cell Non-Hodgkin Lymphoma (NHL)

Molecule 001 is also being tested in a phase 2a study in combination with GemOx in adult patients with histologically confirmed, relapsed or refractory CD20+B-cell NHL. The primary objectives are to determine the safety and tolerability, including the maximum tolerated dose (MTD), of Molecule 001+GemOx. Secondary objectives include determining pharmacokinetics (PK), pharmacodynamics (PD), immunogenicity and tumor response.

The study is being conducted in two parts. Part 1 includes Molecule 001 dose escalation according to a modified 3+3 design to identify the MTD of Molecule 001+GemOx and will enroll up to 24 subjects. Part 2 will confirm the safety and tolerability of Molecule 001+GemOx in the MTD Expansion Cohort and will enroll up to 40 subjects.

Eligible subjects will have histologically confirmed, relapsed or refractory CD20+B-cell NHL (in Part 2, r/rDLBCL only). Subjects must have received at least one approved therapy for NHL and must have measurable disease by Lugano criteria. Subjects who have progressed following CAR T-cell therapy, autologous- or allogeneic stem cell transplant will be also eligible. Serum rituximab level must be below lower limits of quantification (<500 ng/mL) at screening.

The study is being conducted in two sequential parts (Part 1 and Part 2). Part 1 includes a modified 3+3 design to determine the MTD of Molecule 001 in combination with gemcitabine (1000 mg/m²) and oxaliplatin (100 mg/m²) in up to 24 subjects with relapsed or refractory B-Cell NHL. If permitted by the safety results, Molecule 001 dose escalation will proceed in three sequential dose cohorts (Cohorts 1 to 3). The starting dose of Molecule 001 will be 10 μg/kg/dose in Cohort 1, 25 μg/kg/dose in Cohort 2, and 50 μg/kg/dose in Cohort 3. The maximum dose of Molecule 001 given in this study is 50 μg/kg/dose with a maximum total dose of 6000 μg. Molecule 001 dose escalation will not proceed above the 50 μg/kg/dose even if no more than 1 of 6 subjects experiences a dose-limiting toxicity (DLT).

In Part 2, the MTD dose cohort will be expanded to confirm the safety and tolerability of Molecule 001 in combination with GemOx in up to 40 additional subjects with DLBCL. The PK, PD, and immunogenicity and tumor responses of Molecule 001 in combination with GemOx will be more thoroughly evaluated in Part 2.

Molecule 001 was administered to subjects on Days 1, 3, 5, 8, 10 and 12 within 28-day cycles during Cycles 1 and 2 while GemOx (Gemcitabine 1000 mg/m², and Oxaliplatin 100 mg/m²) was administered on Days 2 and 16 during Cycles 1 and 2. During Cycles 3 and beyond, Molecule 001 was to be administered weekly (Days 1, 8, 15, and 21 of a 28-day cycle) with the same dosing of GemOx on Day 2 and 16. Subjects could receive up to 5 treatment cycles in the absence of clear disease progression or toxicity.

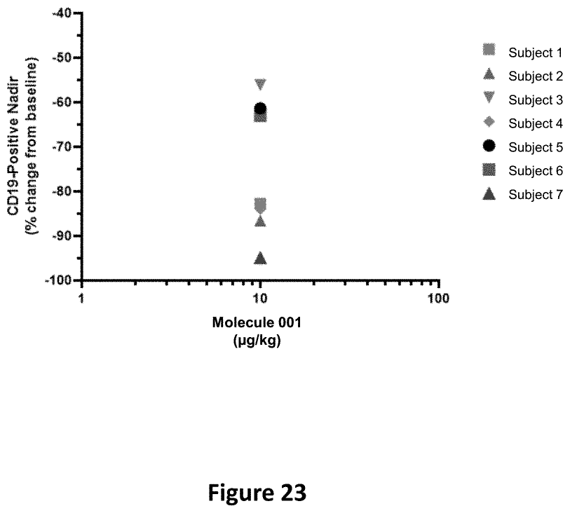

Subjects in Cohort 1 were dosed at 10 μg/kg/dose of Molecule 001 in combination with GemOx (Gemcitabine 1000 mg/m², and Oxaliplatin 100 mg/m²) according to the dosing regimen described above. FIG. 23 shows the minimum observed percent change of CD19+ cells from baseline upon treatment with Molecule 001 and GemOx.

The best overall response was one complete response in a subject with DLBCL, one partial response in a subject with activated B cell DLBCL, one partial response in a subject with germinal center B cell DLBCL, one partial response in a subject with follicular lymphoma, one partial response in a subject with mixed DLBCL and follicular lymphoma, and one partial response in a subject with DLBCL.

All seven subjects in Cohort 1 experienced at least one adverse event (AE) and 3 subjects reported at least one serious adverse event (SAE). Five subjects experienced treatment-related AEs. The most frequent AEs occurring in 3 or more subjects were nausea, peripheral sensory neuropathy, dysgeusia, fatigue, decreased neutrophil count, oedema peripheral, anaemia, increased aspartate aminotransferase, constipation, cough, decreased appetite, diarrhea, hyponatremia, hypotension, and decreased platelet count. Two subjects experienced a treatment-related SAE of Grade 2 capillary leak syndrome (CLS) early in Cycle 1 of treatment. No treatment-related deaths occurred in Cohort 1.

Because of the dose-limiting toxicities in Cohort 1 (Grade 2 CLS), Cycle 1 was amended to a 42-day cycle and all subsequent cycles will be 28 days. In Cycle 1, Molecule 001 will be administered on Day 1, 3, 5, 8, 10, 12 and then weekly on Days 15, 22, 29, and 36 while GemOx will be administered on Days 16 and 30. For subsequent 28-day cycles, Molecule 001 will be administered on Day 1, 8, 15, and 22 while GemOx will be administered on Days 2 and 16. Subjects may receive up to 4 treatment cycles in the absence of clear disease progression or toxicity and additional 28-day cycles may be considered for subjects who experience clinical benefit. The administered dose of Molecule 001 will be capped at 6000 μg/dose.

While some embodiments described herein have been described by way of illustration, it will be apparent that the invention may be put into practice with many modifications, variations and adaptations, and with the use of numerous equivalents or alternative solutions that are within the scope of persons skilled in the art, without departing from the spirit described herein or exceeding the scope of the claims.

All publications, patents, and patent applications are herein incorporated by reference in their entirety to the same extent as if each individual publication, patent or patent application was specifically and individually indicated to be incorporated by reference in its entirety. The patent application publications WO 2005/092917, WO 2007/033497, US 2013/196928, WO 2014/164680, WO 2014/164693, WO 2015/113005, WO 2015/113007, WO 2015/138435, WO 2015/138452, US 2015/0259428, WO 2015/191764, US 2016/0126950 and US 2016/0177284 are each incorporated herein by reference in its entirety. The disclosures of U.S. provisional patent applications 61/777,130, 62/112,314, and 62/249,193 are each incorporated herein by reference in its entirety. The complete disclosures of all electronically available biological sequence information from GenBank (National Center for Biotechnology Information, U.S.) for amino acid and nucleotide sequences cited herein are each incorporated herein by reference in its entirety.

What is claimed is:

1. A method for treating or slowing the progression of a non-Hodgkin's lymphoma;
wherein the method comprises administering to a subject in need thereof:
(i) an effective amount of a CD20-binding molecule; and
(ii) an effective amount of lenalidomide;
wherein the CD20-binding molecule comprises a polypeptide having the sequence of SEQ ID NO: 54.

2. The method of claim 1, wherein the CD20-binding molecule is administered at a dose of about 10 μg/kg, about 20 μg/kg, about 25 μg/kg, about 50 μg/kg, or about 75 μg/kg of the subject's body weight

3. The method of claim 2, wherein the CD20-binding molecule is administered on each of days 1, 3, 5, 8, 10, and 12 of a first 28-day cycle.

4. The method of claim 3, wherein lenalidomide is administered daily on days 1 to 21 of the first 28-day cycle.

5. The method of claim 3, further comprising administering the CD20-binding molecule weekly during a second 28-day cycle following the first 28-day cycle, wherein the CD20-binding molecule is administered on days 1, 8, 15, and 22 of the second 28-day cycle.

6. The method of claim 5, wherein each dose administered during the second 28-day cycle is in an amount of about 10 μg/kg, about 25 μg/kg, about 50 μg/kg, or about 75 μg/kg of the subject's body weight.

7. The method of claim 5, wherein the lenalidomide is administered daily on days 1 to 21 of the second 28-day cycle.

8. The method of claim 5, further comprising administering the CD20-binding molecule weekly during a third 28-day cycle following the first and second 28-day cycles, wherein the CD20-binding molecule is administered on days 1, 8, 15, and 22 of the third 28-day cycle.

9. The method of claim 8, wherein each dose administered during the third 28-day cycle is in an amount of about 10 μg/kg, about 25 μg/kg, about 50 μg/kg, or about 75 μg/kg of the subject's body weight.

10. The method of claim 8, wherein the lenalidomide is administered daily on days 1 to 21 of the third 28-day cycle.

11. The method of claim 8, further comprising administering the CD20-binding molecule for at least one additional 28-day cycle.

12. The method of claim 11, wherein each dose of the CD20-binding molecule administered during the at least one additional 28-day cycle is in an amount of about 10 μg/kg, about 25 μg/kg, about 50 μg/kg, or about 75 μg/kg of the subject's body weight.

13. The method of claim 11, wherein the lenalidomide is administered daily on days 1 to 21 of the at least one additional 28-day cycle.

14. The method of claim 1, wherein the CD20-binding molecule is administered by intravenous infusion.

15. The method of claim 14, wherein the intravenous infusion is administered over about 1 hour.

16. The method of claim 1, wherein the lenalidomide is administered orally.

17. The method of claim 16, wherein the lenalidomide is administered at a dose of about 20 milligram per day.

18. The method of claim 1, wherein the non-Hodgkin's lymphoma is diffuse large B-cell lymphoma, mantle cell lymphoma, or follicular lymphoma.

19. The method of claim 18, wherein the non-Hodgkin's lymphoma is relapsed or refractory to treatment with at least one additional anti-cancer therapy.

20. The method of claim 1, wherein the CD20-binding molecule is a homodimer comprising two identical polypeptides; wherein each identical polypeptide has the amino acid sequence of SEQ ID NO: 54.

21. The method of claim 1, wherein the CD20-binding molecule is provided in a pharmaceutical composition, and wherein the pharmaceutical composition comprises at least one pharmaceutically acceptable excipient or carrier.

22. The method of claim 21, wherein the pharmaceutical composition comprises sorbitol, sodium citrate and polysorbate-20.

23. The method of claim 1, wherein the amount of the CD20-binding molecule administered in a single dose is about 0.5 mg to about 10 mg.

24. The method of claim 1, wherein the amount of the CD20-binding molecule administered over one or more cycles is about 5 mg to about 250 mg.
